(19) United States
(12) Patent Application Publication
(10) Pub. No.: US 2021/0251991 A1
(43) Pub. Date: Aug. 19, 2021

(54) SUBSTITUTED 2,2'-BIPYRIMIDINYL COMPOUNDS, ANALOGUES THEREOF, AND METHODS USING SAME

(71) Applicant: ARBUTUS BIOPHARMA CORPORATION, Burnaby (CA)

(72) Inventors: Shuai Chen, Warrington, PA (US); Andrew G. Cole, Cranbury, NJ (US); Bruce D. Dorsey, Ambler, PA (US); Benjamin J. Dugan, Glen Mills, PA (US); Yi Fan, Doylestown, PA (US); Dimitar B. Gotchev, Hatboro, PA (US); Ramesh Kakarla, Doylestown, PA (US); Jorge Quintero, Sayreville, NJ (US); Michael J. Sofia, Doylestown, PA (US)

(21) Appl. No.: 17/050,238

(22) PCT Filed: May 14, 2019
(86) PCT No.: PCT/US2019/032248

§ 371 Date: Oct. 23, 2020

Related U.S. Application Data
(60) Provisional application No. 62/671,831, filed on May 15, 2018.

(51) Int. Cl. A61K 31/506; A61K 31/713; C07D 401/14; C07D 471/08; C07D 487/08; C07D 403/14; A61K 31/519; C07D 491/048; C07D 513/04; C07D 403/04; C07D 401/04; A61K 31/4725; C07D 471/04; C07D 413/14; A61K 31/553

(52) U.S. Cl. CPC A61K 31/506; A61K 31/553; C07D 401/14; C07D 471/08; C07D 487/08; C07D 403/14; A61K 31/519; C07D 491/048; C07D 513/04; C07D 403/04; C07D 401/04; A61K 31/4725; C07D 471/04; C07D 413/14; A61K 31/713

(57) ABSTRACT

The present disclosure includes substituted 2,2′-bipyrimidinyl compounds, analogues thereof, and compositions comprising the same, which can be used to treat and/or prevent hepatitis B virus (HBV) and/or hepatitis B virus (HBV)-hepatitis D virus (HDV) infection in a patient.

### CROSS REFERENCE TO RELATED APPLICATION

The present application claims priority under 35 U.S.C. § 119(e) to U.S. Provisional Application No. 62/671,831, filed May 15, 2018, which is hereby incorporated by reference herein in its entirety.

### BACKGROUND OF THE INVENTION

Hepatitis B is one of the world's most prevalent diseases. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis, and/or other complications. Hepatitis B is caused by hepatitis B virus (HBV), a noncytopathic, liver tropic DNA virus belonging to Hepadnaviridae family.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit HBV DNA polymerase. At present, the first-line treatment choices are entecavir, tenofovir, or peg-interferon alfa-2a. However, peg-interferon alfa-2a achieves desirable serological milestones in only one third of treated patients, and is frequently associated with severe side effects. Entecavir and tenofovir require long-term or possibly lifetime administration to continuously suppress HBV replication, and may eventually fail due to emergence of drug-resistant viruses.

HBV is an enveloped virus with an unusual mode of replication, centering on the establishment of a covalently closed circular DNA (cccDNA) copy of its genome in the host cell nucleus. Pregenomic (pg) RNA is the template for reverse transcriptional replication of HBV DNA. The encapsidation of pg RNA, together with viral DNA polymerase, into a nucleocapsid is essential for the subsequent viral DNA synthesis.

Aside from being a critical structural component of the virion, the HBV envelope is a major factor in the disease process. In chronically infected individuals, serum levels of HBV surface antigen (HBsAg) can be as high as 400 μg/ml, driven by the propensity for infected cells to secrete non-infectious subviral particles at levels far in excess of infectious (Dane) particles. HBsAg comprises the principal antigenic determinant in HBV infection and is composed of the small, middle and large surface antigens (S, M and L, respectively). These proteins are produced from a single open reading frame as three separate N-glycosylated polypeptides through utilization of alternative transcriptional start sites (for L and M/S mRNAs) and initiation codons (for L, M, and S).

Although the viral polymerase and HBsAg perform distinct functions, both are essential proteins for the virus to complete its life cycle and be infectious. HBV lacking HBsAg is completely defective, and cannot infect or cause infection. HBsAg protects the virus nucleocapsid, begins the infectious cycle, and mediates morphogenesis and secretion of newly forming virus from the infected cell.

People chronically infected with HBV are usually characterized by readily detectable levels of circulating antibody specific to the viral capsid (HBc), with little, if any detectable levels of antibody to HBsAg. There is evidence that chronic carriers produce antibodies to HBsAg, but these antibodies are complexed with the circulating HBsAg, which can be present in mg/mL amounts in a chronic carrier's circulation. Reducing the amount of circulating levels of HBsAg might allow any present anti-HBsA to manage the infection. Further, even if nucleocapsids free of HBsAg were to be expressed or secreted into circulation (perhaps as a result of cell death), the high levels of anti-HBc would quickly complex with them and result in their clearance.

Studies have shown that the presence of subviral particles in a culture of infected hepatocytes may have a transactivating function on viral genomic replication, and the circulating surface antigen suppresses virus-specific immune response. Furthermore, the scarcity of virus-specific cytotoxic T lymphocytes (CTLs), that is a hallmark of chronic HBV infection, may be due to repression of MHC I presentation by intracellular expression of L and M in infected hepatocytes. Existing FDA-approved therapies do not significantly affect HBsAg serum levels.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa, given weekly for 12 to 18 months, is the only licensed treatment for hepatitis D. Response to this therapy is limited-in only about one-quarter of patients is serum HDV RNA undetectable 6 months post therapy.

There is thus a need in the art for novel compounds and/or compositions that can be used to treat and/or prevent HBV and/or HBV-HDV infection in a subject. In certain embodiments, the compounds can be used in patients that are HBV and/or HBV-HDV infected, patients who are at risk of becoming HBV and/or HBV-HDV infected, and/or patients that are infected with drug-resistant HBV and/or HDV. The present invention addresses this need.

### BRIEF SUMMARY OF THE INVENTION

The present disclosure provides a compound of formula (I), or a salt, solvate, geometric isomer, stereoisomer, tautomer, and any mixtures thereof:

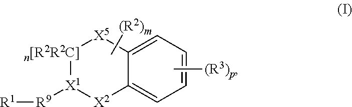

wherein the variables in (I) are defined elsewhere herein.

The present disclosure further provides a compound of formula (Ia), or a salt, solvate, geometric isomer, stereoisomer, tautomer, and any mixtures thereof:

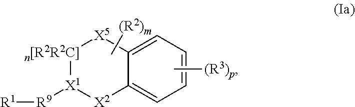

wherein the variables in (Ia) are defined elsewhere herein.

The present disclosure further provides a compound of formula (Ib), or a salt, solvate, geometric isomer, stereoisomer, tautomer, and any mixtures thereof:

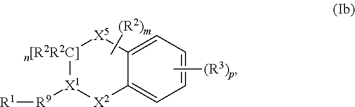

wherein the variables in (Ib) are defined elsewhere herein.

The present disclosure further provides a pharmaceutical composition comprising at least one compound of the disclosure and a pharmaceutically acceptable carrier.

The present disclosure further provides a method of treating, ameliorating, and/or preventing hepatitis virus infection in a subject. In certain embodiments, the method comprises administering to the subject a therapeutically effective amount of at least one compound of the disclosure. In certain embodiments, the method comprises administering to the subject a therapeutically effective amount of at least one composition of the disclosure.

The present disclosure further provides a method of reducing, reversing the increase, and/or minimizing levels of at least one selected from the group consisting of hepatitis B virus surface antigen (HBsAg), hepatitis B e-antigen (HBeAg), hepatitis B core protein, and pregenomic (pg) RNA, in a HBV-infected subject. In certain embodiments, the method comprises administering to the subject a therapeutically effective amount of at least one compound of the disclosure. In certain embodiments, the method comprises administering to the subject a therapeutically effective amount of at least one composition of the disclosure.

### DETAILED DESCRIPTION OF THE INVENTION

The invention relates, in certain aspects, to the discovery of certain substituted polyaromatic compounds that are useful to treat and/or prevent HBV and/or HBV-HDV infection and related conditions in a subject. In certain embodiments, the compounds inhibit and/or reduce HBsAg secretion in an HBV-infected and/or HBV-HDV-infected subject. In other embodiments, the compounds reduce or minimize levels of HBsAg in an HBV-infected and/or HBV-HDV-infected subject. In yet other embodiments, the compounds reduce or minimize levels of HBeAg in an HBV-infected and/or HBV-HDV-infected subject. In yet other embodiments, the compounds reduce or minimize levels of hepatitis B core protein in an HBV-infected and/or HBV-HDV-infected subject. In yet other embodiments, the compounds reduce or minimize levels of pg RNA in an HBV-infected and/or HBV-HDV-infected subject.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Moreover, two or more steps or actions can be conducted simultaneously or not.

The following non-limiting abbreviations are used herein: cccDNA, covalently closed circular DNA; CH₂Cl₂, methylene chloride; DMF, dimethylformamide; DMPA, 4-dimethylamino-pyridine; EtOAc, ethyl acetate; HBc, hepatitis B capsid; HBV, hepatitis B virus; HDV, hepatitis D virus; HBeAg, hepatitis B e-antigen; HBsAg, hepatitis B virus surface antigen; HPLC, high-performance liquid chromatography; IPA, isopropyl alcohol; MeOH, methanol; MTBE, methyl tert-butyl ether; NaHCO₃, sodium bicarbonate; pg RNA, pregenomic RNA; SiO₂, silica; SPhos, 2-dicyclohexylphosphino-2′,6′-dimethoxybiphenyl; THF, tetrahydrofuran; XPhos, 2-dicyclohexylphosphino-2′,4′,6′-triisopropylbiphenyl; XPhos Pd G2, chloro(2-dicyclohexylphosphino-2′,4′,6′-triisopropyl-1,1′-biphenyl)[2-(2′-amino-1,1′-biphenyl)]palladium(II); XPhos Pd G3, [(4,5-bis(diphenylphosphino)-9,9-dimethylxanthene)-2-(2′-amino-1,1′-biphenyl)]palladium(II) methanesulfonate.

As used herein, the articles “a” and “an” refer to one or to more than one (i.e., to at least one) of the grammatical object of the article. By way of example, “an element” means one element or more than one element.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy), and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized R (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one to six carbon alkanediyl chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is [substituted aryl]-(CH₂)—. Similarly, the term “heteroaryl-(C₁-C₆)alkyl” refers to a functional group wherein a one to three carbon alkanediyl chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is [substituted heteroaryl]-(CH₂)—.

In one aspect, the terms “co-administered” and “co-administration” as relating to a subject refer to administering to the subject a compound and/or composition of the invention along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain, or cyclic substituent groups. Examples of (C₃-C₆)cycloalkyl groups are cyclopropyl, cyclobutyl, cyclopentyl, and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include bicyclo-[2.1.1]hexanyl, bicyclo[2.2.1]heptanyl, bicyclo[3.1.1]heptanyl, 1,3-dimethyl[2.2.1] heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates), and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably, and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy, and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido, and nitro. As used herein, where a substituent is an alkyl or alkoxy group, the carbon chain may be branched, straight or cyclic.

Unless otherwise noted, when two substituents are taken together to form a ring having a specified number of ring atoms (e.g., two groups taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

### Compounds

The invention includes certain compound recited herein, as well as any salt, solvate, geometric isomer (such as, in a non-limiting example, any geometric isomer and any mixtures thereof, such as, in a non-limiting example, mixtures in any proportions of any geometric isomers thereof), stereoisomer (such as, in a non-limiting example, any enantiomer or diastereoisomer, and any mixtures thereof, such as, in a non-limiting example, mixtures in any proportions of any enantiomers and/or diastereoisomers thereof), tautomer (such as, in a non-limiting example, any tautomer and any mixtures thereof, such as, in a non-limiting example, mixtures in any proportions of any tautomers thereof), and any mixtures thereof.

The invention includes a compound of formula (I), or a salt, solvate, geometric isomer, stereoisomer, tautomer, and any mixtures thereof:

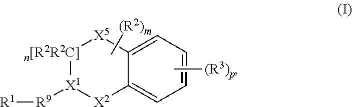

wherein in (I):

X¹ is N and X² is CR²R², or X² is NR⁴ and X¹ is CR⁴;

X⁵ is selected from the group consisting of O and CR²R²,
or one R² group from X⁵ can combine with one R² group of X² to form C₁-C₆ alkylene;
R¹ is selected from the group consisting of:

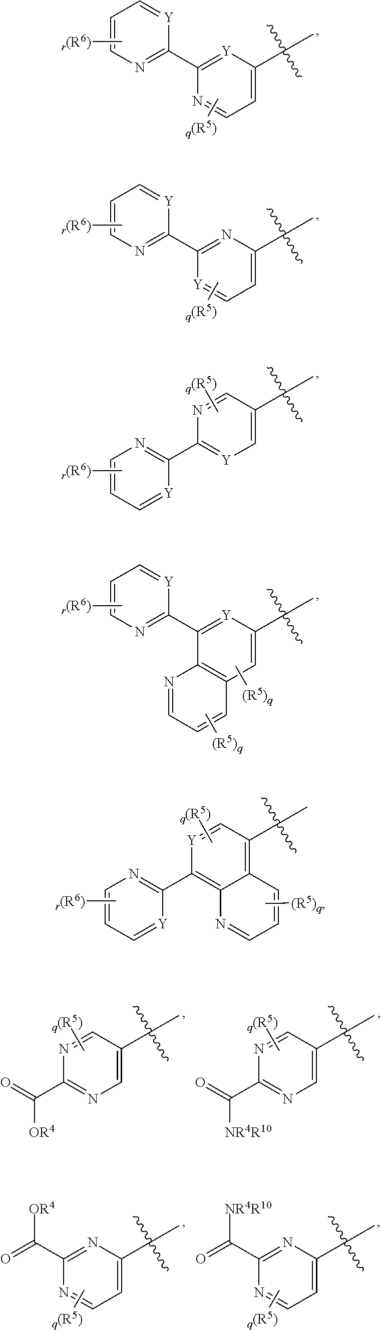

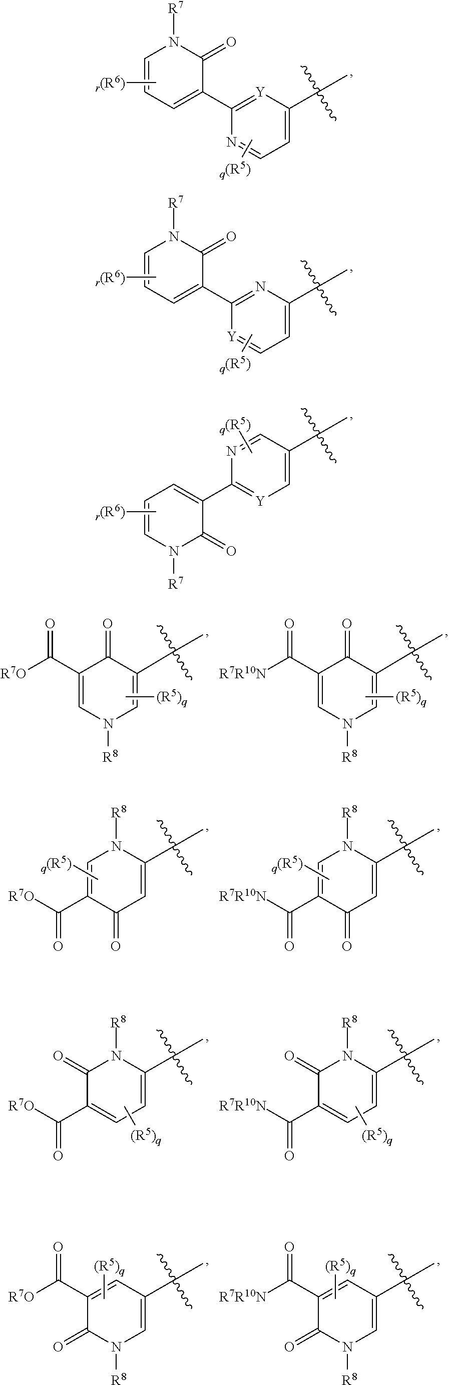

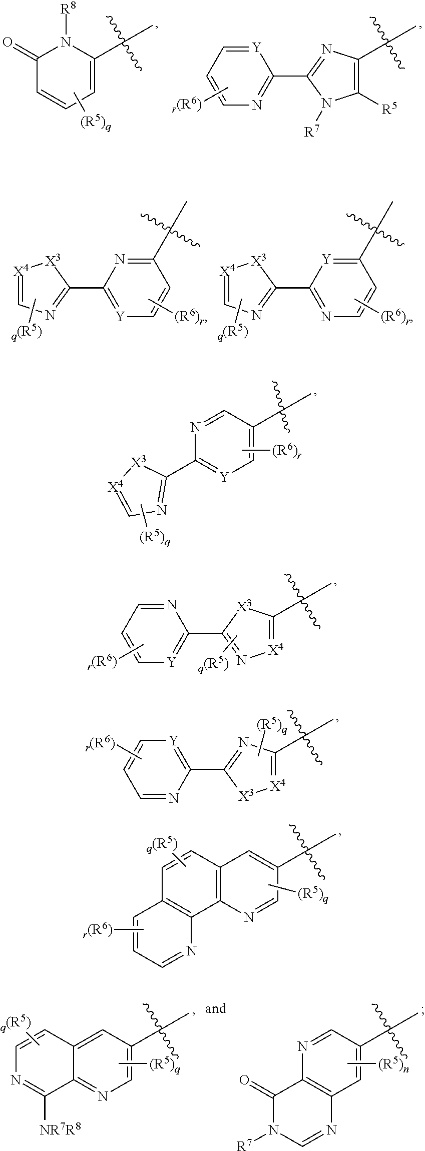

R⁹ is a bond if X¹ is CH, or R⁹ is selected from the group consisting of a bond and —C(═O)— if X¹ is N;

each occurrence of X³ is independently selected from the group consisting of NR⁷, O, and S;

each occurrence of X⁴ is independently selected from the group consisting of NR⁷ and CR⁵;

each occurrence of Y is independently selected from the group consisting of N and CR⁵;

each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)O₂C(═O)OR′, and —N(R′)(R′), wherein each occurrence of R′ is independently selected from the group consisting of H, optionally substituted C₁-C₆ alkyl, and optionally substituted C₃-C₈ cycloalkyl;
or two R² combine with the carbon atom to which both of them are bound to form a substituent selected from the group consisting of C(═O) and optionally substituted 1,1-(C₃-C₈ cycloalkanediyl);
or two R² bound to different carbon atoms combine to form an optionally substituted C₁-C₆ alkanediyl;

each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁴ is independently selected from the group consisting of H, optionally substituted C₁-C₆ alkyl, and optionally substituted C₃-C₈ cycloalkyl;

each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
or two R⁵ bound to adjacent carbon atoms combine to form optionally substituted 5-7 membered carbocyclyl or heterocyclyl;

each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁷ is independently selected from the group consisting of H, optionally substituted C₁-C₆ alkyl, and optionally substituted C₃-C₈ cycloalkyl;

each occurrence of R⁸ is independently selected from the group consisting of H, optionally substituted C₁-C₆ alkyl, and optionally substituted C₃-C₈ cycloalkyl;

each occurrence of R¹⁰ is independently selected from the group consisting of H, optionally substituted C₁-C₆ alkyl, optionally substituted C₃-C₈ cycloalkyl, optionally substituted phenyl, optionally substituted heteroaryl, —S(═O)₂(optionally substituted C₁-C₆ alkyl), and —S(═O)₂(optionally substituted C₃-C₈ cycloalkyl);

m is 0, 1, 2, 3, or 4;

n is 0, 1, or 2;

p is 0, 1, 2, 3, or 4;

q is 0, 1, or 2;

r is 0, 1, 2, or 3.

The invention includes a compound of formula (Ia), or a salt, solvate, geometric isomer, stereoisomer, tautomer, and any mixtures thereof:

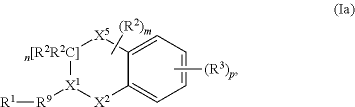

wherein in (Ia):

X¹ is N and X² is CR²R², or X² is NR⁴ and X¹ is CR⁴;

X⁵ is selected from the group consisting of O and CR²R²,
or one R² group from X⁵ can combine with one R² group of X² to form C₁-C₆ alkylene;

R¹ is selected from the group consisting of:

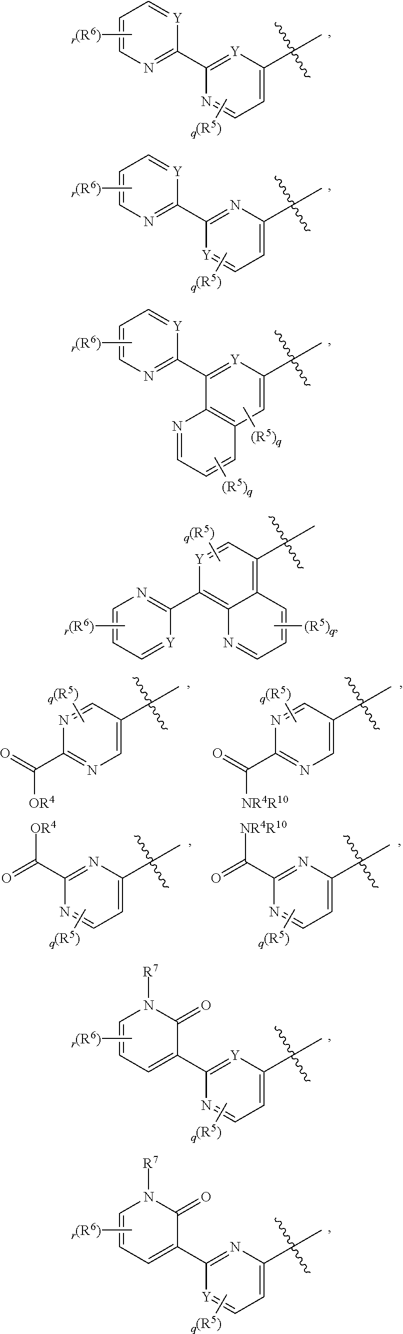

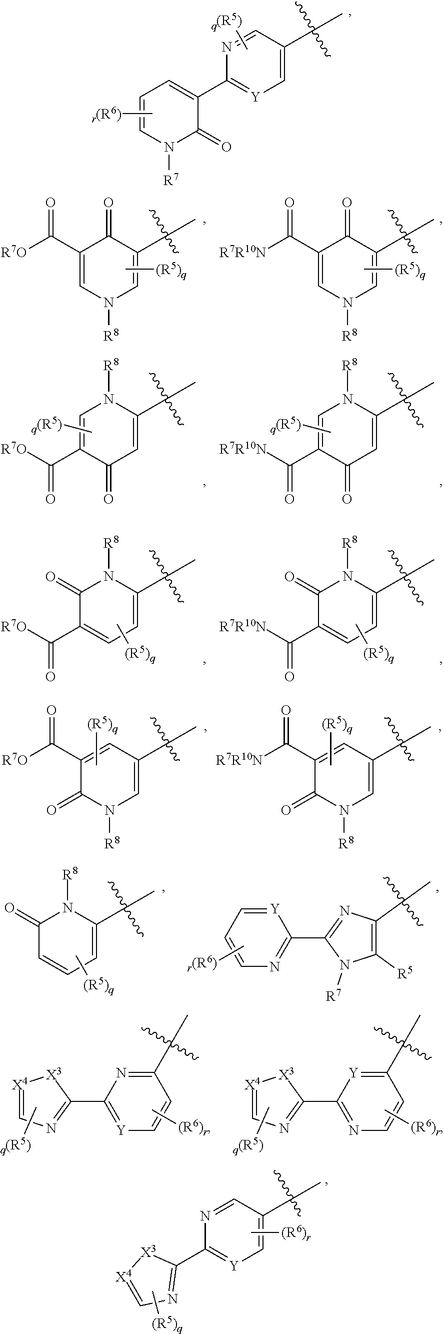

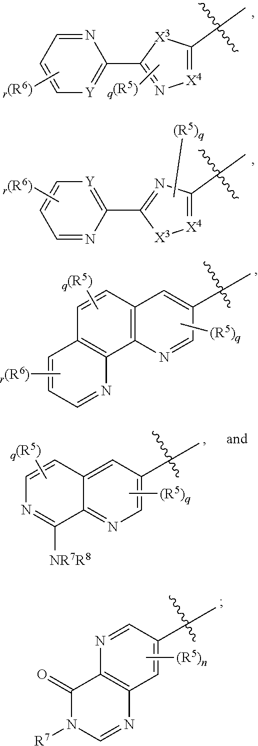

R⁹ is a bond if X¹ is CH, or R⁹ is selected from the group consisting of a bond and —C(═O)— if X¹ is N;

each occurrence of X³ is independently selected from the group consisting of NR⁷, O, and S;

each occurrence of X⁴ is independently selected from the group consisting of NR⁷ and CR⁵;

each occurrence of Y is independently selected from the group consisting of N and CR⁵;

each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)O₂C(═O)OR′, and —N(R′)(R′), wherein each occurrence of R′ is independently selected from the group consisting of H, optionally substituted C₁-C₆ alkyl, and optionally substituted C₃-C₈ cycloalkyl;
or two R² combine with the carbon atom to which both of them are bound to form a substituent selected from the group consisting of C(═O) and optionally substituted 1,1-(C₃-C₈ cycloalkanediyl);
or two R² bound to different carbon atoms combine to form an optionally substituted C₁-C₆ alkanediyl;

each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁴ is independently selected from the group consisting of H, optionally substituted C₁-C₆ alkyl, and optionally substituted C₃-C₈ cycloalkyl;

each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
or two R⁵ bound to adjacent carbon atoms combine to form optionally substituted 5-7 membered carbocyclyl or heterocyclyl;

each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁷ is independently selected from the group consisting of H, optionally substituted C₁-C₆ alkyl, and optionally substituted C₃-C₈ cycloalkyl;

each occurrence of R⁸ is independently selected from the group consisting of H, optionally substituted C₁-C₆ alkyl, and optionally substituted C₃-C₈ cycloalkyl;

each occurrence of R¹⁰ is independently selected from the group consisting of H, optionally substituted C₁-C₆ alkyl, optionally substituted C₃-C₈ cycloalkyl, optionally substituted phenyl, optionally substituted heteroaryl, —S(═O)₂(optionally substituted C₁-C₆ alkyl), and —S(═O)₂(optionally substituted C₃-C₈ cycloalkyl);

m is 0, 1, 2, 3, or 4;

n is 0, 1, or 2;

p is 0, 1, 2, 3, or 4;

q is 0, 1, or 2;

r is 0, 1, 2, or 3.

The invention includes a compound of formula (Ib), or a salt, solvate, geometric isomer, stereoisomer, tautomer, and any mixtures thereof:

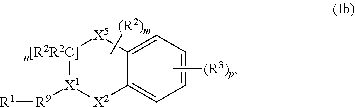

wherein in (Ib):

X¹ is N and X² is CR²R², or X² is NR⁴ and X¹ is CR⁴;

X⁵ is selected from the group consisting of O and CR²R²,
or one R² group from X⁵ can combine with one R² group of X² to form C₁-C₆ alkylene; R¹ is;

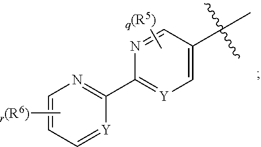

R⁹ is a bond if X¹ is CH, or R⁹ is selected from the group consisting of a bond and —C(═O)— if X¹ is N;
wherein, if R⁹ is a bond, X¹ is N, X² is CHR², and X⁵ is CH₂, then n is not 1;

each occurrence of Y is independently selected from the group consisting of N and CR⁵;

each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)₀₋₂C(═O)OR′, and —N(R′)(R′), wherein each occurrence of R′ is independently selected from the group consisting of H, optionally substituted C₁-C₆ alkyl, and optionally substituted C₃-C₈ cycloalkyl;
or two R² combine with the carbon atom to which both of them are bound to form a substituent selected from the group consisting of C(═O) and optionally substituted 1,1-(C₃-C₈ cycloalkanediyl);
or two R² bound to different carbon atoms combine to form an optionally substituted C₁-C₆ alkanediyl;

each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁴ is independently selected from the group consisting of H, optionally substituted C₁-C₆ alkyl, and optionally substituted C₃-C₈ cycloalkyl;

each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
or two R⁵ bound to adjacent carbon atoms combine to form optionally substituted 5-7 membered carbocyclyl or heterocyclyl;

each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

m is 0, 1, 2, 3, or 4;

n is 0, 1, or 2;

p is 0, 1, 2, 3, or 4;

q is 0, 1, or 2;

r is 0, 1, 2, or 3.

In certain embodiments, if R⁹ is a bond, X¹ is N, X² is CR²R², and X⁵ is CH₂, then n is not 1. In certain embodiments, if R⁹ is a bond, X¹ is N, X² is CHR², and X⁵ is CHR², then n is not 1. In certain embodiments, if R⁹ is a bond, X¹ is N, X² is CHR², and X⁵ is CR²R², then n is not 1. In certain embodiments, if R⁹ is a bond, X¹ is N, X² is CR²R², and X⁵ is CR²R² then n is not 1.

In certain embodiments, the compound of formula (I), (Ia), or (Ib) is:

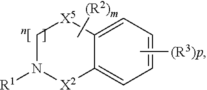

wherein X² is CR²R².

In certain embodiments, the compound of formula (I), (Ia), or (Ib) is:

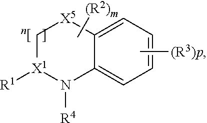

wherein X¹ is CR⁴.

In certain embodiments, each occurrence of R⁴ is independently selected from the group consisting of H, methyl, ethyl, propyl, isopropyl, butyl, isobutyl, sec-butyl, and tert-butyl.

In certain embodiments, R¹ is

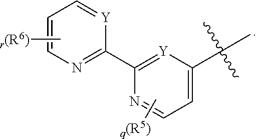

In certain embodiments, R¹ is

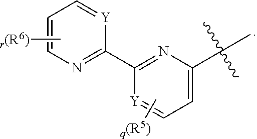

In certain embodiments, R¹ is

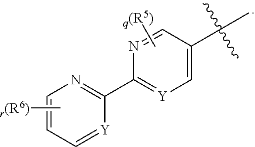

In certain embodiments, R¹ is

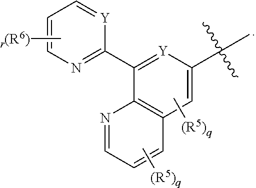

In certain embodiments, R¹ is

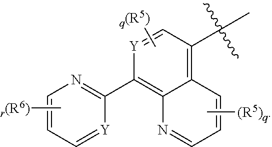

In certain embodiments, R¹ is

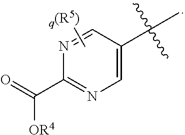

In certain embodiments, R¹ is

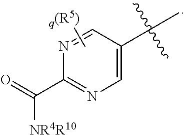

In certain embodiments, R¹ is

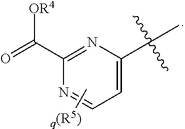

In certain embodiments, R¹ is

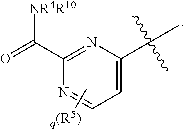

In certain embodiments, R¹ is

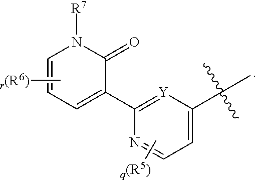

In certain embodiments, R¹ is

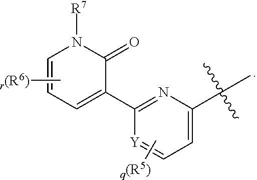

In certain embodiments, R¹ is

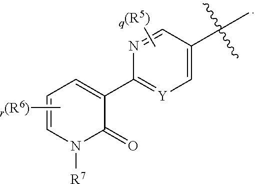

In certain embodiments, R¹ is

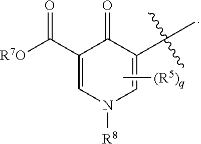

In certain embodiments, R¹ is

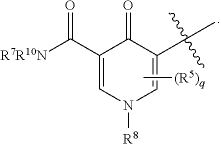

In certain embodiments, R¹ is

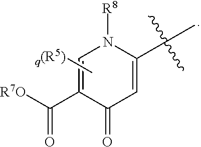

In certain embodiments, R¹ is

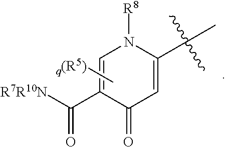

In certain embodiments, R¹ is

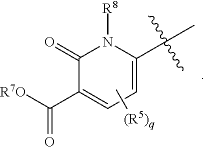

In certain embodiments, R¹ is

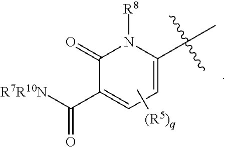

In certain embodiments, R¹ is

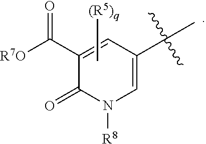

In certain embodiments, R¹ is

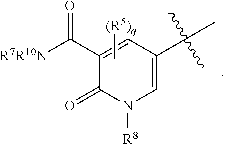

In certain embodiments, R¹ is

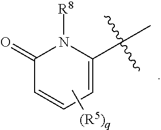

In certain embodiments, R¹ is

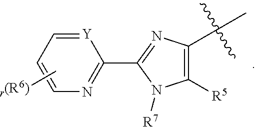

In certain embodiments, R¹ is

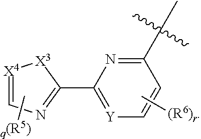

In certain embodiments, R¹ is

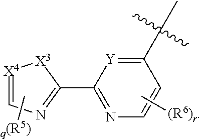

In certain embodiments, R¹ is

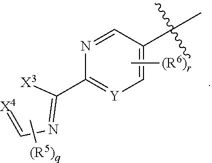

In certain embodiments, R¹ is

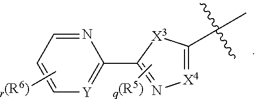

In certain embodiments, R¹ is

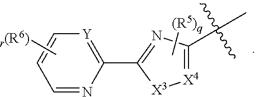

In certain embodiments, R¹ is

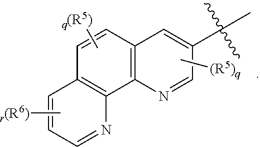

In certain embodiments, R¹ is

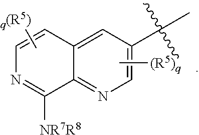

In certain embodiments, R¹ is

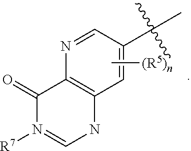

In certain embodiments, R¹ is

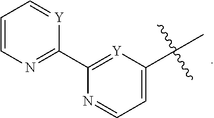

In other embodiments, R¹ is

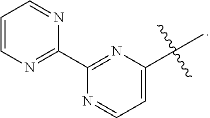 [SMILES: *c1ccnc(-c2ncccn2)n1]

In yet other embodiments, R¹ is

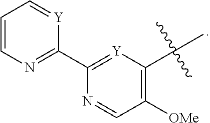

In yet other embodiments, R¹ is

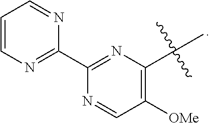 [SMILES: *c1nc(-c2ncccn2)ncc1OC]

In yet other embodiments, R¹ is

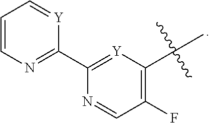

In yet other embodiments, R¹ is

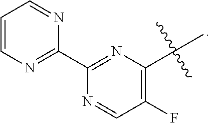 [SMILES: *c1nc(-c2ncccn2)ncc1F]

In yet other embodiments, R¹ is

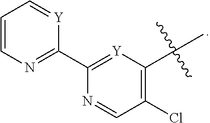

In yet other embodiments, R¹ is

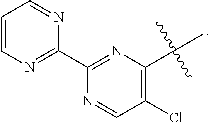 [SMILES: *c1nc(-c2ncccn2)ncc1Cl]

In yet other embodiments, R¹ is

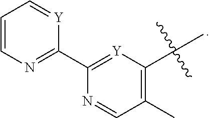

In yet other embodiments, R¹ is

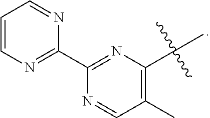 [SMILES: *c1nc(-c2ncccn2)ncc1C]

In yet other embodiments, R¹ is

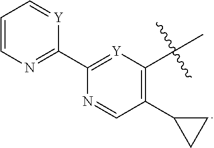

In yet other embodiments, R¹ is

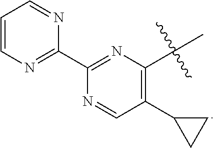 [SMILES: *c1nc(-c2ncccn2)ncc1C1CC1]

In yet other embodiments, R¹ is

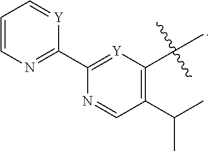

In yet other embodiments, R¹ is

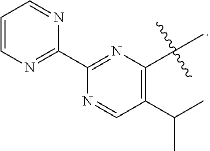 [SMILES: *c1nc(-c2ncccn2)ncc1C(C)C]

In yet other embodiments, R¹ is

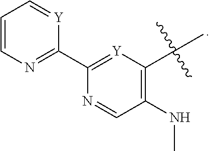

In yet other embodiments, R¹ is

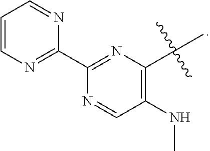 [SMILES: *c1nc(-c2ncccn2)ncc1NC]

In yet other embodiments, R¹ is

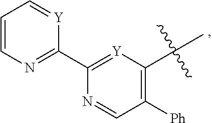

wherein Ph is optionally substituted. In yet other embodiments, R¹ is

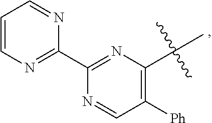

wherein Ph is optionally substituted. In yet other embodiments, R¹ is

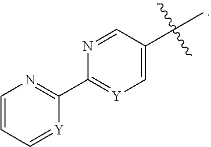

In yet other embodiments, R¹ is

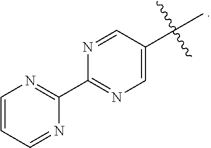 [SMILES: *c1cnc(-c2ncccn2)nc1]

In yet other embodiments, R¹ is

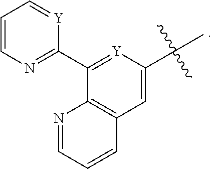

In yet other embodiments, R¹ is

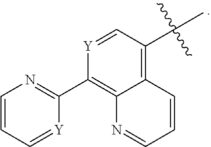

In yet other embodiments, R¹ is

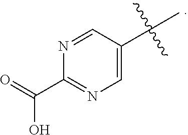 [SMILES: *c1cnc(C(=O)O)nc1]

In yet other embodiments, R¹ is

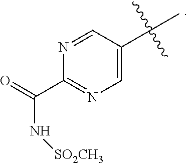 [SMILES: *c1cnc(C(=O)NS(C)(=O)=O)nc1]

In yet other embodiments, R is

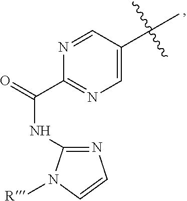

wherein R′″ is H, C₁-C₆ alkyl, or C₃-C₈ cycloalkyl. In yet other embodiments, R¹ is

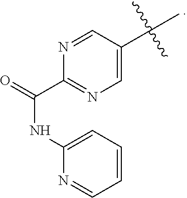 [SMILES: *c1cnc(C(=O)Nc2ccccn2)nc1]

In yet other embodiments, R¹ is

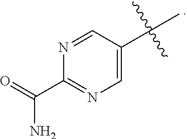 [SMILES: *c1cnc(C(N)=O)nc1]

In yet other embodiments, R¹ is

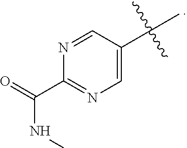 [SMILES: *c1cnc(C(=O)NC)nc1]

In yet other embodiments, R¹ is

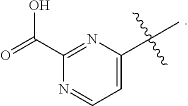 [SMILES: *c1ccnc(C(=O)O)n1]

In yet other embodiments, R¹ is

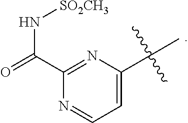 [SMILES: *c1ccnc(C(=O)NS(C)(=O)=O)n1]

In yet other embodiments, R¹ is

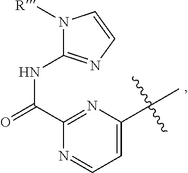

wherein R′″ is H, C₁-C₆ alkyl, or C₃-C₈ cycloalkyl. In yet other embodiments, R¹ is

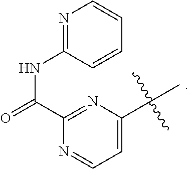 [SMILES: *c1ccnc(C(=O)Nc2ccccn2)n1]

In yet other embodiments, R¹ is

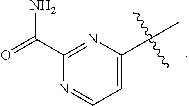 [SMILES: *c1ccnc(C(N)=O)n1]

In yet other embodiments, R¹ is

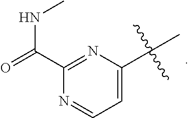 [SMILES: *c1ccnc(C(=O)NC)n1]

In yet other embodiments, R¹ is

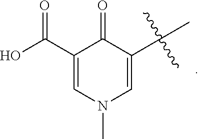 [SMILES: *c1cn(C)cc(C(=O)O)c1=O]

In yet other embodiments, R¹ is

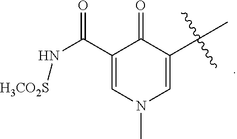

In yet other embodiments, R¹ is

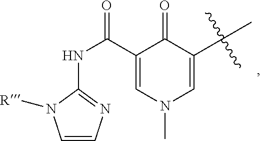

wherein R′″ is H, C₁-C₆ alkyl, or C₃-C₈ cycloalkyl. In yet other embodiments, R¹ is

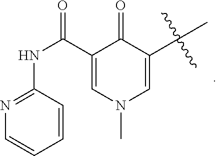 [SMILES: *c1cn(C)cc(C(=O)Nc2ccccn2)c1=O]

In yet other embodiments, R¹ is

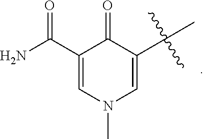 [SMILES: *c1cn(C)cc(C(N)=O)c1=O]

In yet other embodiments, R¹ is

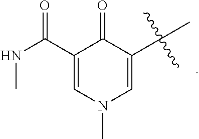 [SMILES: *c1cn(C)cc(C(=O)NC)c1=O]

In yet other embodiments, R¹ is

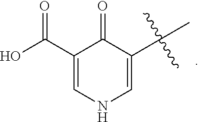 [SMILES: *c1c[nH]cc(C(=O)O)c1=O]

In yet other embodiments R¹ is

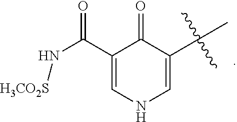

In yet other embodiments, R¹ is

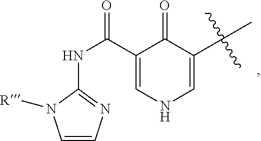

wherein R′″ is H, C₁-C₆ alkyl, or C₃-C₈ cycloalkyl. In yet other embodiments, R¹ is

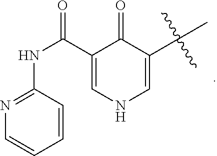 [SMILES: *c1c[nH]cc(C(=O)Nc2ccccn2)c1=O]

In yet other embodiments, R¹ is

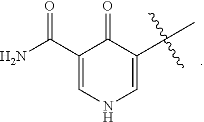 [SMILES: *c1c[nH]cc(C(N)=O)c1=O]

In yet other embodiments, R¹ is

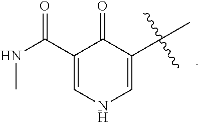 [SMILES: *c1c[nH]cc(C(=O)NC)c1=O]

In yet other embodiments, R¹ is

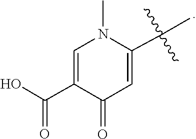 [SMILES: *c1cc(=O)c(C(=O)O)cn1C]

In yet other embodiments, R¹ is

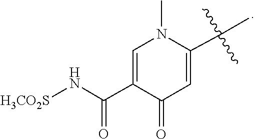

In yet other embodiments, R¹ is

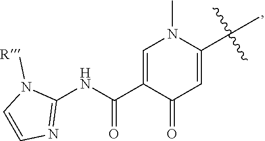

wherein R′″ is H, C₁-C₆ alkyl, or C₃-C₈. In yet other embodiments, R¹ is

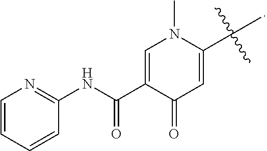 [SMILES: *c1cc(=O)c(C(=O)Nc2ccccn2)cn1C]

In yet other embodiments, R¹ is

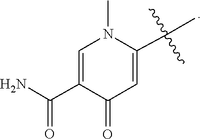 [SMILES: *c1cc(=O)c(C(N)=O)cn1C]

In yet other embodiments, R¹ is

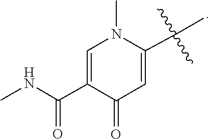 [SMILES: *c1cc(=O)c(C(=O)NC)cn1C]

In yet other embodiments, R¹ is

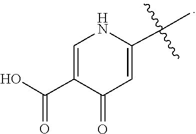 [SMILES: *c1cc(=O)c(C(=O)O)c[nH]1]

In yet other embodiments, R¹ is

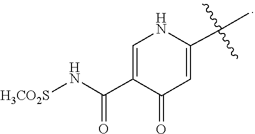

In yet other embodiments, R¹ is

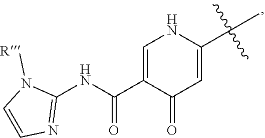

wherein R′″ is H, C₁-C₆ alkyl, or C₃-C₈ cycloalkyl. In yet other embodiments, R¹ is

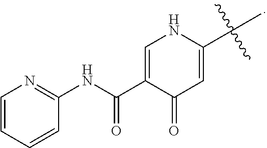 [SMILES: *c1cc(=O)c(C(=O)Nc2ccccn2)c[nH]1]

In yet other embodiments, R¹ is

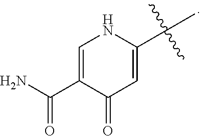 [SMILES: *c1cc(=O)c(C(N)=O)c[nH]1]

In yet other embodiments, R¹ is

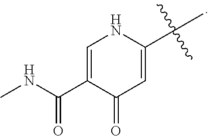 [SMILES: *c1cc(=O)c(C(=O)NC)c[nH]1]

In yet other embodiments, R¹ is

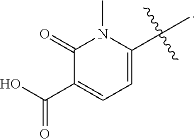 [SMILES: *c1ccc(C(=O)O)c(=O)n1C]

In yet other embodiments, R¹ is

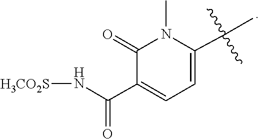

In yet other embodiments, R¹ is

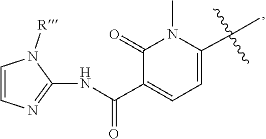

wherein R′″ is H, C₁-C₆ alkyl, or C₃-C₈ cycloalkyl. In yet other embodiments, R¹ is

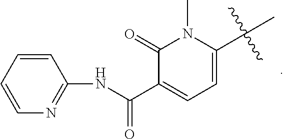 [SMILES: *c1ccc(C(=O)Nc2ccccn2)c(=O)n1C]

In yet other embodiments, R¹ is

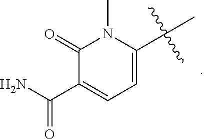 [SMILES: *c1ccc(C(N)=O)c(=O)n1C]

In yet other embodiments, R¹ is

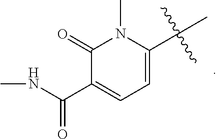 [SMILES: *c1ccc(C(=O)NC)c(=O)n1C]

In yet other embodiments, R¹ is

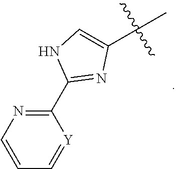

In yet other embodiments, R¹ is

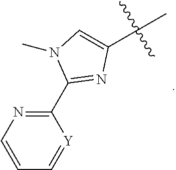

In yet other embodiments, R¹ is

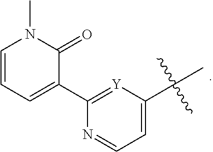

In yet other embodiments, R¹ is

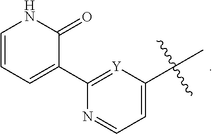

In yet other embodiments, R¹ is

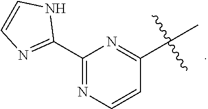 [SMILES: *c1ccnc(-c2ncc[nH]2)n1]

In yet other embodiments, R¹ is

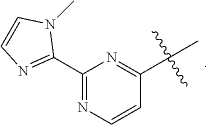 [SMILES: *c1ccnc(-c2nccn2C)n1]

In yet other embodiments, R¹ is

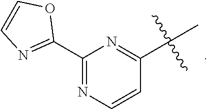 [SMILES: *c1ccnc(-c2ncco2)n1]

In yet other embodiments R¹ is

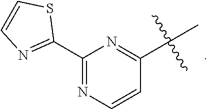 [SMILES: *c1ccnc(-c2nccs2)n1]

In yet other embodiments, R¹ is

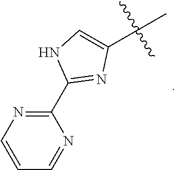 [SMILES: *c1c[nH]c(-c2ncccn2)n1]

In yet other embodiments, R¹ is

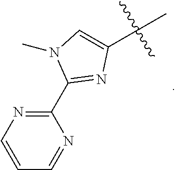 [SMILES: *c1cn(C)c(-c2ncccn2)n1]

In yet other embodiments, R¹ is

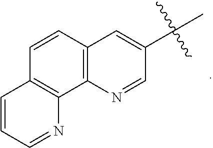 [SMILES: *c1cnc2c(ccc3cccnc32)c1]

In yet other embodiments, R¹ is

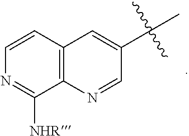

wherein R′″ is H, C₁-C₆ alkyl, or C₃-C₈ cycloalkyl.

In certain embodiments, R⁹ is a bond and X is CH. In certain embodiments, R⁹ is a bond and X¹ is N. In certain embodiments, R⁹ is —C(═O)— and X¹ is N.

In certain embodiments, X³ is NR⁷. In certain embodiments, X³ is O. In certain embodiments, X³ is S.

In certain embodiments, X⁴ is NR⁷. In certain embodiments, X⁴ is CR⁵.

In certain embodiments, Y is N. In certain embodiments, Y is CR⁵.

In certain embodiments, X² is selected from the group consisting of C═O, NH, N(CH₃), N(CH₂CH₃), N(CH(CH₃)₂), CH₂, CH(CH₃), CH(CH₂CH₃), CH(CH₂CH₂CH₃), CHCH(CH₃)₂, C(CH₃)₂, C(CH₃)(CH₂CH₃), C(CH₂CH₃)₂, 1,1-cyclopropanediyl, 1,1-cyclobutanediyl, 1,1-cyclopentanediyl, and 1,1-cyclohexanediyl.

In certain embodiments, each occurrence of R² is independently selected from the group consisting of H and C₁-C₆ alkyl.

In certain embodiments, two R² combine with the carbon atom to which both of them are bound to form a substituent selected from the group consisting of C(═O), 1,1-cyclopropanediyl, 1,1-cyclobutanediyl, 1,1-cyclopentanediyl, and 1,1-cyclohexanediyl.

In certain embodiments, two R² bound to different carbon atoms combine to form —CH₂—, —CH₂CH₂—, —CH(CH₃)CH₂—, —CH₂CH₂CH₂—, or —CH₂CH₂CH₂CH₂—.

In certain embodiments, two R² bound to different carbon atoms combine such that the compound of formula (I), (Ia), or (Ib) is

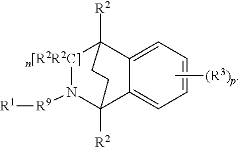

In certain embodiments, two R² bound to different carbon atoms combine such that the compound of formula (I), (Ia), or (Ib) is

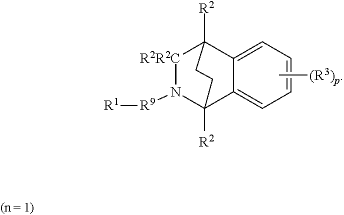

In certain embodiments, two R² bound to different carbon atoms combine such that the compound of formula (I), (Ia), or (Ib) is

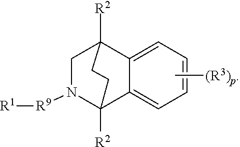

In certain embodiments, two R² bound to different carbon atoms combine such that the compound of formula (I), (Ia), or (Ib) is

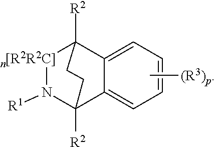

In certain embodiments, two R² bound to different carbon atoms combine such that the compound of formula (I), (Ia), or (Ib) is

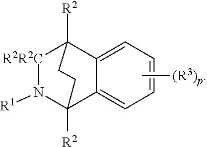

In certain embodiments, two R² bound to different carbon atoms combine such that the compound of formula (I), (Ia), or (Ib) is

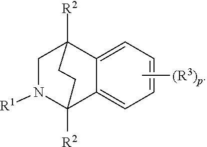

In certain embodiments, two R² bound to different carbon atoms combine such that the compound of formula (I), (Ia), or (Ib) is

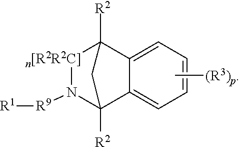

In certain embodiments, two R² bound to different carbon atoms combine such that the compound of formula (I), (Ia), or (Ib) is

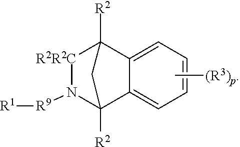

In certain embodiments, two R² bound to different carbon atoms combine such that the compound of formula (I), (Ia), or (Ib) is

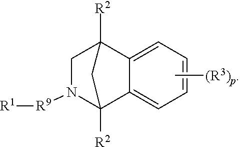

In certain embodiments, two R² bound to different carbon atoms combine such that the compound of formula (I), (Ia), or (Ib) is

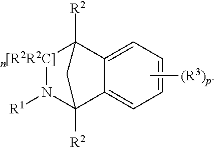

In certain embodiments, two R² bound to different carbon atoms combine such that the compound of formula (I), (Ia), or (Ib) is

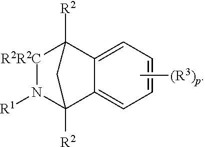

In certain embodiments, two R² bound to different carbon atoms combine such that the compound of formula (I), (Ia), or (Ib) is

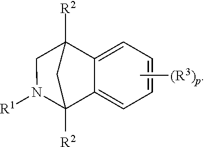

In certain embodiments, two R² bound to different carbon atoms combine such that the compound of formula (I), (Ia), or (Ib) is

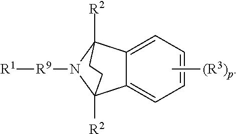

In certain embodiments, two R² bound to different carbon atoms combine such that the compound of formula (I), (Ia), or (Ib) is

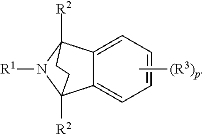

In certain embodiments, each occurrence of R³ is independently selected from the group consisting of H, halo (such as, but not limited to F or Cl), C₁-C₆ alkyl, C₁-C₆ haloalkyl, C₁-C₆ alkoxy, and C₁-C₆ haloalkoxy.

In certain embodiments, each occurrence of R³ is such that the compound of formula (I), (Ia), or (Ib) is

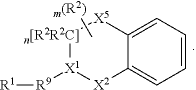

In certain embodiments, each occurrence of R³ is such that the compound of formula (I), (Ia), or (Ib) is

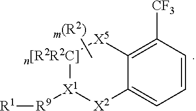

In certain embodiments, each occurrence of R³ is such that the compound of formula (I), (Ia), or (Ib) is

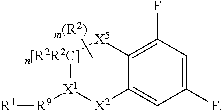

In certain embodiments, each occurrence of R³ is such that the compound of formula (I), (Ia), or (Ib) is

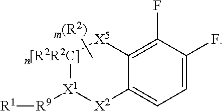

In certain embodiments, each occurrence of R³ is such that the compound of formula (I), (Ia), or (Ib) is

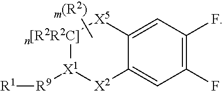

In certain embodiments, each occurrence of R³ is such that the compound of formula (I), (Ia), or (Ib) is

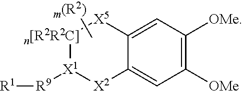

In certain embodiments, each occurrence of R³ is such that the compound of formula (I), (Ia), or (Ib) is

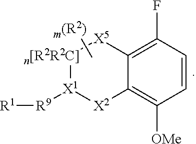

In certain embodiments, each occurrence of R³ is such that the compound of formula (I), (Ia), or (Ib) is

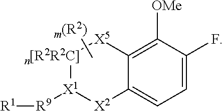

In certain embodiments, each occurrence of R³ is such that the compound of formula (I), (Ia), or (Ib) is

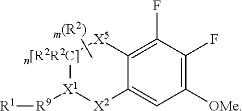

In certain embodiments, each occurrence of R³ is such that the compound of formula (I), (Ia), or (Ib) is

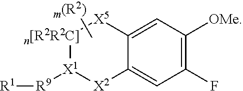

In certain embodiments, each occurrence of R³ is such that the compound of formula (I), (Ia), or (Ib) is

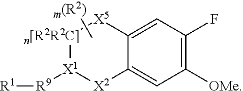

In certain embodiments, each occurrence of R³ is such that the compound of formula (I), (Ia), or (Ib) is

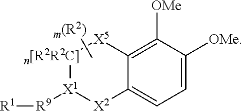

In certain embodiments, each occurrence of R³ is such that the compound of formula (I), (Ia), or (Ib) is

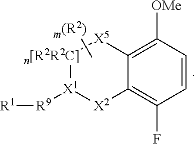

In certain embodiments, each occurrence of R³ is such that the compound of formula
(I), (Ia), or (Ib) is

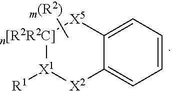

In certain embodiments, each occurrence of R³ is such that the compound of formula (I), (Ia), or (Ib) is

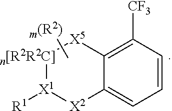

In certain embodiments, each occurrence of R³ is such that the compound of formula (I), (Ia), or (Ib) is

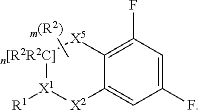

In certain embodiments, each occurrence of R³ is such that the compound of formula (I), (Ia), or (Ib) is

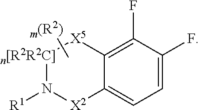

In certain embodiments, each occurrence of R³ is such that the compound of formula (I), (Ia), or (Ib) is

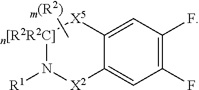

In certain embodiments, each occurrence of R³ is such that the compound of formula (I), (Ia), or (Ib) is

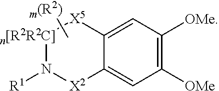

In certain embodiments, each occurrence of R³ is such that the compound of formula (I), (Ia), or (Ib) is

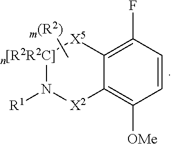

In certain embodiments, each occurrence of R³ is such that the compound of formula (I), (Ia), or (Ib) is

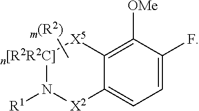

In certain embodiments, each occurrence of R³ is such that the compound of formula (I), (Ia), or (Ib) is

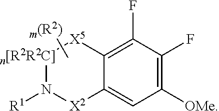

In certain embodiments, each occurrence of R³ is such that the compound of formula (I), (Ia), or (Ib) is

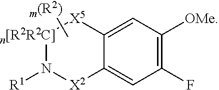

In certain embodiments, each occurrence of R³ is such that the compound of formula (I), (Ia), or (Ib) is

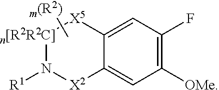

In certain embodiments, each occurrence of R³ is such that the compound of formula (I), (Ia), or (Ib) is

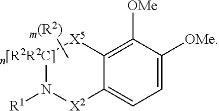

In certain embodiments, each occurrence of R³ is such that the compound of formula (I), (Ia), or (Ib) is

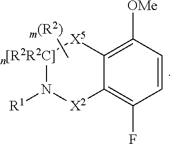

In certain embodiments, each occurrence of R³ is such that the

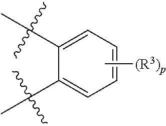

ring in (I), (Ia), or (Ib) is

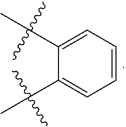 [SMILES: *c1ccccc1*]

In certain embodiments, each occurrence of R³ is such that the

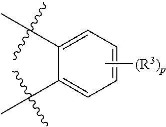

ring in (I), (Ia), or (Ib) is

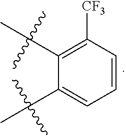 [SMILES: *c1cccc(C(F)(F)F)c1*]

In certain embodiments, each occurrence of R³ is such that the

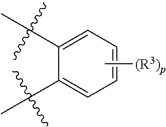

ring in (I), (Ia), or (Ib) is

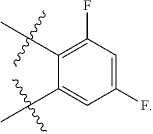 [SMILES: *c1cc(F)cc(F)c1*]

In certain embodiments, each occurrence of R³ is such that the

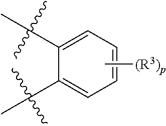

ring in (I), (Ia), or (Ib) is

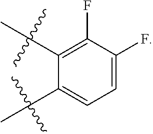 [SMILES: *c1ccc(F)c(F)c1*]

In certain embodiments, each occurrence of R³ is such that the

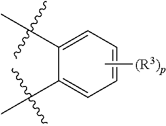

ring in (I), (Ia), or (b) is

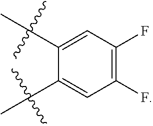 [SMILES: *c1cc(F)c(F)cc1*]

In certain embodiments, each occurrence of R¹ is such that the

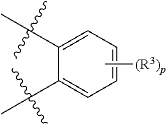

ring in (I), (Ia), or (Ib) is

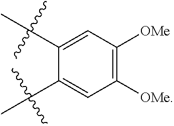 [SMILES: *c1cc(OC)c(OC)cc1*]

In certain embodiments, each occurrence of R³ is such that the

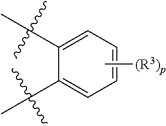

ring in (I), (Ia), or (Ib) is

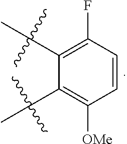 [SMILES: *c1c(F)ccc(OC)c1*]

In certain embodiments, each occurrence of R³ is such that the

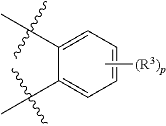

ring in (I), (Ia), or (Ib) is

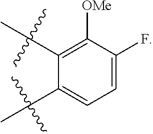 [SMILES: *c1ccc(F)c(OC)c1*]

In certain embodiments, each occurrence of R³ is such that the

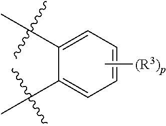

ring in (I), (Ia), or (Ib) is

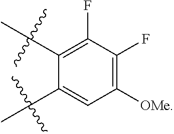 [SMILES: *c1cc(OC)c(F)c(F)c1*]

In certain embodiments, each occurrence of R³ is such that the

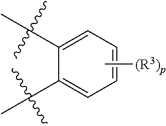

ring in (I), (Ia), or (Ib) is

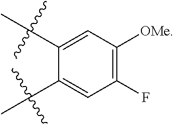 [SMILES: *c1cc(F)c(OC)cc1*]

In certain embodiments, each occurrence of R³ is such that the

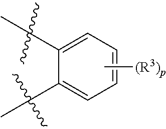

ring in (I), (Ia), or (Ib) is

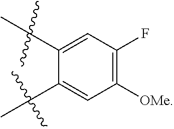 [SMILES: *c1cc(F)c(OC)cc1*]

In certain embodiments, each occurrence of R³ is such that the

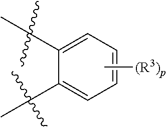

ring in (I), (Ia), or (Ib) is

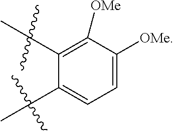 [SMILES: *c1ccc(OC)c(OC)c1*]

In certain embodiments, each occurrence of R³ is such that the

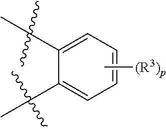

ring in (I), (Ia), or (Ib) is

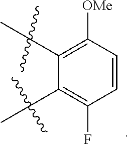 [SMILES: *c1c(F)ccc(OC)c1*]

In certain embodiments, two R⁵ bound to adjacent carbon atoms combine to form optionally substituted 5-membered carbocyclyl or heterocyclyl. In certain embodiments, two R⁵ bound to adjacent carbon atoms combine to form optionally substituted 6-membered carbocyclyl or heterocyclyl. In certain embodiments, two R⁵ bound to adjacent carbon atoms combine to form optionally substituted 7-membered carbocyclyl or heterocyclyl.

In certain embodiments, two R⁵ bound to adjacent carbon atoms combine to form —S—CR′═N—, wherein R′ is H or C₁-C₆ alkyl. In certain embodiments, two R⁵ bound to adjacent carbon atoms combine to form —N═CR′—S—, wherein R′ is H or C₁-C₆ alkyl. In certain embodiments, two R⁵ bound to adjacent carbon atoms combine to form —(CH₂)₃—, wherein each methylene group is optionally substituted with one or two independently selected halo or C₁-C₆ alkyl. In certain embodiments, two R⁵ bound to adjacent carbon atoms combine to form —CH₂OCH₂—, —OCH₂CH₂—, or —CH₂CH₂—, wherein each methylene group is optionally substituted with one or two independently selected halo or C₁-C₆ alkyl. In certain embodiments, two R⁵ bound to adjacent carbon atoms combine to form —OCH═CH— or —CH═CHO—, wherein each CH group is optionally substituted with one independently selected halo or C₁-C₆ alkyl.

In certain embodiments, two R⁵ bound to adjacent carbon atoms combine to form

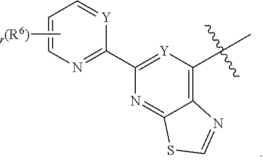

In certain embodiments, two R⁵ bound to adjacent carbon atoms combine to form

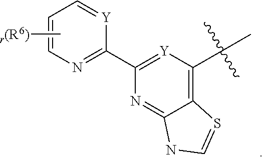

In certain embodiments, two R⁵ bound to adjacent carbon atoms combine to form

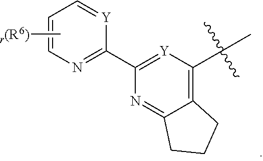

In certain embodiments, two R⁵ bound to adjacent carbon atoms combine to form

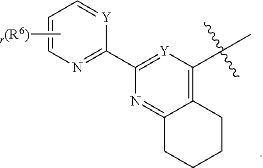

In certain embodiments, two R⁵ bound to adjacent carbon atoms combine to form

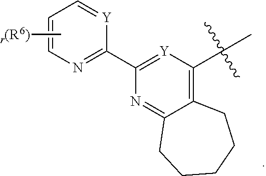

In certain embodiments, two R⁵ bound to adjacent carbon atoms combine to form

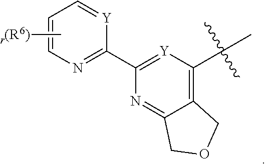

In certain embodiments, two R⁵ bound to adjacent carbon atoms combine to form

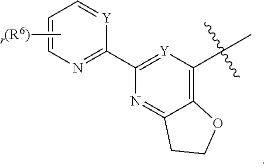

In certain embodiments, two R⁵ bound to adjacent carbon atoms combine to form

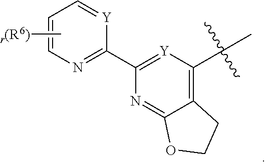

In certain embodiments, two R⁵ bound to adjacent carbon atoms combine to form

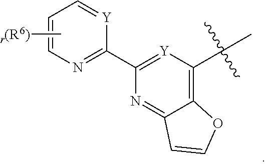

In certain embodiments, two R⁵ bound to adjacent carbon atoms combine to form

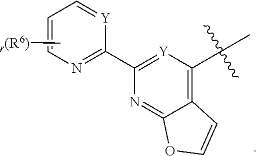

In certain embodiments, m is 0. In certain embodiments, m is 1. In certain embodiments, m is 2. In certain embodiments, m is 3. In certain embodiments, m is 4.

In certain embodiments, n is 0. In certain embodiments, n is 1. In certain embodiments, n is 2.

In certain embodiments, p is 0. In certain embodiments, p is 1. In certain embodiments, p is 2. In certain embodiments, p is 3. In certain embodiments, p is 4.

In certain embodiments, q is 0. In certain embodiments, q is 1. In certain embodiments, q is 2.

In certain embodiments, r is 0. In certain embodiments, r is 1. In certain embodiments, r is 2. In certain embodiments, r is 3.

In certain embodiments, each occurrence of alkyl, alkylenyl (alkylene), cycloalkyl, heterocyclyl, or carbocyclyl is independently optionally substituted with at least one substituent selected from the group consisting of C₁-C₆ alkyl, halo, —OR″, phenyl (thus yielding, in non-limiting examples, optionally substituted phenyl-(C₁-C₃ alkyl), such as, but not limited to, benzyl or substituted benzyl), and —N(R″)(R″), wherein each occurrence of R″ is independently H, C₁-C₆ alkyl or C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR″, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR″, —N(R″)(R″), and C₁-C₆ alkoxycarbonyl, wherein each occurrence of R″ is independently H, C₁-C₆ alkyl or C₃-C₈ cycloalkyl.

In certain embodiments, the compounds of the invention, or a salt, solvate, stereoisomer (such as, in a non-limiting example, an enantiomer or diastereoisomer thereof), any mixture of one or more stereoisomers (such as, in a non-limiting example, mixtures in any proportion of enantiomers thereof, and/or mixtures in any proportion of diastereoisomers thereof), tautomer, and/or any mixture of tautomers thereof, are recited in Table 1.

The compounds of the invention may possess one or more stereocenters, and each stereocenter may exist independently in either the (R) or (S) configuration. In certain embodiments, compounds described herein are present in optically active or racemic forms.

The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, 3-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the invention are useful within the methods of the invention in combination with one or more additional agents useful for treating HBV infections. These additional agents may comprise compounds or compositions identified herein, or compounds (e.g., commercially available compounds) known to treat, prevent, or reduce the symptoms of HBV infections.

Non-limiting examples of one or more additional agents useful for treating HBV infections include: (a) reverse transcriptase inhibitors; (b) capsid inhibitors; (c) cccDNA formation inhibitors; (d) sAg secretion inhibitors; (e) oligomeric nucleotides targeted to the Hepatitis B genome; and (f) immunostimulators.

### (a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

### (b) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921 and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO2017048954, WO 2017048962, WO 2017064156 and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA.

For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

### (d) sAg Secretion Inhibitors

As described herein, the term “sAg secretion inhibitor” includes compounds that are capable of inhibiting, either directly or indirectly, the secretion of sAg (S, M and/or L surface antigens) bearing subviral particles and/or DNA containing viral particles from HBV-infected cells. For example, in certain embodiments, the inhibitor detectably inhibits the secretion of sAg as measured, e.g., using assays known in the art or described herein, e.g., ELISA assay or by Western Blot. In certain embodiments, the inhibitor inhibits the secretion of sAg by at least 5%, at least 10%, at least 20%, at least 50%, at least 75%, or at least 90%. In certain embodiments, the inhibitor reduces serum levels of sAg in a patient by at least 5%, at least 10%, at least 20%, at least 50%, at least 75%, or at least 90%.

Reported sAg secretion inhibitors include compounds described in U.S. Pat. No. 8,921,381, as well as compounds described in U.S. Patent Application Publication Nos. US 2015/0087659 and US 2013/0303552, all of which are incorporated herein in their entireties by reference.

In addition, reported sAg secretion inhibitors include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821 and are incorporated herein in their entirety by reference.

### (e) Immunostimulators

The term “immunostimulator” includes compounds that are capable of modulating an immune response (e.g., stimulate an immune response (e.g., an adjuvant)).

Immunostimulators include, but are not limited to, polyinosinic:poly cytidylic acid (poly I:C) and interferons.

Reported immunostimulators include, but are not limited to, agonists of stimulator of IFN genes (STING) and interleukins. Reported immunostimulators further include, but are not limited to, HBsAg release inhibitors, TLR-7 agonists (such as, but not limited to, GS-9620, RG-7795), T-cell stimulators (such as, but not limited to, GS-4774), RIG-1 inhibitors (such as, but not limited to, SB-9200), and SMAC-mimetics (such as, but not limited to, Birinapant).

### (f) Oligomeric Nucleotides

Reported oligomeric nucleotides targeted to the Hepatitis B genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the Hepatitis B genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

A synergistic effect may be calculated, for example, using suitable methods such as, for example, the Sigmoid-Eₘₐₓ equation (Holford & Schemer, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present invention further provides methods of preparing the compounds of the present invention. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It should be contemplated that the invention includes each and every one of the synthetic schemes described and/or depicted herein.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

In certain embodiments, a compound of the invention can be prepared, for example, according to the illustrative synthetic methods outlined in Scheme I:

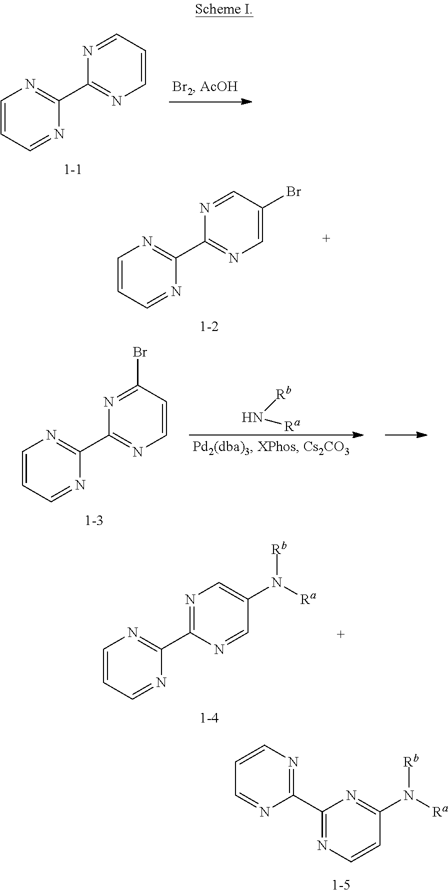

In certain embodiments, a compound of the invention can be prepared, for example, according to the illustrative synthetic methods outlined in Scheme II:

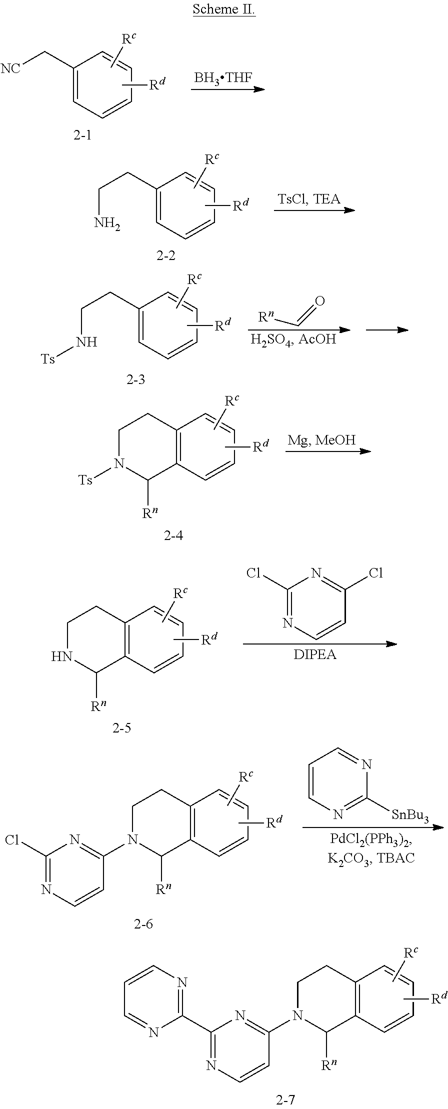

In certain embodiments, a compound of the invention can be prepared, for example, according to the illustrative synthetic methods outlined in Scheme III:

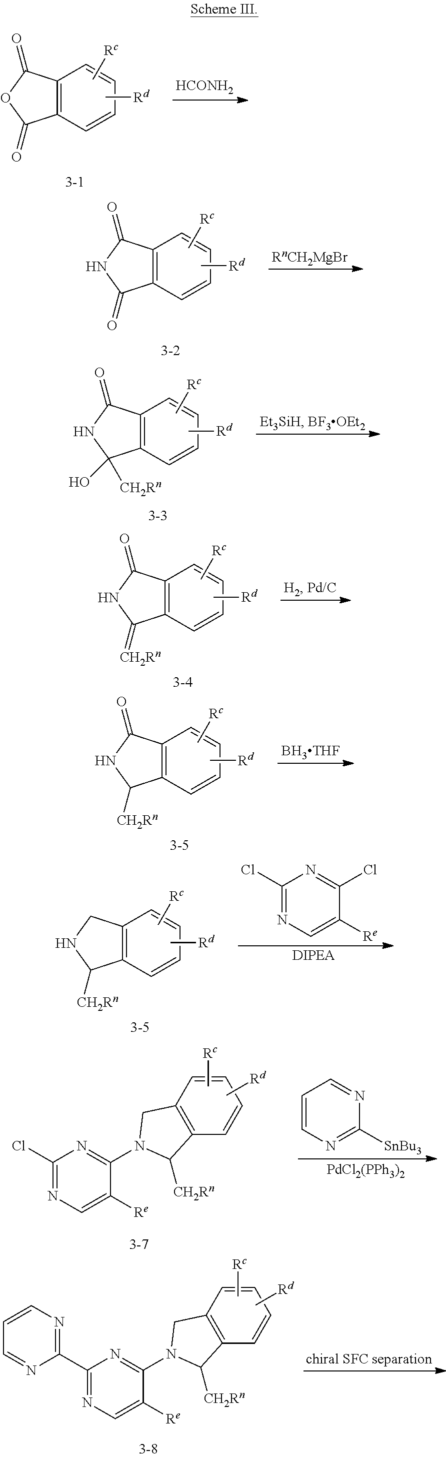

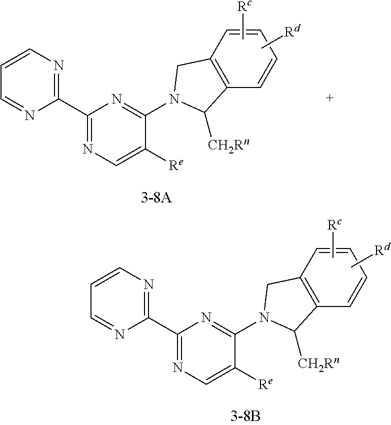

In certain embodiments, a compound of the invention can be prepared, for example, according to the illustrative synthetic methods outlined in Scheme IV:

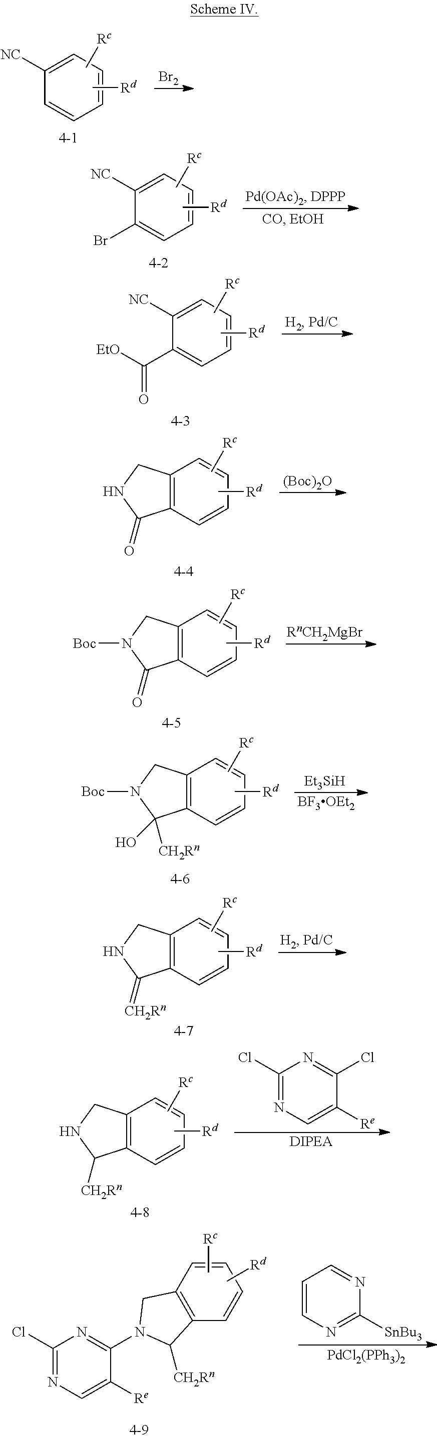

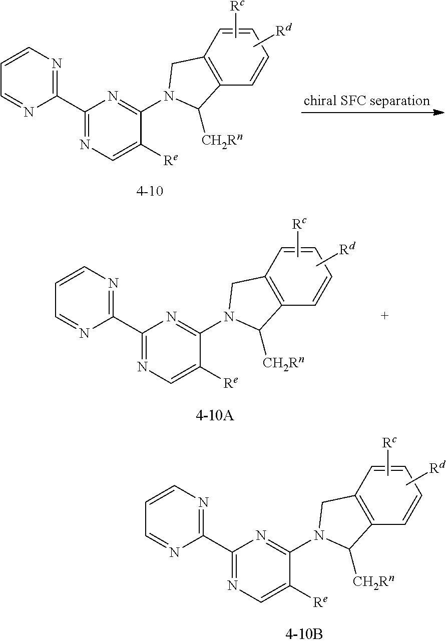

In certain embodiments, a compound of the invention can be prepared, for example, according to the illustrative synthetic methods outlined in Scheme V:

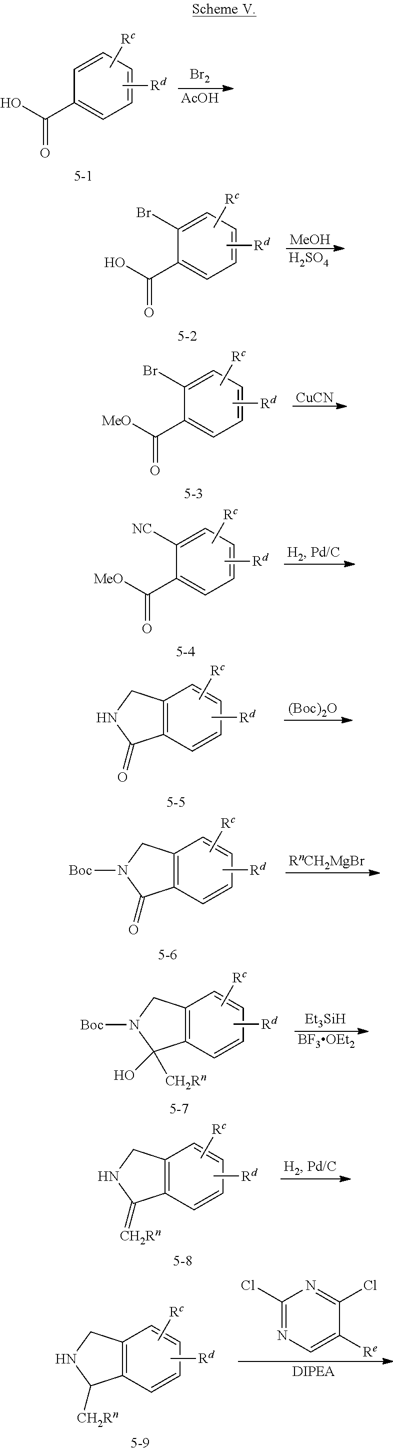

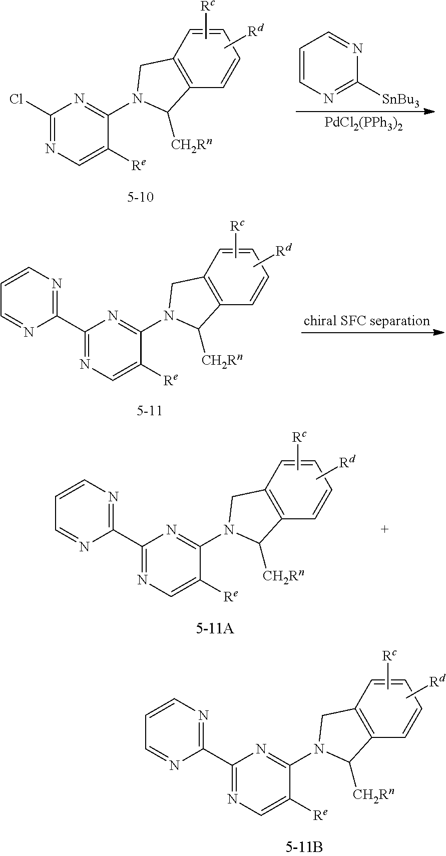

In certain embodiments, a compound of the invention can be prepared, for example, according to the illustrative synthetic methods outlined in Scheme VI:

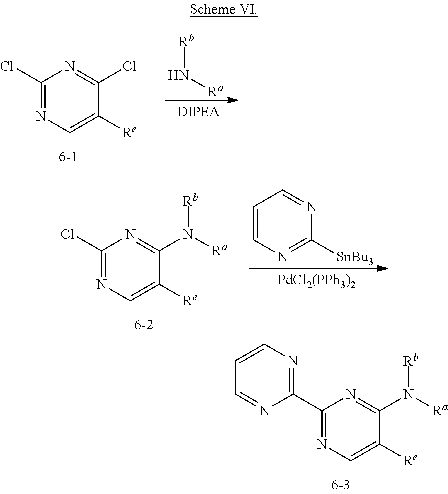

In certain embodiments, a compound of the invention can be prepared, for example, according to the illustrative synthetic methods outlined in Scheme VII:

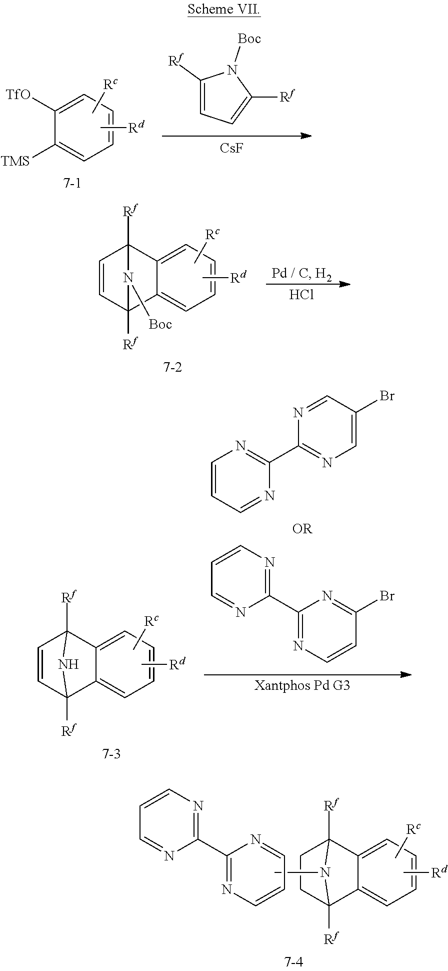

In certain embodiments, a compound of the invention can be prepared, for example, according to the illustrative synthetic methods outlined in Scheme VIII:

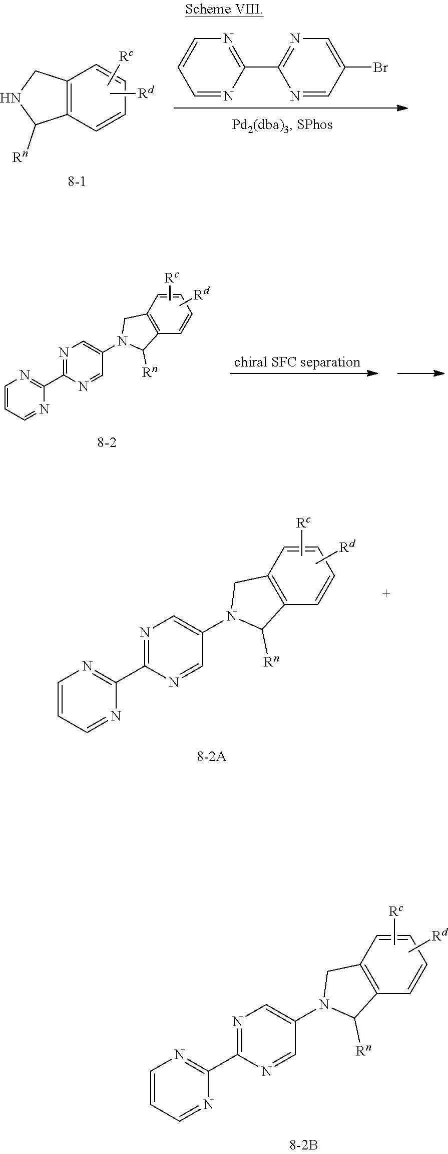

In certain embodiments, a compound of the invention can be prepared, for example, according to the illustrative synthetic methods outlined in Scheme IX:

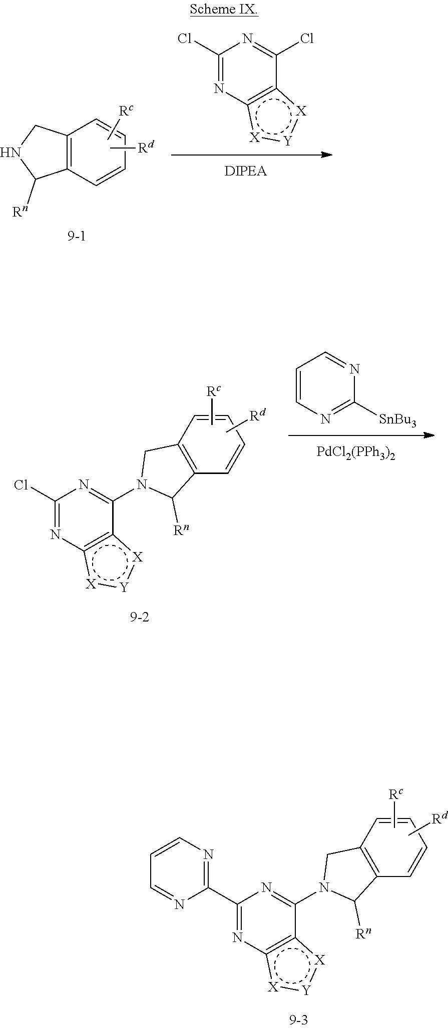

In certain embodiments, a compound of the invention can be prepared, for example, according to the illustrative synthetic methods outlined in Scheme X:

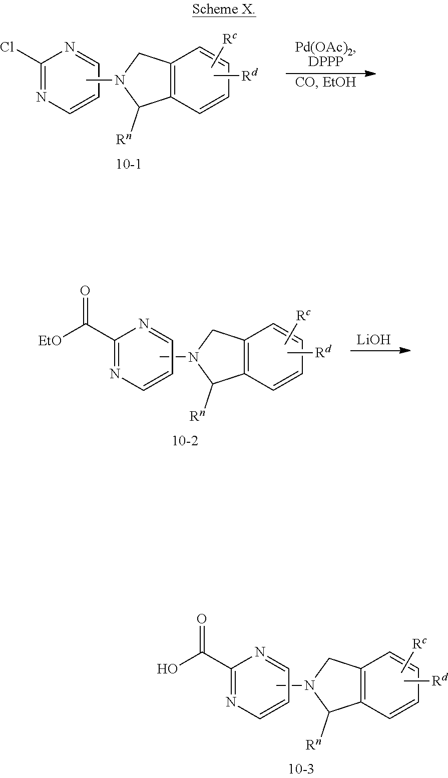

In certain embodiments, a compound of the invention can be prepared, for example, according to the illustrative synthetic methods outlined in Scheme XI:

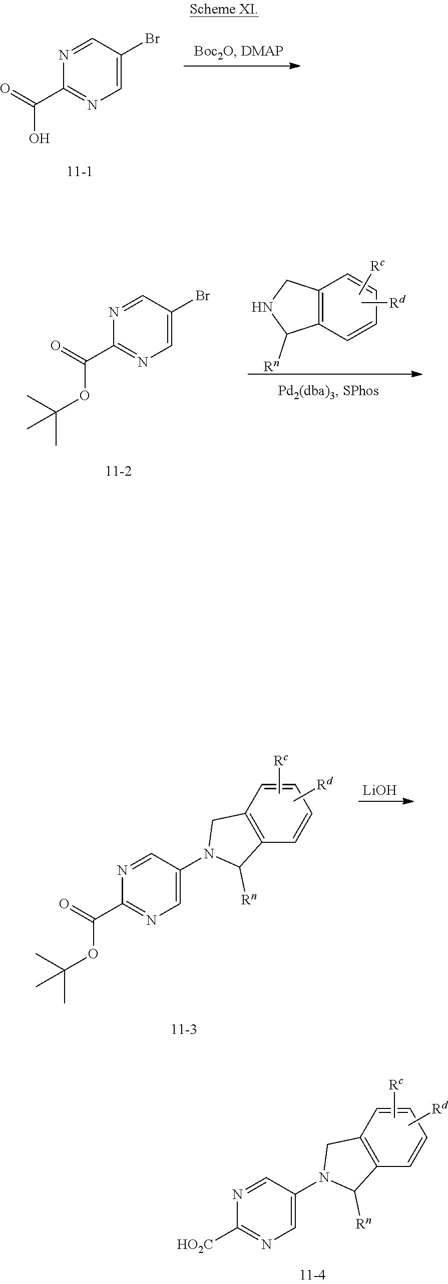

In certain embodiments, a compound of the invention can be prepared, for example, according to the illustrative synthetic methods outlined in Scheme XII:

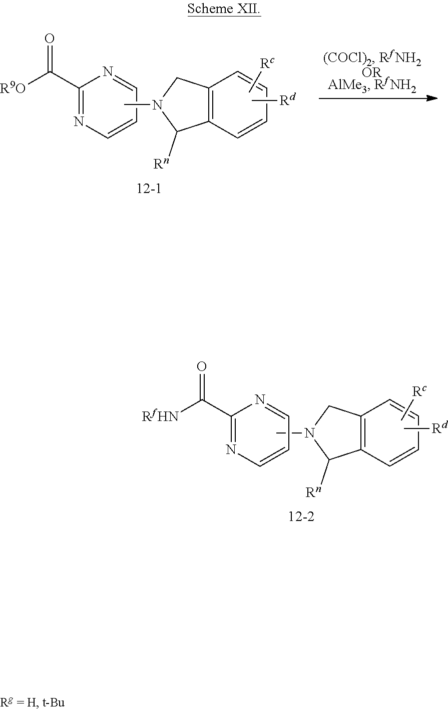

In certain embodiments, a compound of the invention can be prepared, for example, according to the illustrative synthetic methods outlined in Scheme XIII:

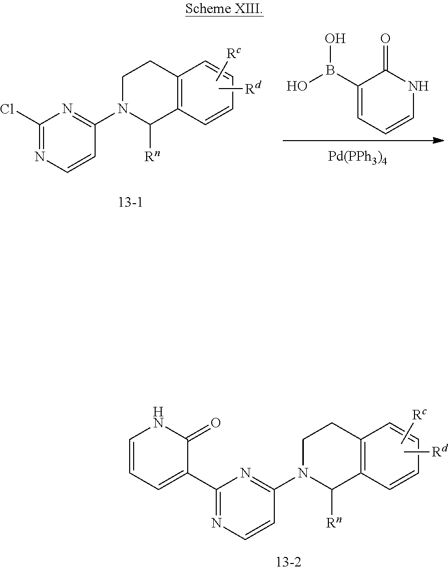

In certain embodiments, a compound of the invention can be prepared, for example, according to the illustrative synthetic methods outlined in Scheme XIV:

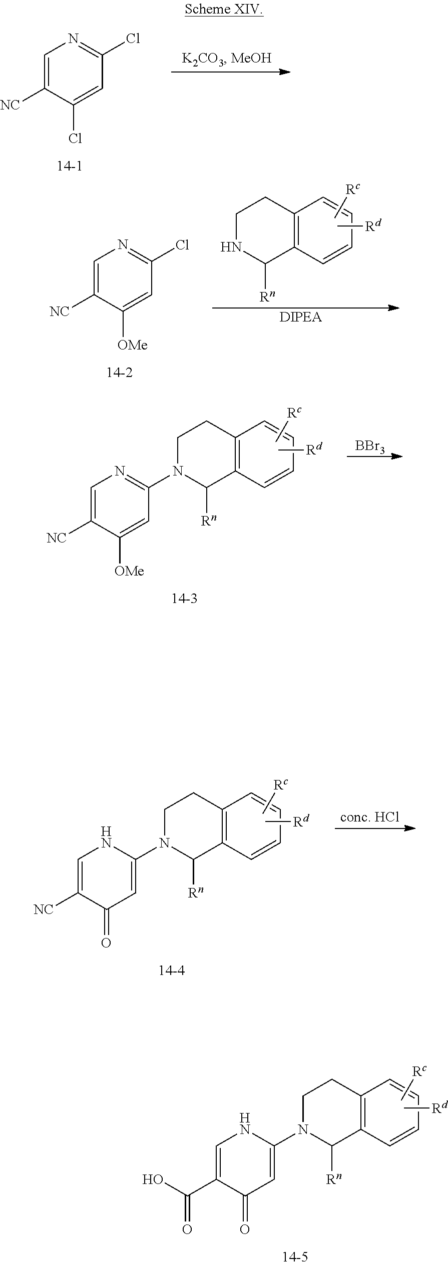

In certain embodiments, a compound of the invention can be prepared, for example, according to the illustrative synthetic methods outlined in Scheme XV:

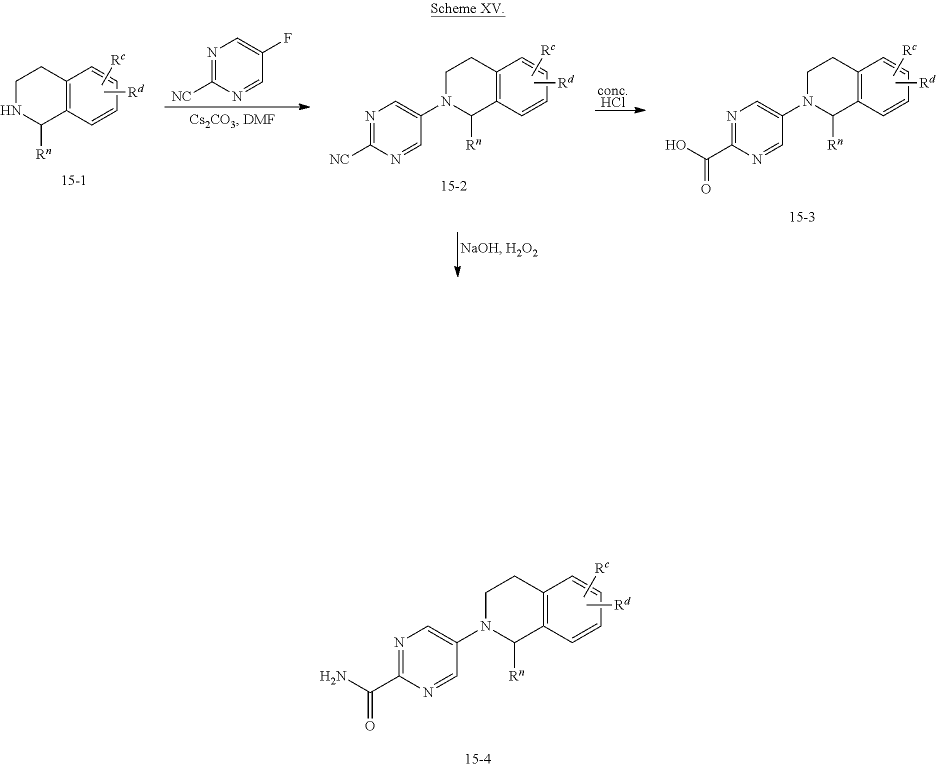

In certain embodiments, a compound of the invention can be prepared, for example, according to the illustrative synthetic methods outlined in Scheme XVI:

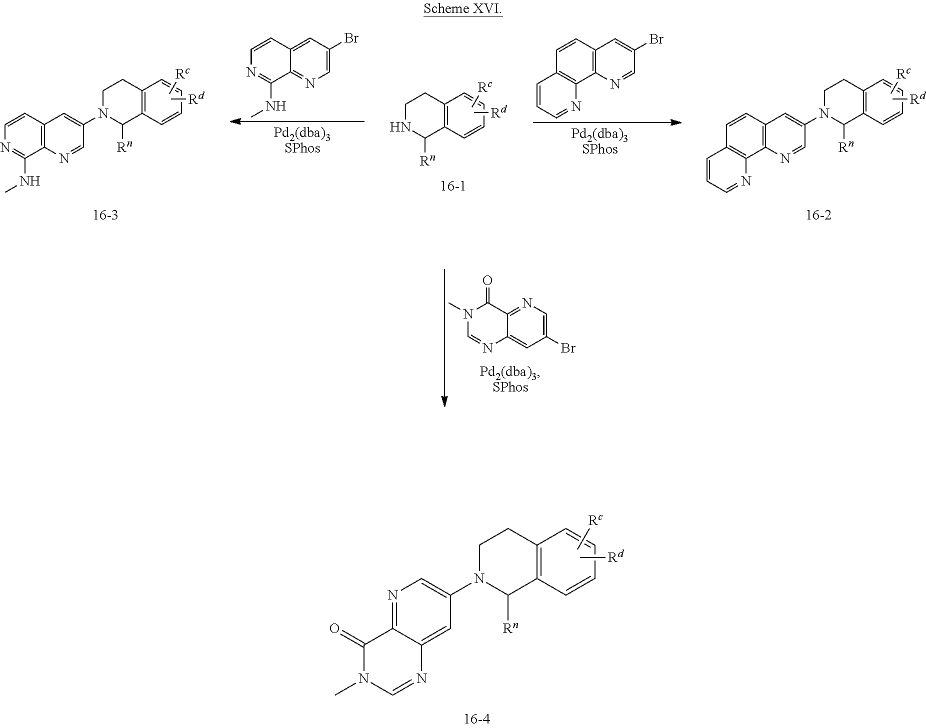

In certain embodiments, a compound of the invention can be prepared, for example, according to the illustrative synthetic methods outlined in Scheme XVII:

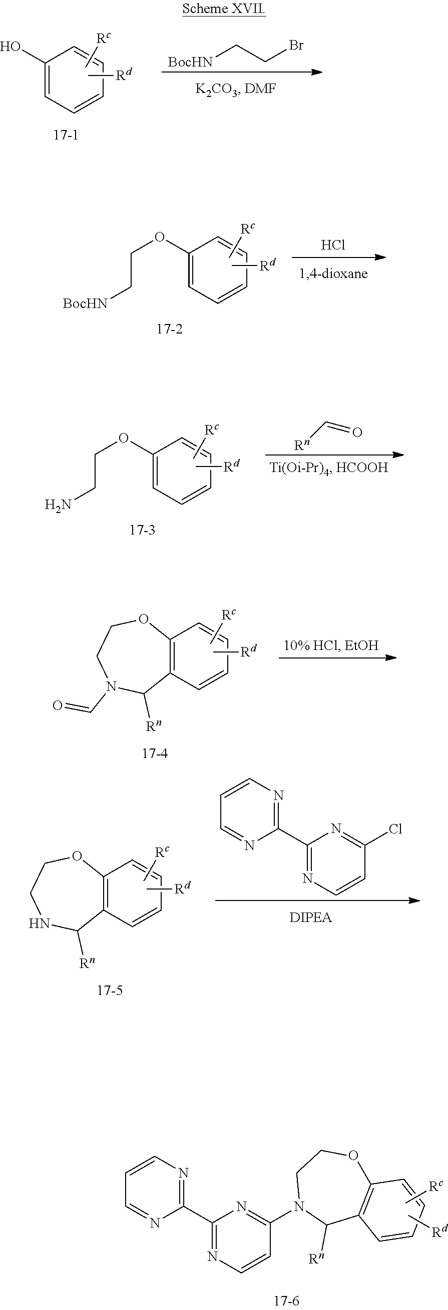

In certain embodiments, a compound of the invention can be prepared, for example, according to the illustrative synthetic methods outlined in Scheme XVIII:

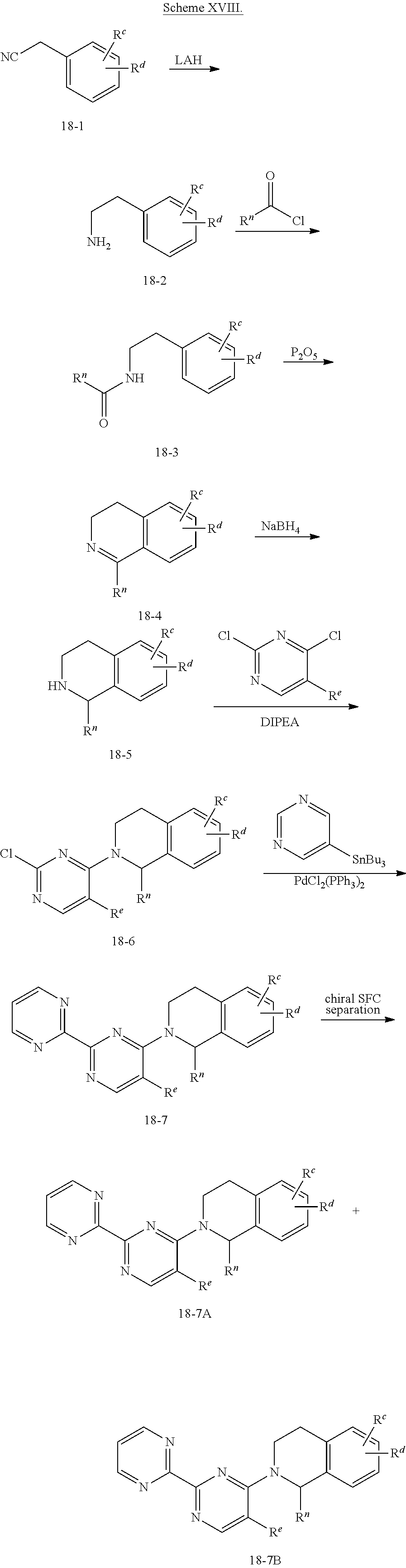

In certain embodiments, a compound of the invention can be prepared, for example, according to the illustrative synthetic methods outlined in Scheme XIX:

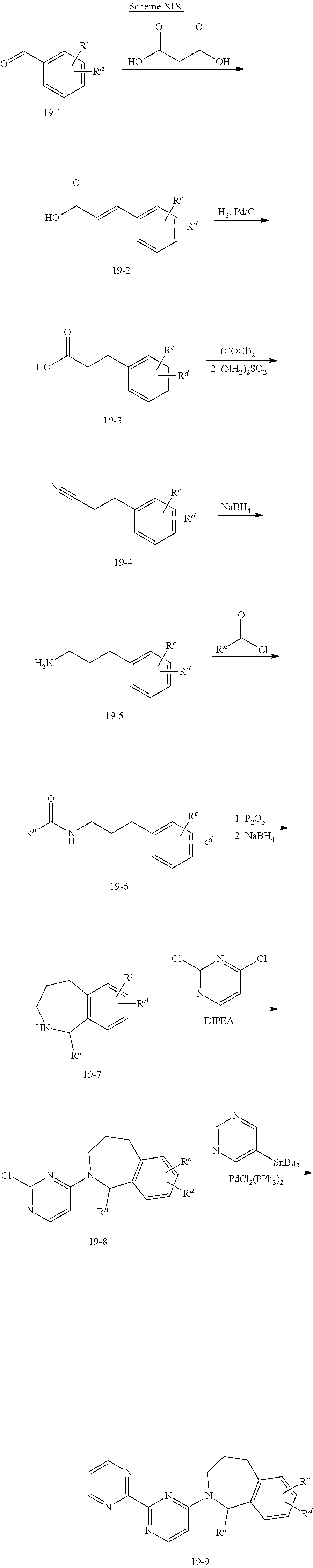

### Methods

The invention provides a method of treating or preventing hepatitis virus infection in a subject. In certain embodiments, the virus comprises hepatitis B virus (HBV). In other embodiments, the virus comprises hepatitis D virus (HDV). In yet other embodiments, the virus comprises HBV and HDV. In yet other embodiments, the method comprises administering to the subject in need thereof a therapeutically effective amount of at least one compound of the invention. In yet other embodiments, the compound of the invention is the only antiviral agent administered to the subject. In yet other embodiments, the at least one compound is administered to the subject in a pharmaceutically acceptable composition. In yet other embodiments, the subject is further administered at least one additional agent useful for treating the hepatitis virus infection. In yet other embodiments, the at least one additional agent comprises at least one selected from the group consisting of reverse transcriptase inhibitor; capsid inhibitor; cccDNA formation inhibitor; sAg secretion inhibitor; oligomeric nucleotide targeted to the Hepatitis B genome; and immunostimulator. In yet other embodiments, the subject is co-administered the at least one compound and the at least one additional agent. In yet other embodiments, the at least one compound and the at least one additional agent are coformulated.

The invention further provides a method of inhibiting and/or reducing HBV surface antigen (HBsAg) secretion either directly or indirectly in a subject. The invention further provides a method of reducing or minimizing levels of HBsAg in an HBV-infected subject. The invention further provides a method of reducing or minimizing levels of HBeAg in an HBV-infected subject. The invention further provides a method of reducing or minimizing levels of hepatitis B core protein in an HBV-infected subject. The invention further provides a method of reducing or minimizing levels of pg RNA in an HBV-infected subject.

In certain embodiments, the method comprises administering to the subject in need thereof a therapeutically effective amount of at least one compound of the invention. In other embodiments, the at least one compound is administered to the subject in a pharmaceutically acceptable composition. In yet other embodiments, the compound of the invention is the only antiviral agent administered to the subject. In yet other embodiments, the subject is further administered at least one additional agent useful for treating HBV infection. In yet other embodiments, the at least one additional agent comprises at least one selected from the group consisting of reverse transcriptase inhibitor; capsid inhibitor; cccDNA formation inhibitor; sAg secretion inhibitor; oligomeric nucleotide targeted to the Hepatitis B genome; and immunostimulator. In yet other embodiments, the subject is co-administered the at least one compound and the at least one additional agent. In yet other embodiments, the at least one compound and the at least one additional agent are coformulated.

In certain embodiments, the subject is a subject in need thereof.

In certain embodiments, the subject is a mammal. In other embodiments, the mammal is a human.

### Pharmaceutical Compositions and Formulations

The invention provides pharmaceutical compositions comprising at least one compound of the invention or a salt or solvate thereof, which are useful to practice methods of the invention. Such a pharmaceutical composition may consist of at least one compound of the invention or a salt or solvate thereof, in a form suitable for administration to a subject, or the pharmaceutical composition may comprise at least one compound of the invention or a salt or solvate thereof, and one or more pharmaceutically acceptable carriers, one or more additional ingredients, or some combination of these. At least one compound of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier.

Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid.

The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and nonionic surfactants, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds of the invention for administration may be in the range of from about 1 μg to about 7,500 mg, about 20 g to about 7,000 mg, about 40 g to about 6,500 mg, about 80 g to about 6,000 mg, about 100 g to about 5,500 mg, about 200 g to about 5,000 mg, about 400 g to about 4,000 mg, about 800 g to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition.

Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Administration Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds useful within the methods of the invention, and a further layer providing for the immediate release of one or more compounds useful within the methods of the invention. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the invention may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the invention may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the invention includes additional modifications of these and other formulations not described herein, but which are known to those of skill in the art.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the compositions and/or formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials & Methods

The following procedures can be utilized in preparing and/or testing exemplary compounds of the invention.

As described herein, “Enantiomer I” refers to the first enantiomer eluded from the chiral column under the specific chiral analytical conditions detailed for examples provided elsewhere herein; and “Enantiomer II” refers to the second enantiomer eluded from the chiral column under the specific chiral analytical conditions detailed for examples provided elsewhere herein. Such nomenclature does not imply or impart any particular relative and/or absolute configuration for these compounds.

### Example 1: 2-([2,2′-Bipyrimidin]-5-yl)-5,7-difluoro-1,2,3,4-tetrahydroisoquinoline

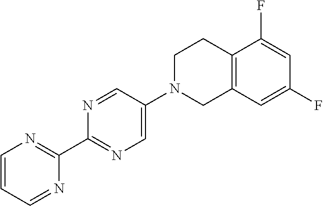 [SMILES: Fc1cc(F)c2c(c1)CN(c1cnc(-c3ncccn3)nc1)CC2]

### Example 2: 2-([2,2′-Bipyrimidin]-4-yl)-5,7-difluoro-1,2,3,4-tetrahydroisoquinoline

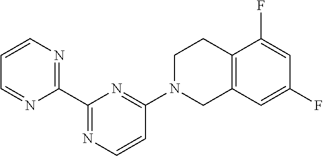 [SMILES: Fc1cc(F)c2c(c1)CN(c1ccnc(-c3ncccn3)n1)CC2]

To a solution of 2,2′-bipyrimidine (5 g, 31.6 mmol) in glacial acetic acid (30 mL) was added bromine (1.95 mL, 37.9 mmol) and the reaction was stirred at 50° C. for 4 hours. The solvent was removed under reduced pressure. The residue was dissolved in CH₂Cl₂ (50 mL), neutralized with saturated aqueous NaHCO₃ solution (50 mL), dried over sodium sulfate, and concentrated under reduced pressure. The residue was purified by normal phase SiO₂ chromatography (0% to 5% MeOH/CH₂Cl₂) to afford a mixture of 5-bromo-2-pyrimidin-2-yl-pyrimidine and 4-bromo-2-pyrimidin-2-yl-pyrimidine as a light brown solid, which was used without further purification (6.1 g, 67% yield).

To the above mixture of 5-bromo-2-pyrimidin-2-yl-pyrimidine and 4-bromo-2-pyrimidin-2-yl-pyrimidine (50 mg, 0.2 mmol) in toluene (2 mL) was added 5,7-difluoro-1,2,3,4-tetrahydroisoquinoline (42 mg, 0.25 mmol), followed by cesium carbonate (137 mg, 0.4 mmol). The solution was purged with nitrogen for 2 minutes. Tris(dibenzylideneacetone) dipalladium(0) (19 mg, 0.02 mmol) and Xphos (30 mg, 0.06 mmol) were added. The reaction vessel was sealed and heated to 110° C. in a microwave reactor for 1 hour. The reaction mixture was cooled to room temperature and water (2 mL) was added, followed by EtOAc (2 mL). The layers were separated, and the aqueous phase was extracted with additional EtOAc (3×2 mL). The combined organic layer was concentrated under reduced pressure. The residue was purified by reverse phase HPLC to afford 5,7-difluoro-2-(2-pyrimidin-2-ylpyrimidin-5-yl)-3,4-dihydro-1H-isoquinoline as a yellow foam (3.2 mg, 4.7% yield) and 5,7-difluoro-2-(2-pyrimidin-2-ylpyrimidin-4-yl)-3,4-dihydro-1H-isoquinoline as a white foam (3.0 mg, 4.4% yield).

### Example 1: 2-([2,2′-Bipyrimidin]-5-yl)-5,7-difluoro-1,2,3,4-tetrahydroisoquinoline

m/z: 326 [M+H]⁺ observed. ¹H NMR (400 MHz, CDCl₃) δ 9.03 (d, J=4.9 Hz, 2H), 8.70 (s, 2H), 7.46 (t, J=4.8 Hz, 1H), 6.86-6.65 (m, 2H), 4.61 (s, 2H), 3.79 (t, J=5.9 Hz, 2H), 3.09-2.93 (m, 2H).

### Example 2: 2-([2,2′-Bipyrimidin]-4-yl)-5,7-difluoro-1,2,3,4-tetrahydroisoquinoline

m/z: 326 [M+H]⁺ observed. ¹H NMR (400 MHz, CDCl₃) δ 9.07 (d, J=4.9 Hz, 2H), 8.81 (d, J=7.1 Hz, 1H), 7.56 (t, J=4.9 Hz, 1H), 6.92 (d, J=7.2 Hz, 1H), 6.86 (d, J=8.5 Hz, 1H), 6.79 (td, J=9.0, 2.5 Hz, 1H), 5.07 (s, 2H), 4.12 (s, 2H), 3.04 (t, J=6.0 Hz, 2H).

The following examples were prepared in a similar manner as 5,7-difluoro-2-(2-pyrimidin-2-ylpyrimidin-5-yl)-3,4-dihydro-1H-isoquinoline and 2-([2,2′-bipyrimidin]-4-yl)-5,7-difluoro-1,2,3,4-tetrahydroisoquinoline from 4-bromo-2,2′-bipyrimidine or 5-bromo-2,2′-bipyrimidine, and an appropriate amine.

### Example 3: 2-([2,2′-Bipyrimidin]-5-yl)-5,6-difluoro-1,2,3,4-tetrahydroisoquinoline

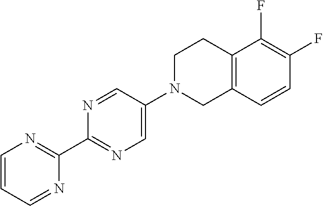 [SMILES: Fc1ccc2c(c1F)CCN(c1cnc(-c3ncccn3)nc1)C2]

m/z: 326 [M+H]⁺ observed. ¹H NMR (400 MHz, CDCl₃) δ 8.96 (d, J=4.9 Hz, 2H), 8.63 (s, 2H), 7.36 (t, J=4.9 Hz, 1H), 7.13-6.92 (m, 2H), 4.56 (s, 2H), 3.75 (t, J=6.0 Hz, 2H), 3.08 (t, J=6.0 Hz, 2H).

### Example 4: 2-([2,2′-Bipyrimidin]-4-yl)-5,6-difluoro-1,2,3,4-tetrahydroisoquinoline

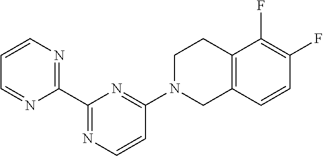 [SMILES: Fc1ccc2c(c1F)CCN(c1ccnc(-c3ncccn3)n1)C2]

m/z: 326 [M+H]⁺ observed. ¹H NMR (400 MHz, CDCl₃) δ 9.07 (d, J=4.9 Hz, 2H), 8.80 (d, J=7.2 Hz, 1H), 7.57 (t, J=4.9 Hz, 1H), 7.17-7.07 (m, 1H), 7.04 (dd, J=8.8, 4.4 Hz, 1H), 6.93 (d, J=7.2 Hz, 1H), 5.05 (s, 2H), 4.17 (s, 2H), 3.13 (t, J=6.0 Hz, 2H).

### Example 5: 2-([2,2′-Bipyrimidin]-5-yl)-4-methyl-1,2,3,4-tetrahydroisoquinoline

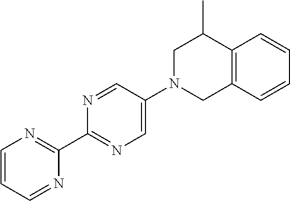 [SMILES: CC1CN(c2cnc(-c3ncccn3)nc2)Cc2ccccc21]

m/z: 304 [M+H]⁺ observed. ¹H NMR (400 MHz, CDCl₃) δ 8.95 (d, J=4.8 Hz, 2H), 8.58 (s, 2H), 7.37-7.26 (m, 5H), 4.72-4.48 (m, 2H), 3.74-3.48 (m, 2H), 3.28-3.12 (m, 1H), 1.42 (d, J=7.0 Hz, 3H).

### Example 6: 1-Methyl-2-(2-pyrimidin-2-ylpyrimidin-5-yl)-3,4-dihydro-1H-isoquinoline

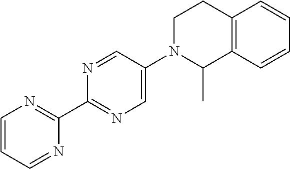 [SMILES: CC1c2ccccc2CCN1c1cnc(-c2ncccn2)nc1]

m/z: 304 [M+H]⁺ observed. ¹H NMR (400 MHz, CDCl₃) δ 8.94 (d, J=4.8 Hz, 2H), 8.57 (s, 2H), 7.31 (t, J=4.8 Hz, 1H), 7.25-7.20 (m, 4H), 5.01 (q, J=6.7 Hz, 1H), 3.92-3.57 (m, 2H), 3.22-2.92 (m, 2H), 1.56 (d, J=6.7 Hz, 3H).

### Example 7: 2-([2,2′-Bipyrimidin]-4-yl)-1-methyl-1,2,3,4-tetrahydroisoquinoline

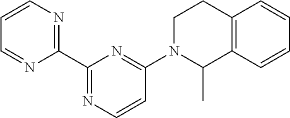 [SMILES: CC1c2ccccc2CCN1c1ccnc(-c2ncccn2)n1]

m/z: 304 [M+H]⁺ observed. ¹H NMR (400 MHz, CDCl₃) δ 8.98 (d, J=4.8 Hz, 2H), 8.48 (d, J=6.2 Hz, 1H), 7.39 (t, J=4.8 Hz, 1H), 7.28-7.17 (m, 4H), 6.66 (d, J=6.2 Hz, 1H), 3.68-3.52 (m, 1H), 3.09-2.87 (m, 2H), 1.60-1.57 (m, 2H), 1.56 (d, J=6.7 Hz, 3H).

### Example 8: 2-([2,2′-Bipyrimidin]-4-yl)-3-ethyl-1,2,3,4-tetrahydroisoquinoline

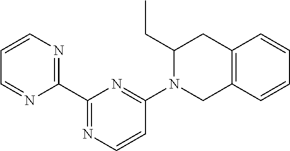 [SMILES: CCC1Cc2ccccc2CN1c1ccnc(-c2ncccn2)n1]

m/z: 318 [M+H]⁺ observed. ¹H NMR (400 MHz, CDCl₃) δ 8.99 (d, J=4.8 Hz, 2H), 8.47 (d, J=6.2 Hz, 1H), 7.39 (t, J=4.8 Hz, 1H), 7.26-7.14 (m, 4H), 6.65 (d, J=6.2 Hz, 1H), 4.57 (s, 1H), 3.28-2.74 (m, 2H), 1.60-1.41 (m, 4H), 0.89 (t, J=7.4 Hz, 3H).

### Example 9: 2-([2,2′-Bipyrimidin]-4-yl)-1,2,3,4-tetrahydroisoquinoline

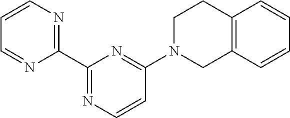 [SMILES: c1cnc(-c2nccc(N3CCc4ccccc4C3)n2)nc1]

m/z: 290 [M+H]⁺ observed. ¹H NMR (400 MHz, CDCl₃) δ 9.05 (d, J=4.9 Hz, 2H), 8.80 (d, J=7.0 Hz, 1H), 7.52 (t, J=4.9 Hz, 1H), 7.29-7.24 (m, 4H), 6.82 (d, J=7.0 Hz, 1H), 5.18-4.89 (m, 2H), 4.26-3.99 (m, 2H), 3.08 (t, J=5.9 Hz, 2H).

### Example 10: 2-([2,2′-Bipyrimidin]-5-yl)-1-ethyl-1,2,3,4-tetrahydroisoquinoline

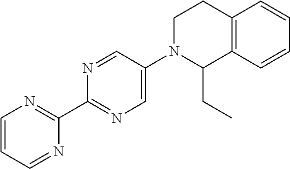 [SMILES: CCC1c2ccccc2CCN1c1cnc(-c2ncccn2)nc1]

m/z: 318 [M+H]⁺ observed. ¹H NMR (400 MHz, CDCl₃) δ 8.93 (d, J=4.8 Hz, 2H), 8.53 (s, 2H), 7.30 (t, J=4.8 Hz, 1H), 7.26-7.15 (m, 4H), 4.67 (dd, J=7.9, 6.2 Hz, 1H), 3.81-3.55 (m, 2H), 3.08 (t, J=6.3 Hz, 2H), 2.08-1.73 (m, 2H), 1.02 (t, J=7.4 Hz, 3H).

5 mg of the mixture of enantiomers was separated by SFC (supercritical fluid chromatography) on a CHIRALCEL® AD column using liquid CO₂ and IPA (35%; 0.1% diethylamine as modifier) to give 2-([2,2′-bipyrimidin]-5-yl)-1-ethyl-1,2,3,4-tetrahydroisoquinoline (single enantiomer I) as a yellow foam (faster eluting enantiomer, 1.8 mg, 36%, m/z: 318 [M+H]⁺ observed), and 2-([2,2′-bipyrimidin]-5-yl)-1-ethyl-1,2,3,4-tetrahydroisoquinoline (single enantiomer II) as a yellow foam (slower eluting enantiomer, 1.8 mg, 36%, m/z: 318 [M+H]⁺ observed).

### Example 11: 2-([2,2′-Bipyrimidin]-5-yl)-1-ethyl-1,2,3,4-tetrahydroisoquinoline (Single Enantiomer I)

m/z: 318 [M+H]⁺ observed. ¹H NMR (400 MHz, CDCl₃) δ 8.93 (d, J=4.8 Hz, 2H), 8.53 (s, 2H), 7.30 (t, J=4.8 Hz, 1H), 7.26-7.15 (m, 4H), 4.67 (dd, J=7.9, 6.2 Hz, 1H), 3.81-3.55 (m, 2H), 3.08 (t, J=6.3 Hz, 2H), 2.08-1.73 (m, 2H), 1.02 (t, J=7.4 Hz, 3H).

### Example 12: 2-([2,2′-Bipyrimidin]-5-yl)-1-ethyl-1,2,3,4-tetrahydroisoquinoline (Single Enantiomer II)

m/z: 318 [M+H]⁺ observed. ¹H NMR (400 MHz, CDCl₃) δ 8.93 (d, J=4.8 Hz, 2H), 8.53 (s, 2H), 7.30 (t, J=4.8 Hz, 1H), 7.26-7.15 (m, 4H), 4.67 (dd, J=7.9, 6.2 Hz, 1H), 3.81-3.55 (m, 2H), 3.08 (t, J=6.3 Hz, 2H), 2.08-1.73 (m, 2H), 1.02 (t, J=7.4 Hz, 3H).

### Example 13: 2-([2,2′-Bipyrimidin]-4-yl)-1-ethyl-1,2,3,4-tetrahydroisoquinoline

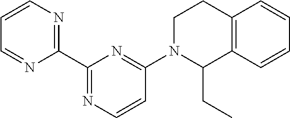 [SMILES: CCC1c2ccccc2CCN1c1ccnc(-c2ncccn2)n1]

m/z: 318 [M+H]⁺ observed. ¹H NMR (400 MHz, CDCl₃) δ 8.97 (d, J=4.8 Hz, 2H), 8.44 (d, J=6.2 Hz, 1H), 7.38 (t, J=4.8 Hz, 1H), 7.21-7.15 (m, 4H), 6.64 (d, J=6.3 Hz, 1H), 3.75 (dt, J=13.1, 6.6 Hz, 1H), 3.06-2.88 (m, 2H), 2.09-1.76 (m, 3H), 1.37-1.14 (m, 1H), 0.99 (t, J=7.4 Hz, 3H).

### Example 14: 2-([2,2′-Bipyrimidin]-5-yl)-4-(trifluoromethyl)isoindoline

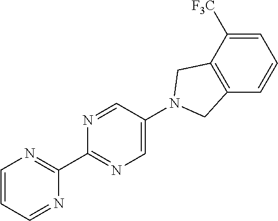 [SMILES: FC(F)(F)c1cccc2c1CN(c1cnc(-c3ncccn3)nc1)C2]

m/z: 344 [M+H]⁺ observed. ¹H NMR (400 MHz, CD₃OD) δ 8.89 (d, J=4.8 Hz, 1H), 8.41 (s, 2H), 7.84-7.01 (m, 5H), 5.00 (m, 2H), 4.83 (m, 2H).

### Example 15: 2-([2,2′-Bipyrimidin]-4-yl)-4-methyl-1,2,3,4-tetrahydroisoquinoline

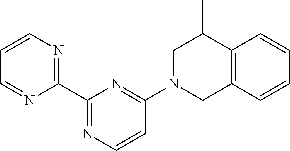 [SMILES: CC1CN(c2ccnc(-c3ncccn3)n2)Cc2ccccc21]

m/z: 304 [M+H]⁺ observed. ¹H NMR (400 MHz, CDCl₃) δ 9.01 (d, J=4.9 Hz, 2H), 8.67 (s, 2H), 7.42 (t, J=4.9 Hz, 1H), 7.32-7.30 (m, 2H), 7.28 (d, J=3.9 Hz, 1H), 7.21 (q, J=3.4 Hz, 1H), 4.77-4.47 (m, 3H), 3.70 (m, 2H), 1.42 (d, J=7.0 Hz, 3H).

### Example 16: 2-([2,2′-Bipyrimidin]-5 yl)-4-methyl-3,4-dihydroisoquinolin-1(2H)-one

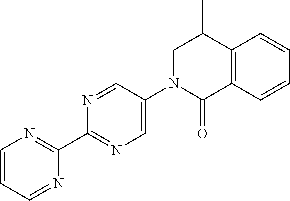 [SMILES: CC1CN(c2cnc(-c3ncccn3)nc2)C(=O)c2ccccc21]

m/z: 318 [M+H]⁺ observed. ¹H NMR (400 MHz, CDCl₃) δ 9.21 (s, 2H), 9.08 (d, J=4.9 Hz, 2H), 8.21-8.13 (m, 1H), 7.59 (td, J=7.5, 1.4 Hz, 1H), 7.51 (t, J=4.9 Hz, 1H), 7.43 (t, J=7.5 Hz, 1H), 7.34 (d, J=7.6 Hz, 1H), 4.22 (dd, J=11.7, 4.4 Hz, 1H), 3.88 (dd, J=11.7, 6.6 Hz, 1H), 3.44-3.33 (m, 1H), 1.48 (d, J=7.0 Hz, 3H).

### Example 17: 2-([2,2′-Bipyrimidin]-4-yl)-6,7-difluoro-1,2,3,4-tetrahydroisoquinoline

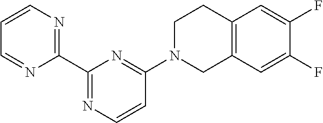 [SMILES: Fc1cc2c(cc1F)CN(c1ccnc(-c3ncccn3)n1)CC2]

m/z: 325 [M+H]⁺ observed. ¹H NMR (400 MHz, DMSO-d₆) δ 8.94 (d, J=4.8 Hz, 2H), 8.40 (d, J=6 Hz, 1H), 7.59 (t, J=5.2 Hz, 1H), 7.41-7.28 (m, 2H), 8.96-8.94 (d, J=6 Hz, 1H), 4.79 (s, 2H), 3.89-3.88 (m, 2H), 2.88 (t, J=5.6 Hz, 2H).

### Example 18: 2′-([2,2′-Bipyrimidin]-5-yl)-6′,7′-dimethoxy-3′,4′-dihydro-2′H-spiro[cyclobutane-1,1′-isoquinoline]

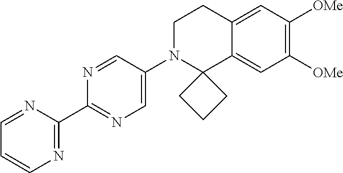 [SMILES: COc1cc2c(cc1OC)C1(CCC1)N(c1cnc(-c3ncccn3)nc1)CC2]

m/z: 390 [M+H]⁺ observed. ¹H NMR (400 MHz, CD₃OD) δ 8.96 (d, J=4.7 Hz, 2H), 8.43 (s, 2H), 7.56 (t, J=4.9 Hz, 1H), 7.25 (s, 1H), 6.63 (s, 1H), 3.94 (d, J=3.6 Hz, 5H), 3.76 (s, 3H), 2.72-2.48 (m, 6H), 2.26-1.92 (m, 2H).

### Example 19: 2-([2,2′-Bipyrimidin]-5-yl)-1-ethylisoindoline

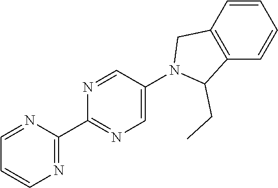 [SMILES: CCC1c2ccccc2CN1c1cnc(-c2ncccn2)nc1]

m/z: 304 [M+H]⁺ observed. ¹H NMR (400 MHz, CD₃OD) δ 8.97 (d, J=4.9 Hz, 2H), 8.51 (s, 2H), 7.55 (t, J=4.9 Hz, 1H), 7.49-7.26 (m, 5H), 5.52-5.34 (m, 1H), 4.92-4.67 (m, 2H), 2.27 (ddd, J=14.6, 7.3, 5.4 Hz, 1H), 1.99 (ddd, J=14.6, 7.4, 2.5 Hz, 1H), 0.63 (t, J=7.4 Hz, 3H).

### Example 20: 10-([2,2′-Bipyrimidin]-5-yl)-1,2,3,4-tetrahydro-1,4-(epiminomethano) naphthalene

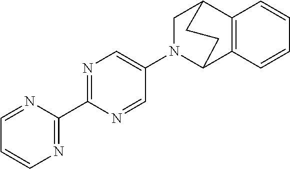 [SMILES: c1cnc(-c2ncc(N3CC4CCC3c3ccccc34)cn2)nc1]

m/z: 316 [M+H]⁺ observed. ¹H NMR (400 MHz, CDCl₃) δ 9.13-8.75 (m, 2H), 8.60-8.31 (m, 2H), 7.32-7.27 (m, 5H), 5.13-5.06 (m, 1H), 3.67-3.62 (m, 1H), 3.46-3.40 (m, 1H), 3.19-3.11 (m, 1H), 2.37-2.24 (m, 1H), 2.07-1.95 (m, 1H), 1.78-1.60 (m, 2H).

### Example 21: 2-([2,2′-Bipyrimidin]-5-yl)-1,2,3,4-tetrahydro-1,4-methanoisoquinoline

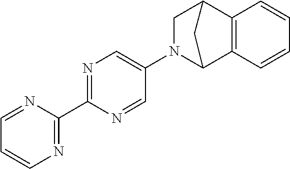 [SMILES: c1cnc(-c2ncc(N3CC4CC3c3ccccc34)cn2)nc1]

m/z: 302 [M+H]⁺ observed. ¹H NMR (400 MHz, CDCl₃) δ 8.91 (s, 2H), 8.36 (s, 2H), 7.37-7.27 (m, 3H), 7.18-7.12 (m, 1H), 7.12-7.06 (m, 1H), 5.17 (s, 1H), 3.96 (dd, J=8.2, 3.1 Hz, 1H), 3.83 (s, 1H), 2.61 (d, J=8.0 Hz, 1H), 2.26-2.20 (m, 1H), 2.12 (d, J=9.3 Hz, 1H).

### Example 22: 9-([2,2′-Bipyrimidin]-5-yl)-1,2,3,4-tetrahydro-1,4-epiminonaphthalene

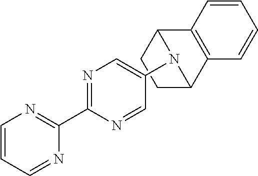 [SMILES: c1cnc(-c2ncc(N3C4CCC3c3ccccc34)cn2)nc1]

m/z: 302 [M+H]⁺ observed. ¹H NMR (400 MHz, DMSO-d₆) δ 9.69-7.94 (m, 4H), 7.69-7.40 (m, 1H), 7.33 (dd, J=5.2, 3.1 Hz, 2H), 7.12 (dd, J=5.3, 3.0 Hz, 2H), 5.53 (s, 2H), 2.12 (d, J=8.7 Hz, 2H), 1.35-1.26 (m, 2H).

### Example 23: 2-([2,2′-Bipyrimidin]-4-yl)-1-propyl-1,2,3,4-tetrahydroisoquinoline

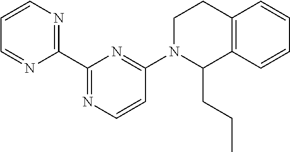 [SMILES: CCCC1c2ccccc2CCN1c1ccnc(-c2ncccn2)n1]

m/z: 332 [M+H]⁺ observed. ¹H NMR (400 MHz, CDCl₃) δ 8.96 (d, J=4.9 Hz, 2H), 8.43 (d, J=6.2 Hz, 1H), 7.38 (t, J=4.9 Hz, 1H), 7.24-7.09 (m, 4H), 6.63 (d, J=6.3 Hz, 1H), 4.63-4.61 (m, 2H), 3.73 (dt, J=13.1, 6.6 Hz, 1H), 3.02 (dt, J=14.1, 6.7 Hz, 2H), 2.01-1.90 (m, 2H), 1.49-1.33 (m, 2H), 0.93 (t, J=7.3 Hz, 3H).

### Example 24: 2-([2,2′-Bipyrimidin]-5-yl)-5,6-difluoro-1-methyl-1,2,3,4-tetrahydroisoquinoline

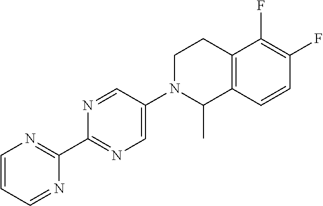 [SMILES: CC1c2ccc(F)c(F)c2CCN1c1cnc(-c2ncccn2)nc1]

m/z: 340 [M+H]⁺ observed. ¹H NMR (400 MHz, CDCl₃) δ 8.94 (d, J=4.8 Hz, 2H), 8.58 (s, 2H), 7.32 (t, J=4.8 Hz, 1H), 7.11-6.99 (m, 1H), 6.94 (ddd, J=8.4, 4.4, 1.5 Hz, 1H), 5.01 (q, J=6.7 Hz, 1H), 3.86 (dt, J=12.8, 5.3 Hz, 1H), 3.58 (ddd, J=13.2, 8.9, 4.9 Hz, 1H), 3.17-2.93 (m, 2H), 1.52 (d, J=6.7 Hz, 3H).

### Example 25: 2-([2,2′-Bipyrimidin]-5-yl)-1-ethyl-6,7-difluoro-1,2,3,4-tetrahydroisoquinoline

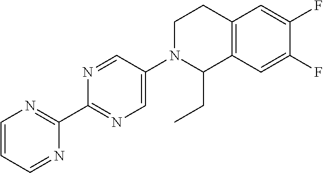 [SMILES: CCC1c2cc(F)c(F)cc2CCN1c1cnc(-c2ncccn2)nc1]

m/z: 354 [M+H]⁺ observed. ¹H NMR (400 MHz, CDCl₃) δ 8.93 (d, J=4.9 Hz, 2H), 8.53 (s, 2H), 7.31 (td, J=4.8, 0.5 Hz, 1H), 7.01 (ddd, J=10.6, 7.7, 4.9 Hz, 2H), 4.63 (t, J=7.1 Hz, 1H), 3.76-3.59 (m, 2H), 3.00 (q, J=6.4 Hz, 2H), 2.09-1.91 (m, 1H), 1.79 (dt, J=14.4, 7.3 Hz, 1H), 1.02 (t, J=7.4 Hz, 3H).

### Example 26: Methyl 2-(2-([2,2′-bipyrimidin]-5-yl)-6,7-dimethoxy-1,2,3,4-tetrahydroisoquinolin-1-yl) acetate

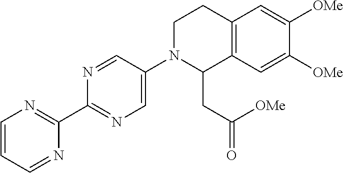 [SMILES: COC(=O)CC1c2cc(OC)c(OC)cc2CCN1c1cnc(-c2ncccn2)nc1]

m/z: 422 [M+H]⁺ observed. ¹H NMR (400 MHz, CDCl₃) δ 9.07 (d, J=5.0 Hz, 2H), 8.91 (s, 2H), 7.53 (t, J=5.0 Hz, 1H), 6.69 (d, J=14.0 Hz, 2H), 5.48 (dd, J=8.0, 6.0 Hz, 1H), 3.87-3.85 (m, 7H), 3.78 (dd, J=8.6, 4.8 Hz, 1H), 3.69 (s, 3H), 3.18-2.99 (m, 2H), 2.89 (ddd, J=21.9, 16.1, 5.6 Hz, 2H).

### Example 27: 2-(2-([2,2′-Bipyrimidin]-5-yl)-6,7-dimethoxy-1,2,3,4-tetrahydroisoquinolin-1-yl) acetic Acid

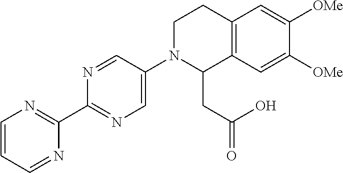 [SMILES: COc1cc2c(cc1OC)C(CC(=O)O)N(c1cnc(-c3ncccn3)nc1)CC2]

m/z: 408 [M+H]⁺ observed. ¹H NMR (400 MHz, CDCl₃) δ 9.03 (d, J=5.0 Hz, 2H), 8.94 (s, 2H), 7.51 (s, 1H), 6.73 (s, 1H), 6.66 (s, 1H), 5.52 (dd, J=8.7, 5.1 Hz, 1H), 3.97-3.89 (m, 1H), 3.87 (s, 3H), 3.85 (s, 3H), 3.78 (ddd, J=13.7, 9.3, 4.6 Hz, 1H), 3.17-3.04 (m, 2H), 2.93 (dt, J=16.3, 5.6 Hz, 2H).

### Example 28: 2-([2,2′-Bipyrimidin]-5-yl)-1-ethyl-5,6-dimethoxy-1,2,3,4 Tetrahydroisoquinoline

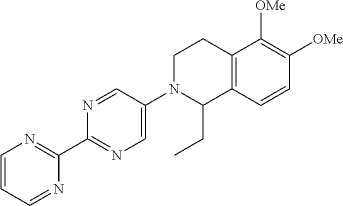 [SMILES: CCC1c2ccc(OC)c(OC)c2CCN1c1cnc(-c2ncccn2)nc1]

m/z: 378 [M+H]⁺ observed. ¹H NMR (400 MHz, CDCl₃) δ 8.92 (d, J=4.8 Hz, 2H), 8.51 (s, 2H), 7.28 (t, J=4.8 Hz, 1H), 6.88 (d, J=8.4 Hz, 1H), 6.79 (d, J=8.4 Hz, 1H), 4.60 (dd, J=7.7, 6.4 Hz, 1H), 3.86 (s, 3H), 3.82 (s, 3H), 3.75-3.66 (m, 1H), 3.58 (ddd, J=12.5, 8.1, 5.1 Hz, 1H), 3.24 (ddd, J=16.3, 6.4, 5.1 Hz, 1H), 2.98-2.82 (m, 1H), 1.98 (ddd, J=14.0, 7.5, 6.4 Hz, 1H), 1.74 (dt, J=13.9, 7.4 Hz, 1H), 0.99 (t, J=7.4 Hz, 3H).

### Example 29: 1-Ethyl-5,6-difluoro-7-methoxy-2-(2-pyrimidin-2-ylpyrimidin-5-yl)-3,4-dihydro-1H-isoquinoline

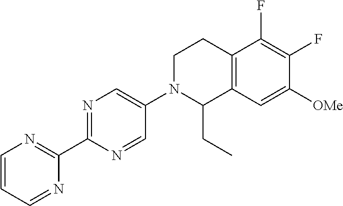 [SMILES: CCC1c2cc(OC)c(F)c(F)c2CCN1c1cnc(-c2ncccn2)nc1]

m/z: 384 [M+H]⁺ observed. ¹H NMR (400 MHz, DMSO-d₆) δ 8.90 (d, J=5.6 Hz, 2H), 8.66 (s, 2H), 7.49 (t, J=5.2 Hz, 1H), 6.97 (d, J=7.2 Hz, 1H), 5.10-5.06 (m, 1H), 4.03-3.97 (m, 1H), 3.87 (s, 3H), 3.62-3.55 (m, 1H), 2.96-2.88 (m, 1H), 2.78-2.73 (m, 1H), 1.98-1.83 (m, 2H), 0.98 (t, J=7.2 Hz, 3H).

### Example 30: 1-Ethyl-5,6-difluoro-7-methoxy-2-(2-pyrimidin-2-ylpyrimidin-5-yl)-3,4-dihydro-1H-isoquinoline (Single Enantiomer I)

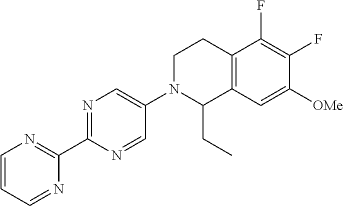 [SMILES: CCC1c2cc(OC)c(F)c(F)c2CCN1c1cnc(-c2ncccn2)nc1]

m/z: 384 [M+H]⁺ observed. ¹H NMR (400 MHz, DMSO-d₆) δ 8.90 (d, J=5.6 Hz, 2H), 8.66 (s, 2H), 7.49 (t, J=5.2 Hz, 1H), 6.97 (d, J=7.2 Hz, 1H), 5.10-5.06 (m, 1H), 4.03-3.97 (m, 1H), 3.87 (s, 3H), 3.62-3.55 (m, 1H), 2.96-2.88 (m, 1H), 2.78-2.73 (m, 1H), 1.98-1.83 (m, 2H), 0.98 (t, J=7.2 Hz, 3H).

### Example 31: 1-Ethyl-5,6-difluoro-7-methoxy-2-(2-pyrimidin-2-ylpyrimidin-5-yl)-3,4-dihydro-1H-isoquinoline (Single Enantiomer II)

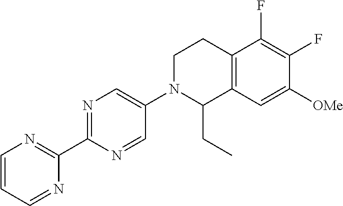 [SMILES: CCC1c2cc(OC)c(F)c(F)c2CCN1c1cnc(-c2ncccn2)nc1]

m/z: 384 [M+H]⁺ observed. ¹H NMR (400 MHz, DMSO-d₆) δ 8.90 (d, J=5.6 Hz, 2H), 8.66 (s, 2H), 7.49 (t, J=5.2 Hz, 1H), 6.97 (d, J=7.2 Hz, 1H), 5.10-5.06 (m, 1H), 4.03-3.97 (m, 1H), 3.87 (s, 3H), 3.62-3.55 (m, 1H), 2.96-2.88 (m, 1H), 2.78-2.73 (m, 1H), 1.98-1.83 (m, 2H), 0.98 (t, J=7.2 Hz, 3H).

### Example 32: 1-Ethyl-5,6-difluoro-2-(2-pyrimidin-2-ylpyrimidin-5-yl)-3,4-dihydro-1H-isoquinoline

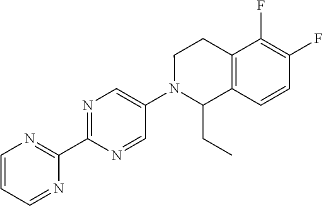 [SMILES: CCC1c2ccc(F)c(F)c2CCN1c1cnc(-c2ncccn2)nc1]

m/z: 354 [M+H]⁺ observed. ¹H NMR (400 MHz, DMSO-d₆) δ 8.88 (d, J=4.8 Hz, 2H), 8.66 (s, 2H), 4.78 (d, J=4.8 Hz, 1H), 7.47-7.25 (m, 1H), 7.14-7.11 (m, 1H), 5.13 (t, J=6.8 Hz, 1H), 3.99 (t, J=8.8 Hz, 1H), 3.61-3.56 (m, 1H), 3.00-2.98 (m, 1H), 2.86 (m, 1H), 1.94-1.88 (m, 1H), 1.80-1.76 (m 1H), 0.95 (t, J=7.2 Hz, 3H).

### Example 33: 1-Ethyl-5,6-difluoro-2-(2-pyrimidin-2-ylpyrimidin-5-yl)-3,4-dihydro-1H-isoquinoline (Single Enantiomer I)

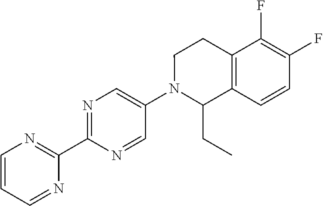 [SMILES: CCC1c2ccc(F)c(F)c2CCN1c1cnc(-c2ncccn2)nc1]

m/z: 354 [M+H]⁺ observed. ¹H NMR (400 MHz, DMSO-d₆) δ 8.88 (d, J=4.8 Hz, 2H), 8.66 (s, 2H), 4.78 (d, J=4.8 Hz, 1H), 7.47-7.25 (m, 1H), 7.14-7.11 (m, 1H), 5.13 (t, J=6.8 Hz, 1H), 3.99 (t, J=8.8 Hz, 1H), 3.61-3.56 (m, 1H), 3.00-2.98 (m, 1H), 2.86 (m, 1H), 1.94-1.88 (m, 1H), 1.80-1.76 (m 1H), 0.95 (t, J=7.2 Hz, 3H).

### Example 34: 1-Ethyl-5,6-difluoro-2-(2-pyrimidin-2-ylpyrimidin-5-yl)-3,4-dihydro-1H-isoquinoline (Single Enantiomer II)

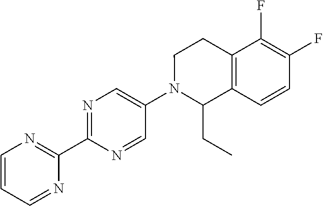 [SMILES: CCC1c2ccc(F)c(F)c2CCN1c1cnc(-c2ncccn2)nc1]

m/z: 354[M+H]⁺ observed. ¹H NMR (400 MHz, DMSO-d₆) δ 8.88 (d, J=4.8 Hz, 2H), 8.66 (s, 2H), 4.78 (d, J=4.8 Hz, 1H), 7.47-7.25 (m, 1H), 7.14-7.11 (m, 1H), 5.13 (t, J=6.8 Hz, 1H), 3.99 (t, J=8.8 Hz, 1H), 3.61-3.56 (m, 1H), 3.00-2.98 (m, 1H), 2.86 (m, 1H), 1.94-1.88 (m, 1H), 1.80-1.76 (m 1H), 0.95 (t, J=7.2 Hz, 3H).

### Example 35: 1-Ethyl-5-fluoro-8-methoxy-2-(2-pyrimidin-2-ylpyrimidin-5-yl)-3,4-dihydro-1H-isoquinoline

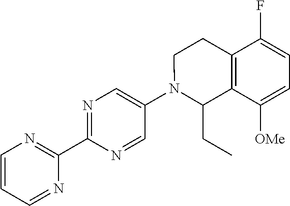 [SMILES: CCC1c2c(OC)ccc(F)c2CCN1c1cnc(-c2ncccn2)nc1]

m/z: 366 [M+H]⁺ observed. ¹H NMR (400 MHz, DMSO-d₆) δ 8.92-8.89 (m, 2H), 8.64 (s, 2H), 7.52-7.48 (m, 1H), 7.06-7.02 (m, 1H), 6.89-6.87 (m, 1H), 5.04-5.00 (m, 1H), 4.01-3.97 (m, 1H), 3.83 (s, 3H), 3.67-3.65 (m, 1H), 2.94-2.91 (m, 1H), 2.79 (s, 1H), 1.92-1.82 (m, 2H), 0.99-0.96 (m, 3H).

### Example 36: 2-([2,2′-Bipyrimidin]-4-yl)-5,6-difluoro-1-methyl-1,2,3,4-tetrahydroisoquinoline

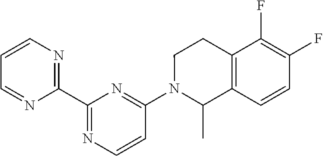 [SMILES: CC1c2ccc(F)c(F)c2CCN1c1ccnc(-c2ncccn2)n1]

### 2-(2,3-Difluorophenyl)ethan-1-amine

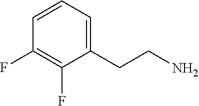 [SMILES: NCCc1cccc(F)c1F]

To a solution of (2,3-difluorophenyl) acetonitrile (10 g, 65.3 mmol) in THF (100 mL) was added borane solution (1M in THF, 310 mL, 314 mmol) at 0° C. The mixture was then warmed to 80° C. and stirred for 10 hr. The mixture was cooled to 0° C. and then aqueous HCl solution (2.6 M, 1.2 L) was carefully added at 0° C. The mixture was carefully warmed to 80° C. and stirred for 1 h. The mixture was concentrated under reduced pressure, the residue was dissolved in aqueous HCl solution (2.6 M, 100 mL), and the pH was adjusted to 10˜11 with 1N aqueous NaOH solution. The resulting mixture was extracted with EtOAc (4×150 mL). The combined organic layers were washed with saturated aqueous brine solution (300 mL), dried over sodium sulfate, filtered, and concentrated under reduced pressure. The residue was purified by normal phase SiO₂ chromatography (5% to 50% EtOAc/petroleum ether) to afford 2-(2,3-difluorophenyl)ethan-1-amine as a light yellow solid (7.9 g, 77% yield, m/z: 158 [M+H]⁺ observed).

### N-(2,3-Difluorophenethyl)-4-methylbenzenesulfonamide

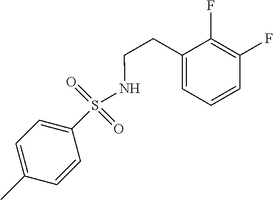 [SMILES: Cc1ccc(S(=O)(=O)NCCc2cccc(F)c2F)cc1]

To a solution of 2-(2,3-difluorophenyl)ethan-1-amine (5.9 g, 37.5 mmol) and triethylamine (5.5 mL, 39.7 mmol) in CH₂Cl₂ (150 mL) was added 4-methylbenzenesulfonyl chloride (6.8 g, 35.7 mmol, 0.95 eq) at 0° C. The mixture was stirred at room temperature for 15 hr. The reaction mixture was quenched with 1N aqueous HCl solution (150 mL) and extracted with CH₂Cl₂ (4×50 mL). The combined organic fractions were washed with saturated aqueous brine solution (100 mL), followed by 1N aqueous HCl solution (100 mL) and saturated aqueous NaHCO₃ solution (100 mL). The solution was dried over sodium sulfate, filtered, and concentrated under reduced pressure. The residue was purified by normal phase SiO₂ chromatography (0% to 15% EtOAc/petroleum ether) to afford N-(2,3-difluorophenethyl)-4-methylbenzenesulfonamide as a white solid (4.9 g, 42% yield, m/z: 312 [M+H]⁺ observed). ¹H NMR (400 MHz, CDCl₃) δ 7.62 (d, J=8 Hz, 2H), 7.21 (m, 2H), 6.96-6.92 (m, 2H), 6.90-6.81 (m, 1H), 4.42 (m 1H), 3.18-3.13 (m, 2H), 2.77 (t, J=6.95 Hz, 2H), 2.35 (s, 3H).

### 5,6-Difluoro-1-methyl-2-tosyl-1,2,3,4-tetrahydroisoquinoline

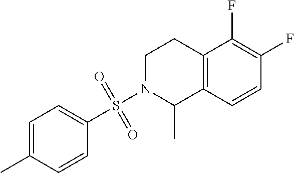 [SMILES: Cc1ccc(S(=O)(=O)N2CCc3c(ccc(F)c3F)C2C)cc1]

A mixture of N—[2-(2,3-difluorophenyl)ethyl]-4-methyl-benzenesulfonamide (6.5 g, 20.9 mmol) and acetaldehyde (1.3 mL, 23.8 mmol) in concentrated H₂SO₄/glacial AcOH mixture (2:1, 70 mL) was degassed with vacuum/nitrogen cycle (3 times), and then the mixture was stirred at room temperature for 30 hr under N₂ atmosphere. The reaction mixture was quenched with ice water (40 mL) and extracted with EtOAc (3×30 mL). The combined organic layers were washed with H₂O (50 mL), saturated aqueous NaHCO₃ solution (50 mL), and saturated aqueous brine solution (50 mL). The solution was dried over sodium sulfate, filtered, and concentrated under reduced pressure. The residue was purified by normal phase SiO₂ chromatography (0% to 5% EtOAc/petroleum ether) to afford 5,6-difluoro-1-methyl-2-tosyl-1,2,3,4-tetrahydroisoquinoline as a white solid, which was used without further purification (2.35 g, 22% yield, 66% purity, m/z: 338 [M+H]⁺ observed). ¹H NMR (400 MHz, CDCl₃) δ 7.58 (d, J=7.6 Hz, 2H) 7.14 (d, J=8 Hz, 2H) 6.90 (q, J=8.75 Hz, 1H) 6.74-6.72 (t, J=8, 4.63 Hz, 1H) 5.09-5.05 (m 1H) 3.94-3.89 (m, 1H) 3.30-3.23 (m, 1H) 2.65 (d, J=16.8 Hz 1H) 2.53-2.48 (m, 1H) 2.31 (s, 3H) 1.36 (d, J=6.8 Hz, 3H).

### 5,6-Difluoro-1-methy-1,2,3,4-tetrahydroisoquinoline

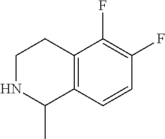

A mixture of 5,6-difluoro-1-methyl-2-tosyl-1,2,3,4-tetrahydroisoquinoline (66% purity, 2.2 g, 6.52 mmol) and Mg (1.58 g, 65.2 mmol) in MeOH (25 mL) was degassed with vacuum/nitrogen cycle (3 times), and then the mixture was stirred at room temperature for 10 hr under N₂ atmosphere. The reaction mixture was quenched with saturated aqueous NH₄Cl (50 mL) and extracted with EtOAc (5×20 mL). The combined organic layers were washed with saturated aqueous brine solution (40 mL), dried over sodium sulfate, filtered and concentrated under reduced pressure. The mixture was dissolved in MTBE (10 mL) and the pH was adjusted to 1-2 by the addition of HCl solution (6M in 1,4-dioxane, 8 mL) until pH 1-2. The resultant mixture was filtered through CELITE®, and the filter was evaporated under vacuum to give 5,6-difluoro-1-methyl-1,2,3,4-tetrahydroisoquinoline, hydrochloride salt as a yellow solid (1 g, 45% yield, m/z: 184 [M+H]⁺ observed). ¹H NMR (400 MHz, DMSO-d₆) δ 10.04 (s, 1H), 9.64 (s, 1H), 7.41-7.36 (m, 1H), 7.22-7.19 (m, 1H), 4.53 (s, 1H), 3.43 (d, J=6 Hz, 1H), 3.30-3.27 (m, 1H), 3.06-2.97 (m, 2H), 1.58 (d, J=6.8 Hz, 3H).

### 2-(2-Chloropyrimidin-4-yl)-5,6-difluoro-1-methyl-1,2,3,4-tetrahydroisoquinoline

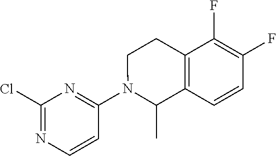 [SMILES: CC1c2ccc(F)c(F)c2CCN1c1ccnc(Cl)n1]

A mixture of 5,6-difluoro-1-methyl-1,2,3,4-tetrahydroisoquinoline, hydrochloride salt (500 mg, 2.28 mmol), 2,4-dichloropyrimidine (340 mg, 2.28 mmol), and N,N-diisopropylethylamine (1 mL, 5.7 mmol) in CH₂Cl₂ (10 mL) was degassed with vacuum/nitrogen cycle (3 times) and then the mixture was stirred at room temperature for 16 hr under N₂ atmosphere. The reaction mixture was concentrated under reduced pressure. The residue was purified by normal phase SiO₂ chromatography (0% to 20% EtOAc/petroleum ether) to afford 2-(2-chloropyrimidin-4-yl)-5,6-difluoro-1-methyl-1,2,3,4-tetrahydroisoquinoline as a yellow solid (0.51 g, 76% yield, m/z: 296 [M+H]⁺ observed).

### 2-([2,2′-Bipyrimidin]-4-yl)-5,6-difluoro-1-methyl-1,2,3,4-tetrahydroisoquinoline

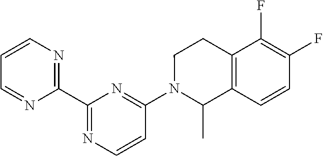 [SMILES: CC1c2ccc(F)c(F)c2CCN1c1ccnc(-c2ncccn2)n1]

A mixture of 2-(2-chloropyrimidin-4-yl)-5,6-difluoro-1-methyl-1,2,3,4-tetrahydroisoquinoline (50 mg, 0.169 mmol), 2-(tributylstannyl)pyrimidine (188 mg, 0.508 mmol), potassium carbonate (47 mg, 339 mmol), bis(triphenylphosphine)palladium(II) dichloride (12 mg, 0.017 mmol), and tert-butyl acetate (39 mg, 0.338 mmol) in DMF (2 mL) was degassed with vacuum/nitrogen cycle (3 times), and then the mixture was stirred at 80° C. for 10 hr under N₂ atmosphere. The reaction mixture was quenched with saturated aqueous KF solution (10 mL) and extracted with EtOAc (4×5 mL). The combined organic layers were washed with saturated aqueous brine solution (10 mL), dried over sodium sulfate, filtered, and concentrated under reduced pressure. The residue was purified by reverse phase HPLC to afford 2-([2,2′-bipyrimidin]-4-yl)-5,6-difluoro-1-methyl-1,2,3,4-tetrahydroisoquinoline as a white solid (17 mg, 30% yield, m/z: 340 [M+H]⁺ observed). H NMR (400 MHz, CDCl₃) δ 8.91 (d, J=4.8 Hz, 2H), 8.43 (d, J=6 Hz, 1H), 7.32 (t, J=4.7 Hz, 1H), 6.99-6.95 (m, 1H), 6.91-6.90 (m, 1H), 6.59 (d, J=6.4 Hz, 1H), 3.41-3.36 (m, 1H), 2.98-2.87 (m, 2H), 1.53 (br s, 2H), 1.47 (d, J=7.2 Hz, 3H).

### Example 37: 2-([2,2′-Bipyrimidin]-4-yl)-5,6-difluoro-1-methyl-1,2,3,4-tetrahydroisoquinoline (Single Enantiomer I)

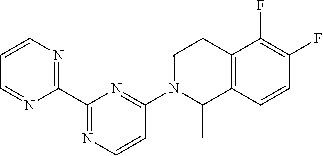 [SMILES: CC1c2ccc(F)c(F)c2CCN1c1ccnc(-c2ncccn2)n1]

### Example 38: 2-([2,2′-Bipyrimidin]-4-yl)-5,6-difluoro-1-methyl-1,2,3,4-tetrahydroisoquinoline (Single Enantiomer II)

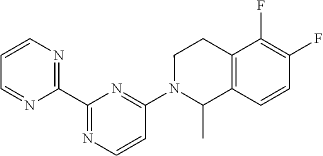 [SMILES: CC1c2ccc(F)c(F)c2CCN1c1ccnc(-c2ncccn2)n1]

180 mg of the mixture of enantiomers was separated by SFC (supercritical fluid chromatography) on a CHIRALCEL® OD-H column using liquid CO₂ and EtOH (44%; 0.1% aqueous NH₃ as modifier) to give 2-([2,2′-bipyrimidin]-4-yl)-5,6-difluoro-1-methyl-1,2,3,4-tetrahydroisoquinoline (Single Enantiomer I) as a light red solid (faster eluting enantiomer, 52 mg, 29%, m/z: 340 [M+H]⁺ observed) and 2-([2,2′-bipyrimidin]-4-yl)-5,6-difluoro-1-methyl-1,2,3,4-tetrahydroisoquinoline (Single Enantiomer II) as a light pink solid (slower eluting enantiomer, 52 mg, 29%, m/z: 340 [M+H]⁺ observed).

### Example 37: 2-([2,2′-Bipyrimidin]-4-yl)-5,6-difluoro-1-methyl-1,2,3,4-tetrahydroisoquinoline (Single Enantiomer I)

m/z: 340 [M+H]⁺ observed. ¹H NMR (400 MHz, CDCl₃) δ 8.91 (d, J=4.8 Hz, 2H), 8.43 (d, J=6 Hz, 1H), 7.32 (t, J=4.7 Hz, 1H), 6.99-6.95 (m, 1H), 6.91-6.90 (m, 1H), 6.59 (d, J=6.4 Hz, 1H), 3.41-3.36 (m, 1H), 2.98-2.87 (m, 2H), 1.53 (br s, 2H), 1.47 (d, J=7.2 Hz, 3H).

### Example 38: 2-([2,2′-Bipyrimidin]-4-yl)-5,6-difluoro-1-methyl-1,2,3,4-tetrahydroisoquinoline (Single Enantiomer II)

m/z: 340 [M+H]⁺ observed. ¹H NMR (400 MHz, CDCl₃) δ 8.91 (d, J=4.8 Hz, 2H), 8.43 (d, J=6 Hz, 1H), 7.32 (t, J=4.7 Hz, 1H), 6.99-6.95 (m, 1H), 6.91-6.90 (m, 1H), 6.59 (d, J=6.4 Hz, 1H), 3.41-3.36 (m, 1H), 2.98-2.87 (m, 2H), 1.53 (br s, 2H), 1.47 (d, J=7.2 Hz, 3H).

The following examples were prepared in a similar manner as 2-([2,2′-bipyrimidin]-4-yl)-5,6-difluoro-1-methyl-1,2,3,4-tetrahydroisoquinoline from 4-bromo-2,2′-bipyrimidine and an appropriate amine.

### Example 39: 1-Ethyl-5,6-difluoro-2-(2-pyrimidin-2-ylpyrimidin-4-yl)-3,4-dihydro-1H-isoquinoline

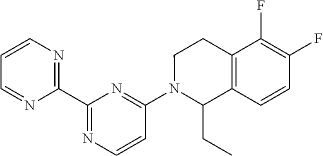 [SMILES: CCC1c2ccc(F)c(F)c2CCN1c1ccnc(-c2ncccn2)n1]

m/z: 354 [M+H]⁺ observed. ¹H NMR (400 MHz, DMSO-d₆) δ 9.05 (m, 2H), 8.42 (s, 1H), 7.62 (t, J=5.2 Hz, 1H), 7.31-7.26 (m, 2H), 7.07 (d, J=6.4 Hz 1H), 3.62-3.52 (m, 1H), 2.96-2.93 (m, 2H), 1.98-1.91 (m, 2H), 1.29 (s, 2H), 0.96 (t, J=7.2 Hz, 3H).

### Example 40: 1-Ethyl-5,6-difluoro-2-(2-pyrimidin-2-ylpyrimidin-4-yl)-3,4-dihydro-1H-isoquinoline (Single Enantiomer I)

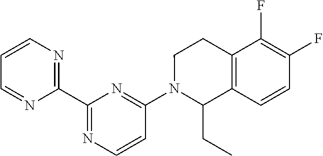 [SMILES: CCC1c2ccc(F)c(F)c2CCN1c1ccnc(-c2ncccn2)n1]

m/z: 354 [M+H]⁺ observed. ¹H NMR (400 MHz, DMSO-d₆) δ 9.05 (m, 2H), 8.42 (s, 1H), 7.62 (t, J=5.2 Hz, 1H), 7.31-7.26 (m, 2H), 7.07 (d, J=6.4 Hz 1H), 3.62-3.52 (m, 1H), 2.96-2.93 (m, 2H), 1.98-1.91 (m, 2H), 1.29 (s, 2H), 0.96 (t, J=7.2 Hz, 3H).

### Example 41: 1-Ethyl-5,6-difluoro-2-(2-pyrimidin-2-ylpyrimidin-4-yl)-3,4-dihydro-1H-isoquinoline (Single Enantiomer II)

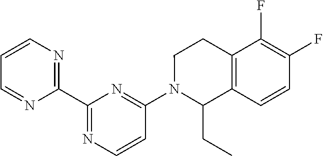 [SMILES: CCC1c2ccc(F)c(F)c2CCN1c1ccnc(-c2ncccn2)n1]

m/z: 354 [M+H]⁺ observed. ¹H NMR (400 MHz, DMSO-d₆) δ 9.05 (m, 2H), 8.42 (s, 1H), 7.62 (t, J=5.2 Hz, 1H), 7.31-7.26 (m, 2H), 7.07 (d, J=6.4 Hz 1H), 3.62-3.52 (m, 1H), 2.96-2.93 (m, 2H), 1.98-1.91 (m, 2H), 1.29 (s, 2H), 0.96 (t, J=7.2 Hz, 3H).

### Example 42: 1-Ethyl-5-fluoro-8-methoxy-2-(2-pyrimidin-2-ylpyrimidin-4-yl)-3,4-dihydro-1H-isoquinoline

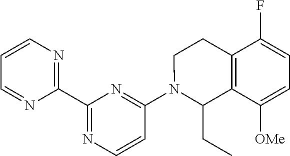 [SMILES: CCC1c2c(OC)ccc(F)c2CCN1c1ccnc(-c2ncccn2)n1]

m/z: 366 [M+H]⁺ observed. ¹H NMR (400 MHz, CDCl₃) δ 9.15-9.11 (m, 2H), 8.80 (s, 1H), 7.62-7.57 (m, 1H), 7.00-6.95 (m, 2H), 6.76-6.58 (m, 1H), 5.40-5.25 (m, 2H), 3.90 (s, 3H), 3.89-3.67 (m, 1H), 3.06-3.02 (m, 2H), 2.23-2.21 (m, 1H), 1.94-1.92 (s, 1H), 1.02-0.96 (m, 3H).

### Example 43: 1-Ethyl-5-fluoro-8-methoxy-2-(2-pyrimidin-2-ylpyrimidin-4-yl)-3,4-dihydro-1H-isoquinoline (Single Enantiomer I)

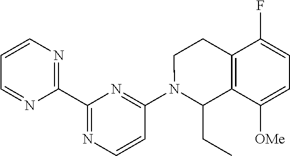 [SMILES: CCC1c2c(OC)ccc(F)c2CCN1c1ccnc(-c2ncccn2)n1]

m/z: 366 [M+H]⁺ observed. ¹H NMR (400 MHz, CDCl₃) δ 9.15-9.11 (m, 2H), 8.80 (s, 1H), 7.62-7.57 (m, 1H), 7.00-6.95 (m, 2H), 6.76-6.58 (m, 1H), 5.40-5.25 (m, 2H), 3.90 (s, 3H), 3.89-3.67 (m, 1H), 3.06-3.02 (m, 2H), 2.23-2.21 (m, 1H), 1.94-1.92 (s, 1H), 1.02-0.96 (m, 3H).

### Example 44: 1-Ethyl-5-fluoro-8-methoxy-2-(2-pyrimidin-2-ylpyrimidin-4-yl)-3,4-dihydro-1H-isoquinoline (Single Enantiomer II)

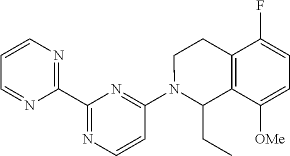 [SMILES: CCC1c2c(OC)ccc(F)c2CCN1c1ccnc(-c2ncccn2)n1]

m/z: 366 [M+H]⁺ observed. ¹H NMR (400 MHz, CDCl₃) δ 9.15-9.11 (m, 2H), 8.80 (s, 1H), 7.62-7.57 (m, 1H), 7.00-6.95 (m, 2H), 6.76-6.58 (m, 1H), 5.40-5.25 (m, 2H), 3.90 (s, 3H), 3.89-3.67 (m, 1H), 3.06-3.02 (m, 2H), 2.23-2.21 (s, 1H), 1.94-1.92 (s, 1H), 1.02-0.96 (m, 3H).

### Example 45: 5-Fluoro-8-methoxy-2-(2-pyrimidin-2-ylpyrimidin-4-yl)-3,4-dihydro-1H-isoquinoline

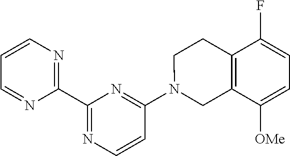 [SMILES: COc1ccc(F)c2c1CN(c1ccnc(-c3ncccn3)n1)CC2]

m/z: 338 [M+H]⁺ observed. ¹H NMR (400 MHz, CDCl₃) δ 9.14-9.13 (m, 2H), 8.90 (s, 1H), 7.59-7.58 (m, 1H), 7.13-6.97 (m, 2H), 6.77-6.73 (m, 1H), 4.73 (s, 2H), 4.52-4.50 (m, 2H), 3.90-3.88 (m, 3H), 3.09-3.05 (m, 2H).

### Example 46: 1-Ethyl-6-fluoro-5-methoxy-2-(2-pyrimidin-2-ylpyrimidin-4-yl)-3,4-dihydro-1H-isoquinoline

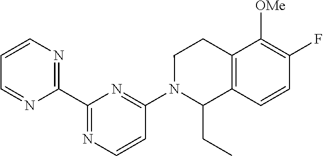 [SMILES: CCC1c2ccc(F)c(OC)c2CCN1c1ccnc(-c2ncccn2)n1]

m/z: 366 [M+H]⁺ observed. ¹H NMR (400 MHz, DMSO-d₆) δ 9.14 (s, 2H), 8.38 (s, 1H), 7.82-7.80 (m, 1H), 7.35 (d, J=6.8 Hz, 1H), 7.17-7.08 (m, 2H), 5.78 (s, 1H), 3.88 (s, 3H), 3.87-3.73 (m, 2H), 3.06-2.97 (m, 2H), 2.02-1.97 (m, 2H), 0.95 (t, J=7.2 Hz, 3H).

### Example 47: 1-Ethyl-5,6-difluoro-7-methoxy-2-(2-pyrimidin-2-ylpyrimidin-4-yl)-3,4-dihydro-1H-isoquinoline

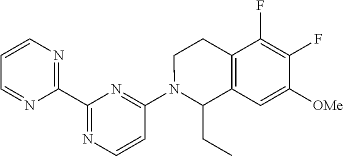 [SMILES: CCC1c2cc(OC)c(F)c(F)c2CCN1c1ccnc(-c2ncccn2)n1]

m/z: 384 [M+H]⁺ observed. ¹H NMR (400 MHz, DMSO-d₆) δ 9.18-9.17 (m, 2H), 8.44 (d, J=7.6 Hz, 1H), 7.89-7.84 (m, 1H), 7.45-7.43 (m, 1H), 7.15-6.98 (m, 1H), 6.20-6.18 (m, 0.5H), 5.63-5.11 (m, 0.5H), 3.89 (s, 3H), 3.73-3.57 (m, 2H), 3.02-2.72 (m, 2H), 2.19-1.92 (m, 2H), 0.93 (t, J=7.2 Hz, 3H).

### Example 48: 2-([2,2′-Bipyrimidin]-4-yl)-1-ethyl-5,6-difluoroisoindoline

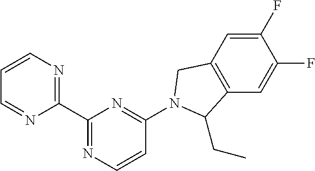 [SMILES: CCC1c2cc(F)c(F)cc2CN1c1ccnc(-c2ncccn2)n1]

### 5,6-Difluoroisoindoline-1,3-dione

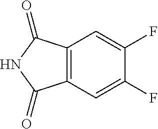 [SMILES: O=C1NC(=O)c2cc(F)c(F)cc21]

A solution of 5,6-difluoroisobenzofuran-1,3-dione (7.5 g, 41 mmol) in formamide (40 mL) was stirred at 130° C. for 2 h. The mixture was poured into ice water and stirred for 30 min. The white precipitate was filtered and dried to give 5,6-difluoroisoindoline-1,3-dione as an off-white solid (7.5 g, 94% yield, m/z: 203 [M+H+H₂O]⁺ observed). ¹H NMR (300 MHz, DMSO-d₆) δ 1.55 (s, 1H), 8.02-7.95 (m, 2H).

### 3-Ethyl-5,6-difluoro-3-hydroxyisoindolin-1-one

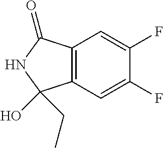

To a solution of 5,6-difluoroisoindoline-1,3-dione (7.5 g, 41 mmol) in CH₂Cl₂ (150 mL) at 5° C., was added dropwise ethylmagnesium bromide (3.0M in Et₂O, 41.0 mL, 123 mmol) under N₂ and the mixture was stirred at 5° C. for 3 hr. The reaction was quenched with saturated aqueous NH₄Cl solution (100 mL). The resulting mixture was extracted with CH₂Cl₂ (2×300 mL). The combined organic phase was washed with saturated aqueous brine solution (100 mL), dried over Na₂SO₄, and evaporated to dryness under reduced pressure. The residue was triturated with n-pentane to give 3-ethyl-5,6-difluoro-3-hydroxyisoindolin-1-one as a white solid (7.1 g, 81% yield, m/z: 214 [M+H]⁺ observed). ¹H NMR (300 MHz, DMSO-d₆) δ 8.94 (s, 1H), 7.69-7.60 (m, 2H), 6.30 (s, 1H), 1.98-1.88 (m, 2H), 0.68 (t, 3H).

### 3-Ethylidene-5,6-difluoroisoindolin-1-one

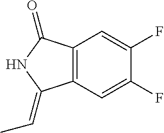

To a solution of 3-ethyl-5,6-difluoro-3-hydroxyisoindolin-1-one (7.1 g, 33 mmol) in CH₂Cl₂ (100 mL) at −15° C. was added triethylsilane (42 mL, 266 mmol) and boron trifluoride diethyl etherate (8.3 mL, 67 mmol). The reaction mixture was stirred at rt for 24 h. The reaction mixture was quenched with saturated aqueous NaHCO₃ solution, then extracted with CH₂Cl₂ (2×200 mL). The organic layer was washed with saturated aqueous brine solution (100 mL), dried over Na₂SO₄, filtered and evaporated to dryness under reduced pressure to give 3-ethylidene-5,6-difluoroisoindolin-1-one as a white solid, which was used without further purification (6.1 g, 94% yield, m/z: 196 [M+H]⁺ observed).

### 3-Ethyl-5,6-difluoroisoindolin-1-one

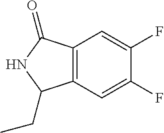

To a solution of (Z)-3-ethylidene-5,6-difluoroisoindolin-1-one (6.1 g, 31 mmol) in MeOH (100 mL) was added palladium on carbon (10 wt. % loading on carbon, 1.2 g, 1.1 mmol). The reaction mixture was stirred under H₂ atmosphere (balloon) at rt for 16 h. The reaction was degassed, then filtered through a CELITE® pad and washed with MeOH (2×50 mL). The combined organic layer was evaporated to dryness under reduced pressure to give 3-ethyl-5,6-difluoroisoindolin-1-one as a white solid (4.1 g, 67% yield, m/z: 198 [M+H]⁺ observed). ¹H NMR (300 MHz, DMSO-d₆) δ 8.99 (s, 1H), 7.77-7.63 (m, 2H), 4.54 (t, 1H), 1.99-1.88 (m, 1H), 1.62-1.53 (m, 1H), 0.80 (t, 3H).

### 1-Ethyl-5,6-difluoroisoindoline Hydrochloride

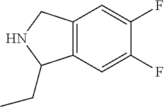

To a solution of 3-ethyl-5,6-difluoroisoindolin-1-one (4.1 g, 21 mmol) in THF (100 mL) 0° C. was added borane (1M solution in THF, 83 mL, 83 mmol). The reaction mixture was stirred at reflux for 48 h. The reaction mixture was cooled to rt and quenched by the slow addition of ice-cold H₂O (50 mL), followed by the addition of aqueous sodium hydroxide (1.0 M solution in water, 50 mL). The mixture was extracted with EtOAc (2×200 mL). The organic layer was washed with saturated aqueous brine solution (100 mL), dried over anhydrous sodium sulfate, filtered and evaporated to dryness. To the residue was added HCl (4.0M solution in 1,4-dioxane, 20 mL, 80 mmol) and the solvent evaporated. The residue was triturated with diethyl ether and evaporated under reduced pressure to give 1-ethyl-5,6-difluoroisoindoline as a white solid (HCl salt, 3.1 g, 68% yield, m/z: 184 [M+H]⁺ observed). ¹H NMR (400 MHz, DMSO-d₆) δ 10.16 (br s, 2H), 7.56-7.50 (m, 2H), 4.75 (q, 1H), 4.49-4.38 (q, 2H), 2.11-2.05 (m, 1H), 1.90-1.82 (m, 1H), 1.02 (t, 3H).

### 2-(2-Chloropyrimidin-4-yl)-1-ethyl-5,6-difluoroisoindoline

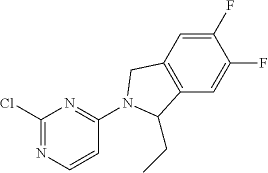 [SMILES: CCC1c2cc(F)c(F)cc2CN1c1ccnc(Cl)n1]

To a solution of 1-ethyl-5,6-difluoroisoindoline, hydrochloride salt (0.60 g, 2.7 mmol.) in THF (10 mL) was added N,N-diisopropylethylamine (1.5 mL, 8.2 mmol) and 2,4-dichloropyrimidine (0.45 g, 3.0 mmol) and the reaction mixture was stirred at rt for 2 h. The reaction mixture was diluted with water (100 mL) and extracted with EtOAc (2×100 mL). The combined organic layer was washed with saturated aqueous brine solution (100 mL), dried over anhydrous sodium sulfate, filtered and evaporated under reduced pressure. The residue was purified normal phase SiO₂ chromatography (0-20% EtOAc/petroleum ether) to give 2-(2-chloropyrimidin-4-yl)-1-ethyl-5,6-difluoroisoindoline as a white solid (0.41 g, 50% yield, m/z: 296 [M+H]⁺ observed). ¹H NMR (400 MHz, CDCl₃) δ 8.1 (d, 1H), 7.15-7.04 (m, 2H), 6.38-6.30 (m, 1H), 5.10 (br s, 1H), 4.64 (br s, 2H), 2.04 (br s, 1H), 1.90-1.63 (m, 1H), 0.60 (s, 3H).

### 2-([2,2′-Bipyrimidin]-4-yl)-1-ethyl-5,6-difluoroisoindoline

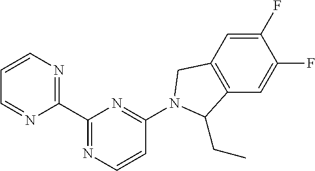 [SMILES: CCC1c2cc(F)c(F)cc2CN1c1ccnc(-c2ncccn2)n1]

To a solution of 2-(2-chloropyrimidin-4-yl)-1-ethyl-5,6-difluoroisoindoline (0.41 g, 1.38 mmol) in DMF (5 mL) was added 2-(tributylstannyl)pyrimidine (0.51 g, 1.4 mmol), tetraethylammonium chloride (0.23 g, 1.4 mmol), and potassium carbonate (0.39 g, 2.8 mmol). The mixture was degassed with N₂ for 10 min. Then bis(triphenylphosphine) palladium(II) dichloride (9.7 mg, 0.013 mmol) was added and the solution was degassed with N₂ for 5 min. The reaction mixture was stirred at 100° C. for 24 h, cooled to rt, diluted with water (100 mL) and extracted with EtOAc (2×100 mL). The combined organic layer was washed with saturated aqueous brine solution (100 mL), dried over anhydrous sodium sulfate, filtered and evaporated under reduced pressure. The crude residue was purified by reverse phase HPLC to give 2-(2,2′-bipyrimidin-4-yl)-1-ethyl-5,6-difluoroisoindoline as a white solid (0.12 g, 25% yield, m/z: 340 [M+H]⁺ observed). ¹H NMR (300 MHz, DMSO-d₆, heated to 90° C.) δ 8.92 (d, 2H), 8.40 (d, 1H), 7.54 (t, 1H), 7.49-7.39 (m, 2H), 6.73 (d, 1H), 5.44 (s, 1H), 4.85 (q, 2H), 2.38-2.34 (m, 1H), 1.92-1.84 (m, 1H), 0.58 (t, 3H).

### Example 49: 2-([2,2′-Bipyrimidin]-4-yl)-1-ethyl-5,6-difluoroisoindoline (Single Enantiomer I)

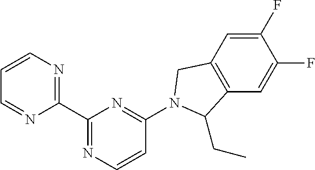 [SMILES: CCC1c2cc(F)c(F)cc2CN1c1ccnc(-c2ncccn2)n1]

### Example 50: 2-([2,2′-Bipyrimidin]-4-yl)-1-ethyl-5,6-difluoroisoindoline (Single Enantiomer II)

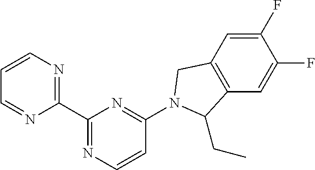 [SMILES: CCC1c2cc(F)c(F)cc2CN1c1ccnc(-c2ncccn2)n1]

A mixture of enantiomers (54 mg) was separated by chiral chromatography on a CHIRALCEL® OD-H column using 50% ethanol in n-hexane to give 2-([2,2′-bipyrimidin]-4-yl)-1-ethyl-5,6-difluoroisoindoline (Single Enantiomer I) as a pale yellow solid (faster eluting enantiomer, 25 mg, 46% yield, m/z: 340 [M+H]⁺ observed), and 2-([2,2′-bipyrimidin]-4-yl)-1-ethyl-5,6-difluoroisoindoline (Single Enantiomer II) as a pale yellow solid (slower eluting enantiomer, 27 mg, 53% yield, m/z: 340 [M+H]⁺ observed).

### Example 49: 2-([2,2′-Bipyrimidin]-4-yl)-1-ethyl-5,6-difluoroisoindoline (Single Enantiomer I)

m/z: 340 [M+H]⁺ observed. ¹H NMR (300 MHz, DMSO-d₆, heated to 90° C.) δ 8.92 (d, 2H), 8.40 (d, 1H), 7.54 (t, 1H), 7.49-7.39 (m, 2H), 6.73 (d, 1H), 5.44 (s, 1H), 4.85 (q, 2H), 2.38-2.34 (m, 1H), 1.92-1.84 (m, 1H), 0.58 (t, 3H).

### Example 50: 2-([2,2′-Bipyrimidin]-4-yl)-1-ethyl-5,6-difluoroisoindoline (Single Enantiomer II)

m/z: 340 [M+H]⁺ observed. ¹H NMR (300 MHz, DMSO-d₆, heated to 90° C.) δ 8.92 (d, 2H), 8.40 (d, 1H), 7.54 (t, 1H), 7.49-7.39 (m, 2H), 6.73 (d, 1H), 5.44 (s, 1H), 4.85 (q, 2H), 2.38-2.34 (m, 1H), 1.92-1.84 (m, 1H), 0.58 (t, 3H).

The following examples were prepared in a similar manner as 2-([2,2′-bipyrimidin]-4-yl)-1-ethyl-5,6-difluoroisoindoline from 1-ethyl-5,6-difluoroisoindoline and an appropriately 5-substituted 2,4-dichloropyrimidine, followed by coupling with 2-(tributylstannyl)pyrimidine.

### Example 51: 1-Ethyl-5,6-difluoro-2-(5-fluoro-[2,2′-bipyrimidin]-4-yl)isoindoline

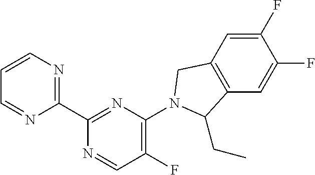 [SMILES: CCC1c2cc(F)c(F)cc2CN1c1nc(-c2ncccn2)ncc1F]

m/z: 358 [M+H]⁺ observed. ¹H NMR (400 MHz, CDCl₃) δ 8.98 (d, J=4.8 Hz, 2H), 8.35 (d, J=5.3 Hz, 1H), 7.39 (t, J=4.8 Hz, 1H), 7.10 (dt, J=22.4, 8.3 Hz, 2H), 5.55 (bs, 1H), 5.08 (d, J=15.6 Hz, 2H), 2.24 (bs, 1H), 1.90 (ddd, J=14.3, 7.3, 2.7 Hz, 1H), 0.71 (t, J=7.4 Hz, 3H).

### Example 52: 1-Ethyl-5,6-difluoro-2-(5-methyl-[2,2′-bipyrimidin]-4-yl)isoindoline

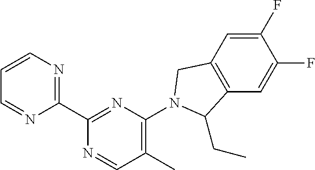 [SMILES: CCC1c2cc(F)c(F)cc2CN1c1nc(-c2ncccn2)ncc1C]

m/z: 354 [M+H]⁺ observed. ¹H NMR (400 MHz, CDCl₃) 8.98 (s, 2H), 8.33 (s, 1H), 7.38 (s, 1H), 7.14-7.01 (m, 2H), 5.91 (s, 1H), 5.25 (d, J=14.3 Hz, 1H), 4.92 (d, J=14.3 Hz, 1H), 2.48 (s, 3H), 2.17-2.01 (m, 1H), 1.90-1.75 (m, 1H), 0.71 (t, J=7.4 Hz, 3H).

### Example 53: 2-(5-Chloro-[2,2′-bipyrimidin]-4-yl)-1-ethyl-5,6-difluoroisoindoline

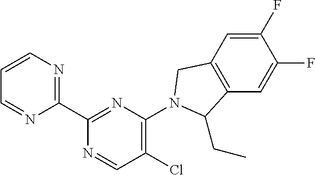 [SMILES: CCC1c2cc(F)c(F)cc2CN1c1nc(-c2ncccn2)ncc1Cl]

m/z: 374 [M+H]⁺ observed. ¹H NMR (400 MHz, CDCl₃) δ 8.98 (s, 2H), 8.49 (s, 1H), 7.42 (s, 1H), 7.19-7.03 (m, 2H), 6.16-6.04 (m, 1H), 5.34 (d, J=15.2 Hz, 1H), 5.12 (d, J=15.2 Hz, 1H), 2.29-2.12 (m, 1H), 1.96-1.74 (m, 1H), 0.72 (t, J=7.4 Hz, 3H).

### Example 54: 2-(5-Cyclopropyl-[2,2′-bipyrimidin]-4-yl)-1-ethyl-5,6-difluoroisoindoline

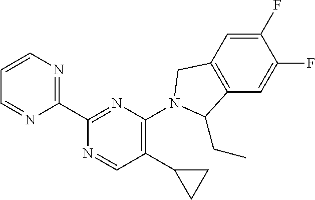 [SMILES: CCC1c2cc(F)c(F)cc2CN1c1nc(-c2ncccn2)ncc1C1CC1]

m/z: 380 [M+H]⁺ observed. ¹H NMR (400 MHz, CDCl₃) δ 9.01 (s, 2H), 8.56 (s, 1H), 7.42 (t, J=5.0 Hz, 1H), 7.16-7.05 (m, 2H), 6.14 (s, 1H), 5.46 (d, J=14.8 Hz, 1H), 5.22 (d, J=14.7 Hz, 1H), 2.29 (s, 1H), 2.10 (s, 1H), 1.87 (d, J=20.3 Hz, 1H), 1.17 (m, 2H), 0.96 (s, 2H), 0.72 (t, J=7.4 Hz, 3H).

The following examples were prepared in a similar manner as 2-([2,2′-bipyrimidin]-4-yl)-1-ethyl-5,6-difluoroisoindoline from 5,6-dimethoxyisobenzofuran-1,3-dione and formamide.

### Example 55: 2-([2,2′-Bipyrimidin]-4-yl)-1-ethyl-5,6-dimethoxyisoindoline

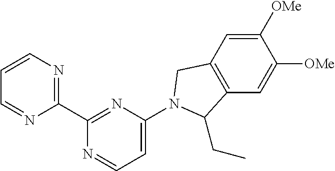 [SMILES: CCC1c2cc(OC)c(OC)cc2CN1c1ccnc(-c2ncccn2)n1]

m/z: 364 [M+H]⁺ observed. ¹H NMR (400 MHz, DMSO-d₆) δ 8.97 (d, 2H), 8.39 (br s, 1H), 7.60 (t, 1H), 7.00 (br s, 1H), 6.96 (s, 1H), 6.83-6.68 (m, 1H), 5.48-5.23 (m, 1H), 4.67 (br s, 2H), 3.77 (s, 6H), 2.60 (br s, 1H), 1.85 (br s, 1H), 0.56-0.48 (m, 3H).

### Example 56: 2-([2,2′-Bipyrimidin]-4-yl)-1-ethyl-5,6-dimethoxyisoindoline (Single Enantiomer I)

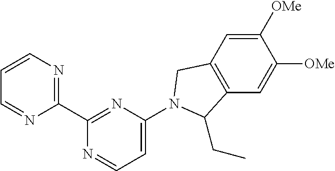 [SMILES: CCC1c2cc(OC)c(OC)cc2CN1c1ccnc(-c2ncccn2)n1]

m/z: 364 [M+H]⁺ observed. ¹H NMR (400 MHz, DMSO-d₆) δ 8.97 (d, 2H), 8.39 (br s, 1H), 7.60 (t, 1H), 7.00 (br s, 1H), 6.96 (s, 1H), 6.83-6.68 (m, 1H), 5.48-5.23 (m, 1H), 4.67 (br s, 2H), 3.77 (s, 6H), 2.60 (br s, 1H), 1.85 (br s, 1H), 0.56-0.48 (m, 3H).

### Example 57: 2-([2,2′-Bipyrimidin]-4-yl)-1-ethyl-5,6-dimethoxyisoindoline (Single Enantiomer II)

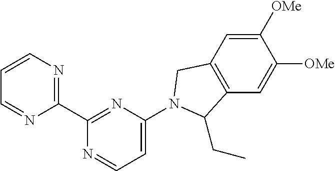 [SMILES: CCC1c2cc(OC)c(OC)cc2CN1c1ccnc(-c2ncccn2)n1]

m/z: 364 [M+H]⁺ observed. ¹H NMR (400 MHz, DMSO-d₆) δ 8.97 (d, 2H), 8.39 (br s, 1H), 7.60 (t, 1H), 7.00 (br s, 1H), 6.96 (s, 1H), 6.83-6.68 (m, 1H), 5.48-5.23 (m, 1H), 4.67 (br s, 2H), 3.77 (s, 6H), 2.60 (br s, 1H), 1.85 (br s, 1H), 0.56-0.48 (m, 3H).

The following examples were prepared in a similar manner as 2-([2,2′-bipyrimidin]-4-yl)-1-ethyl-5,6-difluoroisoindoline from 1-ethyl-5,6-dimethoxyisoindoline and an appropriately 5-substituted 2,4-dichloropyrimidine, followed by coupling with 2-(tributylstannyl)pyrimidine.

### Example 58: 1-Ethyl-5,6-dimethoxy-2-(5-methyl-[2,2′-bipyrimidin]-4-yl)isoindoline

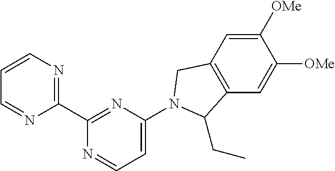 [SMILES: CCC1c2cc(OC)c(OC)cc2CN1c1ccnc(-c2ncccn2)n1]

m/z: 378 [M+H]⁺ observed. ¹H NMR (400 MHz, CDCl₃) δ 9.04 (d, J=4.9 Hz, 2H), 8.51 (s, 1H), 7.58-7.48 (m, 1H), 6.83 (s, 1H), 6.78 (s, 1H), 6.03 (bs, 1H), 5.39-5.22 (m, 2H), 3.91 (d, J=1.7 Hz, 6H), 2.65 (s, 3H), 2.49-2.03 (m, 1H), 1.96 (ddd, J=14.3, 7.4, 2.9 Hz, 1H), 0.74 (t, J=7.4 Hz, 3H).

### Example 59: 4-(1-Ethyl-5,6-dimethoxyisoindolin-2-yl)-N-methyl-[2,2′-bipyrimidin]-5-amine

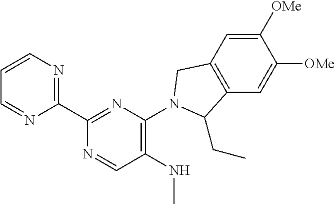 [SMILES: CCC1c2cc(OC)c(OC)cc2CN1c1nc(-c2ncccn2)ncc1NC]

m/z: 393 [M+H]⁺ observed. ¹H NMR (400 MHz, CDCl₃) δ 8.93 (d, J=4.8 Hz, 2H), 8.05 (s, 1H), 7.28 (t, J=4.8 Hz, 1H), 6.79 (s, 1H), 6.76 (s, 1H), 5.93-5.88 (m, 1H), 5.30 (dd, J=13.7, 2.6 Hz, 1H), 4.61 (d, J=13.7 Hz, 1H), 3.90 (s, 3H), 3.89 (s, 3H), 3.76 (s, 1H), 2.99 (s, 3H), 1.97-1.86 (m, 1H), 1.79-1.68 (m, 1H), 0.68 (t, J=7.4 Hz, 3H).

### Example 60: 2-(5-Chloro-[2,2′-bipyrimidin]-4-yl)-1-ethyl-5,6-dimethoxyisoindoline

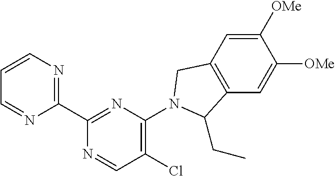 [SMILES: CCC1c2cc(OC)c(OC)cc2CN1c1nc(-c2ncccn2)ncc1Cl]

m/z: 398 [M+H]⁺ observed. ¹H NMR (400 MHz, CDCl₃) δ 8.98 (d, J=4.8 Hz, 2H), 8.46 (s, 1H), 7.39 (t, J=4.8 Hz, 1H), 6.83 (s, 1H), 6.76 (s, 1H), 6.12-6.05 (m, 1H), 5.38-5.29 (m, 1H), 5.11 (d, J=14.7 Hz, 1H), 3.91-3.89 (m, 6H), 2.33-2.15 (m, 1H), 1.95-1.82 (m, 1H), 0.71 (t, J=7.4 Hz, 3H).

### Example 61: 2-(5-Cyclopropyl-[2,2′-bipyrimidin]-4-yl)-1-ethyl-5,6-dimethoxyisoindoline

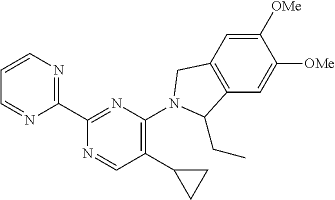 [SMILES: CCC1c2cc(OC)c(OC)cc2CN1c1nc(-c2ncccn2)ncc1C1CC1]

m/z: 404 [M+H]⁺ observed. ¹H NMR (400 MHz, CDCl₃) δ 9.02 (d, J=4.9, 0.4 Hz, 2H), 8.67 (s, 1H), 7.49 (t, J=5.1, 4.6 Hz, 1H), 6.80 (d, J=15.6 Hz, 2H), 6.18 (s, 1H), 5.51 (d, J=14.6 Hz, 1H), 5.40 (d, J=14.6 Hz, 1H), 3.91 (d, J=1.5 Hz, 6H), 2.45-2.35 (m, 1H), 2.23-2.14 (m, 1H), 2.02-1.87 (m, 1H), 1.30-1.18 (m, 2H), 1.13-0.98 (m, 2H), 0.73 (t, J=7.4 Hz, 3H).

### Example 62: 1-Ethyl-2-(5-isopropyl-[2,2′-bipyrimidin]-4-yl)-5,6-dimethoxyisoindoline

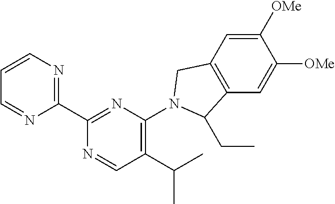 [SMILES: CCC1c2cc(OC)c(OC)cc2CN1c1nc(-c2ncccn2)ncc1C(C)C]

m/z found 406.3 [M+H]⁺. ¹H NMR (400 MHz, CDCl₃) δ 8.96 (d, J=4.8 Hz, 2H), 8.50 (s, 1H), 7.36 (t, J=4.8 Hz, 1H), 6.79 (d, J=14.8 Hz, 2H), 6.01-5.94 (m, 1H), 5.21 (d, J=13.6, 2.5 Hz, 1H), 4.77 (d, J=13.4 Hz, 1H), 3.90 (s, 6H), 3.41-3.27 (m, 1H), 2.25-2.07 (m, 1H), 1.89-1.74 (m, 1H), 1.49 (d, J=6.7 Hz, 3H), 1.19 (d, J=6.8 Hz, 3H), 0.68 (t, J=7.4 Hz, 3H).

### Example 63: 2-([2,2′-Bipyrimidin]-4-yl)-1-ethyl-6-fluoro-5-methoxyisoindoline

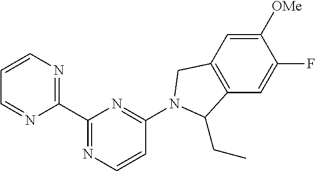 [SMILES: CCC1c2cc(F)c(OC)cc2CN1c1ccnc(-c2ncccn2)n1]

### 2-Bromo-4-fluoro-5-methoxybenzonitrile

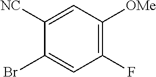 [SMILES: COc1cc(C#N)c(Br)cc1F]

To a solution of 4-fluoro-3-methoxybenzonitrile (10 g, 66.2 mmol) in AcOH/H₂O (1:1, 100 mL) was added dropwise bromine (7.5 mL, 146 mmol) at rt and the reaction mixture was heated at 50° C. for 16 h. The mixture was cooled to rt and poured into ice-cold water (100 mL) and stirred for 30 min. The resulting white precipitate was filtered and dried under vacuum to give 2-bromo-4-fluoro-5-methoxybenzonitrile as an off-white solid (11.5 g, 76% yield). ¹H NMR (400 MHz, CDCl₃): δ 7.38 (d, 1H), 7.21 (d, 1H), 3.19 (s, 3H).

### Ethyl 2-cyano-5-fluoro-4-methoxybenzoate

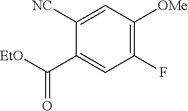 [SMILES: CCOC(=O)c1cc(F)c(OC)cc1C#N]

To a solution of 2-bromo-4-fluoro-5-methoxybenzonitrile (11 g, 48.2 mmol) in EtOH (240 mL) was added triethylamine (20 mL, 144 mmol) at rt in a steel bomb. The reaction mixture was then degassed with argon for 10-15 min. To the reaction mixture was added 1,3-bis(diphenylphosphino)propane (3.0 g, 7.3 mmol) and Pd(OAc)₂ (1.1 g, 4.8 mmol) with continued degassing for 10 min. The reaction mixture was stirred under CO pressure (200 psi) at 100° C. for 16 h. The mixture was concentrated under reduced pressure, diluted with water (50 mL), and extracted with EtOAc (2×350 mL). The combined organic layer was washed with saturated aqueous brine solution (100 mL), dried over anhydrous sulfate, filtered and evaporated under reduced pressure. The residue was purified by normal phase SiO₂ chromatography (0-20% EtOAc/petroleum ether) to give ethyl 2-cyano-5-fluoro-4-methoxybenzoate (8.5 g, 79% yield, m/z: 224 [M+H]⁺ observed). ¹H NMR (400 MHz, CDCl₃): δ 7.85 (d, 1H), 7.31 (d, 1H), 4.44 (q, 2H), 3.99 (s, 3H), 1.44 (t, 3H).

### 6-Fluoro-5-methoxyisoindolin-1-one

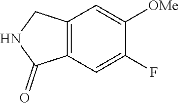 [SMILES: COc1cc2c(cc1F)C(=O)NC2]

To a solution of ethyl 2-cyano-5-fluoro-4-methoxybenzoate (8.5 g, 38 mmol) in EtOH (200 mL) was added palladium (10 wt. % loading on carbon, 4.0 g, 3.8 mmol) at rt and stirred under H₂ pressure (200 psi) in a steel bomb at room temperature for 16 h. The reaction mixture was degassed and back filled with nitrogen, filtered through CELITE® and washed with MeOH (100 mL). The filtrate was evaporated under reduced pressure to give crude 6-fluoro-5-methoxyisoindolin-1-one as a white solid, which was used in the next step without further purification (6.1 g, 88% yield, m/z: 182 [M+H]⁺ observed).

### tert-Butyl 6-fluoro-5-methoxy-1-oxoisoindoline-2-carboxylate

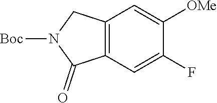 [SMILES: COc1cc2c(cc1F)C(=O)N(C(=O)OC(C)(C)C)C2]

To a solution of crude 6-fluoro-5-methoxyisoindolin-1-one (6.0 g, 33.1 mmol) in THF (60 mL) was added triethylamine (14 mL, 99.4 mmol), di-tert-butyl dicarbonate (8.7 g, 40 mmol) and DMAP (0.4 g, 3.31 mmol) and the mixture was stirred at rt for 6 h. The reaction mixture was diluted with water (200 mL) and extracted with EtOAc (2×200 mL). The combined organic layer was washed with saturated aqueous brine solution (100 mL), dried over anhydrous sodium sulfate, filtered and evaporated under reduced pressure. The crude compound was purified by normal phase SiO₂ chromatography (0-30% EtOAc/petroleum ether) to give tert-butyl 6-fluoro-5-methoxy-1-oxoisoindoline-2-carboxylate as a white solid (6.1 g, 65% yield, m/z: 226 [M-(tert-Butyl)+H]⁺ observed). ¹H NMR (400 MHz, CDCl₃): δ 7.55 (d, 1H), 6.99 (d, 1H), 4.69 (s, 2H), 3.97 (s, 3H), 1.59 (s, 9H).

### tert-Butyl 1-ethyl-6-fluoro-1-hydroxy-5-methoxyisoindoline-2-carboxylate

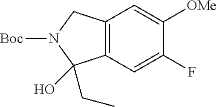 [SMILES: CCC1(O)c2cc(F)c(OC)cc2CN1C(=O)OC(C)(C)C]

To a cooled solution of tert-butyl 6-fluoro-5-methoxy-1-oxoisoindoline-2-carboxylate (6.0 g, 21 mmol) in THF (60 mL) was added dropwise ethyl magnesium bromide (3.0 M solution in Et₂O, 21.5 mL, 64.5 mmol) at 0° C. under an inert atmosphere for 10 min. The reaction mixture was slowly warmed to rt and stirred for 3 h. The reaction mixture was cooled to 0° C., quenched with saturated aqueous ammonium chloride solution (100 mL) and the resulting mixture was extracted with CH₂Cl₂ (2×200 mL). The combined organic layer was washed with saturated aqueous brine solution (100 mL), dried over anhydrous sodium sulfate, filtered and evaporated under reduced pressure. The residue was triturated with n-pentane (50 mL), filtered and dried under vacuum to give tert-butyl 1-ethyl-6-fluoro-1-hydroxy-5-methoxyisoindoline-2-carboxylate as a reddish gummy solid, which was used in the next step without further purification (4.2 g, 63% yield, m/z: 312 [M+H]⁺ observed).

### 1-Ethylidene-6-fluoro-5-methoxyisoindoline

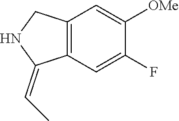

To a solution of crude tert-butyl 1-ethyl-6-fluoro-1-hydroxy-5-methoxyisoindoline-2-carboxylate (4.1 g, 13.1 mmol) in CH₂Cl₂ (50 mL) at −15° C. was added triethylsilane (17 mL, 105 mmol), followed by borontrifluoride-diethyl ether complex (3.2 mL, 26 mmol) under an inert atmosphere. The reaction mixture was slowly warmed to rt and stirred for 24 h. The reaction mixture was cooled to 0° C. and basified with saturated sodium bicarbonate solution. The resulting mixture was extracted with CH₂Cl₂ (2×200 mL). The combined organic layer was washed with saturated aqueous brine solution (100 mL), dried over anhydrous sodium sulfate, filtered and evaporated under reduced pressure to give crude 1-ethylidene-6-fluoro-5-methoxyisoindoline, which was used in the next step without further purification (2.2 g, 87% yield, m/z: 194 [M+H]⁺ observed). 1-Ethyl-6-fluoro-5-methoxyisoindoline:

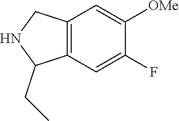

To a solution of crude 1-ethylidene-6-fluoro-5-methoxyisoindoline (2.2 g, 11.3 mmol) in MeOH (100 mL) was added palladium (10 wt. % loading on carbon, 1.0 g, 0.9 mmol) at rt and stirred under H₂ atmosphere (balloon) for 4 h. The reaction mixture was degassed with nitrogen, filtered through CELITE® and washed with MeOH (100 mL). The filtrate was evaporated under reduced pressure to give crude 1-ethyl-6-fluoro-5-methoxyisoindoline as an orange gummy solid, which was used in the next step without further purification (1.1 g, 50% yield, m/z: 196 [M+H]⁺ observed).

### 2-(2-Chloropyrimidin-4-yl)-1-ethyl-6-fluoro-5-methoxyisoindoline

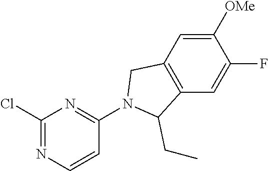 [SMILES: CCC1c2cc(F)c(OC)cc2CN1c1ccnc(Cl)n1]

To a solution of crude 1-ethyl-6-fluoro-5-methoxyisoindoline (1.0 g, 5.13 mmol) in THF (10 mL) was added N,N-diisopropylethylamine (2.7 mL, 15.4 mmol) and 2,4-dichloropyrimidine (0.83 g, 5.6 mmol) at rt and stirred for 2 h. The reaction mixture was diluted with water (100 mL) and extracted with EtOAc (2×200 mL). The combined organic layer was washed with saturated aqueous brine solution (100 mL), dried over anhydrous sulfate, filtered and evaporated under reduced pressure. The crude compound was purified by normal phase SiO₂ chromatography (0-30% EtOAc/petroleum ether) to give 2-(2-chloropyrimidin-4-yl)-1-ethyl-6-fluoro-5-methoxyisoindoline as an orange solid (0.51 g, 32% yield, m/z: 308 [M+H]⁺ observed). ¹H NMR (400 MHz, DMSO-d₆) δ 8.15-8.12 (m, 1H), 7.27-7.18 (m, 2H), 6.77-6.60 (m, 1H), 5.37-5.28 (m, 1H), 4.68-4.62 (m, 2H), 3.85 (s, 3H), 2.33-2.13 (m, 1H), 1.85-1.79 (m, 1H), 0.53-0.50 (m, 3H).

### 2-([2,2′-Bipyrimidin]-4-yl)-1-ethyl-6-fluoro-5-methoxyisoindoline

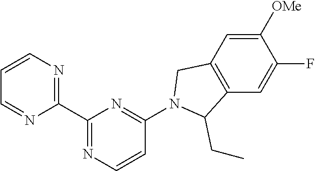 [SMILES: CCC1c2cc(F)c(OC)cc2CN1c1ccnc(-c2ncccn2)n1]

To a solution of 2-(2-chloropyrimidin-4-yl)-1-ethyl-6-fluoro-5-methoxyisoindoline (0.5 g, 1.6 mmol) in DMF (10 mL) was added 2-(tributylstannyl)pyrimidine (0.6 g, 1.6 mmol), tetraethylammonium chloride (0.27 g, 1.6 mmol) and potassium carbonate (0.45 g, 3.2 mmol) at rt. The reaction mixture was degassed with N₂ gas for 10 min. To this, PdCl₂(PPh₃)₂ (0.11 g, 0.16 mmol) was added and degassing with N₂ gas was continued for 10 min. The reaction mixture was stirred at 90° C. for 12 h, cooled to rt, diluted with water (100 mL), and extracted with EtOAc (2×100 mL). The organic layer was washed with saturated aqueous brine solution (100 mL), dried over anhydrous sodium sulfate, filtered and evaporated under reduced pressure. The residue was purified by reverse phase HPLC to give 2-([2,2′-bipyrimidin]-4-yl)-1-ethyl-6-fluoro-5-methoxyisoindoline as a white solid (0.24 g, 42% yield, m/z: 352 [M+H]⁺ observed). ¹H NMR (400 MHz, DMSO-d₆ at 90° ° C.) δ 8.91 (d, 2H), 8.39 (d, 1H), 7.54-7.52 (m, 1H), 7.21-7.17 (m, 2H), 6.71 (d, 1H), 5.34-5.38 (m, 1H), 4.81-4.61 (m, 2H), 3.86 (s, 3H), 2.35-2.31 (m, 1H), 1.89-1.83 (m, 1H), 0.58 (t, 3H).

### Example 64: 2-([2,2′-Bipyrimidin]-4-yl)-1-ethyl-6-fluoro-5-methoxyisoindoline (Single Enantiomer I)

### Example 65: 2-([2,2′-Bipyrimidin]-4-yl)-1-ethyl-6-fluoro-5-methoxyisoindoline (Single Enantiomer II)

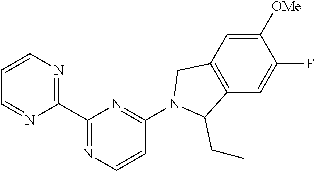 [SMILES: CCC1c2cc(F)c(OC)cc2CN1c1ccnc(-c2ncccn2)n1]

A mixture of enantiomers (190 mg) was separated by SFC (supercritical fluid chromatography) on a CHIRALCEL OD-H column using 30% MeOH to give 2-([2,2′-bipyrimidin]-4-yl)-1-ethyl-6-fluoro-5-methoxyisoindoline (single enantiomer I) as a white solid (faster eluting enantiomer, 53 mg, 28% yield, m/z: 352 [M+H]⁺ observed), and 2-([2,2′-bipyrimidin]-4-yl)-1-ethyl-6-fluoro-5-methoxyisoindoline (single enantiomer II) as a white solid (slower eluting enantiomer, 31 mg, 16% yield, m/z: 352 [M+H]⁺ observed).

### Example 64: 2-([2,2′-Bipyrimidin]-4-yl)-1-ethyl-6-fluoro-5-methoxyisoindoline (Single Enantiomer I)

m/z: 352 [M+H]⁺ observed. ¹H NMR (400 MHz, DMSO-d₆ at 90° C.) δ 8.91 (d, 2H), 8.39 (d, 1H), 7.54-7.52 (m, 1H), 7.21-7.17 (m, 2H), 6.71 (d, 1H), 5.34-5.38 (m, 1H), 4.81-4.61 (m, 2H), 3.86 (s, 3H), 2.35-2.31 (m, 1H), 1.89-1.83 (m, 1H), 0.58 (t, 3H).

### Example 65: 2-([2,2′-Bipyrimidin]-4-yl)-1-ethyl-6-fluoro-5-methoxyisoindoline (single enantiomer II)

m/z: 352 [M+H]⁺ observed. ¹H NMR (400 MHz, DMSO-d₆ at 90° ° C.) δ 8.91 (d, 2H), 8.39 (d, 1H), 7.54-7.52 (m, 1H), 7.21-7.17 (m, 2H), 6.71 (d, 1H), 5.34-5.38 (m, 1H), 4.81-4.61 (m, 2H), 3.86 (s, 3H), 2.35-2.31 (m, 1H), 1.89-1.83 (m, 1H), 0.58 (t, 3H).

The following examples were prepared in a similar manner as 2-([2,2′-bipyrimidin]-4-yl)-1-ethyl-6-fluoro-5-methoxyisoindoline from 1-ethyl-6-fluoro-5-methoxyisoindoline and an appropriately 5-substituted 2,4-dichloropyrimidine, followed by coupling with 2-(tributylstannyl)pyrimidine.

### Example 66: 1-Ethyl-6-fluoro-5-methoxy-2-(5-phenyl-[2,2′-bipyrimidin]-4-yl)isoindoline (Single Enantiomer I)

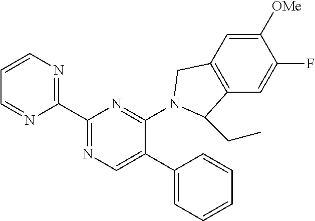 [SMILES: CCC1c2cc(F)c(OC)cc2CN1c1nc(-c2ncccn2)ncc1-c1ccccc1]

m/z: 428 [M+H]⁺ observed. ¹H NMR (400 MHz, DMSO-d₆) δ 8.87-8.82 (m, 2H), 8.64 (s, 1H), 7.53-7.50 (m, 1H), 7.25-7.18 (m, 5H), 7.05-7.02 (m, 2H), 5.42-5.36 (m, 1H), 4.98-4.67 (m, 2H), 3.85 (s, 3H), 2.43-2.41 (m, 1H), 2.07-1.78 (m, 1H), 0.59-052 (m, 3H).

### Example 67: 1-Ethyl-6-fluoro-5-methoxy-2-(5-phenyl-[2,2′-bipyrimidin]-4-yl)isoindoline (Single Enantiomer II)

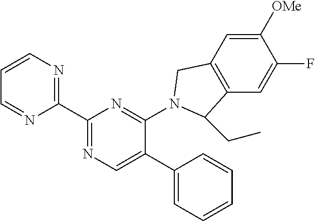 [SMILES: CCC1c2cc(F)c(OC)cc2CN1c1nc(-c2ncccn2)ncc1-c1ccccc1]

m/z: 428 [M+H]⁺ observed. ¹H NMR (400 MHz, DMSO-d₆) δ 8.87-8.82 (m, 2H), 8.64 (s, 1H), 7.53-7.50 (m, 1H), 7.25-7.18 (m, 5H), 7.05-7.02 (m, 2H), 5.42-5.36 (m, 1H), 4.98-4.67 (m, 2H), 3.85 (s, 3H), 2.43-2.41 (m, 1H), 2.07-1.78 (m, 1H), 0.59-052 (m, 3H).

### Example 68: 1-Ethyl-6-fluoro-5-methoxy-2-(5-methyl-[2,2′-bipyrimidin]-4-yl)isoindoline (Single Enantiomer I)

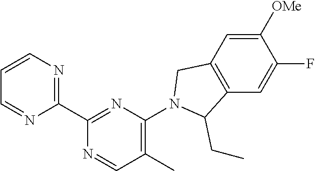 [SMILES: CCC1c2cc(F)c(OC)cc2CN1c1nc(-c2ncccn2)ncc1C]

m/z: 366 [M+H]⁺ observed. ¹H NMR (400 MHz, DMSO-d₆) δ 8.95-8.94 (m, 2H), 8.23 (s, 1H), 7.59-7.56 (m, 1H), 7.28-7.25 (m, 1H), 7.21-7.19 (m, 1H), 5.79-5.77 (m, 1H), 5.20-5.18 (m, 1H), 5.02-4.88 (m, 1H), 3.84 (s, 3H), 2.49 (s, 3H), 2.20-2.10 (m, 1H), 1.80-1.75 (m, 1H), 0.58 (t, 3H).

### Example 69: 1-Ethyl-6-fluoro-5-methoxy-2-(5-methyl-[2,2′-bipyrimidin]-4-yl)isoindoline (Single Enantiomer II)

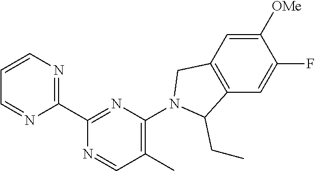 [SMILES: CCC1c2cc(F)c(OC)cc2CN1c1nc(-c2ncccn2)ncc1C]

m/z: 366 [M+H]⁺ observed. ¹H NMR (400 MHz, DMSO-d₆) δ 8.95-8.94 (m, 2H), 8.23 (s, 1H), 7.59-7.56 (m, 1H), 7.28-7.25 (m, 1H), 7.21-7.19 (m, 1H), 5.79-5.77 (m, 1H), 5.20-5.18 (m, 1H), 5.02-4.88 (m, 1H), 3.84 (s, 3H), 2.49 (s, 3H), 2.20-2.10 (m, 1H), 1.80-1.75 (m, 1H), 0.58 (t, 3H).

### Example 70: 1-Ethyl-6-fluoro-2-(5-fluoro-[2,2′-bipyrimidin]-4-yl)-5-methoxyisoindoline

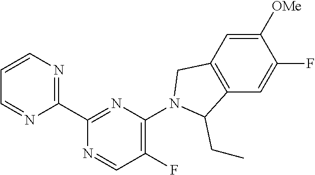 [SMILES: CCC1c2cc(F)c(OC)cc2CN1c1nc(-c2ncccn2)ncc1F]

m/z: 370 [M+H]⁺ observed. ¹H NMR (400 MHz, DMSO-d₆) δ 8.97-8.96 (m, 2H), 8.45-8.44 (m, 1H), 7.61-7.59 (m, 1H), 7.30-7.26 (m, 2H), 5.62 (m, 1H), 5.07-4.95 (m, 2H), 3.96 (s, 3H), 2.33-2.32 (m, 1H), 1.88-1.83 (m, 1H), 0.59-0.55 (m, 3H).

### Example 71: 1-Ethyl-6-fluoro-2-(5-fluoro-[2,2′-bipyrimidin]-4-yl)-5-methoxyisoindoline (Single Enantiomer I)

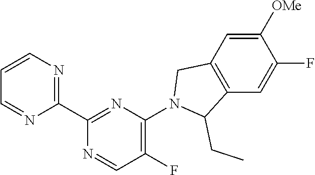 [SMILES: CCC1c2cc(F)c(OC)cc2CN1c1nc(-c2ncccn2)ncc1F]

m/z: 370 [M+H]⁺ observed. ¹H NMR (400 MHz, DMSO-d₆) δ 8.97-8.96 (m, 2H), 8.45-8.44 (m, 1H), 7.61-7.59 (m, 1H), 7.30-7.26 (m, 2H), 5.62 (m, 1H), 5.07-4.95 (m, 2H), 3.96 (s, 3H), 2.33-2.32 (m, 1H), 1.88-1.83 (m, 1H), 0.59-0.55 (m, 3H).

### Example 72: 1-Ethyl-6-fluoro-2-(5-fluoro-[2,2′-bipyrimidin]-4-yl)-5-methoxyisoindoline (Single Enantiomer II)

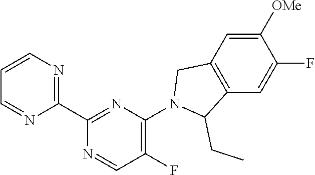 [SMILES: CCC1c2cc(F)c(OC)cc2CN1c1nc(-c2ncccn2)ncc1F]

m/z: 370 [M+H]⁺ observed. ¹H NMR (400 MHz, DMSO-d₆) δ 8.97-8.96 (m, 2H), 8.45-8.44 (m, 1H), 7.61-7.59 (m, 1H), 7.30-7.26 (m, 2H), 5.62 (m, 1H), 5.07-4.95 (m, 2H), 3.96 (s, 3H), 2.33-2.32 (m, 1H), 1.88-1.83 (m, 1H), 0.59-0.55 (m, 3H).

### Example 73: 1-Ethyl-6-fluoro-5-methoxy-2-(5-methoxy-[2,2′-bipyrimidin]-4-yl)isoindoline (Single Enantiomer I)

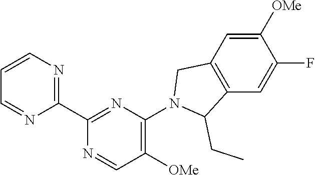 [SMILES: CCC1c2cc(F)c(OC)cc2CN1c1nc(-c2ncccn2)ncc1OC]

m/z: 382 [M+H]⁺ observed. ¹H NMR (400 MHz, DMSO-d₆) δ 8.94-8.93 (m, 2H), 8.16 (s, 1H), 7.55 (t, 1H), 7.27-7.24 (m, 2H), 5.73 (s, 1H), 5.07-4.96 (m, 2H), 3.96 (s, 3H), 3.84 (s, 3H), 2.32-2.18 (m, 1H), 1.83-1.78 (m, 1H), 0.57-0.53 (m, 3H).

### Example 74: 1-Ethyl-6-fluoro-5-methoxy-2-(5-methoxy-[2,2′-bipyrimidin]-4-yl)isoindoline (Single Enantiomer II)

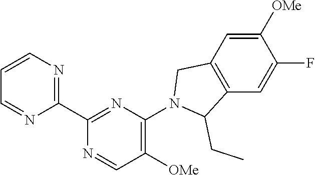 [SMILES: CCC1c2cc(F)c(OC)cc2CN1c1nc(-c2ncccn2)ncc1OC]

m/z: 382 [M+H]⁺ observed. ¹H NMR (400 MHz, DMSO-d₆) δ 8.94-8.93 (m, 2H), 8.16 (s, 1H), 7.55 (t, 1H), 7.27-7.24 (m, 2H), 5.73 (s, 1H), 5.07-4.96 (m, 2H), 3.96 (s, 3H), 3.84 (s, 3H), 2.32-2.18 (m, 1H), 1.83-1.78 (m, 1H), 0.57-0.53 (m, 3H).

### Example 75: 1-Ethyl-6-fluoro-2-(5-fluoro-[2,2′-bipyrimidin]-4-yl)isoindolin-5-ol

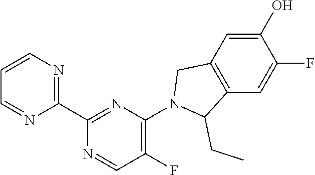 [SMILES: CCC1c2cc(F)c(O)cc2CN1c1nc(-c2ncccn2)ncc1F]

m/z: 356 [M+H]⁺ observed. ¹H NMR (400 MHz, CDCl₃) δ 8.98 (dt, J=4.9, 1.0 Hz, 2H), 8.32 (dt, J=5.5, 1.0 Hz, 1H), 7.41 (tt, J=4.9, 1.0 Hz, 1H), 6.89 (dd, J=8.9, 6.7 Hz, 2H), 6.79 (s, 1H), 5.63 (s, 1H), 4.98 (s, 2H), 2.17 (d, J=0.9 Hz, 1H), 1.83 (dd, J=13.4, 7.6 Hz, 1H), 0.68 (t, J=7.4 Hz, 3H).

### Example 76: 2-([2,2′-Bipyrimidin]-4-yl)-1-ethyl-5-fluoro-6-methoxyisoindoline

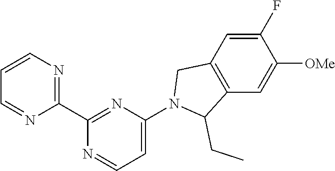 [SMILES: CCC1c2cc(OC)c(F)cc2CN1c1ccnc(-c2ncccn2)n1]

### 2-Bromo-4-fluoro-5-methoxybenzoic Acid

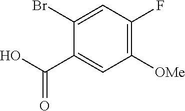 [SMILES: COc1cc(C(=O)O)c(Br)cc1F]

To a solution of 4-fluoro-3-methoxybenzoic acid (10 g, 58.8 mmol) in AcOH/H₂O (1:1, 100 mL) was added dropwise bromine (6.6 mL, 129 mmol) at rt, then the reaction mixture was heated at 50° C. for 16 h. The mixture was cooled to rt and poured into ice-cold water (100 mL) and stirred for 30 min. The white precipitate was filtered and dried under vacuum to give 2-bromo-4-fluoro-5-methoxybenzoic acid as an off-white solid (10.5 g, 72% yield). ¹H NMR (400 MHz, CDCl₃) δ 7.67 (d, 1H), 7.43 (d, 1H), 3.93 (s, 3H).

### Methyl 2-bromo-4-fluoro-5-methoxybenzoate

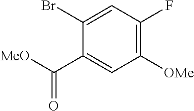 [SMILES: COC(=O)c1cc(OC)c(F)cc1Br]

To a solution of 2-bromo-4-fluoro-5-methoxybenzoic acid (10.5 g, 42.3 mmol) in MeOH (100 mL) was added concentrated sulfuric acid (1 mL), and the mixture was stirred at 60° C. for 16 h. The solvent was evaporated, and the residue was diluted with EtOAc (300 mL). The organic layer was washed with saturated aqueous bicarbonate solution (2×100 mL), dried over anhydrous sodium sulfate, filtered and evaporated reduced pressure to give methyl 2-bromo-4-fluoro-5-methoxybenzoate (10 g, 90% yield, m/z: 263 [M+H]⁺ observed). ¹H NMR (400 MHz, CDCl₃) δ 7.47 (d, 1H), 7.38 (d, 1H), 3.93 (s, 3H), 3.91 (s, 3H).

### Methyl 2-cyano-4-fluoro-5-methoxybenzoate

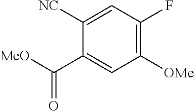 [SMILES: COC(=O)c1cc(OC)c(F)cc1C#N]

To a solution of methyl 2-bromo-4-fluoro-5-methoxybenzoate (10 g, 38.2 mmol) in DMF (50 mL) was added CuCN (5.15 g, 57.5 mmol). The reaction was heated at 150° C. and stirred for 4 h. The mixture was cooled to rt and poured into ice-cold water (50 mL) and stirred for 30 min. The white precipitate was filtered and dried under vacuum to give methyl 2-cyano-4-fluoro-5-methoxybenzoate (6.5 g, 81% yield, m/z: 210 [M+H]⁺ observed). ¹H NMR (400 MHz, CDCl₃) δ 7.71 (d, 1H), 7.47 (d, 1H), 4.02 (s, 3H), 4.01 (s, 3H).

### 5-Fluoro-6-methoxyisoindolin-1-one

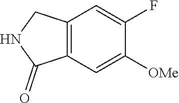 [SMILES: COc1cc2c(cc1F)CNC2=O]

To a solution of methyl 2-cyano-4-fluoro-5-methoxybenzoate (6.5 g, 31 mmol) in EtOH (200 mL) was added palladium (10 wt. % loading on carbon, 3.0 g, 3.0 mmol) and stirred under H₂ pressure (55 psi) in a steel bomb at rt for 16 h. The reaction mixture was degassed and back-filled with nitrogen, filtered through CELITE® and washed with MeOH (2×100 mL). The filtrate was evaporated under reduced pressure to give crude 5-fluoro-6-methoxyisoindolin-1-one as a white solid, which was used in the next step without further purification (4.5 g, 80% yield, m/z: 182 [M+H]⁺ observed).

### tert-Butyl 5-fluoro-6-methoxy-1-oxoisoindoline-2-carboxylate

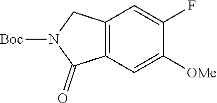 [SMILES: COc1cc2c(cc1F)CN(C(=O)OC(C)(C)C)C2=O]

To a solution of crude 5-fluoro-6-methoxyisoindolin-1-one (4.5 g, 24.8 mmol) in CH₂Cl₂ (50 mL) was added triethylamine (10.4 mL, 74.6 mmol), di-tert-butyl dicarbonate (6.50 g, 29.8 mmol) and DMAP (0.30 g, 2.5 mmol). The reaction mixture was stirred at rt for 16 h. The reaction mixture was diluted with water (200 mL) and extracted with CH₂Cl₂ (2×200 mL). The combined organic layer was washed with saturated aqueous brine solution (100 mL), dried over anhydrous sodium sulfate, filtered and evaporated under reduced pressure. The crude compound was purified by normal phase SiO₂ chromatography (0-20% EtOAc/petroleum ether) to give tert-butyl 5-fluoro-6-methoxy-1-oxoisoindoline-2-carboxylate as a white solid (4.1 g, 59% yield, m/z: 226 [M-(t-Butyl)+H]⁺ observed). ¹H NMR (400 MHz, CDCl₃) δ 7.44 (d, 1H), 7.16 (d, 1H), 4.67 (s, 2H), 3.94 (s, 3H), 1.59 (s, 9H).

### tert-Butyl 1-ethyl-5-fluoro-1-hydroxy-6-methoxyisoindoline-2-carboxylate

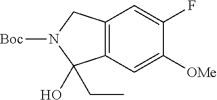 [SMILES: CCC1(O)c2cc(OC)c(F)cc2CN1C(=O)OC(C)(C)C]

To a solution of tert-butyl 5-fluoro-6-methoxy-1-oxoisoindoline-2-carboxylate (4.1 g, 14.6 mmol) in THF (60 mL) was added dropwise ethylmagnesium bromide (3.0M solution in Et₂O, 14.6 mL, 43.8 mmol) at 0° C. under an inert atmosphere over 10 min. The reaction mixture was warmed to rt over 30 min and stirred for 3 h. The reaction mixture was cooled to 0° C. and quenched with saturated aqueous ammonium chloride solution (100 mL). The resulting mixture was extracted with EtOAc (2×200 mL). The combined organic layer was washed with saturated aqueous brine solution (100 mL), dried over anhydrous sodium sulfate, filtered and evaporated under reduced pressure to give crude tert-butyl 1-ethyl-5-fluoro-1-hydroxy-6-methoxyisoindoline-2-carboxylate as reddish gummy solid, which was used in the next step without further purification (3.2 g, 70% yield, m/z: 294 [(M−H₂O)+H]⁺ observed). 1-Ethylidene-5-fluoro-6-methoxyisoindoline:

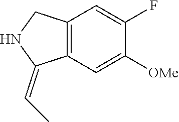

To a solution of crude tert-butyl 1-ethyl-5-fluoro-1-hydroxy-6-methoxyisoindoline-2-carboxylate (3.2 g, 10.3 mmol) in CH₂Cl₂ (50 mL) at −15° C. was added triethylsilane (13 mL, 82 mmol) followed by borontrifluoride-diethyl ether complex (2.5 mL, 20.6 mmol) under an inert atmosphere. The reaction mixture was slowly warmed to rt and stirred for 24 h, cooled to 0° C., and basified with saturated aqueous sodium bicarbonate solution. The resulting mixture was extracted with CH₂Cl₂ (2×200 mL). The combined organic layer was washed with saturated aqueous brine solution (100 mL), dried over anhydrous sodium sulfate, filtered and evaporated under reduced pressure to give crude 1-ethylidene-5-fluoro-6-methoxyisoindoline, which was used in the next step without further purification (2.1 g, >100% yield, 194 [M+H]⁺ observed).

### 1-Ethyl-5-fluoro-6-methoxyisoindoline

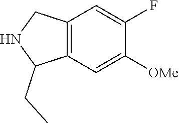

To a solution of crude 1-ethylidene-5-fluoro-6-methoxyisoindoline (2.0 g, 10.4 mmol) in MeOH (100 mL) was added palladium (10 wt. % loading on carbon, 1.0 g, 1.0 mmol) at rt and stirred under H₂ atmosphere (balloon) for 16 h. The reaction mixture was degassed and back-filled with N₂, filtered through the CELITE® and washed with MeOH (100 mL). The filtrate was evaporated under reduced pressure to give 1-ethyl-5-fluoro-6-methoxyisoindoline as an orange gummy solid, which was used in the next step without further purification (1.3 g, 64% yield, 196 [M+H]⁺ observed).

### 2-(2-Chloropyrimidin-4-yl)-1-ethyl-5-fluoro-6-methoxyisoindoline

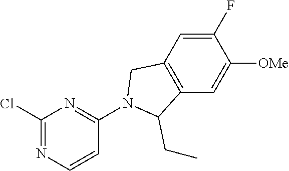 [SMILES: CCC1c2cc(OC)c(F)cc2CN1c1ccnc(Cl)n1]

To a solution of crude 1-ethyl-5-fluoro-6-methoxyisoindoline (1.2 g, 6.15 mmol) in THF 10 mL was added N,N-diisopropylethylamine (3.2 mL, 18.5 mmol), 2,4-dichloropyrimidine (1.0 g, 6.8 mmol) at rt and the reaction stirred for 2 h. The reaction mixture was diluted with water (100 mL) and extracted with EtOAc (2×200 mL). The combined organic layer was washed with saturated aqueous brine solution (100 mL), dried over anhydrous sodium sulfate, filtered and evaporated under reduced pressure. The crude compound was purified by normal phase SiO₂ chromatography (0-30% EtOAc/petroleum ether) to give 2-(2-chloropyrimidin-4-yl)-1-ethyl-5-fluoro-6-methoxyisoindoline as an orange solid (0.70 g, 37% yield, 308 [M+H]⁺ observed). ¹H NMR (400 MHz, DMSO-d₆) δ 8.12 (br s, 1H), 7.27-7.16 (m, 2H), 6.77-6.58 (m, 1H), 5.39-5.25 (m, 1H), 4.84-4.58 (m, 2H), 3.85 (s, 3H), 2.43-2.06 (m, 1H), 1.91-1.81 (m, 1H), 0.52-0.49 (m, 3H).

### 2-([2,2′-Bipyrimidin]-4-yl)-1-ethyl-5-fluoro-6-methoxyisoindoline

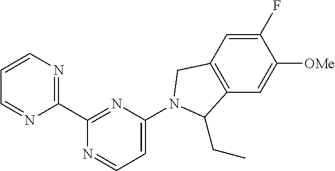 [SMILES: CCC1c2cc(OC)c(F)cc2CN1c1ccnc(-c2ncccn2)n1]

To a solution of 2-(2-chloropyrimidin-4-yl)-1-ethyl-5-fluoro-6-methoxyisoindoline (0.50 g, 1.6 mmol) in DMF (10 mL) was added 2-(tributylstannyl)pyrimidine (0.60 g, 1.6 mmol), tetraethyl ammonium chloride (0.27 g, 1.6 mmol) and potassium carbonate (0.45 g, 3.2 mmol) at rt and degassed with N₂ for 10 min. To this, PdCl₂(PPh₃)₂ (0.11 g, 0.16 mmol) was added and degassing with N₂ continued for 10 min. The reaction mixture was stirred at 90° C. for 12 h, cooled to rt, diluted with water (100 mL) and extracted with EtOAc (2×100 mL). The combined organic layer was washed with saturated aqueous brine solution (100 mL), dried over anhydrous sodium sulfate, filtered and evaporated under reduced pressure. The crude residue was purified by purified by reverse phase HPLC to give 2-([2,2′-bipyrimidin]-4-yl)-1-ethyl-5-fluoro-6-methoxyisoindoline as an off-white solid (0.28 g, 49% yield, 352 [M+H]⁺ observed). ¹H NMR (400 MHz, DMSO-d₆ at 90° C.) δ 8.93 (d, 2H), 8.40 (d, 1H), 7.55 (t, 1H), 7.26-7.14 (m, 2H), 6.72 (d, 1H), 5.42 (br s, 1H), 4.84-4.68 (m, 2H), 3.88 (s, 3H), 2.39-2.32 (m, 1H), 1.95-1.89 (m, 1H), 0.61 (t, 3H).

### Example 77: 2-([2,2′-Bipyrimidin]-4-yl)-1-ethyl-5-fluoro-6-methoxyisoindoline (Single Enantiomer I)

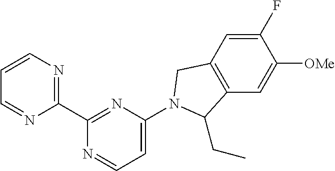 [SMILES: CCC1c2cc(OC)c(F)cc2CN1c1ccnc(-c2ncccn2)n1]

### Example 78: 2-([2,2′-Bipyrimidin]-4-yl)-1-ethyl-5-fluoro-6-methoxyisoindoline (Single Enantiomer II)

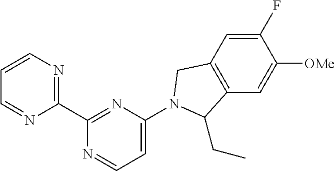 [SMILES: CCC1c2cc(OC)c(F)cc2CN1c1ccnc(-c2ncccn2)n1]

A mixture of enantiomers (260 mg) was separated by SFC (supercritical fluid chromatography) on a CHIRALCEL OD-H column using 25% MeOH (0.5% ammonia as modifier) to give 2-([2,2′-bipyrimidin]-4-yl)-1-ethyl-5-fluoro-6-methoxyisoindoline (single enantiomer I) as a white solid (faster eluting enantiomer, 30 mg, 12% yield, 352 [M+H]⁺ observed), and 2-([2,2′-bipyrimidin]-4-yl)-1-ethyl-5-fluoro-6-methoxyisoindoline (single enantiomer II) as a white solid (slower eluting enantiomer, 30 mg, 12% yield, 352 [M+H]⁺ observed).

### Example 77: 2-([2,2′-Bipyrimidin]-4-yl)-1-ethyl-5-fluoro-6-methoxyisoindoline (Single Enantiomer I)

m/z: 352 [M+H]⁺ observed. ¹H NMR (400 MHz, DMSO-d₆ at 90° ° C.) δ 8.93 (d, 2H), 8.40 (d, 1H), 7.55 (t, 1H), 7.26-7.14 (m, 2H), 6.72 (d, 1H), 5.42 (br s, 1H), 4.84-4.68 (m, 2H), 3.88 (s, 3H), 2.39-2.32 (m, 1H), 1.95-1.89 (m, 1H), 0.61 (t, 3H).

### Example 78: 2-([2,2′-Bipyrimidin]-4-yl)-1-ethyl-5-fluoro-6-methoxyisoindoline (Single Enantiomer II)

m/z: 352 [M+H]⁺ observed. ¹H NMR (400 MHz, DMSO-d₆ at 90° C.) δ 8.93 (d, 2H), 8.40 (d, 1H), 7.55 (t, 1H), 7.26-7.14 (m, 2H), 6.72 (d, 1H), 5.42 (br s, 1H), 4.84-4.68 (m, 2H), 3.88 (s, 3H), 2.39-2.32 (m, 1H), 1.95-1.89 (m, 1H), 0.61 (t, 3H).

### Example 79: 10-([2,2′-Bipyrimidin]-4-yl)-1,2,3,4-tetrahydro-1,4-(epiminomethano)naphthalene

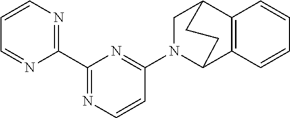 [SMILES: c1cnc(-c2nccc(N3CC4CCC3c3ccccc34)n2)nc1]

### 10-(2-Chloropyrimidin-4-yl)-1,2,3,4-tetrahydro-1,4-(epiminomethano)naphthalene

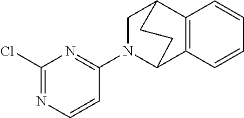 [SMILES: Clc1nccc(N2CC3CCC2c2ccccc23)n1]

To a solution of 2,4-dichloropyrimidine (57 mg, 0.38 mmol) in dry THF (1 mL) were added 1,2,3,4-tetrahydro-10λ²-1,4-(epaminomethano)naphthalene, hydrochloride salt (75 mg, 0.38 mmol), and N,N-diisopropylethylamine (170 uL, 0.96 mmol) and the reaction stirred at room temperature overnight. The reaction mixture was diluted with water (10 mL) and extracted with EtOAc (10 mL). The organic phase was dried over MgSO₄, filtered and the filtrate was evaporated under reduced pressure. The residue was purified using normal phase SiO₂ chromatography (0-50% EtOAc/Hexanes) to give 10-(2-chloropyrimidin-4-yl)-1,2,3,4-tetrahydro-1,4-(epiminomethano)naphthalene as a clear resin (90.7 mg, 87% yield, m/z: 272 [M+H]⁺ observed). ¹H NMR (400 MHz, CDCl₃) δ 7.94 (d, J=6.0 Hz, 1H), 7.35-7.19 (m, 4H), 6.14-5.91 (m, 2H), 3.54-3.26 (m, 2H), 3.12-2.97 (m, 1H), 2.24-2.12 (m, 1H), 1.99-1.86 (m, 1H), 1.69-1.55 (m, 2H).

### 10-([2,2′-Bipyrimidin]-4-yl)-1,2,3,4-tetrahydro-1,4-(epiminomethano)naphthalene

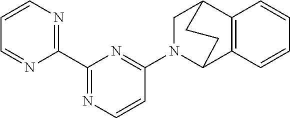 [SMILES: c1cnc(-c2nccc(N3CC4CCC3c3ccccc34)n2)nc1]

A microwave vial with stir bar was charged with 10-(2-chloropyrimidin-4-yl)-1,2,3,4-tetrahydro-1,4-(epiminomethano)naphthalene (90.7 mg, 0.33 mmol), bis(triphenylphosphine) palladium(II) dichloride (46.9 mg, 0.070 mmol), tributyl(pyrimidin-2-yl)stannane (265 μL, 0.83 mmol) and dry 1,4-dioxane (1 mL). The vial was back-flushed with nitrogen, sealed and heated at 110° C. in a reaction block behind a blast shield for 18 hours. The reaction mixture was cooled to rt and the volatiles were evaporated. The residue was partitioned between acetonitrile (10 mL) and hexane (25 mL). The acetonitrile lower layer (contained product by LC/MS) was collected and evaporated to dryness. The residue was purified by reverse phase HPLC. The desired fractions were combined and basified with saturated aqueous sodium bicarbonate solution to adjust pH to 9. The aqueous layer was extracted with CH₂Cl₂ (3×10 mL). The combined organic layer was dried over anhydrous sodium sulfate and filtered, and the filtrate was evaporated to give 10-([2,2′-bipyrimidin]-4-yl)-1,2,3,4-tetrahydro-1,4-(epiminomethano)naphthalene as a light brown foam (23.2 mg, 22% yield, m/z: 316 [M+H]⁺ observed). ¹H NMR (400 MHz, DMSO-d₆ at 50° C.) δ 9.06-8.80 (m, 2H), 8.36-8.21 (m, 1H), 7.64-7.48 (m, 1H), 7.39-7.11 (m, 4H), 6.53-6.31 (m, 1H), 6.23-5.97 (m, 1H), 3.70-3.49 (m, 1H), 3.48-3.38 (m, 1H), 3.13-3.00 (m, 1H), 2.21-2.05 (m, 1H), 2.03-1.82 (m, 1H), 1.66-1.37 (m, 2H).

The following examples were prepared in a similar manner as 10-([2,2′-bipyrimidin]-4-yl)-1,2,3,4-tetrahydro-1,4-(epiminomethano)naphthalene from an appropriately substituted 2,4-dichloropyrimidine and an appropriate amine.

### Example 80: 10-(5-Fluoro-[2,2′-bipyrimidin]-4-yl)-1,2,3,4-tetrahydro-1,4-(epiminomethano)naphthalene

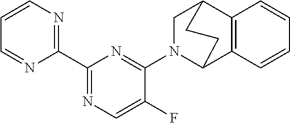 [SMILES: Fc1cnc(-c2ncccn2)nc1N1CC2CCC1c1ccccc12]

m/z: 334 [M+H]⁺ observed. ¹H NMR (400 MHz, CDCl₃) δ 8.94 (d, J=4.9 Hz, 2H), 8.21 (d, J=6.1 Hz, 1H), 7.35 (t, J=4.9 Hz, 1H), 7.30-7.26 (m, 4H), 4.01-3.91 (m, 1H), 3.64-3.52 (m, 1H), 3.41-3.34 (m, 1H), 2.40-2.30 (m, 1H), 2.05-1.96 (m, 1H), 1.73-1.58 (m, 3H).

### Example 81: 10-(5-Methyl-[2,2′-bipyrimidin]-4-yl)-1,2,3,4-tetrahydro-1,4-(epiminomethano)naphthalene

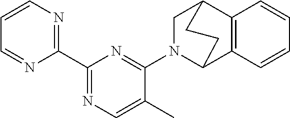 [SMILES: Cc1cnc(-c2ncccn2)nc1N1CC2CCC1c1ccccc12]

m/z: 330 [M+H]⁺ observed. ¹H NMR (400 MHz, DMSO-d₆) δ 9.06-8.98 (m, 2H), 8.15 (s, 1H), 7.66 (s, 1H), 7.34-7.30 (m, 2H), 7.29-7.18 (m, 2H), 5.96 (s, 1H), 4.01-3.90 (m, 1H), 3.51-3.36 (m, 2H), 2.36 (s, 3H), 2.32-2.21 (m, 1H), 2.01-1.90 (m, 1H), 1.58-1.37 (m, 2H).

### Example 82: 9-([2,2′-Bipyrimidin]-4-yl)-1,2,3,4-tetrahydro-1,4-epiminonaphthalene

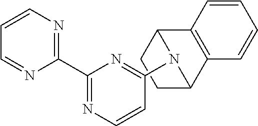 [SMILES: c1cnc(-c2nccc(N3C4CCC3c3ccccc34)n2)nc1]

A mixture of 1,2,3,4-tetrahydro-1,4-epiminonaphthalene hydrochloride (50.0 mg, 0.28 mmol), 4-chloro-2-pyrimidin-2-yl-pyrimidine (53.0 mg, 0.28 mmol), and N,N-diisopropylethylamine (144 μL, 0.83 mmol) in dry CH₃CN/THF (1:1, 2 mL) was heated at 50° C. for 48 hours. The volatiles were evaporated under reduced pressure and the residue was purified by reverse phase HPLC. The desired fractions were poured into saturated NaHCO₃ solution to adjust the pH to 9. The aqueous phase was extracted with CH₂Cl₂ (3×10 mL). The combined organic was dried over anhydrous sodium sulfate, filtered and evaporated under reduced pressure to give 9-([2,2′-bipyrimidin]-4-yl)-1,2,3,4-tetrahydro-1,4-epiminonaphthalene as an off-white solid (38.4 mg, 44% yield, m/z: 302 [M+H]⁺ observed). ¹H NMR (400 MHz, CDCl₃) δ 8.99 (d, J=4.8 Hz, 2H), 8.48 (d, J=5.8 Hz, 1H), 7.39 (t, J=4.8 Hz, 1H), 7.32-7.26 (m, 2H), 7.18-7.12 (m, 2H), 6.64 (d, J=5.9 Hz, 1H), 5.64 (s, 2H), 2.16 (d, J=8.5 Hz, 2H), 1.43 (d, J=7.6 Hz, 2H).

The following examples were prepared in a similar manner as 9-([2,2′-bipyrimidin]-4-yl)-1,2,3,4-tetrahydro-1,4-epiminonaphthalene from 4-chloro-2,2′-bipyrimidine and an appropriate amine.

### Example 83: 2-([2,2′-Bipyrimidin]-4-yl)-1,2,3,4-tetrahydro-1,4-methanoisoquinoline

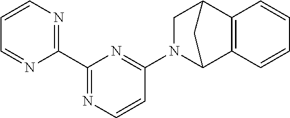 [SMILES: c1cnc(-c2nccc(N3CC4CC3c3ccccc34)n2)nc1]

m/z: 302 [M+H]⁺ observed. ¹H NMR (400 MHz, CDCl₃) δ 9.08-5.92 (m, 9H), 5.09 (s, 1H), 4.07-3.65 (m, 2H), 3.41-2.55 (m, 1H), 2.27-2.02 (m, 2H).

### Example 84: 9-([2,2′-Bipyrimidin]-5-yl)-6,7-dimethoxy-1,2,3,4-tetrahydro-1,4-epiminonaphthalene

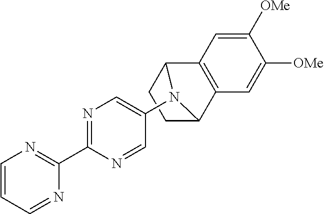 [SMILES: COc1cc2c(cc1OC)C1CCC2N1c1cnc(-c2ncccn2)nc1]

### tert-Butyl 6,7-dimethoxy-1,4-dihydro-1,4-epiminonaphthalene-9-carboxylate

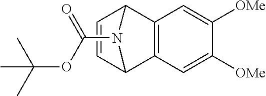 [SMILES: COc1cc2c(cc1OC)C1C=CC2N1C(=O)OC(C)(C)C]

To a stirred solution of (4,5-dimethoxy-2-trimethylsilyl-phenyl) trifluoromethanesulfonate (2.0 g, 5.6 mmol) and tert-butyl pyrrole-1-carboxylate (0.93 mL, 5.6 mmol) in dry acetonitrile (10 mL) was added anhydrous cesium fluoride (0.93 g, 6.1 mmol). The suspension was stirred at rt under N₂ atmosphere for 18 h. The reaction mixture was warmed to 50° C. and stirred for 7 h. The mixture was cooled to rt, poured into water (25 mL) and extracted with EtOAc (2×25 mL). The combined organic layer was washed with water (2×15 mL) and saturated aqueous brine solution (10 mL). The organic layer was dried over magnesium sulfate, filtered and evaporated under reduced pressure. The residue was purified via normal phase SiO₂ chromatography (0-20% EtOAc/hexanes) to give tert-butyl 6,7-dimethoxy-1,4-dihydro-1,4-epiminonaphthalene-9-carboxylate as a pale yellow oil (1.4 g, 82% yield, 248 [(M-tButyl)+H]⁺ observed). ¹H NMR (400 MHz, CDCl₃) δ 7.11-6.84 (m, 4H), 5.52-5.33 (m, 2H), 3.84 (s, 6H), 1.37 (s, 9H).

### tert-Butyl 6,7-dimethoxy-1,2,3,4-tetrahydro-1,4-epiminonaphthalene-9-carboxylate

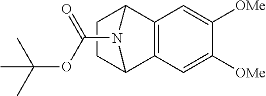 [SMILES: COc1cc2c(cc1OC)C1CCC2N1C(=O)OC(C)(C)C]

A flask was charged with palladium (10 wt. % loading on carbon, 250 mg, 0.23 mmol) and to this was carefully added a solution of tert-butyl 6,7-dimethoxy-1,4-dihydro-1,4-epiminonaphthalene-9-carboxylate (252 mg, 0.83 mmol) in EtOAc (15 mL). The suspension was stirred under an atmosphere of H₂ (balloon). The reaction mixture was stirred for 18 hours. The mixture was degassed and back-flushed with N₂. The suspension was filtered through a plug of CELITE®, rinsed with CH₂Cl₂ (2×20 mL), and the filtrate was evaporated under reduced pressure to give crude tert-butyl 6,7-dimethoxy-1,2,3,4-tetrahydro-1,4-epiminonaphthalene-9-carboxylate as a red-orange resin, which was used in the next step without further purification (193 mg, 76%, 250 [(M-tButyl)+H]⁺ observed). ¹H NMR (400 MHz, CDCl₃) δ 6.86 (s, 2H), 5.05 (s, 2H), 3.86 (s, 6H), 2.08 (d, J=8.9 Hz, 2H), 1.40 (s, 9H), 1.23 (d, J=7.8 Hz, 2H).

### 6,7-Dimethoxy-1,2,3,4-tetrahydro-1,4-epiminonaphthalene

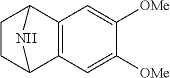 [SMILES: COc1cc2c(cc1OC)C1CCC2N1]

To a solution of crude tert-butyl 6,7-dimethoxy-1,2,3,4-tetrahydro-1,4-epiminonaphthalene-9-carboxylate (515 mg, 1.7 mmol) in dry Et₂O (5 mL) at 5° C. under a nitrogen atmosphere was added dropwise hydrogen chloride (1.0M solution in Et₂O, 3.4 mL, 3.4 mmol). The mixture was slowly warmed to rt and stirred for 24 h. The precipitate was filtered, rinsed with Et₂O and dried under high vacuum to give 6,7-dimethoxy-1,2,3,4-tetrahydro-1,4-epiminonaphthalene hydrochloride as a tan solid, which was used in the next step without further purification (154 mg, 38% yield, 189 [(M-NH₂)+H]⁺ observed).

### 9-([2,2′-Bipyrimidin]-5-yl-6,7-dimethoxy-1,2,3,4-tetrahydro-1,4-epiminonaphthalene

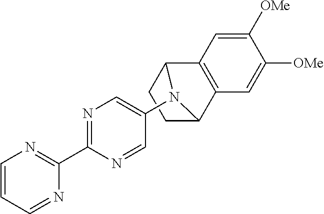 [SMILES: COc1cc2c(cc1OC)C1CCC2N1c1cnc(-c2ncccn2)nc1]

A microwave vial with a stir bar was charged with crude 6,7-dimethoxy-1,2,3,4-tetrahydro-1,4-epiminonaphthalene (152 mg, 0.17 mmol), 5-bromo-2-pyrimidin-2-yl-pyrimidine (44 mg, 0.19 mmol), cesium carbonate (166 mg, 0.51 mmol), Xantphos Pd G3 (9.8 mg, 0.02 mmol) and dry 1,4-dioxane (1 mL). The vial was backflushed with N₂, sealed and heated at 120° C. for 18 hours. The mixture was cooled to rt, diluted with CH₂Cl₂ (10 mL) and filtered through a plug of CELITE®, rinsed with CH₂Cl₂ (10 mL) and the filtrate was evaporated under reduced pressure. The residue was purified by reverse phase HPLC. The desired fractions were collected, poured into saturated aqueous sodium bicarbonate solution to adjust the pH to 9 and extracted with CH₂Cl₂ (3×10 mL). The combined organic layer was dried over anhydrous sodium sulfate, filtered and evaporated under reduced pressure to give 9-([2,2′-bipyrimidin]-5-yl)-6,7-dimethoxy-1,2,3,4-tetrahydro-1,4-epiminonaphthalene as an off-white solid (28 mg, 45% yield, 362 [M+H]⁺ observed). ¹H NMR (400 MHz, CDCl₃) δ 8.93 (d, J=4.8 Hz, 2H), 8.53 (s, 2H), 7.32 (t, J=4.8 Hz, 1H), 6.89 (s, 2H), 5.23-4.98 (m, 2H), 3.84 (s, 6H), 2.30-2.17 (m, 2H), 1.48-1.37 (m, 2H).

The following examples were prepared in a similar manner as 9-([2,2′-bipyrimidin]-5-yl)-6,7-dimethoxy-1,2,3,4-tetrahydro-1,4-epiminonaphthalene from 1,2,3,4-tetrahydro-1,4-epiminonaphthalene and an appropriately 5-substituted 2,4-dichloropyrimidine, followed by coupling with 2-(tributylstannyl)pyrimidine.

### Example 85: 9-(5-Methyl-[2,2′-bipyrimidin]-4-yl)-1,2,3,4-tetrahydro-1,4-epiminonaphthalene

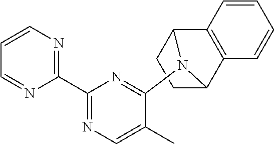 [SMILES: Cc1cnc(-c2ncccn2)nc1N1C2CCC1c1ccccc12]

m/z: 316 [M+H]⁺ observed. ¹H NMR (400 MHz, CDCl₃) δ 8.97 (d, J=4.8 Hz, 2H), 8.33 (s, 1H), 7.36 (t, J=4.8 Hz, 1H), 7.29 (dd, J=5.3, 3.1 Hz, 2H), 7.15 (dd, J=5.3, 3.0 Hz, 2H), 5.79-5.73 (m, 2H), 2.40 (s, 3H), 2.21-2.10 (m, 2H), 1.44-1.37 (m, 2H).

### Example 86: 9-(5-Fluoro-[2,2′-bipyrimidin]-4-yl)-1,2,3,4-tetrahydro-1,4-epiminonaphthalene

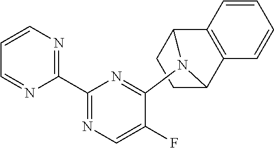 [SMILES: Fc1cnc(-c2ncccn2)nc1N1C2CCC1c1ccccc12]

m/z: 320 [M+H]⁺ observed. ¹H NMR (400 MHz, CDCl₃) δ 8.97 (d, J=4.7 Hz, 2H), 8.32 (s, 1H), 7.38 (t, J=4.9 Hz, 1H), 7.30 (dd, J=5.3, 3.1 Hz, 2H), 7.16 (dd, J=5.3, 3.1 Hz, 2H), 6.05-5.75 (m, 2H), 2.29-2.10 (m, 2H), 1.48-1.42 (m, 2H).

The following examples were prepared in a similar manner as 9-([2,2′-bipyrimidin]-5-yl)-6,7-dimethoxy-1,2,3,4-tetrahydro-1,4-epiminonaphthalene from 6,7-dimethoxy-1,2,3,4-tetrahydro-1,4-epiminonaphthalene and an appropriately 5-substituted 2,4-dichloropyrimidine, followed by coupling with 2-(tributylstannyl)pyrimidine.

### Example 87: 9-(5-Fluoro-[2,2′-bipyrimidin]-4-yl)-6,7-dimethoxy-1,2,3,4-tetrahydro-1,4-epiminonaphthalene

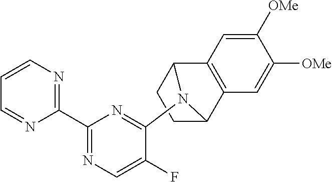 [SMILES: COc1cc2c(cc1OC)C1CCC2N1c1nc(-c2ncccn2)ncc1F]

m/z: 380 [M+H]⁺ observed. ¹H NMR (400 MHz, CDCl₃) δ 8.97 (s, 2H), 8.32 (s, 1H), 7.40 (s, 1H), 6.91 (s, 2H), 5.83 (s, 2H), 3.86 (s, 6H), 2.16 (d, J=8.6 Hz, 2H), 1.40 (d, J=9.1 Hz, 2H).

### Example 88: 6,7-Dimethoxy-9-(5-methyl-[2,2′-bipyrimidin]-4-yl)-1,2,3,4-tetrahydro-1,4-epiminonaphthalene

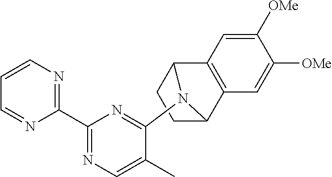 [SMILES: COc1cc2c(cc1OC)C1CCC2N1c1nc(-c2ncccn2)ncc1C]

m/z: 376 [M+H]⁺ observed. ¹H NMR (400 MHz, CDCl₃) δ 8.97 (d, J=4.8 Hz, 2H), 8.33 (s, 1H), 7.37 (t, J=4.8 Hz, 1H), 6.91 (s, 2H), 5.72 (s, 2H), 3.86 (s, 6H), 2.40 (s, 3H), 2.13 (d, J=8.7 Hz, 2H), 1.36 (d, J=7.5 Hz, 2H).

### Example 89: 2-([2,2′-Bipyrimidin]-5-yl)-1-ethyl-6-fluoro-5-methoxyisoindoline (Single Enantiomer I)

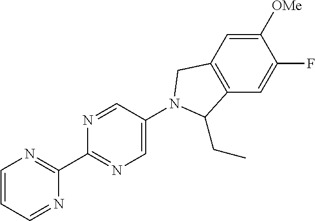 [SMILES: CCC1c2cc(F)c(OC)cc2CN1c1cnc(-c2ncccn2)nc1]

### Example 90: 2-([2,2′-Bipyrimidin]-5-yl)-1-ethyl-6-fluoro-5-methoxyisoindoline (Single Enantiomer II)

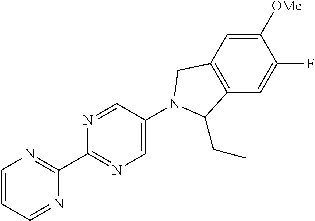 [SMILES: CCC1c2cc(F)c(OC)cc2CN1c1cnc(-c2ncccn2)nc1]

To a solution of 5-bromo-2,2′-bipyrimidine (400 mg, 1.69 mmol) in DMF/toluene (1:1, 8 mL) was added 1-ethyl-6-fluoro-5-methoxyisoindoline (396 mg, 2.03 mmol) and Cs₂CO₃ (1.1 g, 3.38 mmol) at rt. The reaction mixture was degassed with argon for 5 minutes. To the obtained mixture was added Pd₂(dba)₃ (155 mg, 0.169 mmol) and SPhos (173 mg, 0.42 mmol). The reaction mixture was heated at 150° C. in a microwave reactor for 1 h, cooled to rt, diluted with EtOAc (50 mL) and filtered through CELITE®. The filtrate was washed with ice-cold saturated aqueous brine solution (2×20 mL), dried with anhydrous sodium sulfate and evaporated under reduced pressure. The crude residue was purified by reverse phase HPLC to give 2-([2,2′-bipyrimidin]-5-yl)-1-ethyl-6-fluoro-5-methoxyisoindoline as a white solid (70 mg, 12% yield, m/z: 352 [M+H]⁺ observed). ¹H NMR (400 MHz, DMSO-d₆) δ 8.92-8.91 (m, 2H), 8.44 (s, 2H), 7.51-7.49 (m, 1H), 7.31-7.23 (m, 2H), 5.37 (s, 1H), 4.83-4.67 (m, 2H), 3.87 (s, 3H), 2.15-2.12 (m, 1H), 1.87-1.85 (m, 1H), 0.55-0.52 (m, 3H).

The mixture of enantiomers (70 mg) was separated by SFC (supercritical fluid chromatography) on a Chiralcel OD-H using 30% EtOH to give 2-([2,2′-bipyrimidin]-5-yl)-1-ethyl-6-fluoro-5-methoxyisoindoline (single enantiomer I) as a white solid (faster eluting enantiomer, 18 mg, 26% yield, m/z: 352 [M+H]⁺ observed), and 2-([2,2′-bipyrimidin]-5-yl)-1-ethyl-6-fluoro-5-methoxyisoindoline (single enantiomer II) as a white solid (slower eluting enantiomer, 20 mg, 29% yield, m/z: 352 [M+H]⁺ observed).

### Example 89: 2-([2,2′-Bipyrimidin]-5-yl)-1-ethyl-6-fluoro-5-methoxyisoindoline (Single Enantiomer I)

m/z: 352 [M+H]⁺ observed. ¹H NMR (400 MHz, DMSO-d₆) δ 8.92-8.91 (m, 2H), 8.44 (s, 2H), 7.51-7.49 (m, 1H), 7.31-7.23 (m, 2H), 5.37 (s, 1H), 4.83-4.67 (m, 2H), 3.87 (s, 3H), 2.15-2.12 (m, 1H), 1.87-1.85 (m, 1H), 0.55-0.52 (m, 3H).

### Example 90: 2-([2,2′-Bipyrimidin]-5-yl)-1-ethyl-6-fluoro-5-methoxyisoindoline (Single Enantiomer II)

m/z: 352 [M+H]⁺ observed. ¹H NMR (400 MHz, DMSO-d₆) δ 8.92-8.91 (m, 2H), 8.44 (s, 2H), 7.51-7.49 (m, 1H), 7.31-7.23 (m, 2H), 5.37 (s, 1H), 4.83-4.67 (m, 2H), 3.87 (s, 3H), 2.17-2.10 (m, 1H), 1.87-1.84 (m, 1H), 0.55-0.52 (m, 3H).

### Example 91: 4-(1-Ethyl-5,6-dimethoxyisoindolin-2-yl)-2-(pyrimidin-2-yl)furo[3,2-d]pyrimidine

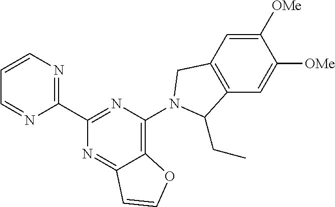 [SMILES: CCC1c2cc(OC)c(OC)cc2CN1c1nc(-c2ncccn2)nc2ccoc12]

### 2-Chloro-4-(1-ethyl-5,6-dimethoxyisoindolin-2-yl)furo[3,2-d]pyrimidine

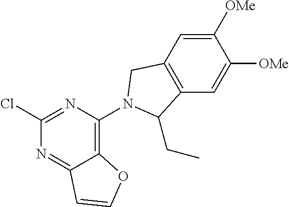 [SMILES: CCC1c2cc(OC)c(OC)cc2CN1c1nc(Cl)nc2ccoc12]

To a solution of 1-ethyl-5,6-dimethoxyisoindoline, hydrochloride salt (0.1 g, 0.41 mmol.) in THF (5 mL) was added N,N-diisopropylethylamine (0.17 mL, 1.23 mmol), 2,4-dichlorofuro[3,2-d]pyrimidine (90 mg, 0.45 mmol) and the reaction mixture was stirred at rt for 8 h. The reaction mixture was diluted with H₂O (25 mL) and extracted with EtOAc (2×25 mL). The combined organic layer was washed with saturated aqueous brine solution (25 mL), dried over anhydrous sodium sulfate, filtered and evaporated under reduced pressure. The residue was purified normal phase SiO₂ chromatography (0-50% EtOAc/hexanes) to give 2-chloro-4-(1-ethyl-5,6-dimethoxyisoindolin-2-yl)furo[3,2-d]pyrimidine as a white solid (120 mg, 69% yield, m/z: 360 [M+H]⁺ observed).

### 4-(1-Ethyl-5,6-dimethoxyisoindolin-2-yl)-2-(pyrimidin-2-yl)furo[3,2-d]pyrimidine

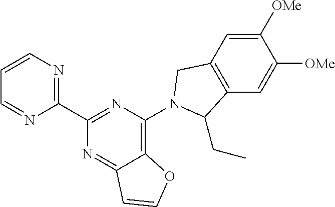 [SMILES: CCC1c2cc(OC)c(OC)cc2CN1c1nc(-c2ncccn2)nc2ccoc12]

To a solution of 2-chloro-4-(1-ethyl-5,6-dimethoxyisoindolin-2-yl)furo[3,2-d]pyrimidine (0.12 g, 0.33 mmol) in DMF (5 mL) was added 2-(tributylstannyl)pyrimidine (0.25 g, 1.4 mmol), followed by potassium carbonate (0.14 g, 1 mmol). The mixture was degassed with N₂ for 10 min. Then bis(triphenylphosphine)palladium(II) dichloride (23 mg, 0.033 mmol) was added and the solution was degassed with N₂ for 5 min. The reaction mixture was stirred at 100° C. for 24 h. The reaction mixture was cooled to rt, diluted with H₂O (25 mL) and extracted with EtOAc (2×25 mL). The combined organic layer was washed with saturated aqueous brine solution (25 mL), dried over anhydrous sodium sulfate, filtered and evaporated under reduced pressure. The crude residue was purified by reverse phase HPLC to give 4-(1-ethyl-5,6-dimethoxyisoindolin-2-yl)-2-(pyrimidin-2-yl)furo[3,2-d]pyrimidine as a white solid (22 mg, 17% yield, m/z: 404 [M+H]⁺ observed). ¹H NMR (400 MHz, CDCl₃) δ 9.00 (d, J=4.9 Hz, 2H), 7.85 (s, 1H), 7.38 (t, J=4.8 Hz, 1H), 7.06 (s, 1H), 6.87 (s, 1H), 6.80 (s, 1H), 5.90 (s, 1H), 5.34 (d, J=14.6 Hz, 1H), 5.14 (s, 1H), 3.92 (d, J=2.0 Hz, 6H), 2.20-2.30 (m, 1H), 2.00 (d, J=10.1 Hz, 1H), 0.73 (s, 3H).

The following examples were prepared in a similar manner as 4-(1-ethyl-5,6-dimethoxyisoindolin-2-yl)-2-(pyrimidin-2-yl)furo[3,2-d]pyrimidine from 1-ethyl-5,6-dimethoxyisoindoline and an appropriately substituted 2,4-dichloropyrimidine, followed by coupling with 2-(tributylstannyl)pyrimidine.

### Example 92: 4-(1-Ethyl-5,6-dimethoxyisoindolin-2-yl)-2-(pyrimidin-2-yl)-5,7-dihydrofuro[3,4-d]pyrimidine

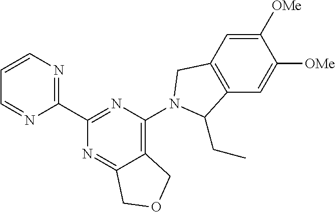 [SMILES: CCC1c2cc(OC)c(OC)cc2CN1c1nc(-c2ncccn2)nc2c1COC2]

m/z: 406 [M+H]⁺ observed. ¹H NMR (400 MHz, CDCl₃) δ 8.99 (d, J=4.8 Hz, 2H), 7.39 (m, 1H), 6.82 (s, 1H), 6.76 (s, 1H), 5.51 (d, J=21.1 Hz, 2H), 5.18-4.95 (m, 5H), 3.90 (d, J=1.6 Hz, 6H), 1.95-1.82 (m, 1H), 1.60 (s, 1H), 0.70 (s, 3H).

### Example 93: 7-(1-Ethyl-5,6-dimethoxy-isoindolin-2-yl)-5-pyrimidin-2-yl-thiazolo[5,4-d]pyrimidine

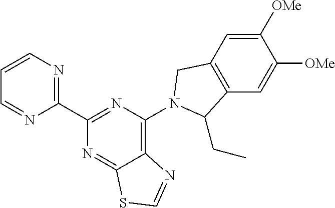 [SMILES: CCC1c2cc(OC)c(OC)cc2CN1c1nc(-c2ncccn2)nc2scnc12]

m/z: 421 [M+H]⁺ observed. ¹H NMR (400 MHz, CDCl₃) δ 9.03 (m, 2H), 8.69 (d, J=10.4 Hz, 1H), 7.40 (d, J=4.8 Hz, 1H), 6.89 (s, 1H), 6.81 (d, J=7.2 Hz, 1H), 6.35 (s, 0.5H), 6.07 (s, 0.5H), 5.61 (d, J=16 Hz, 0.5H), 5.40 (m, 1H), 5.15 (d, J=16 Hz, 0.5H), 3.92 (s, 3H), 3.91 (s, 3H), 2.80-2.70 (m, 0.5H), 2.27-2.19 (m, 0.5H), 2.10-1.90 (m, 1H), 0.77-0.66 (m, 3H).

### Example 94: 4-(1-Ethyl-5,6-dimethoxy-isoindolin-2-yl)-2-pyrimidin-2-yl-6,7-dihydro-5H-cyclopenta[d]pyrimidine

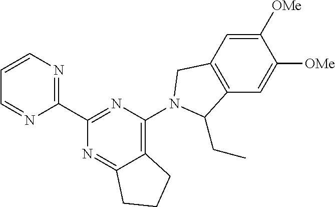 [SMILES: CCC1c2cc(OC)c(OC)cc2CN1c1nc(-c2ncccn2)nc2c1CCC2]

m/z: 404 [M+H]⁺ observed. ¹H NMR (400 MHz, CDCl₃) δ 8.96 (br s, 2H), 7.34 (d, J=3.2 Hz, 1H), 6.81 (s, 1H), 6.75 (d, J=3.2 Hz, 1H), 5.80-5.60 (m, 1H), 5.08 (brs, 2H), 3.89 (s, 6H), 3.85-3.20 (m, 2H), 3.10-3.00 (m, 2H), 2.20-2.05 (m, 3H), 1.90-1.80 (m, 1H), 0.70-0.67 (m, 3H).

### Example 95: 4-(1-Ethyl-5,6-difluoroisoindolin-2-yl)pyrimidine-2-carboxylic Acid

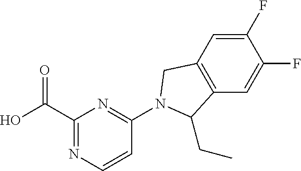 [SMILES: CCC1c2cc(F)c(F)cc2CN1c1ccnc(C(=O)O)n1]

### Ethyl 4-(1-ethyl-5,6-difluoroisoindolin-2-yl)pyrimidine-2-carboxylate

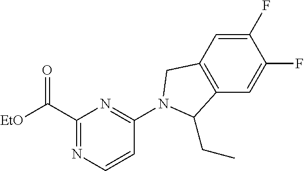 [SMILES: CCOC(=O)c1nccc(N2Cc3cc(F)c(F)cc3C2CC)n1]

To a solution of 2-(2-chloropyrimidin-4-yl)-1-ethyl-5,6-difluoroisoindoline (0.50 g, 1.7 mmol) in EtOH (10 mL) was added triethylamine (0.7 mL, 5 mmol) at rt in a steel bomb. The reaction mixture was purged with argon gas for 10 min, followed by the addition of diphenyl phosphoryl azide (70 mg, 0.25 mmol) and Pd(OAc)₂ (36 mg, 0.16 mmol). The reaction mixture was purged with argon gas for 5 min. The mixture was then stirred under CO pressure (200 psi) at 100° C. for 16 h. The reaction mixture was cooled to rt, filtered through a CELITE® and washed with EtOAc (20 mL). The filtrate was evaporated under reduced pressure to give crude ethyl 4-(1-ethyl-5,6-difluoroisoindolin-2-yl)pyrimidine-2-carboxylate as a sticky liquid, which was used in the next step without further purification (0.5 g, 89% yield, m/z: 334 [M+H]⁺ observed).

### 4-(1-Ethyl-5,6-difluoroisoindolin-2-yl)pyrimidine-2-carboxylic Acid

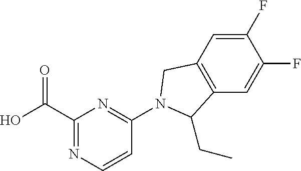 [SMILES: CCC1c2cc(F)c(F)cc2CN1c1ccnc(C(=O)O)n1]

To a solution of crude ethyl 4-(1-ethyl-5,6-difluoroisoindolin-2-yl)pyrimidine-2-carboxylate (0.5 g, 1.5 mmol) in THF/EtOH (2:1; 15 mL) was added a solution of lithium hydroxide monohydrate (0.25 g, 6 mmol) of in H₂O (5 mL) at rt. The reaction mixture was stirred for 4 h and then concentrated under reduced pressure. The aqueous layer was acidified using HCl (10% solution in H₂O) and extracted with CH₂Cl₂/MeOH (9:1, 2×30 mL). The combined organic layer was washed with saturated aqueous brine solution (10 mL), dried over anhydrous sodium sulfate, filtered and evaporated to dryness. The residue was purified by purified by reverse phase HPLC to give 4-(1-ethyl-5,6-difluoroisoindolin-2-yl)pyrimidine-2-carboxylic acid as an off-white solid (0.135 g, 26% yield, m/z: 306 [M+H]⁺ observed). ¹H NMR (300 MHz, DMSO-d₆, at 90° C.) δ 8.31 (d, 1H), 7.48-7.39 (m, 2H), 6.74 (d, 1H), 5.43-5.41 (m, 1H), 4.86-4.68 (m, 2H), 2.30-2.25 (m, 1H), 1.92-1.83 (m, 1H), 0.58 (t, 3H).

The following examples were prepared in a similar manner as 4-(1-ethyl-5,6-difluoroisoindolin-2-yl)pyrimidine-2-carboxylic acid from 1-ethyl-6-fluoro-5-methoxyisoindoline and 2,4-dicholoropyrimidine.

### Example 96: 4-(1-Ethyl-6-fluoro-5-methoxyisoindolin-2-yl)pyrimidine-2-carboxylic Acid

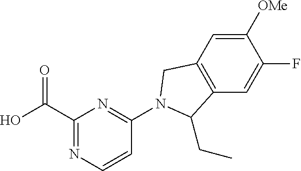 [SMILES: CCC1c2cc(F)c(OC)cc2CN1c1ccnc(C(=O)O)n1]

m/z: 318 [M+H]⁺ observed. ¹H NMR (400 MHz, DMSO-d₆) δ 11.98 (br s, 1H), 8.31 (br s, 1H), 7.29-7.26 (m, 1H), 7.22 (br s, 1H), 6.83-6.70 (m, 1H) 5.47-5.25 (m, 1H), 4.69 (s, 2H), 3.05 (s, 3H), 2.42-2.45 (m, 1H), 1.90-1.84 (m, 1H), 0.52-0.48 (m, 3H).

### Example 97: 5-(1-Ethyl-5,6-dimethoxyisoindolin-2-yl)pyrimidine-2-carboxylic Acid

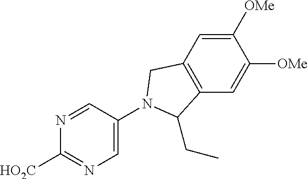 [SMILES: CCC1c2cc(OC)c(OC)cc2CN1c1cnc(C(=O)O)nc1]

### tert-Butyl 5-bromopyrimidine-2-carboxylate

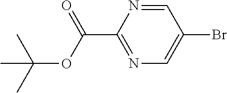

To a stirred solution of 5-bromopyrimidine-2-carboxylic acid (5 g, 25 mmol) in tert-butanol (50 mL) was added di-tert-butyl dicarbonate (11 g, 50 mmol) and DMAP (0.3 g, 2.48 mmol) at rt under inert atmosphere. The reaction mixture was stirred at 60° C. for 7 h. The reaction mixture was cooled to rt and diluted with aqueous ammonium chloride solution (50 mL) and extracted with EtOAc (3×100 mL). The combined organic layer was dried over anhydrous sodium sulfate and concentrated under reduced pressure. The residue was purified via normal phase SiO₂ chromatography (0-10% EtOAc/petroleum ether) to give tert-butyl 5-bromopyrimidine-2-carboxylate as a white solid (5.1 g, 79% yield, 258 [M+H]⁺ observed).

### tert-Butyl 5-(1-ethyl-5,6-dimethoxyisoindolin-2-yl)pyrimidine-2-carboxylate

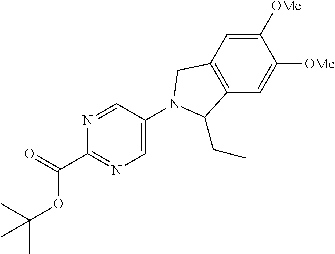 [SMILES: CCC1c2cc(OC)c(OC)cc2CN1c1cnc(C(=O)OC(C)(C)C)nc1]

To a solution of 1-ethyl-5,6-dimethoxyisoindoline, hydrochloride salt (1.0 g, 4.11 mmol) in DMF/toluene (1:1, 5 mL) was added tert-butyl 5-bromopyrimidine-2-carboxylate (1.59 g, 6.17 mmol), potassium carbonate (1.1 g, 8.2 mmol) at rt and the reaction mixture was degassed with argon for 5 minutes. To this mixture, Pd₂(dba)₃ (375 mg, 0.41 mmol) and SPhos (421 mg, 1.02 mmol) were added and stirred at 120° C. for 16 h. The reaction mixture cooled to rt, diluted with EtOAc (200 mL) and filtered through CELITE®. The filtrate was washed with ice-cold aqueous brine solution (2×20 mL), dried with anhydrous sodium sulfate and concentrated under reduced pressure. The residue was purified via normal phase SiO₂ chromatography (0-60% EtOAc/petroleum ether) to give tert-butyl 5-(1-ethyl-5,6-dimethoxyisoindolin-2-yl)pyrimidine-2-carboxylate as an orange-red solid, which was used without further purification (0.41 g, 22% yield, 386 [M+H]⁺ observed).

### 5-(1-Ethyl-5,6-dimethoxyisoindolin-2-yl)pyrimidine-2-carboxylic Acid

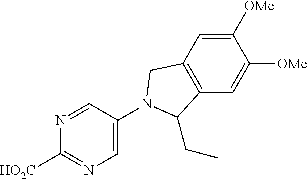 [SMILES: CCC1c2cc(OC)c(OC)cc2CN1c1cnc(C(=O)O)nc1]

To a solution of tert-butyl 5-(1-ethyl-5,6-dimethoxyisoindolin-2-yl)pyrimidine-2-carboxylate (400 mg, 1.04 mmol) in 1,4-dioxane/water (1:1, 20 mL) at rt was added lithium hydroxide monohydrate (130 mg, 3.09 mmol) and the reaction mixture was stirred at rt for 16 h. The reaction mixture was diluted with H₂O (30 mL) and extracted with EtOAc (50 mL). The aqueous layer was acidified with HCl (10% aqueous solution) and extracted with CH₂Cl₂ (2×100 mL). The combined organic layer was dried over anhydrous sodium sulfate and concentrated under reduced pressure. The residue was purified by purified by reverse phase HPLC to give 5-(1-ethyl-5,6-dimethoxyisoindolin-2-yl)pyrimidine-2-carboxylic acid as a brown solid (21 mg, 6% yield, m/z: 330 [M+H]⁺ observed). ¹H NMR (400 MHz, DMSO-d₆) δ 8.27 (s, 2H), 6.97 (d, 2H), 5.27 (s, 1H), 4.71 (d, 1H), 4.57 (d, 1H), 3.78 (s, 6H), 2.09-2.07 (m, 1H), 1.88-1.83 (m, 1H), 0.50 (t, 3H).

The following examples were prepared in a similar manner as 5-(1-ethyl-5,6-dimethoxyisoindolin-2-yl)pyrimidine-2-carboxylic acid from 1-ethyl-6-fluoro-5-methoxyisoindoline and tert-butyl 5-bromopyrimidine-2-carboxylate.

### Example 98: 5-(1-Ethyl-6-fluoro-5-methoxyisoindolin-2-yl)pyrimidine-2-carboxylic Acid

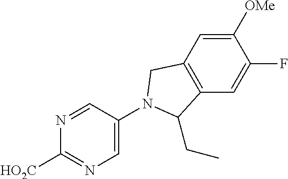 [SMILES: CCC1c2cc(F)c(OC)cc2CN1c1cnc(C(=O)O)nc1]

m/z: 318 [M+H]⁺ observed. ¹H NMR (400 MHz, DMSO-d₆) δ 8.32 (s, 2H), 7.30-7.22 (m, 2H), 5.33 (s, 1H), 4.79-4.63 (m, 2H), 3.86 (s, 3H), 2.12-2.05 (m, 1H), 1.86-1.80 (m, 1H), 0.51 (t, 3H).

### Example 99: 5-(1-Ethyl-6-fluoro-5-methoxyisoindolin-2-yl)pyrimidine-2-carboxylic Acid (Single Enantiomer I)

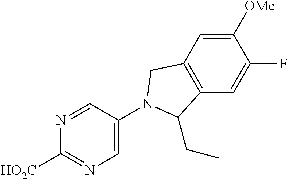 [SMILES: CCC1c2cc(F)c(OC)cc2CN1c1cnc(C(=O)O)nc1]

m/z: 318 [M+H]⁺ observed. ¹H NMR (400 MHz, DMSO-d₆) δ 8.32 (s, 2H), 7.30-7.22 (m, 2H), 5.33 (s, 1H), 4.79-4.63 (m, 2H), 3.86 (s, 3H), 2.12-2.05 (m, 1H), 1.86-1.80 (m, 1H), 0.49 (t, 3H).

### Example 100: 5-(1-Ethyl-6-fluoro-5-methoxyisoindolin-2-yl)pyrimidine-2-carboxylic Acid (Single Enantiomer II)

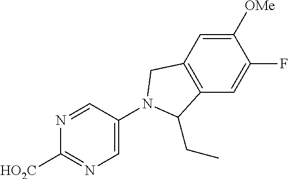 [SMILES: CCC1c2cc(F)c(OC)cc2CN1c1cnc(C(=O)O)nc1]

m/z: 318 [M+H]⁺ observed. ¹H NMR (400 MHz, DMSO-d₆) δ 8.32 (s, 2H), 7.30-7.22 (m, 2H), 5.33 (s, 1H), 4.79-4.63 (m, 2H), 3.86 (s, 3H), 2.12-2.05 (m, 1H), 1.86-1.80 (m, 1H), 0.49 (t, 3H).

### Example 101: 5-(1-Ethyl-6-fluoro-5-methoxyisoindolin-2-yl)-N-(methylsulfonyl)pyrimidine-2-carboxamide

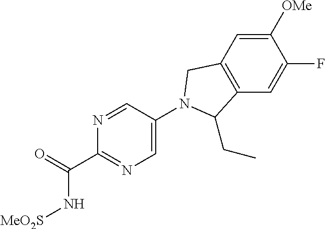 [SMILES: CCC1c2cc(F)c(OC)cc2CN1c1cnc(C(=O)NS(C)(=O)=O)nc1]

To a solution of 5-(1-ethyl-6-fluoro-5-methoxyisoindolin-2-yl)pyrimidine-2-carboxylic acid (200 mg, 0.63 mmol) in CH₂Cl₂ (5 mL) was added oxalyl chloride (0.06 mL, 0.75 mmol) at 0° C. The reaction was slowly warmed to rt and stirred for 2 h. The reaction mixture was concentrated under reduced pressure. The crude acid chloride was diluted with CH₂Cl₂ (5 mL) followed by the addition of DIPEA (0.33 mL, 1.9 mmol) and methanesulfonamide (89 mg, 0.94 mmol) at 0° C. The reaction mixture was warmed to rt and stirred for 4 h. The mixture was diluted with H₂O (30 mL) and extracted with CH₂Cl₂ (2×50 mL). The combined organic layer was dried over anhydrous sodium sulfate, filtered and concentrated under reduced pressure. The residue was purified by purified by reverse phase HPLC to give 5-(1-ethyl-6-fluoro-5-methoxyisoindolin-2-yl)-N-(methylsulfonyl)pyrimidine-2-carboxamide as an off-white solid (45 mg, 18% yield, 395 [M+H]⁺ observed). ¹H NMR (400 MHz, DMSO-d₆) δ 12.51 (br s, 1H), 8.32 (br s, 2H), 7.29-7.21 (m, 2H), 5.34 (s, 1H), 4.80-4.63 (m, 2H), 3.32 (s, 3H), 2.99 (s, 3H), 2.49-2.32 (m, 1H), 2.08-2.07 (m, 1H), 0.52-0.49 (m, 3H).

### Example 102: 5-(1-Ethyl-6-fluoro-5-methoxyisoindolin-2-yl)-N-(methylsulfonyl)pyrimidine-2-carboxamide (Single Enantiomer I)

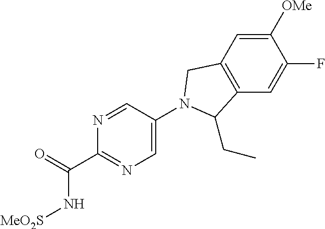 [SMILES: CCC1c2cc(F)c(OC)cc2CN1c1cnc(C(=O)NS(C)(=O)=O)nc1]

### Example 103: 5-(1-Ethyl-6-fluoro-5-methoxyisoindolin-2-yl)-N-(methylsulfonyl)pyrimidine-2-carboxamide (Single Enantiomer II)

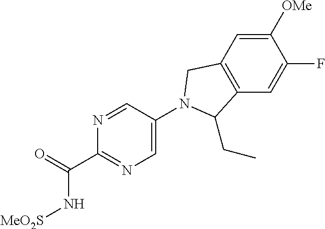 [SMILES: CCC1c2cc(F)c(OC)cc2CN1c1cnc(C(=O)NS(C)(=O)=O)nc1]

A mixture of enantiomers (35 mg) was separated by chiral HPLC on a Chiralpak IC® column using 55% EtOH/0.2% TFA in n-hexanes to give 5-(1-ethyl-6-fluoro-5-methoxyisoindolin-2-yl)-N-(methylsulfonyl)pyrimidine-2-carboxamide (single enantiomer I) as an off-white solid (faster eluting enantiomer, 5 mg, 14% yield, 395 [M+H]⁺ observed), and 5-(1-ethyl-6-fluoro-5-methoxyisoindolin-2-yl)-N-(methylsulfonyl)pyrimidine-2-carboxamide (single enantiomer II) as an off-white solid (slower eluting enantiomer, 7 mg, 20% yield, 395 [M+H]⁺ observed).

### Example 102: 5-(1-Ethyl-6-fluoro-5-methoxyisoindolin-2-yl)-N-(methylsulfonyl)pyrimidine-2-carboxamide (Single Enantiomer I)

m/z: 395 [M+H]⁺ observed. ¹H NMR (400 MHz, DMSO-d₆) δ 12.51 (br s, 1H), 8.32 (br s, 2H), 7.29-7.21 (m, 2H), 5.34 (s, 1H), 4.80-4.63 (m, 2H), 3.32 (s, 3H), 2.99 (s, 3H), 2.49-2.32 (m, 1H), 2.08-2.07 (m, 1H), 0.52-0.49 (m, 3H).

### Example 103: 5-(1-Ethyl-6-fluoro-5-methoxyisoindolin-2-yl)-N-(methylsulfonyl)pyrimidine-2-carboxamide (Single Enantiomer II)

m/z: 395 [M+H]⁺ observed. ¹H NMR (400 MHz, DMSO-d₆) δ 12.51 (br s, 1H), 8.32 (br s, 2H), 7.29-7.21 (m, 2H), 5.34 (s, 1H), 4.80-4.63 (m, 2H), 3.32 (s, 3H), 2.99 (s, 3H), 2.49-2.32 (m, 1H), 2.08-2.07 (m, 1H), 0.52-0.49 (m, 3H).

The following examples were prepared in a similar manner as 5-(1-ethyl-6-fluoro-5-methoxyisoindolin-2-yl)-N-(methylsulfonyl)pyrimidine-2-carboxamide from an appropriately substituted pyrimidine-2-carboxylic acid and an appropriate amine.

### Example 104: 4-(1-Ethyl-6-fluoro-5-methoxyisoindolin-2-yl)-N-(methylsulfonyl)pyrimidine-2-carboxamide

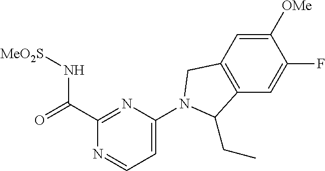 [SMILES: CCC1c2cc(F)c(OC)cc2CN1c1ccnc(C(=O)NS(C)(=O)=O)n1]

m/z: 395 [M+H]⁺ observed. ¹H NMR (400 MHz, DMSO-d₆) δ 8.31 (br s, 1H), 7.27 (d, 2H), 6.65 (br s, 1H), 5.49-4.68 (m, 3H), 3.80 (s, 3H), 3.08 (s, 3H), 2.42-2.45 (m, 1H), 1.90-1.87 (m, 1H), 0.52-0.49 (m, 3H).

### Example 105: 5-(1-Ethyl-6-fluoro-5-methoxyisoindolin-2-yl)-N-(1-methyl-1H-imidazol-2-yl)pyrimidine-2-carboxamide

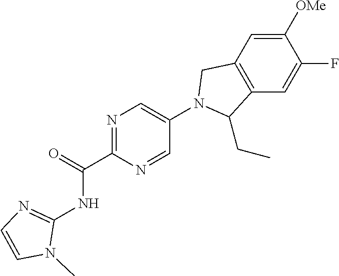 [SMILES: CCC1c2cc(F)c(OC)cc2CN1c1cnc(C(=O)Nc2nccn2C)nc1]

m/z: 397 [M+H]⁺ observed. ¹H NMR (400 MHz, DMSO-d₆) δ 10.32 (s, 1H), 8.40 (br s, 2H), 7.31 (d, 1H), 7.24 (d, 1H), 7.12 (s, 1H), 6.81 (d, 1H), 5.40 (s, 1H), 4.84-4.69 (m, 2H), 3.87 (s, 3H), 3.47 (s, 3H), 2.15-2.08 (m, 1H), 2.07-1.82 (m, 1H), 0.54 (t, 3H).

### Example 106: 5-(1-Ethyl-6-fluoro-5-methoxyisoindolin-2-yl)-N-(pyridin-2-yl)pyrimidine-2-carboxamide

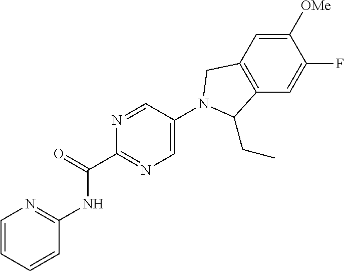 [SMILES: CCC1c2cc(F)c(OC)cc2CN1c1cnc(C(=O)Nc2ccccn2)nc1]

To a solution of tert-butyl 5-(1-ethyl-6-fluoro-5-methoxyisoindolin-2-yl)pyrimidine-2-carboxylate (400 mg, 1.1 mmol) in toluene (10 mL) was added 2-aminopyridine (151 mg, 1.60 mmol) and trimethylaluminum (1.0 M in toluene, 2.2 mL, 2.2 mmol) at rt. The reaction mixture was stirred at 100° C. for 2 h, cooled to rt, diluted with H₂O (50 mL) and extracted with EtOAc (2×50 mL). The combined organic layer was dried over anhydrous sodium sulfate and concentrated under reduced pressure. The residue was purified by reverse phase HPLC to give 5-(1-ethyl-6-fluoro-5-methoxyisoindolin-2-yl)-N-(pyridin-2-yl)pyrimidine-2-carboxamide as an off-white solid (45 mg, 11% yield, 394 [M+H]⁺ observed). ¹H NMR (400 MHz, DMSO-d₆) δ 10.22 (s, 1H), 8.46 (s, 2H), 8.38-8.37 (m, 1H), 8.28 (d, 1H), 7.90-7.86 (m, 1H), 7.31 (d, 1H), 7.24 (d, 1H), 7.19-7.17 (m, 1H), 5.40 (s, 1H), 4.85-4.70 (m, 2H), 3.87 (s, 3H), 2.18-2.11 (m, 1H), 1.89-1.83 (m, 1H), 0.59-0.56 (m, 3H).

### Example 107: 5-(1-Ethyl-6-fluoro-5-methoxyisoindolin-2-yl)-N-(pyridin-2-yl)pyrimidine-2-carboxamide (Single Enantiomer I)

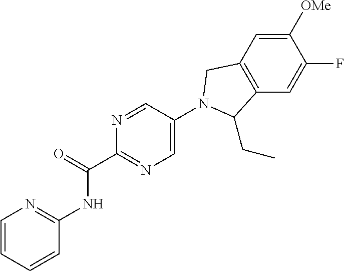 [SMILES: CCC1c2cc(F)c(OC)cc2CN1c1cnc(C(=O)Nc2ccccn2)nc1]

### Example 108: 5-(1-Ethyl-6-fluoro-5-methoxyisoindolin-2-yl)-N-(pyridin-2-yl)pyrimidine-2-carboxamide (Single Enantiomer II)

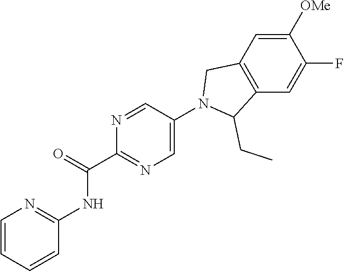 [SMILES: CCC1c2cc(F)c(OC)cc2CN1c1cnc(C(=O)Nc2ccccn2)nc1]

A mixture of enantiomers (45 mg) was separated by SFC (supercritical fluid chromatography) on a (R,R)Whelk-01 column using 50% MeOH to give 5-(1-ethyl-6-fluoro-5-methoxyisoindolin-2-yl)-N-(pyridin-2-yl)pyrimidine-2-carboxamide (single enantiomer I) as a white solid (faster eluting enantiomer, 5 mg, 11% yield, 394 [M+H]⁺ observed), and 5-(1-ethyl-6-fluoro-5-methoxyisoindolin-2-yl)-N-(pyridin-2-yl)pyrimidine-2-carboxamide (single enantiomer II) as a white solid (slower eluting enantiomer, 5 mg, 11% yield, 394 [M+H]⁺ observed).

### Example 107: 5-(1-Ethyl-6-fluoro-5-methoxyisoindolin-2-yl)-N-(pyridin-2-yl)pyrimidine-2-carboxamide (Single Enantiomer I)

m/z: 394 [M+H]⁺ observed. ¹H NMR (400 MHz, DMSO-d₆) δ 10.22 (s, 1H), 8.46 (s, 2H), 8.38-8.37 (m, 1H), 8.28 (d, 1H), 7.90-7.86 (m, 1H), 7.31 (d, 1H), 7.24 (d, 1H), 7.19-7.17 (m, 1H), 5.40 (s, 1H), 4.85-4.70 (m, 2H), 3.87 (s, 3H), 2.18-2.11 (m, 1H), 1.89-1.83 (m, 1H), 0.59-0.56 (m, 3H).

### Example 108: 5-(1-Ethyl-6-fluoro-5-methoxyisoindolin-2-yl)-N-(pyridin-2-yl)pyrimidine-2-carboxamide (Single Enantiomer II)

m/z: 394 [M+H]⁺ observed. ¹H NMR (400 MHz, DMSO-d₆) δ 10.22 (s, 1H), 8.46 (s, 2H), 8.38-8.37 (m, 1H), 8.28 (d, 1H), 7.90-7.86 (m, 1H), 7.31 (d, 1H), 7.24 (d, 1H), 7.19-7.17 (m, 1H), 5.40 (s, 1H), 4.85-4.70 (m, 2H), 3.87 (s, 3H), 2.18-2.11 (m, 1H), 1.89-1.83 (m, 1H), 0.59-0.56 (m, 3H).

The following examples were prepared in a similar manner as 5-(1-ethyl-6-fluoro-5-methoxyisoindolin-2-yl)-N-(pyridin-2-yl)pyrimidine-2-carboxamide from an appropriately substituted pyrimidine-2-carboxylate and an appropriate amine.

### Example 109: 4-(1-Ethyl-6-fluoro-5-methoxyisoindolin-2-yl)-N-methylpyrimidine-2-carboxamide (Single Enantiomer I)

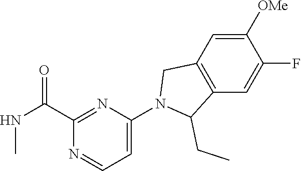 [SMILES: CCC1c2cc(F)c(OC)cc2CN1c1ccnc(C(=O)NC)n1]

m/z: 331 [M+H]⁺ observed. ¹H NMR (400 MHz, DMSO-d₆, at 90° C.) δ 8.31-8.29 (m, 1H), 8.26 (s, 1H), 7.20-7.17 (m, 2H), 6.71-6.69 (m, 1H), 5.39 (s, 1H), 4.81-4.69 (m, 2H), 3.86 (s, 3H), 2.82-2.81 (m, 3H), 2.30-2.24 (m, 1H), 1.88-1.84 (m, 1H), 0.59-0.56 (m, 3H).

### Example 110: 4-(1-Ethyl-6-fluoro-5-methoxyisoindolin-2-yl)-N-methylpyrimidine-2-carboxamide (Single Enantiomer II)

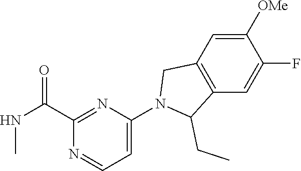 [SMILES: CCC1c2cc(F)c(OC)cc2CN1c1ccnc(C(=O)NC)n1]

m/z: 331 [M+H]⁺ observed. ¹H NMR (400 MHz, DMSO-d₆, at 90° C.) δ 8.31-8.29 (m, 1H), 8.26 (s, 1H), 7.20-7.17 (m, 2H), 6.71-6.69 (m, 1H), 5.39 (s, 1H), 4.81-4.69 (m, 2H), 3.86 (s, 3H), 2.82-2.81 (m, 3H), 2.30-2.24 (m, 1H), 1.88-1.84 (m, 1H), 0.59-0.56 (m, 3H).

### Example 111: 3-[4-(6,7-Difluoro-3,4-dihydro-1H-isoquinolin-2-yl)pyrimidin-2-yl]pyridin-2-ol

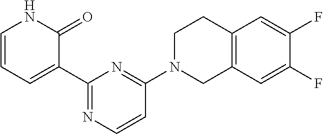 [SMILES: O=c1[nH]cccc1-c1nccc(N2CCc3cc(F)c(F)cc3C2)n1]

A mixture of 2-(2-chloropyrimidin-4-yl)-6,7-difluoro-3,4-dihydro-1H-isoquinoline (400 mg, 1.42 mmol), (2-oxo-1,2-dihydropyridin-3-yl) boronic acid (296 mg, 2.13 mmol), Na₂CO₃ (451 mg, 4.26 mmol) and Pd(PPh₃)₄ (164 mg, 0.142 mmol) in MeOH/toluene (2:1, 15 mL) was degassed and then heated to 120° C. for 2 hours in a sealed tube under N₂. The reaction mixture was poured into H₂O (20 mL) and extracted with EtOAc (3×20 mL). The combined organic phase was washed with saturated aqueous brine solution (30 mL), dried over anhydrous sodium sulfate, filtered and evaporated under reduced pressure. The crude product was purified by neutral prep-HPLC to afford 3-[4-(6,7-difluoro-3,4-dihydro-1H-isoquinolin-2-yl)pyrimidin-2-yl]pyridine-2-ol as a light yellow solid (197 mg, 38% yield, m/z: 341 [M+H]⁺ observed). ¹H NMR (400 MHz, DMSO-d₆) δ 14.93 (s, 1H), 8.78-8.63 (m, 1H), 8.33 (d, J=6.4 Hz, 1H), 8.28-8.08 (m, 1H), 7.42-7.37 (m, 1H), 7.34-7.27 (m, 1H), 7.11-6.79 (m, 2H), 4.86-4.71 (m, 2H), 4.00-3.76 (m, 2H), 2.91 (t, J=5.6 Hz, 2H).

### Example 112: 6-(1-Ethyl-5,6-difluoro-3,4-dihydro-1H-isoquinolin-2-yl)-4-hydroxy-pyridine-3-carboxylic Acid

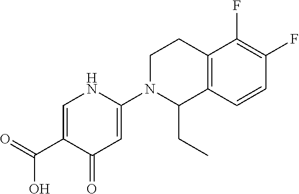 [SMILES: CCC1c2ccc(F)c(F)c2CCN1c1cc(=O)c(C(=O)O)c[nH]1]

### 6-Chloro-4-methoxy-pyridine-3-carbonitrile

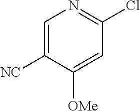 [SMILES: COc1cc(Cl)ncc1C#N]

A mixture of 4,6-dichloropyridine-3-carbonitrile (8 g, 46.2 mmol) and K₂CO₃ (6.39 g, 46.2 mmol) in MeOH (300 mL) was stirred at rt for 24 hr. The reaction mixture was concentrated under reduced pressure. The residue was diluted with H₂O (300 mL) and extracted with EtOAc (3×200 mL). The combined organic layer was dried over anhydrous sodium sulfate, filtered and concentrated under reduced pressure. The residue was purified by normal phase SiO₂ chromatography (0% to 40% EtOAc/petroleum ether) to afford 6-chloro-4-methoxynicotinonitrile as a white solid (5 g, 64% yield). ¹H NMR (400 MHz, CDCl3): δ 8.40 (s, 1H), 6.89 (s, 1H), 3.95 (s, 3H).

### 6-(1-Ethyl-5,6-difluoro-3,4-dihydroisoquinolin-2(1H)-yl)-4-hydroxynicotinonitrile

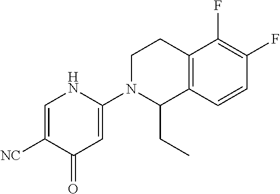 [SMILES: CCC1c2ccc(F)c(F)c2CCN1c1cc(=O)c(C#N)c[nH]1]

To a solution of 6-chloro-4-methoxy-pyridine-3-carbonitrile (1 g, 5.93 mmol) in methylpyrrolidone (4 mL) was added 1-ethyl-5,6-difluoro-1,2,3,4-tetrahydroisoquinoline (1.29 g, 6.53 mmol) and N,N-diisopropylethylamine (4.1 mL, 23.7 mmol) under N₂. Then the reaction was stirred for 1 hour at 140° C. under microwave irradiation. The reaction was cooled to rt, diluted with H₂O (20 mL) and extracted with EtOAc (3×20 mL). The combined organic layer was washed with saturated aqueous brine solution (2×50 mL), dried over anhydrous sodium sulfate, filtered and concentrated under reduced pressure. The residue was purified by normal phase SiO₂ chromatography (0% to 60% EtOAc/petroleum ether) to afford 6-(1-ethyl-5,6-difluoro-3,4-dihydro-1H-isoquinolin-2-yl)-4-methoxy-pyridine-3-carbonitrile as an orange oil, which was used in the next step without further purification (1 g, 51% yield).

To a solution of 6-(1-ethyl-5,6-difluoro-3,4-dihydro-1H-isoquinolin-2-yl)-4-methoxy-pyridine-3-carbonitrile (0.91 g, 2.76 mmol) in CH₂Cl₂ (20 mL) at −78° C. was added dropwise boron tribromide (1.6 mL, 16.6 mmol) and the mixture stirred at rt for 12 h. The reaction was quenched with H₂O (100 ml) and extracted with CH₂Cl₂ (3×50 mL). The combined organic layer was washed with saturated aqueous sodium bicarbonate solution (2×100 mL), saturated aqueous brine solution (200 mL), dried over anhydrous sodium sulfate, filtered and concentrated under reduced pressure. The residue was purified by normal phase SiO₂ chromatography (0% to 65% EtOAc/petroleum ether) to give 6-(1-ethyl-5,6-difluoro-3,4-dihydro-1H-isoquinolin-2-yl)-4-hydroxy-pyridine-3-carbonitrile as a yellow solid (0.2 g, 20% yield, m/z: 316 [M+H]⁺ observed).

### 6-(1-Ethyl-5,6-difluoro-3,4-dihydro-1H-isoquinolin-2-yl)-4-hydroxy-pyridine-3-carboxylic Acid

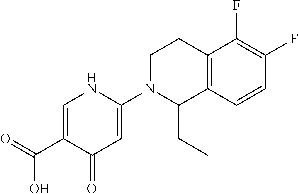 [SMILES: CCC1c2ccc(F)c(F)c2CCN1c1cc(=O)c(C(=O)O)c[nH]1]

A solution of 6-(1-ethyl-5,6-difluoro-3,4-dihydro-1H-isoquinolin-2-yl)-4-hydroxy-pyridine-3-carbonitrile (0.15 g, 0.48 mmol) in concentrated HCl (1 mL) was stirred for 16 h at 80° C. The reaction was cooled to rt and concentrated under reduced pressure. The residue was purified by reverse phase HPLC to give 6-(1-ethyl-5,6-difluoro-3,4-dihydro-H-isoquinolin-2-yl)-4-hydroxy-pyridine-3-carboxylic acid as a white solid (58 mg, 37% yield, m/z: 335 [M+H]⁺ observed). ¹H NMR (400 MHz, DMSO-d₆) δ 8.27 (s, 1H), 7.31-7.25 (m, 1H), 7.14-7.11 (m, 1H), 6.25 (s, 1H), 5.34 (s, 1H), 4.14-4.11 (m, 1H), 2.92-2.89 (m, 3H), 1.93-1.77 (m, 2H), 0.91-0.87 (m, 3H).

### Example 113: 5-(1-Ethyl-5,6-difluoro-3,4-dihydro-1H-isoquinolin-2-yl)pyrimidine-2-carboxylic Acid

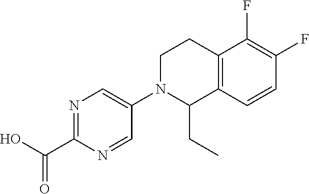 [SMILES: CCC1c2ccc(F)c(F)c2CCN1c1cnc(C(=O)O)nc1]

### 5-(1-Ethyl-5,6-difluoro-3,4-dihydro-1H-isoquinolin-2-yl)pyrimidine-2-carbonitrile

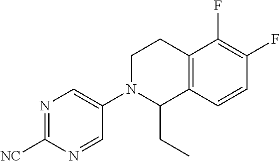 [SMILES: CCC1c2ccc(F)c(F)c2CCN1c1cnc(C#N)nc1]

To a mixture of 1-ethyl-5,6-difluoro-1,2,3,4-tetrahydroisoquinoline, hydrochloride salt (500 mg, 2.15 mmol) and 5-fluoropyrimidine-2-carbonitrile (395 mg, 3.21 mmol) in DMF (5 mL) was added cesium carbonate (2.09 g, 6.42 mmol) under N₂ and stirred at 100° C. for 2 hours. The reaction mixture was cooled to rt, H₂O (50 mL) was added and the aqueous phase extracted with EtOAc (3×20 mL). The combined organic phase was washed with saturated aqueous brine solution (20 mL), dried with anhydrous sodium sulfate, filtered and concentrated in vacuum to get 5-(1-ethyl-5,6-difluoro-3,4-dihydro-1H-isoquinolin-2-yl)pyrimidine-2-carbonitrile as a yellow solid, which was used directly without purification (500 mg, 65% yield, m/z: 301 [M+H]⁺ observed).

### 5-(1-Ethyl-5,6-difluoro-3,4-dihydro-1H-isoquinolin-2-yl)pyrimidine-2-carboxylic Acid

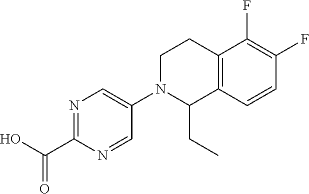 [SMILES: CCC1c2ccc(F)c(F)c2CCN1c1cnc(C(=O)O)nc1]

A mixture of 5-(1-ethyl-5,6-difluoro-3,4-dihydro-1H-isoquinolin-2-yl)pyrimidine-2-carbonitrile (0.5 g, 1.66 mmol) in concentrated HCl (5 mL) was stirred at 80° C. for 2 hours. The mixture was concentrated in vacuum. Saturated aqueous sodium carbonate was added to adjust the pH to 6. The resulting mixture was purified directly by reverse phase HPLC to get 5-(1-ethyl-5,6-difluoro-3,4-dihydroisoquinolin-2(1H)-yl)pyrimidine-2-carboxylic acid as a light yellow solid (18 mg, 3% yield, m/z: 320 [M+H]⁺ observed). ¹H NMR (400 MHz, DMSO-d₆) δ 8.51 (s, 2H), 7.31-7.26 (m, 1H), 7.14-7.11 (m, 1H), 5.07 (t, J=7.6 Hz, 1H), 3.96-3.90 (m, 1H), 3.60-3.53 (m, 1H), 3.02-2.93 (m, 1H), 2.87-2.82 (m, 1H), 1.94-1.86 (m, 1H), 1.80-1.71 (m, 1H), 0.94 (t, J=7.2 Hz, 3H).

The following examples were prepared in a similar manner as 5-(1-ethyl-5,6-difluoro-3,4-dihydroisoquinolin-2(1H)-yl)pyrimidine-2-carboxylic acid from 5-fluoropyrimidine-2-carbonitrile and an appropriate amine.

### Example 114: 5-(1-Ethyl-7-fluoro-6-methoxy-3,4-dihydro-1H-isoquinolin-2-yl)pyrimidine-2-carboxylic Acid (Single Enantiomer I)

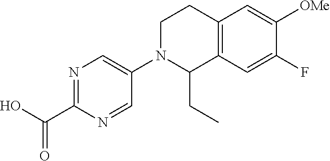 [SMILES: CCC1c2cc(F)c(OC)cc2CCN1c1cnc(C(=O)O)nc1]

m/z: 332 [M+H]⁺ observed. ¹H NMR (400 MHz, CDCl₃) δ 8.36 (s, 2H), 6.87 (d, J=11.6 Hz, 1H), 6.75 (d, J=8 Hz, 1H), 4.52 (s, 1H), 3.87 (s, 3H), 3.61-3.53 (m, 2H), 2.94 (s, 2H), 1.89-1.87 (m, 1H), 1.70-1.69 (m, 1H), 0.95-0.92 (m, 3H).

### Example 115: 5-(1-Ethyl-7-fluoro-6-methoxy-3,4-dihydro-1H-isoquinolin-2-yl)pyrimidine-2-carboxylic Acid (Single Enantiomer II)

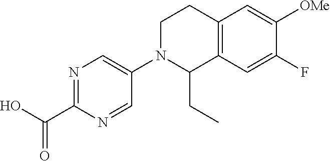 [SMILES: CCC1c2cc(F)c(OC)cc2CCN1c1cnc(C(=O)O)nc1]

m/z: 332 [M+H]⁺ observed. ¹H NMR (400 MHz, CDCl₃) δ 8.36 (s, 2H), 6.87 (d, J=11.6 Hz, 1H), 6.75 (d, J=8 Hz, 1H), 4.52 (s, 1H), 3.87 (s, 3H), 3.61-3.53 (m, 2H), 2.94 (s, 2H), 1.89-1.87 (m, 1H), 1.70-1.69 (m, 1H), 0.95-0.92 (m, 3H).

### Example 116: 5-(1-Ethyl-5,6-difluoroisoindolin-2-yl)pyrimidine-2-carboxylic Acid (Single Enantiomer I)

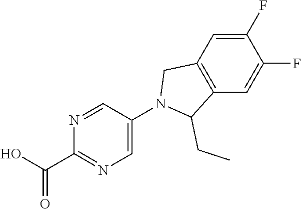 [SMILES: CCC1c2cc(F)c(F)cc2CN1c1cnc(C(=O)O)nc1]

m/z: 306 [M+H]⁺ observed. ¹H NMR (400 MHz, DMSO-d₆) δ 8.28 (s, 2H), 7.54 (t, J=8.8 Hz, 2H), 5.37 (s, 1H), 4.81-4.76 (m, 1H), 4.66 (d, J=10.4 Hz, 1H), 2.12-2.11 (m, 1H), 1.88-1.87 (m, 1H), 0.52 (t, J=7.6 Hz, 3H).

### Example 117: 5-(1-Ethyl-5,6-difluoroisoindolin-2-yl)pyrimidine-2-carboxylic Acid (Single Enantiomer II)

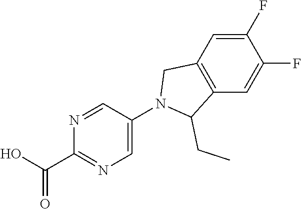 [SMILES: CCC1c2cc(F)c(F)cc2CN1c1cnc(C(=O)O)nc1]

m/z: 306 [M+H]⁺ observed. ¹H NMR (400 MHz, DMSO-d₆) δ 8.28 (s, 2H), 7.54 (t, J=8.8 Hz, 2H), 5.37 (s, 1H), 4.81-4.76 (m, 1H), 4.66 (d, J=10.4 Hz, 1H), 2.12-2.11 (m, 1H), 1.88-1.87 (m, 1H), 0.52 (t, J=7.6 Hz, 3H).

### Example 118: 5-(1-Ethyl-7-fluoro-6-methoxy-3,4-dihydro-1H-isoquinolin-2-yl)pyrimidine-2-carboxamide

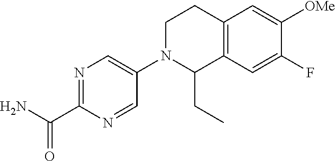 [SMILES: CCC1c2cc(F)c(OC)cc2CCN1c1cnc(C(N)=O)nc1]

### 5-(1-Ethyl-7-fluoro-6-methoxy-3,4-dihydro-1H-isoquinolin-2-yl)pyrimidine-2-carbonitrile

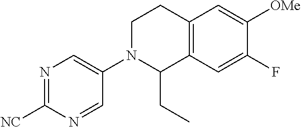 [SMILES: CCC1c2cc(F)c(OC)cc2CCN1c1cnc(C#N)nc1]

To a mixture of 1-ethyl-7-fluoro-6-methoxy-1,2,3,4-tetrahydroisoquinoline (0.3 g, 1.43 mmol) and 5-fluoropyrimidine-2-carbonitrile (0.26 g, 2.15 mmol) in DMF (8 mL) was added cesium carbonate (1.4 g, 4.3 mmol) under N₂. The mixture was stirred at 100° C. for 12 hours. The reaction mixture was cooled to rt, diluted with H₂O (30 mL) and extracted with EtOAc (3×30 mL). The combined organic phase was washed with saturated aqueous brine solution (30 mL), dried over anhydrous sulfate, filtered and concentrated in vacuum. The residue was purified via normal phase SiO₂ chromatography (0-20% EtOAc/petroleum ether) to give 5-(1-ethyl-7-fluoro-6-methoxy-3,4-dihydro-1H-isoquinolin-2-yl) pyrimidine-2-carbonitrile as a yellow solid (0.2 g, 45% yield, m/z: 313 [M+H]⁺ observed). ¹H NMR (400 MHz, CDCl₃) δ 8.31 (s, 2H), 6.89 (d, J=10.8 Hz, 1H), 6.80 (d, J=8 Hz, 1H), 4.54 (t, J=7.6 Hz, 1H), 3.90 (s, 3H), 3.73-3.67 (m, 1H), 3.61-3.54 (m, 1H), 3.03 (t, J=6 Hz, 2H), 2.00-1.93 (m, 1H), 1.81-1.74 (m, 1H), 1.01 (t, J=7.6 Hz, 3H).

### 5-(1-Ethyl-7-fluoro-6-methoxy-3,4-dihydro-1H-isoquinolin-2-yl)pyrimidine-2-carboxamide

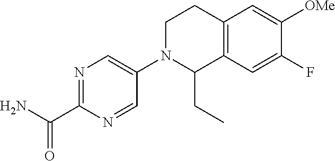 [SMILES: CCC1c2cc(F)c(OC)cc2CCN1c1cnc(C(N)=O)nc1]

To a mixture of 5-(1-ethyl-7-fluoro-6-methoxy-3,4-dihydro-1H-isoquinolin-2-yl) pyrimidine-2-carbonitrile (200 mg, 0.64 mmol) in MeOH (4 mL) was added NaOH (1 M in H₂O, 1.9 mL, 1.9 mmol) and H₂O₂ (30% solution in H₂O, 200 uL, 1.92 mmol) under N₂. The mixture was stirred at rt for 16 hours. The reaction mixture was quenched by the addition of saturated sodium sulfite solution (5 mL) and filtered. The pH of the filtrate was adjusted to 7 with glacial HOAc. The mixture was purified directly by reverse phase HPLC to give 5-(1-ethyl-7-fluoro-6-methoxy-3,4-dihydro-1H-isoquinolin-2-yl)pyrimidine-2-carboxamide as a white solid (77 mg, 36% yield, m/z: 331 [M+H]⁺ observed). ¹H NMR (400 MHz, DMSO-d₆) δ 8.50 (s, 2H), 7.83 (s, 1H), 7.44 (s, 1H), 7.10 (d, J=12 Hz, 1H), 7.01 (d, J=8.8 Hz, 1H), 4.97-4.94 (m, 1H), 3.81 (s, 3H), 3.78-3.75 (m, 1H), 3.62-3.60 (m, 1H), 2.95-2.88 (m, 2H), 1.89-1.71 (m, 2H), 0.91 (t, J=7.2 Hz, 3H).

The following examples were prepared in a similar manner as 5-(1-ethyl-7-fluoro-6-methoxy-3,4-dihydroisoquinolin-2(1H)-yl)pyrimidine-2-carboxamide from 5-fluoropyrimidine-2-carbonitrile and an appropriate amine.

### Example 119: 5-(1-Ethyl-5,6-difluoroisoindolin-2-yl)pyrimidine-2-carboxamide

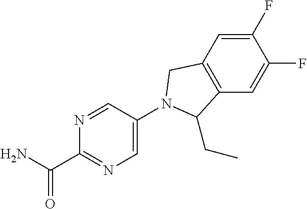 [SMILES: CCC1c2cc(F)c(F)cc2CN1c1cnc(C(N)=O)nc1]

m/z: 305 [M+H]⁺ observed. ¹H NMR (400 MHz, DMSO-d₆) δ 8.35 (s, 2H), 7.87 (br s, 1H), 7.55 (t, J=9.2 Hz, 2H), 7.44 (br s, 1H), 5.42 (br s, 1H), 4.83-4.79 (m, 1H), 4.71-4.68 (m, 1H), 2.14-2.08 (m, 1H), 1.90-1.86 (m, 1H), 0.52 (t, J=7.2 Hz, 3H).

### Example 120: 4-(1-Ethyl-7-fluoro-6-methoxy-3,4-dihydro-1H-isoquinolin-2-yl)pyrimidine-2-carboxamide

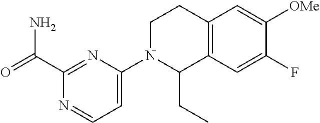 [SMILES: CCC1c2cc(F)c(OC)cc2CCN1c1ccnc(C(N)=O)n1]

m/z: 331 [M+H]⁺ observed. ¹H NMR (400 MHz, CDCl₃) δ 8.33 (d, J=6 Hz, 1H), 7.70 (s, 1H), 6.92 (d, J=11.6 Hz, 1H), 6.75 (d, J=8.4 Hz, 1H), 6.63 (d, J=6 Hz, 1H), 5.87 (s, 1H), 3.88 (s, 3H), 3.62 (t, J=7.2 Hz, 1H), 2.94 (t, J=7.6 Hz, 2H), 1.98-1.67 (m, 2H), 0.97 (t, J=7.2 Hz, 3H).

### Example 121: 4-(1-Ethyl-5,6-difluoroisoindolin-2-yl)pyrimidine-2-carboxamide

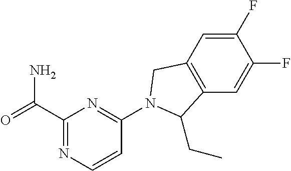 [SMILES: CCC1c2cc(F)c(F)cc2CN1c1ccnc(C(N)=O)n1]

m/z: 305 [M+H]⁺ observed. ¹H NMR (400 MHz, DMSO-d₆) δ 8.35 (br d, J=6.4 Hz, 1H), 7.96 (br s, 1H), 7.62 (br s, 1H), 7.53 (t, J=8.8 Hz, 2H), 6.85-6.70 (m, 1H), 5.62-5.31 (m, 1H), 5.02-4.71 (m, 2H), 1.90-1.87 (m, 1H), 1.86-1.84 (m, 1H), 0.53 (br s, 3H).

### Example 122: 3-(1-Ethyl-3,4-dihydroisoquinolin-2(1H)-yl)-1,10-phenanthroline

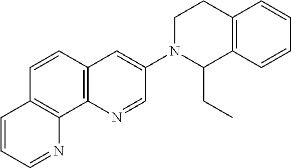 [SMILES: CCC1c2ccccc2CCN1c1cnc2c(ccc3cccnc32)c1]

To a solution of 3-bromo-1,10-phenanthroline (100 mg, 0.39 mmol) in toluene (2 mL) was added 1-ethyl-1,2,3,4-tetrahydroisoquinoline (62 mg, 0.39 mmol), followed by cesium carbonate (150 mg, 0.46 mmol). The solution was purged with nitrogen for 2 minutes. Tris(dibenzylideneacetone)dipalladium(0) (18 mg, 0.02 mmol) and SPhos (24 mg, 0.06 mmol) were added. The reaction vessel was sealed and heated to 110° C. for 16 hours. The reaction mixture was cooled to rt and H₂O (2 mL) was added, followed by EtOAc (2 mL). The layers were separated, and the aqueous phase was extracted with additional EtOAc (3×2 mL). The combined organic layer was concentrated under reduced pressure. The residue was purified by reverse phase HPLC to afford 3-(1-ethyl-3,4-dihydroisoquinolin-2(1H)-yl)-1,10-phenanthroline as a bright yellow solid (36 mg, 27% yield, m/z: 340 [M+H]⁺ observed). ¹H NMR (400 MHz, CDCl₃) δ 9.05 (dd, J=4.4, 1.8 Hz, 1H), 8.97 (d, J=3.0 Hz, 1H), 8.11 (dd, J=8.0, 1.8 Hz, 1H), 7.66-7.58 (m, 2H), 7.45 (dd, J=8.0, 4.4 Hz, 1H), 7.33 (d, J=3.0 Hz, 1H), 7.24-7.13 (m, 4H), 4.78 (t, J=7.1 Hz, 1H), 3.85-3.65 (m, 2H), 3.05 (qdd, J=15.9, 7.1, 5.4 Hz, 2H), 2.07 (ddd, J=14.2, 7.5, 6.7 Hz, 1H), 1.94-1.73 (m, 1H), 1.05 (t, J=7.4 Hz, 3H).

The following examples were prepared in a similar manner as 3-(1-ethyl-3,4-dihydroisoquinolin-2(1H)-yl)-1,10-phenanthroline from 3-bromo-1,10-phenanthroline and an appropriate amine.

### Example 123: 3-(5,6-Difluoro-1-methyl-3,4-dihydroisoquinolin-2(1H)-yl)-1,10-phenanthroline

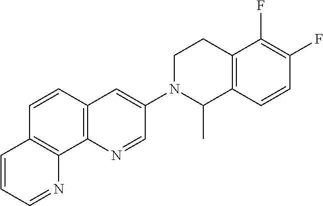 [SMILES: CC1c2ccc(F)c(F)c2CCN1c1cnc2c(ccc3cccnc32)c1]

m/z: 362 [M+H]⁺ observed. ¹H NMR (400 MHz, CDCl₃) δ 9.26 (dd, J=5.4, 1.5 Hz, 1H), 9.09 (d, J=2.9 Hz, 1H), 8.65 (dd, J=8.1, 1.5 Hz, 1H), 7.96-7.76 (m, 3H), 7.51 (d, J=2.9 Hz, 1H), 7.15-6.88 (m, 2H), 5.20 (q, J=6.7 Hz, 1H), 3.96 (dt, J=13.1, 5.4 Hz, 1H), 3.69 (ddd, J=13.3, 8.7, 4.9 Hz, 1H), 3.19-2.94 (m, 2H), 1.60 (d, J=6.8 Hz, 3H).

### Example 124: 3-(1-Ethyl-3,4-dihydroisoquinolin-2(1H)-yl)-N-methyl-1,7-naphthyridin-8-amine

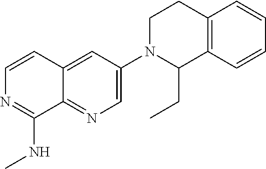 [SMILES: CCC1c2ccccc2CCN1c1cnc2c(NC)nccc2c1]

To a solution of 3-bromo-N-methyl-1,7-naphthyridin-8-amine (100 mg, 0.42 mmol) in toluene (2 mL) was added 1-ethyl-1,2,3,4-tetrahydroisoquinoline (68 mg, 0.42 mmol), followed by cesium carbonate (163 mg, 0.5 mmol). The solution was purged with nitrogen for 2 minutes. Tris(dibenzylideneacetone)dipalladium (0) (18 mg, 0.02 mmol) and SPhos (24 mg, 0.06 mmol) were added. The reaction vessel was sealed and heated to 110° C. for 16 hours. The reaction mixture was cooled to rt and H₂O (2 mL) was added, followed by EtOAc (2 mL). The layers were separated, and the aqueous phase was extracted with additional EtOAc (3×2 mL). The combined organic layer was concentrated under reduced pressure. The residue was purified by reverse phase HPLC to afford 3-(1-ethyl-3,4-dihydroisoquinolin-2(1H)-yl)-N-methyl-1,7-naphthyridin-8-amine as a light yellow solid (15 mg, 11% yield, m/z: 319 [M+H]⁺ observed). ¹H NMR (400 MHz, CDCl₃) δ 8.51 (d, J=2.9 Hz, 1H), 7.88 (d, J=5.9 Hz, 1H), 7.23-7.11 (m, 4H), 7.05-6.94 (m, 1H), 6.63 (d, J=6.0 Hz, 1H), 6.58 (s, 1H), 4.70 (t, J=7.1 Hz, 1H), 3.79-3.58 (m, 2H), 3.16 (d, J=5.1 Hz, 3H), 3.04 (dt, J=13.2, 6.4 Hz, 1H), 2.14-1.71 (m, 2H), 1.03 (t, J=7.4 Hz, 3H).

### Example 125: 7-(1-Ethyl-7-fluoro-6-methoxy-3,4-dihydroisoquinolin-2(1H)-yl)-3-methylpyrido[3,2-d]pyrimidin-4(3H)-one

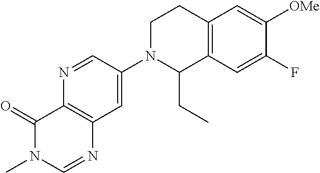 [SMILES: CCC1c2cc(F)c(OC)cc2CCN1c1cnc2c(=O)n(C)cnc2c1]

### 7-Bromo-3-methylpyrido[3,2-d]pyrimidin-4(3H)-one

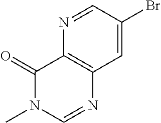

To a solution of 7-bromopyrido[3,2-d]pyrimidin-4-ol (400 mg, 1.78 mmol) in DMF (2 mL) was added cesium carbonate (720 mg, 2.23 mmol) and iodomethane (0.13 mL, 1.78 mmol). The mixture was stirred at rt for 24 h. The mixture was filtered off through CELITE®. The mother liquor was diluted with H₂O (10 mL), extracted with CH₂Cl₂ (2×20 mL), dried with anhydrous sodium sulfate, filtered and concentrated under reduced pressure to give 7-bromo-3-methylpyrido[3,2-d]pyrimidin-4(3H)-one as a white solid (425 mg, 100% yield, m/z: 239 [M]⁺ observed).

### 7-(1-Ethyl-7-fluoro-6-methoxy-3,4-dihydroisoquinolin-2(H)-yl)-3-methylpyrido[3,2-d]pyrimidin-4(3H)-one

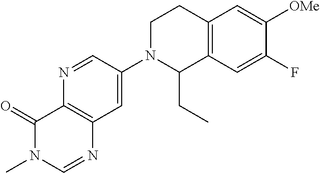 [SMILES: CCC1c2cc(F)c(OC)cc2CCN1c1cnc2c(=O)n(C)cnc2c1]

7-Bromo-3-methylpyrido[3,2-d]pyrimidin-4(3H)-one (150 mg, 0.628 mmol), 1-ethyl-7-fluoro-6-methoxy-1,2,3,4-tetrahydroisoquinoline (197 mg, 0.942 mmol), cesium carbonate (409 mg, 1.26 mmol) were dissolved in toluene/DMF (1:1, 4 mL) in a microwave flask. The reaction mixture was degassed with N₂ gas for 2 min. Pd₂dba₃ (57 mg, 0.063 mmol) and SPhos (77 mg, 0.2 mmol) were added quickly. The reaction vessel was sealed, degassed again with N₂ gas for 2 min and stirred at rt for 10 min. The mixture was heated to 150° C. in a microwave reactor for 30 min. The reaction mixture was filtered through CELITE® and concentrated under reduced pressure. The residue was purified by reverse phase HPLC to give 7-(1-ethyl-7-fluoro-6-methoxy-3,4-dihydroisoquinolin-2(1H)-yl)-3-methylpyrido[3,2-d]pyrimidin-4(3H)-one as a yellow solid (25 mg, 11% yield, m/z: 369 [M+H]⁺ observed). ¹H NMR (400 MHz, CDCl₃) δ 8.74 (d, J=2.8 Hz, 1H), 8.41-8.23 (m, 1H), 7.24 (d, J=2.7 Hz, 1H), 6.91 (d, J=11.4 Hz, 1H), 6.80 (d, J=8.3 Hz, 1H), 4.67 (t, J=7.1 Hz, 1H), 3.89 (s, 3H), 3.82-3.72 (m, 1H), 3.66 (s, 4H), 3.03 (t, J=6.2 Hz, 2H), 2.08-1.93 (m, 1H), 1.89-1.72 (m, 1H), 1.02 (t, J=7.4 Hz, 3H).

### Example 126: 5-Ethyl-8,9-difluoro-4-(2-pyrimidin-2-ylpyrimidin-4-yl)-3,5-dihydro-2H-1,4-benzoxazepine

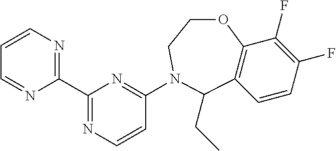 [SMILES: CCC1c2ccc(F)c(F)c2OCCN1c1ccnc(-c2ncccn2)n1]

### tert-Butyl N—[2-(2,3-difluorophenoxy)ethyl]carbamate

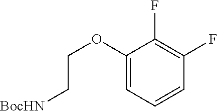 [SMILES: CC(C)(C)OC(=O)NCCOc1cccc(F)c1F]

To a mixture of 2,3-difluorophenol (10 g, 76.9 mmol) and 2-(Boc-amino)ethyl bromide (19 g, 84.6 mmol) in DMF (150 mL) was added potassium carbonate (21.3 g, 154 mmol) and sodium iodide (23 g, 154 mmol) in one portion. The mixture was stirred at 50° C. for 16 hours. The reaction mixture was cooled to rt, quenched with H₂O (500 mL) and extracted with EtOAc (3×200 mL). The combined organic phase was dried over anhydrous sulfate, filtered and concentrated under reduced pressure. The residue was purified by normal phase SiO₂ chromatography (0-10% EtOAc/petroleum ether) to give tert-butyl N—[2-(2,3-difluoro phenoxy)ethyl]carbamate as a yellow solid (13 g, 62% yield, m/z: 218 [(M-tButyl)+H]⁺ observed). ¹H NMR (400 MHz, CDCl₃): δ 6.98-6.96 (m, 1H), 6.79-6.73 (m, 2H), 5.02 (br s, 1H), 4.10 (t, J=5.2 Hz, 2H), 3.56-3.54 (t, J=5.2 Hz, 2H), 1.45 (s, 9H).

### 2-(2,3-Difluorophenoxy)ethanamine

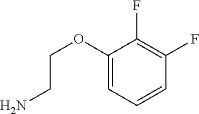 [SMILES: NCCOc1cccc(F)c1F]

A mixture of tert-butyl N—[2-(2,3-difluorophenoxy)ethyl]carbamate (8 g, 29.3 mmol) in HCl (4 M solution in 1,4-dioxane, 110 mL, 439 mmol) was stirred at rt for 6 hours. The resulting white solid was collected by filtration, washed with MTBE (100 mL) and dried under reduced to give 4.1 g of desired product as a hydrochloride salt. The product was combined with another batch at 2.7 g scale, treated with NaOH (1N solution in H₂O, 100 mL) and extracted with EtOAc (3×50 mL). The combined organic phase was washed with saturated aqueous brine solution (100 mL), dried over anhydrous sulfate, filtered and concentrated under reduced pressure to give 2-(2,3-difluoro phenoxy) ethanamine as a light-yellow oil (4.6 g, 68% yield). ¹H NMR (400 MHz, CDCl₃): δ 6.97-6.95 (m, 1H), 6.78-6.74 (m, 2H), 4.06 (t, J=5.2 Hz, 2H), 3.12 (t, J=5.2 Hz, 2H), 1.47 (s, 2H).

### 5-Ethyl-8,9-difluoro-2,3,4,5-tetrahydro-1,4-benzoxazepine

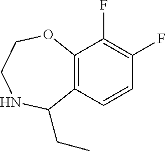

A mixture of 2-(2,3-difluorophenoxy)ethanamine (1.7 g, 9.8 mmol), propanal (0.85 mL, 11.8 mmol) and titanium(IV) isopropoxide (3.5 mL, 11.8 mmol) was heated at 70° C. for 2 h under an argon atmosphere. Meanwhile, a solution of formic acid (18.5 mL, 491 mmol) and acetic anhydride (46 mL, 491 mmol) was stirred for 2 h at rt under an argon atmosphere to prepare acetic-formic anhydride. The pre-formed acetic-formic anhydride solution was added to the reaction at 0° C. and the mixture was heated at 70° C. for 2 h. The reaction mixture was concentrated under reduced pressure. Trifluoroacetic acid (76 mL, 982 mmol) was added and the contents of the flask heated to 70° C. for 16 h. The reaction mixture was cooled to rt and concentrated in vacuum. Saturated aqueous sodium bicarbonate solution was added to adjust the pH to 8. The mixture was extracted with EtOAc (3×50 mL). The combined organic phase was dried over anhydrous sodium sulfate, filtered and concentrated under reduced pressure. The residue was purified by normal phase SiO₂ chromatography (5-30% EtOAc/hexanes) to give 5-ethyl-8,9-difluoro-3,5-dihydro-2H-1,4-benzoxazepine-4-carbaldehyde as a yellow oil, which was used in the next step without further purification (500 mg, 13% yield).

A mixture of crude 5-ethyl-8,9-difluoro-3,5-dihydro-2H-1,4-benzoxazepine-4-carbaldehyde (500 mg, 2.07 mmol) in EtOH (25 mL) and HCl (10% solution in H₂O, 25 mL) was stirred at 80° C. for 16 h. Saturated aqueous sodium carbonate solution was added to adjust the pH to 9. The mixture was extracted with EtOAc (3×20 mL). The combined organic phase was dried over anhydrous sodium sulfate, filtered and concentrated under reduced pressure. The residue was purified by normal phase SiO₂ chromatography (0-10% MeOH/CH₂C₂) to give 5-ethyl-8,9-difluoro-2,3,4,5-tetrahydro-1,4-benzoxazepine as a yellow oil (60 mg, 14% yield, m/z: 214 [M+H]⁺ observed). ¹H NMR (400 MHz, CDCl₃) δ 6.76-6.69 (m, 2H), 4.18-4.15 (m, 1H), 3.92-3.90 (m, 1H), 3.71-3.67 (m, 1H), 3.36-3.35 (m, 1H), 3.06-3.05 (m, 1H), 1.86-1.78 (m, 2H), 0.87 (t, J=7.2 Hz, 3H).

### 5-Ethyl-8,9-difluoro-4-(2-pyrimidin-2-ylpyrimidin-4-yl)-3,5-dihydro-2H-1,4-benzoxazepine

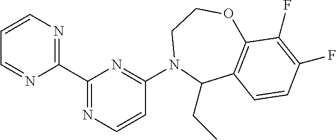 [SMILES: CCC1c2ccc(F)c(F)c2OCCN1c1ccnc(-c2ncccn2)n1]

To a mixture of 5-ethyl-8,9-difluoro-2,3,4,5-tetrahydro-1,4-benzoxazepine (60 mg, 0.28 mmol) and 4-chloro-2-pyrimidin-2-yl-pyrimidine (30 mg, 0.31 mmol) in acetonitrile (1 mL) was added N,N-disopropylethylamine (0.97 mL, 5.6 mmol). The mixture was heated to 80° C. and stirred for 40 hours. The reaction mixture was concentrated under reduced pressure. The residue was purified by reverse phase HPLC to give 5-ethyl-8,9-difluoro-4-(2-pyrimidin-2-ylpyrimidin-4-yl)-3,5-dihydro-2H-1,4-benzoxazepine as hydrochloride salt (10 mg, 10% yield, m/z: 370 [M+H]⁺ observed). ¹H NMR (400 MHz, DMSO-d₆, 80° C.): δ 9.11 (s, 2H), 8.43 (br s, 1H), 7.76 (s, 1H), 7.41-7.36 (m, 2H), 7.12-7.06 (m, 1H), 5.64 (s, 1H), 4.88 (s, 1H), 4.62 (d, J=12.4 Hz, 1H), 4.05-3.91 (m, 2H), 2.30-2.23 (m, 1H), 2.11-2.04 (m, 1H), 0.91 (t, J=7.2 Hz, 3H).

### Example 127: 2-(2,2′-Bipyrimidin-4-yl)-1-ethyl-7-fluoro-6-methoxy-1,2,3,4-tetrahydroisoquinoline

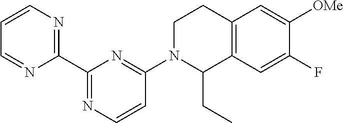 [SMILES: CCC1c2cc(F)c(OC)cc2CCN1c1ccnc(-c2ncccn2)n1]

### 2-(4-Fluoro-3-methoxyphenyl)ethan-1-amine hydrochloride

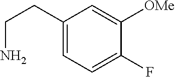 [SMILES: COc1cc(CCN)ccc1F]

To a solution of 2-(4-fluoro-3-methoxyphenyl)acetonitrile (10 g, 61 mmol) in THF (100 mL) was added lithium aluminum hydride (2M solution in THF, 67 mL, 134 mmol) at rt and the reaction was stirred for 2 h. The mixture was cooled to 0° C. and quenched with aqueous potassium hydroxide (50 mL), diluted with EtOAc (500 mL) and filtered through a CELITE® pad. The filtrate was evaporated under reduced pressure, then HCl (4M solution in 1,4-dioxane, 20 mL) was added and the reaction was stirred for 2 h at 0° C. The resulting precipitate was collected by filtration and washed with diethyl ether (2×50 mL) to afford 2-(4-fluoro-3-methoxyphenyl)ethan-1-amine as an off-white solid, hydrochloride salt (8 g, 64% yield, m/z: 170 [M+H]⁺ observed). ¹H NMR (400 MHz, DMSO-d₆) δ 8.11 (br s, 3H), 7.30-7.02 (m, 2H), 6.82-6.79 (m, 1H), 3.85 (s, 3H), 3.06-3.00 (m, 2H), 2.87 (t, 2H).

### N-(4-Fluoro-3-methoxyphenethyl)propionamide

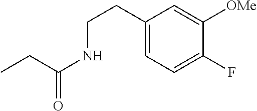 [SMILES: CCC(=O)NCCc1ccc(F)c(OC)c1]

To a solution of 2-(4-fluoro-3-methoxyphenyl)ethan-1-amine hydrochloride (6 g, 29 mmol) in dichloromethane (60 mL) was added N,N-diisopropylethylamine (15 mL, 88 mmol) and propionyl chloride (5 mL, 59 mmol) at 0° C. The reaction was stirred at rt for 2 h. The reaction mixture was diluted with water (100 mL) and extracted with dichloromethane (2×200 mL). The combined organic layer was washed with saturated aqueous brine solution (100 mL), dried with anhydrous sodium sulfate and evaporated under reduced pressure to dryness. The residue was purified by normal phase SiO₂ chromatography (0% to 30% EtOAc/hexanes) to afford N-(4-fluoro-3-methoxyphenethyl)propionamide as a white solid (5.5 g, 84% yield, m/z 226 [M+H]⁺ observed). ¹H NMR (400 MHz, CDCl₃) δ 7.02-6.97 (m, 1H), 6.80-6.78 (m, 1H), 6.71-6.67 (m, 1H), 5.42 (s, 1H), 3.88 (s, 3H), 3.50 (q, 2H), 2.78 (t, 2H), 2.20-2.14 (m, 2H), 1.15 (t, 3H).

### 1-Ethyl-7-fluoro-6-methoxy-3,4-dihydroisoquinoline

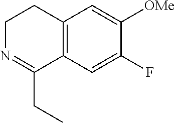

To a solution of N-(4-fluoro-3-methoxyphenethyl)propionamide (2 g, 8.8 mmol) in xylene (20 mL) was added phosphorus pentoxide (5.04 g, 35.5 mmol) and the reaction mixture was heated to 140° C. for 6 h. The reaction mixture was evaporated under reduced pressure, then diluted with ice-cold H₂O (100 mL), basified with saturated sodium bicarbonate solution and extracted with EtOAc (2×100 mL). The combined organic layer was washed with saturated aqueous brine solution (100 mL), dried with anhydrous sodium sulfate and evaporated under reduced pressure. The residue was purified by normal phase SiO₂ chromatography (0% to 70% EtOAc/petroleum ether) to afford 1-ethyl-7-fluoro-6-methoxy-3,4-dihydroisoquinoline (1.1 g, 60%, m/z 208 [M+H]⁺ observed). ¹H NMR (400 MHz, CDCl₃) δ 7.21 (d, 1H), 6.76 (d, 1H), 3.92 (s, 3H), 3.65 (t, 2H), 2.69-2.61 (m, 4H), 1.22 (t, 3H).

### 1-Ethyl-7-fluoro-6-methoxy-1,2,3,4-tetrahydroisoquinoline

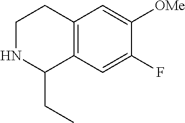

To a solution of ethyl-7-fluoro-6-methoxy-3,4-dihydroisoquinoline (1 g, 4.8 mmol) in methanol (10 mL) was added sodium borohydride (0.54 g, 14.4 mmol). The reaction mixture was stirred at rt for 2 h. The reaction mixture was diluted with water (100 mL) and extracted with EtOAc (2×100 mL). The combined organic layer was washed with saturated aqueous brine solution (100 mL), dried over anhydrous sodium sulfate and evaporated under reduced pressure to afford 1-ethyl-7-fluoro-6-methoxy-1,2,3,4-tetrahydroisoquinoline as a yellow gummy solid (0.9 g, 81%, m/z 210 [M+H]⁺ observed).

### 2-(2-Chloropyrimidin-4-yl)-1-ethyl-7-fluoro-6-methoxy-1,2,3,4-tetrahydroisoquinoline

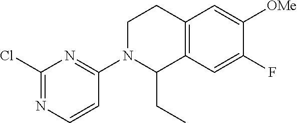 [SMILES: CCC1c2cc(F)c(OC)cc2CCN1c1ccnc(Cl)n1]

To a solution of 1-ethyl-7-fluoro-6-methoxy-1,2,3,4-tetrahydroisoquinoline (0.9 g, 4.3 mmol) in THF (10 mL) was added N,N-diisopropylethylamine (2.2 mL, 12.9 mmol) and 2,4-dichloropyrimidine (0.96 g, 6.45 mmol) at rt and heated to 50° C. for 1 h. The reaction mixture was cooled to rt, diluted with water (100 mL) and extracted with EtOAc (2×100 mL). The combined organic layer was washed with saturated aqueous brine solution (100 mL), dried with anhydrous sodium sulfate and concentrated under reduced pressure. The residue was purified by normal phase SiO₂ chromatography (0% to 20% EtOAc/petroleum ether) to afford 2-(2-chloropyrimidin-4-yl)-1-ethyl-7-fluoro-6-methoxy-1,2,3,4-tetrahydroisoquinoline as a white solid (0.8 g, 58% yield, m/z 322 [M+H]⁺ observed). ¹H NMR (400 MHz, DMSO-d₆) δ 8.78 (d, 1H), 8.08 (d, 1H), 7.01 (d, 1H), 6.89 (d, 1H), 3.93-3.87 (m, 4H), 2.89-2.85 (m, 2H), 2.50-2.48 (m, 2H), 1.83-1.81 (m, 2H), 0.89-0.87 (m, 3H).

### 2-(2,2′-Bipyrimidin-4-yl)-1-ethyl-7-fluoro-6-methoxy-1,2,3,4-tetrahydroisoquinoline

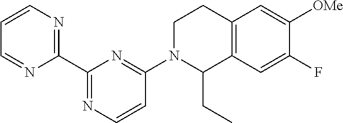 [SMILES: CCC1c2cc(F)c(OC)cc2CCN1c1ccnc(-c2ncccn2)n1]

To a solution of 2-(2-chloropyrimidin-4-yl)-1-ethyl-7-fluoro-6-methoxy-1,2,3,4-tetrahydroisoquinoline (0.8 g, 2.49 mmol) in DMF (10 mL) was added 2-(tributylstannyl) pyrimidine (1.4 g, 3.73 mmol), tetraethylammonium chloride (0.41 g, 2.49 mmol) and potassium carbonate (0.69 g, 4.98 mmol) at rt. The reaction mixture was degassed with N₂ for 10 min. Then bis(triphenylphosphine)palladium(II) dichloride (0.17 g, 0.24 mmol) was added and degassing with N₂ was continued for another 10 min. The reaction mixture was stirred at 100° C. for 24 h, cooled to rt, diluted with water (100 mL) and extracted with EtOAc (2×100 mL). The combined organic layer was washed with saturated aqueous brine solution (100 mL), dried with sodium sulfate and concentrated under reduced pressure. The crude residue was purified by reverse phase HPLC to give 2-(2,2′-bipyrimidin-4-yl)-1-ethyl-7-fluoro-6-methoxy-1,2,3,4-tetrahydroisoquinoline as a white solid (50 mg, 6% yield, m/z 366 [M+H]⁺ observed). ¹H NMR (400 MHz, DMSO-d₆) δ 8.94 (d, 2H), 8.34 (d, 1H), 7.58 (t, 1H), 7.16 (d, 1H), 6.99-6.95 (m, 2H) 3.80 (s, 3H), 3.50 (br s, 2H), 2.84 (br s, 3H), 1.86 (q, 2H), 0.87 (t, 3H).

### Example 128: 2-(2,2′-Bipyrimidin-4-yl)-1-ethyl-7-fluoro-6-methoxy-1,2,3,4-tetrahydroisoquinoline (Single Enantiomer I)

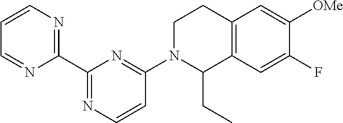 [SMILES: CCC1c2cc(F)c(OC)cc2CCN1c1ccnc(-c2ncccn2)n1]

### Example 129: 2-(2,2′-Bipyrimidin-4-yl)-1-ethyl-7-fluoro-6-methoxy-1,2,3,4-tetrahydroisoquinoline (Single Enantiomer II)

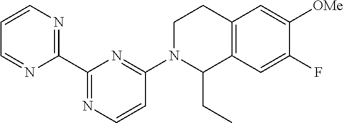 [SMILES: CCC1c2cc(F)c(OC)cc2CCN1c1ccnc(-c2ncccn2)n1]

The mixture of enantiomers was separated by SFC (supercritical fluid chromatography) on a CHIRALCEL® OD-H column using 30% EtOH to give 2-(2,2′-bipyrimidin-4-yl)-1-ethyl-7-fluoro-6-methoxy-1,2,3,4-tetrahydroisoquinoline (single enantiomer I) (faster eluting enantiomer, 9 mg, 25% yield, m/z: 366 [M+H]⁺ observed) and 2-(2,2′-bipyrimidin-4-yl)-1-ethyl-7-fluoro-6-methoxy-1,2,3,4-tetrahydroisoquinoline (single enantiomer II) (slower eluting enantiomer, 10 mg, 27% yield, m/z: 366 [M+H]⁺ observed).

### Example 128: 2-(2,2′-Bipyrimidin-4-yl)-1-ethyl-7-fluoro-6-methoxy-1,2,3,4-tetrahydroisoquinoline (Single Enantiomer I)

m/z 366 [M+H]⁺ observed. ¹H NMR (400 MHz, DMSO-d₆) δ 8.94 (d, 2H), 8.34 (d, 1H), 7.58 (t, 1H), 7.16 (d, 1H), 6.99-6.95 (m, 2H) 3.80 (s, 3H), 3.50 (br s, 2H), 2.84 (br s, 3H), 1.86 (q, 2H), 0.87 (t, 3H).

### Example 129: 2-(2,2′-Bipyrimidin-4-yl)-1-ethyl-7-fluoro-6-methoxy-1,2,3,4-tetrahydroisoquinoline (Single Enantiomer II)

m/z 366 [M+H]⁺ observed. ¹H NMR (400 MHz, DMSO-d₆) δ 8.94 (d, 2H), 8.34 (d, 1H), 7.58 (t, 1H), 7.16 (d, 1H), 6.99-6.95 (m, 2H) 3.80 (s, 3H), 3.50 (br s, 2H), 2.84 (br s, 3H), 1.86 (q, 2H), 0.87 (t, 3H).

The following examples were prepared in a similar manner as 2-([2,2′-bipyrimidin]-4-yl)-1-ethyl-7-fluoro-6-methoxy-1,2,3,4-tetrahydroisoquinoline from 2,4-dichloropyrimidine and an appropriate amine.

### Example 130: 2-([2,2′-Bipyrimidin]-4-yl)-5,6-difluoro-1-propyl-1,2,3,4-tetrahydroisoquinoline (Single Enantiomer I)

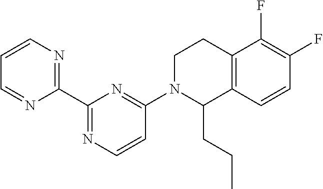 [SMILES: CCCC1c2ccc(F)c(F)c2CCN1c1ccnc(-c2ncccn2)n1]

m/z: 368 [M+H]⁺ observed. ¹H NMR (400 MHz, DMSO-d₆) δ 8.95 (d, 2H), 8.37 (d, 1H), 7.61 (t, 1H), 7.28 (d, 1H), 7.16 (d, 1H), 7.04 (d, 1H), 3.50 (m, 1H), 2.91 (m, 2H), 2.50 (m, 2H), 1.93 (m, 1H), 1.78 (m, 1H), 1.31 (m, 2H), 0.90 (t, 3H).

### Example 131: 2-([2,2′-Bipyrimidin]-4-yl)-5,6-difluoro-1-propyl-1,2,3,4-tetrahydroisoquinoline (Single Enantiomer II)

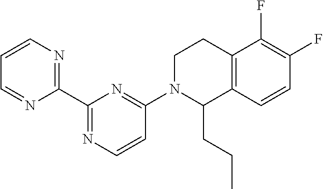 [SMILES: CCCC1c2ccc(F)c(F)c2CCN1c1ccnc(-c2ncccn2)n1]

m/z: 368 [M+H]⁺ observed. ¹H NMR (400 MHz, DMSO-d₆) δ 8.95 (d, 2H), 8.37 (d, 1H), 7.61 (t, 1H), 7.28 (d, 1H), 7.16 (d, 1H), 7.04 (d, 1H), 3.50 (m, 1H), 2.91 (m, 2H), 2.50 (m, 2H), 1.93 (m, 1H), 1.78 (m, 1H), 1.31 (m, 2H), 0.90 (t, 3H).

The following examples were prepared in a similar manner as 2-([2,2′-bipyrimidin]-4-yl)-1-ethyl-7-fluoro-6-methoxy-1,2,3,4-tetrahydroisoquinoline from 5-bromo-2,2′-bipyrimidine and an appropriate amine.

### Example 132: 2-(2,2′-Bipyrimidin-5-yl)-1-ethyl-7-fluoro-6-methoxy-1,2,3,4-tetrahydroisoquinoline

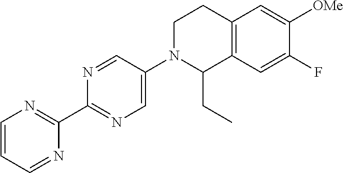 [SMILES: CCC1c2cc(F)c(OC)cc2CCN1c1cnc(-c2ncccn2)nc1]

m/z: 366 [M+H]⁺ observed. ¹H NMR (400 MHz, DMSO-d₆) δ 8.90 (d, 2H), 8.60 (s, 2H), 7.49 (t, 1H), 7.12 (d, 1H), 7.01 (d, 1H), 4.97 (t, 1H), 3.84-3.78 (m, 4H), 3.66-3.59 (m, 1H), 3.02-2.95 (m, 1H), 2.95-2.89 (m, 1H), 1.93-1.86 (m, 1H), 1.80-1.73 (m, 1H), 0.94 (t, 3H).

### Example 133: 2-(2,2′-Bipyrimidin-5-yl)-1-ethyl-7-fluoro-6-methoxy-1,2,3,4-tetrahydroisoquinoline (Single Enantiomer I)

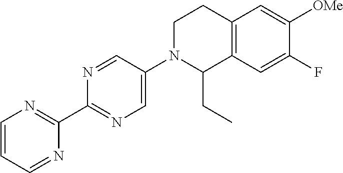 [SMILES: CCC1c2cc(F)c(OC)cc2CCN1c1cnc(-c2ncccn2)nc1]

m/z: 366 [M+H]⁺ observed. ¹H NMR (400 MHz, DMSO-d₆) δ 8.90 (d, 2H), 8.60 (s, 2H), 7.49 (t, 1H), 7.12 (d, 1H), 7.01 (d, 1H), 4.97 (t, 1H), 3.84-3.78 (m, 4H), 3.66-3.59 (m, 1H), 3.02-2.95 (m, 1H), 2.95-2.89 (m, 1H), 1.93-1.86 (m, 1H), 1.80-1.73 (m, 1H), 0.94 (t, 3H).

### Example 134: 2-(2,2′-Bipyrimidin-5-yl)-1-ethyl-7-fluoro-6-methoxy-1,2,3,4-tetrahydroisoquinoline (Single Enantiomer II)

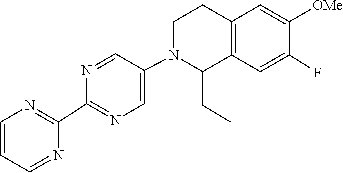 [SMILES: CCC1c2cc(F)c(OC)cc2CCN1c1cnc(-c2ncccn2)nc1]

m/z: 366 [M+H]⁺ observed. ¹H NMR (400 MHz, DMSO-d₆) δ 8.90 (d, 2H), 8.60 (s, 2H), 7.49 (t, 1H), 7.12 (d, 1H), 7.01 (d, 1H), 4.97 (t, 1H), 3.84-3.78 (m, 4H), 3.66-3.59 (m, 1H), 3.02-2.95 (m, 1H), 2.95-2.89 (m, 1H), 1.93-1.86 (m, 1H), 1.80-1.73 (m, 1H), 0.94 (t, 3H).

### Example 135: 2-([2,2′-Bipyrimidin]-4-yl)-1-ethyl-8-fluoro-7-methoxy-2,3,4,5-tetrahydro-1H-benzo[c]azepine

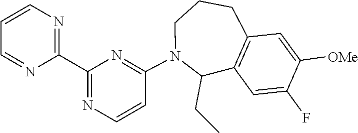 [SMILES: CCC1c2cc(F)c(OC)cc2CCCN1c1ccnc(-c2ncccn2)n1]

### (E)-3-(4-Fluoro-3-methoxyphenyl)acrylic Acid

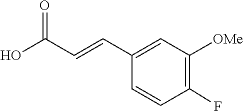 [SMILES: COc1cc(/C=C/C(=O)O)ccc1F]

To a solution of 4-fluoro-3-methoxybenzaldehyde (30 g, 195 mmol) and piperidine (4 ml, 42 mmol) in pyridine (150 mL) was added malonic acid (30.4 g, 293 mmol). The reaction mixture was refluxed for 16 h, cooled to rt and evaporated under reduced pressure. The residue was acidified with hydrochloric acid (1.5N in H₂O). The resulting precipitate was filtered and washed with petroleum ether (2×40 mL) to afford (E)-3-(4-fluoro-3-methoxyphenyl)acrylic acid as a white solid, which was taken to the next step without further purification (37 g, 97% yield, m/z: 197 [M+H]⁺ observed). ¹H NMR (300 MHz, DMSO-d₆) δ 12.55 (br s, 1H), 7.58-7.52 (m, 2H), 7.26-7.20 (m, 2H), 6.56 (d, 1H), 3.89 (s, 3H).

### 3-(4-Fluoro-3-methoxyphenyl)propanoic Acid

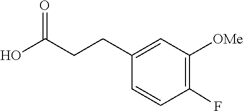 [SMILES: COc1cc(CCC(=O)O)ccc1F]

To a solution of (E)-3-(4-fluoro-3-methoxyphenyl)acrylic acid (30 g, 153 mmol) in methanol (600 mL) was added palladium (10% wt on carbon, 5 G, 4.7 mmol). The reaction mixture was stirred at rt for 24 h under H₂(balloon) pressure. The reaction mixture was filtered through a pad of CELITE® and washed with methanol (2×100 mL). The combined organic layer was evaporated under reduced pressure to afford 3-(4-fluoro-3-methoxyphenyl) propanoic acid as a white solid (24.1 g, 80% yield, m/z: 199 [M+H]⁺ observed). ¹H NMR (400 MHz, CDCl₃): δ 7.01-7.69 (m, 1H), 6.81 (dd, 1H), 6.74-6.70 (m, 1H), 3.87 (s, 3H), 2.92 (t, 2H), 2.67 (t, 2H).

### 3-(4-Fluoro-3-methoxyphenyl)propanenitrile

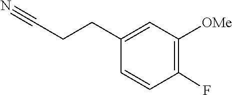 [SMILES: COc1cc(CCC#N)ccc1F]

To a solution of 3-(4-fluoro-3-methoxyphenyl)propanoic acid (20 g, 101 mmol) in dichloromethane (200 mL) at 0° C. was added DMF (0.8 mL, 10.1 mmol) and oxalyl chloride (10 mL, 121 mmol) dropwise and the reaction mixture was stirred at rt for 4 h. The reaction mixture was evaporated under reduced pressure, then dissolved in sulfolane (100 mL). Sulfamide (9 mL, 152 mmol) was added and the reaction mixture was stirred at 130° C. for 2 h. The mixture was cooled to rt, diluted with water (600 mL) and extracted with EtOAc (2×400 mL). The combined organic layer was washed with a saturated aqueous brine solution (2×200 mL), dried over anhydrous sodium sulfate and evaporated under reduced pressure to afford 3-(4-fluoro-3-methoxyphenyl)propanenitrile as an orange gummy solid, which was used stepin the next step without further purification (13.5 g, 75% yield, m/z: 180 [M+H]⁺ observed). ¹H NMR (400 MHz, CDCl₃): δ 7.05-7.00 (m, 1H), 6.84 (dd, 1H), 6.76-6.73 (m, 1H), 3.90 (s, 3H), 2.93-2.90 (t, 2H), 2.63-2.59 (t, 2H).

### 3-(4-Fluoro-3-methoxyphenyl)propan-1-amine Hydrochloride

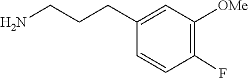 [SMILES: COc1cc(CCCN)ccc1F]

To a solution of 3-(4-fluoro-3-methoxyphenyl)propanenitrile (13 g, 73 mmol) in methanol (400 mL) was added di-tert-butyl dicarbonate (47.5 g, 217 mmol) and nickel(II)chloride hexahydrate (17.3 g, 72.6 mmol) at 0° C. To the mixture was added NaBH₄ (16.5 g, 435 mmol) portionwise over 30 minutes at 0° C. and then stirred at rt for 16 h. The mixture was filtered through a pad of CELITE® and then evaporated under reduced pressure. The residue was dissolved in EtOAc (700 mL) and washed with water (2×200 mL), dried over anhydrous sodium sulfate, filtered & evaporated under reduced pressure. The crude residue was diluted with hydrochloric acid (4N in 1,4-dioxane, 130 mL) at 0° C. and stirred at rt for 4 h. The reaction mixture was evaporated under reduced pressure and triturated with Et₂O (200 mL) to afford 3-(4-fluoro-3-methoxyphenyl)propan-1-amine, hydrochloride salt as an off-white solid (10.3 g, 65% yield, m/z 184 [M+H]⁺ observed).

### N-(3-(4-Fluoro-3-methoxyphenyl)propyl)propionamide

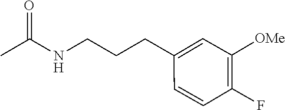 [SMILES: COc1cc(CCCNC(C)=O)ccc1F]

To a solution of 3-(4-fluoro-3-methoxyphenyl)propan-1-amine hydrochloride (10 g, 46 mmol) in CH₂Cl₂ (500 mL) was added N,N-diisopropylethylamine (24 mL, 136 mmol) and propionyl chloride (4.8 mL, 54.8 mmol) at 0° C. and stirred at rt for 2 h. The reaction mixture was diluted with water (400 mL) and extracted with dichloromethane (2×200 mL). The combined organic layer was washed with saturated aqueous brine solution (200 mL), dried over anhydrous sodium sulfate and evaporated under reduced pressure. The residue was purified by normal phase SiO₂ chromatography (0-30% EtOAc/petroleum ether) to afford N-(3-(4-fluoro-3-methoxyphenyl)propyl) propionamide as an orange liquid (7.2 g, 66% yield, m/z 240 [M+H]⁺ observed). ¹H NMR (400 MHz, CDCl₃) δ 6.99-6.94 (m, 1H), 6.78 (dd, 1H), 6.70-6.67 (m, 1H), 5.44 (br s, 1H), 3.87 (s, 3H), 3.31-3.26 (m, 2H), 2.60 (t, 2H), 2.20-2.15 (m, 2H), 1.85-1.78 (m, 2H), 1.14 (t, 3H).

### 1-Ethyl-8-fluoro-7-methoxy-2,3,4,5-tetrahydro-1H-benzo[c]azepine

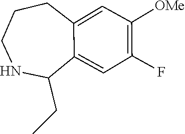 [SMILES: CCC1NCCCc2cc(OC)c(F)cc21]

To a solution of N-(3-(4-fluoro-3-methoxyphenyl)propyl) propionamide (7 g, 29.3 mmol) in xylene (200 mL) was added phosphorus pentoxide (16.6 g, 117 mmol) and the reaction mixture was heated to 140° C. for 6 h. The reaction was cooled to rt and evaporated under reduced pressure, then diluted with ice-cold water (200 mL), basified with saturated aqueous sodium bicarbonate solution and extracted with EtOAc (3×200 mL). The combined organic phase was washed with saturated aqueous brine solution (200 mL), dried over anhydrous sodium sulfate and evaporated under reduced pressure. The crude residue was dissolved in methanol (100 mL) and sodium borohydride (3.32 g, 87.8 mmol) was added at 0° C. The reaction mixture was stirred at rt for 2 h. The mixture was diluted with water (100 mL) and extracted with dichloromethane (2×100 mL). The combined organic phase was washed with saturated aqueous brine solution (100 mL), dried over anhydrous sodium sulfate and evaporated under reduced pressure to afford 1-ethyl-8-fluoro-7-methoxy-2,3,4,5-tetrahydro-1H-benzo[c]azepine as an orange oil, which was used in the next step without further purification (0.750 g, 11% yield, m/z 224 [M+H]⁺ observed).

### 2-(2-Chloropyrimidin-4-yl)-1-ethyl-8-fluoro-7-methoxy-2,3,4,5-tetrahydro-1H-benzo[c]azepine

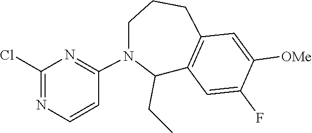 [SMILES: CCC1c2cc(F)c(OC)cc2CCCN1c1ccnc(Cl)n1]

To a solution of 1-ethyl-8-fluoro-7-methoxy-2,3,4,5-tetrahydro-1H-benzo[c]azepine (0.75 g, 3.36 mmol) in THF (20 mL) was added N,N-diisopropylethylamine (1.8 mL, 10 mmol) and 2,4-dichloropyrimidine (0.55 g, 3.69 mmol) at rt and the reaction stirred at rt for 2 h. The reaction mixture was diluted with water (100 mL) and extracted with EtOAc (2×100 mL). The combined organic layer was washed with saturated aqueous brine solution (100 mL), dried with anhydrous sodium sulfate and evaporated to dryness. The residue was purified by normal phase SiO₂ chromatography (0-30% EtOAc/petroleum ether) to afford 2-(2-chloropyrimidin-4-yl)-1-ethyl-8-fluoro-7-methoxy-2,3,4,5-tetrahydro-1H-benzo[c]azepine as an orange oil (0.36 g, 32% yield, m/z 336 [M+H]⁺ observed). ¹H NMR (400 MHz, DMSO-d₆) δ 8.03 (d, 1H), 7.42 (d, 1H), 7.08-6.84 (m, 2H), 4.81-4.70 (m, 1H), 4.00-3.82 (m, 1H), 3.76 (s, 3H), 3.16-3.13 (m, 1H), 2.78 (m, 1H), 2.20-2.14 (m, 1H), 1.96-1.82 (m, 3H), 1.58 (m, 1H), 0.82 (q, 3H).

### 2-(2,2′-Bipyrimidin-4-yl)-1-ethyl-8-fluoro-7-methoxy-2,3,4,5-tetrahydro-1H-benzo[c]azepine

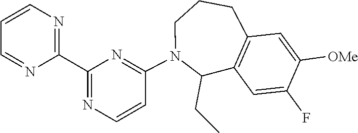 [SMILES: CCC1c2cc(F)c(OC)cc2CCCN1c1ccnc(-c2ncccn2)n1]

To a solution of 2-(2-chloropyrimidin-4-yl)-1-ethyl-8-fluoro-7-methoxy-2,3,4,5-tetrahydro-1H-benzo[c]azepine (0.35 g, 1.04 mmol)) in N,N-dimethylacetamide (4 mL) was added 2-(tributylstannyl)pyrimidine (0.33 mL, 1.04 mmol), tetraethyl ammonium chloride (0.172 g, 1.04 mmol) and potassium carbonate (0.288 g, 2.08 mmol) at rt. The reaction mixture was degassed with N₂ for 10 min. Then bis(triphenylphosphine)palladium(II) dichloride (0.073 g, 0.104 mmol) was added and the degassing with N₂ continued for 10 min. The reaction mixture was stirred at 110° C. for 32 h. The mixture was cooled to rt, diluted with water (100 mL) and extracted with EtOAc (2×100 mL). The combined organic phase was washed with saturated aqueous brine solution (100 mL), dried with anhydrous sodium sulfate and evaporated under reduced pressure. The crude residue was purified by reverse phase HPLC to give 2-(2,2′-bipyrimidin-4-yl)-1-ethyl-8-fluoro-7-methoxy-2,3,4,5-tetrahydro-1H-benzo[c]azepine as a white solid (0.035 g, 9% yield, m/z 380 [M+H]⁺ observed). ¹H NMR (400 MHz, DMSO-d₆ at 90° C.) δ 8.90 (br s, 2H), 8.27 (d, 1H), 7.52 (br s, 1H), 7.25 (br s, 1H), 6.88-6.86 (m, 2H), 5.24 (br s, 1H), 4.42 (br s, 1H), 3.76 (s, 3H), 3.32-3.58 (m, 1H), 3.04-2.49 (m, 2H), 2.30-2.21 (m, 1H), 2.08-1.98 (m, 1H), 1.89-1.70 (m, 2H), 0.93-0.88 (t, 3H).

### Example 136: Biological Examples

### HBsAg Assay

Inhibition of HBsAg was determined in HepG2.2.15 cells. Cells were maintained in culture medium containing 10% fetal calf serum, G414, Glutamine, penicillin/streptomycin. Cells were seeded in 96-well collagen-coated plate at a density of 30,000 cells/well. Serially diluted compounds were added to cells next day at the final DMSO concentration of 0.5%. Cells were incubated with compounds for 2-3 days, after which medium was removed. Fresh medium containing compounds was added to cells for additional 3-4 days. At day 6 after exposure of compounds, supernatant was collected, the HBsAg immunoassay (microplate-based chemiluminescence immunoassay kits, CLIA, Autobio Diagnosics Co., Zhengzhou, China, Catalog #CL0310-2) was used to determine the level of HBsAg according to manufactory instruction. Dose-response curves were generated and the EC₅₀ value (effective concentrations that achieved 50% inhibitory effect) were determined using XLfit software. In addition, cells were seeded at a density of 5,000 cells/well for determination of cell viability in the presence and absence of compounds by using CellTiter-Glo reagent (Promega).

Table 1 shows EC₅₀ values obtained by the HBsAg assay for selected compounds.

TABLE 1

| | | | sAg |
|---|---|---|---|
| Ex. | | | EC₅₀, |
| No. | Structure | Nomenclature | μM |
| 1 | 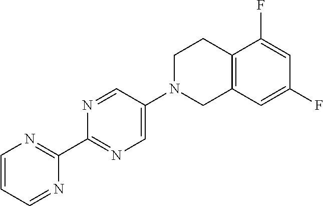 [SMILES: Fc1cc(F)c2c(c1)CN(c1cnc(-c3ncccn3)nc1)CC2] | 2-([2,2′-bipyrimidin]-5-yl)-5,7- difluoro-1,2,3,4-tetrahydroisoquinoline | 1.9 |
| 2 | 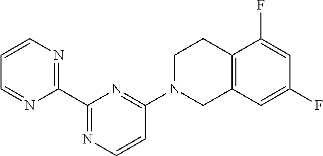 [SMILES: Fc1cc(F)c2c(c1)CN(c1ccnc(-c3ncccn3)n1)CC2] | 2-([2,2′-bipyrimidin]-4-yl)-5,7- difluoro-1,2,3,4-tetrahydroisoquinoline | 1.6 |
| 3 | 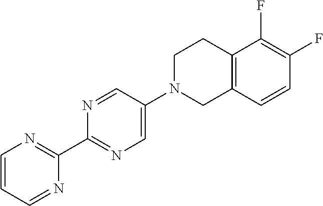 [SMILES: Fc1ccc2c(c1F)CCN(c1cnc(-c3ncccn3)nc1)C2] | 2-([2,2′-bipyrimidin]-5-yl)-5,6- difluoro-1,2,3,4-tetrahydroisoquinoline | 3.4 |
| 4 | 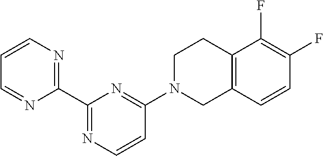 [SMILES: Fc1ccc2c(c1F)CCN(c1ccnc(-c3ncccn3)n1)C2] | 2-([2,2′-bipyrimidin]-4-yl)-5,6- difluoro-1,2,3,4-tetrahydroisoquinoline | 0.90 |
| 5 | 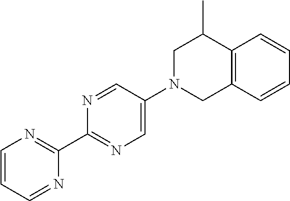 [SMILES: CC1CN(c2cnc(-c3ncccn3)nc2)Cc2ccccc21] | 2-([2,2′-bipyrimidin]-5-yl)-4-methyl- 1,2,3,4-tetrahydroisoquinoline | 34 |
| 6 | 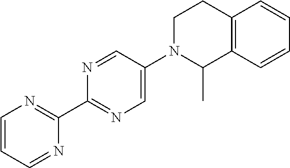 [SMILES: CC1c2ccccc2CCN1c1cnc(-c2ncccn2)nc1] | 1-methyl-2-(2-pyrimidin-2- ylpyrimidin-5-yl)-3,4-dihydro-1H- isoquinoline | 1.4 |
| 7 | 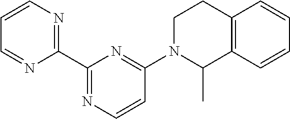 [SMILES: CC1c2ccccc2CCN1c1ccnc(-c2ncccn2)n1] | 2-([2,2′-bipyrimidin]-4-yl)-1-methyl- 1,2,3,4-tetrahydroisoquinoline | 1.9 |
| 8 | 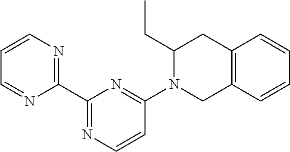 [SMILES: CCC1Cc2ccccc2CN1c1ccnc(-c2ncccn2)n1] | 2-([2,2′-bipyrimidin]-4-yl)-3-ethyl- 1,2,3,4-tetrahydroisoquinoline | 2.3 |
| 9 | 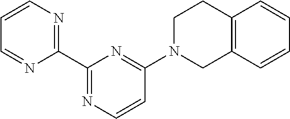 [SMILES: c1cnc(-c2nccc(N3CCc4ccccc4C3)n2)nc1] | 2-([2,2′-bipyrimidin]-4-yl)-1,2,3,4- tetrahydroisoquinoline | 8.5 |
| 10 | 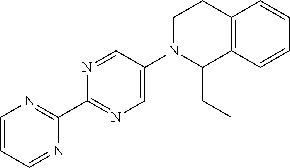 [SMILES: CCC1c2ccccc2CCN1c1cnc(-c2ncccn2)nc1] | 2-([2,2′-bipyrimidin]-5-yl)-1-ethyl- 1,2,3,4-tetrahydroisoquinoline | 0.25 |
| 11 | 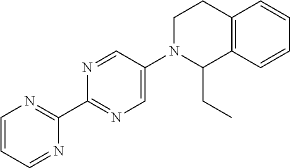 [SMILES: CCC1c2ccccc2CCN1c1cnc(-c2ncccn2)nc1] | 2-([2,2′-bipyrimidin]-5-yl)-1-ethyl- 1,2,3,4-tetrahydroisoquinoline (single enantiomer I) | 1.4 |
| 12 | 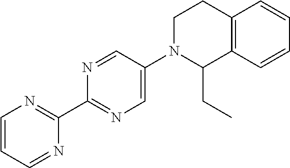 [SMILES: CCC1c2ccccc2CCN1c1cnc(-c2ncccn2)nc1] | 2-([2,2′-bipyrimidin]-5-yl)-1-ethyl- 1,2,3,4-tetrahydroisoquinoline (single enantiomer II) | 0.19 |
| 13 | 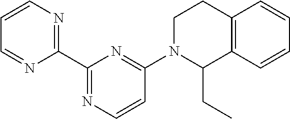 [SMILES: CCC1c2ccccc2CCN1c1ccnc(-c2ncccn2)n1] | 2-([2,2′-bipyrimidin]-4-yl)-1-ethyl- 1,2,3,4-tetrahydroisoquinoline | 0.50 |
| 14 | 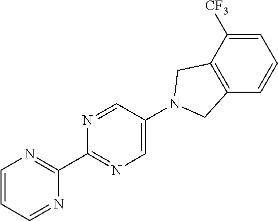 [SMILES: FC(F)(F)c1cccc2c1CN(c1cnc(-c3ncccn3)nc1)C2] | 2-([2,2′-bipyrimidin]-5-yl)-4- (trifluoromethyl)isoindoline | 25 |
| 15 | 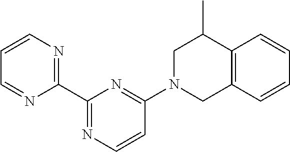 [SMILES: CC1CN(c2ccnc(-c3ncccn3)n2)Cc2ccccc21] | 2-([2,2′-bipyrimidin]-4-yl)-4-methyl- 1,2,3,4-tetrahydroisoquinoline | 1.1 |
| 16 | 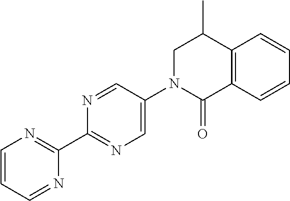 [SMILES: CC1CN(c2cnc(-c3ncccn3)nc2)C(=O)c2ccccc21] | 2-([2,2′-bipyrimidin]-5-yl)-4-methyl- 3,4-dihydroisoquinolin-1(2H)-one | 9.1 |
| 17 | 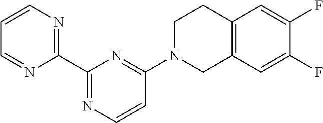 [SMILES: Fc1cc2c(cc1F)CN(c1ccnc(-c3ncccn3)n1)CC2] | 2-([2,2′-bipyrimidin]-4-yl)-6,7- difluoro-1,2,3,4-tetrahydroisoquinoline | 0.61 |
| 18 | 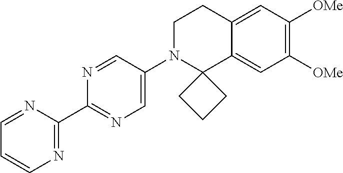 [SMILES: COc1cc2c(cc1OC)C1(CCC1)N(c1cnc(-c3ncccn3)nc1)CC2] | 2′-([2,2′-bipyrimidin]-5-yl)-6′,7′- dimethoxy-3′,4′-dihydro-2′H- spiro[cyclobutane-1,1′-isoquinoline] | 50 |
| 19 | 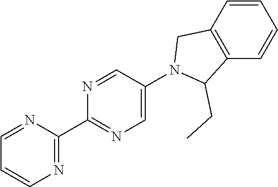 [SMILES: CCC1c2ccccc2CN1c1cnc(-c2ncccn2)nc1] | 2-([2,2′-bipyrimidin]-5-yl)-1- ethylisoindoline | 0.80 |
| 20 | 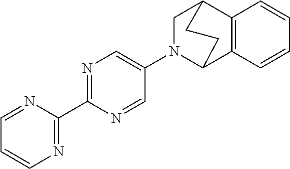 [SMILES: c1cnc(-c2ncc(N3CC4CCC3c3ccccc34)cn2)nc1] | 10-([2,2′-bipyrimidin]-5-yl)-1,2,3,4- tetrahydro-1,4- (epiminomethano)naphthalene | 32 |
| 21 | 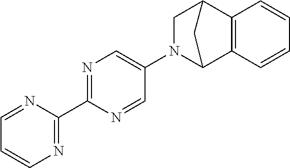 [SMILES: c1cnc(-c2ncc(N3CC4CC3c3ccccc34)cn2)nc1] | 2-([2,2′-bipyrimidin]-5-yl)-1,2,3,4- tetrahydro-1,4-methanoisoquinoline | 27 |
| 22 | 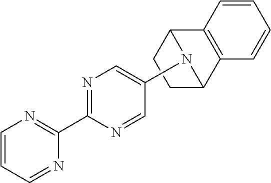 [SMILES: c1cnc(-c2ncc(N3C4CCC3c3ccccc34)cn2)nc1] | 9-([2,2′-bipyrimidin]-5-yl)-1,2,3,4- tetrahydro-1,4-epiminonaphthalene | 21 |
| 23 | 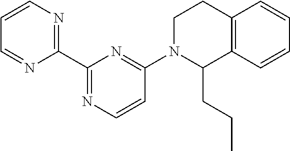 [SMILES: CCCC1c2ccccc2CCN1c1ccnc(-c2ncccn2)n1] | 2-([2,2′-bipyrimidin]-4-yl)-1-propyl- 1,2,3,4-tetrahydroisoquinoline | 1.0 |
| 24 | 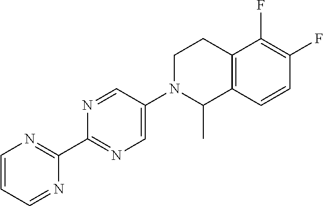 [SMILES: CC1c2ccc(F)c(F)c2CCN1c1cnc(-c2ncccn2)nc1] | 2-([2,2′-bipyrimidin]-5-yl)-5,6- difluoro-1-methyl-1,2,3,4- tetrahydroisoquinoline | 0.34 |
| 25 | 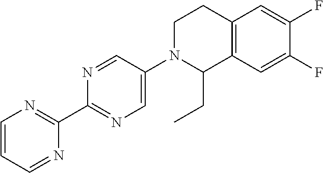 [SMILES: CCC1c2cc(F)c(F)cc2CCN1c1cnc(-c2ncccn2)nc1] | 2-([2,2′-bipyrimidin]-5-yl)-1-ethyl-6,7- difluoro-1,2,3,4-tetrahydroisoquinoline | 0.085 |
| 26 | 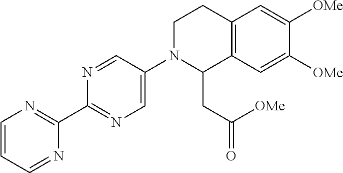 [SMILES: COC(=O)CC1c2cc(OC)c(OC)cc2CCN1c1cnc(-c2ncccn2)nc1] | methyl 2-(2-([2,2′-bipyrimidin]-5-yl)- 6,7-dimethoxy-1,2,3,4- tetrahydroisoquinolin-1-yl)acetate | 1.2 |
| 27 | 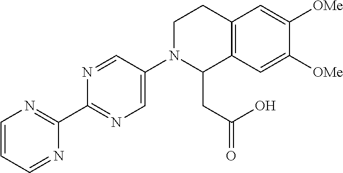 [SMILES: COc1cc2c(cc1OC)C(CC(=O)O)N(c1cnc(-c3ncccn3)nc1)CC2] | 2-(2-([2,2′-bipyrimidin]-5-yl)-6,7- dimethoxy-1,2,3,4- tetrahydroisoquinolin-1-yl)acetic acid | 28 |
| 28 | 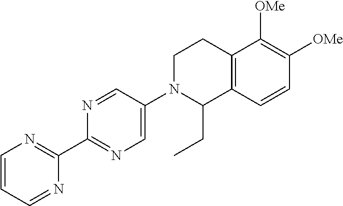 [SMILES: CCC1c2ccc(OC)c(OC)c2CCN1c1cnc(-c2ncccn2)nc1] | 2-([2,2′-bipyrimidin]-5-yl)-1-ethyl-5,6- dimethoxy-1,2,3,4- tetrahydroisoquinoline | 0.18 |
| 29 | 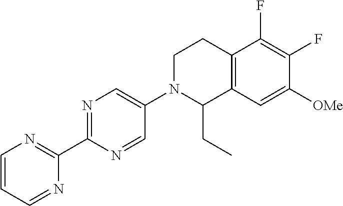 [SMILES: CCC1c2cc(OC)c(F)c(F)c2CCN1c1cnc(-c2ncccn2)nc1] | 2-([2,2′-bipyrimidin]-5-yl)-1-ethyl-5,6- difluoro-7-methoxy-1,2,3,4- tetrahydroisoquinoline | 0.17 |
| 30 | 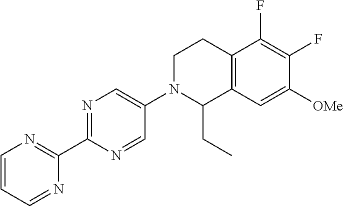 [SMILES: CCC1c2cc(OC)c(F)c(F)c2CCN1c1cnc(-c2ncccn2)nc1] | 2-([2,2′-bipyrimidin]-5-yl)-1-ethyl-5,6- difluoro-7-methoxy-1,2,3,4- tetrahydroisoquinoline (single enantiomer I) | 0.13 |
| 31 | 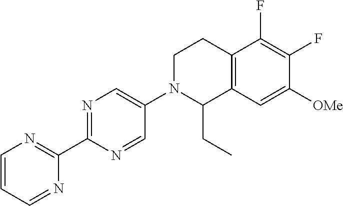 [SMILES: CCC1c2cc(OC)c(F)c(F)c2CCN1c1cnc(-c2ncccn2)nc1] | 2-([2,2′-bipyrimidin]-5-yl)-1-ethyl-5,6- difluoro-7-methoxy-1,2,3,4- tetrahydroisoquinoline (single enantiomer II) | 1.2 |
| 32 | 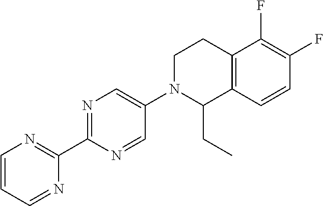 [SMILES: CCC1c2ccc(F)c(F)c2CCN1c1cnc(-c2ncccn2)nc1] | 2-([2,2′-bipyrimidin]-5-yl)-1-ethyl-5,6- difluoro-1,2,3,4-tetrahydroisoquinoline | 2.0 |
| 33 | 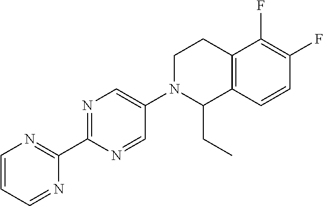 [SMILES: CCC1c2ccc(F)c(F)c2CCN1c1cnc(-c2ncccn2)nc1] | 2-([2,2′-bipyrimidin]-5-yl)-1-ethyl-5,6- difluoro-1,2,3,4-tetrahydroisoquinoline (single enantiomer I) | 0.39 |
| 34 | 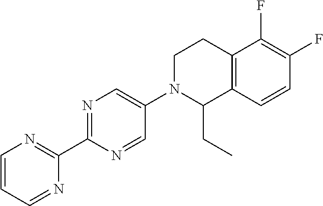 [SMILES: CCC1c2ccc(F)c(F)c2CCN1c1cnc(-c2ncccn2)nc1] | 2-([2,2′-bipyrimidin]-5-yl)-1-ethyl-5,6- difluoro-1,2,3,4-tetrahydroisoquinoline (single enantiomer II) | 0.16 |
| 35 | 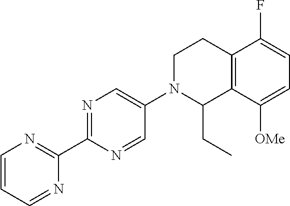 [SMILES: CCC1c2c(OC)ccc(F)c2CCN1c1cnc(-c2ncccn2)nc1] | 2-([2,2′-bipyrimidin]-5-yl)-1-ethyl-5- fluoro-8-methoxy-1,2,3,4- tetrahydroisoquinoline | 0.62 |
| 36 | 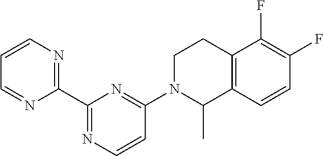 [SMILES: CC1c2ccc(F)c(F)c2CCN1c1ccnc(-c2ncccn2)n1] | 2-([2,2′-bipyrimidin]-4-yl)-5,6- difluoro-1-methyl-1,2,3,4- tetrahydroisoquinoline | 0.48 |
| 37 | 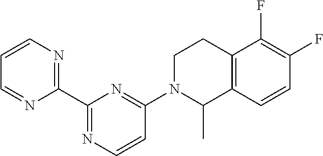 [SMILES: CC1c2ccc(F)c(F)c2CCN1c1ccnc(-c2ncccn2)n1] | 2-([2,2′-bipyrimidin]-4-yl)-5,6- difluoro-1-methyl-1,2,3,4- tetrahydroisoquinoline (single enantiomer I) | 0.31 |
| 38 | 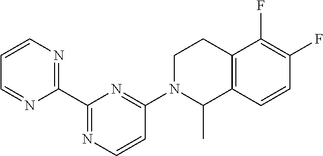 [SMILES: CC1c2ccc(F)c(F)c2CCN1c1ccnc(-c2ncccn2)n1] | 2-([2,2′-bipyrimidin]-4-yl)-5,6- difluoro-1-methyl-1,2,3,4- tetrahydroisoquinoline (single enantiomer II) | 1.4 |
| 39 | 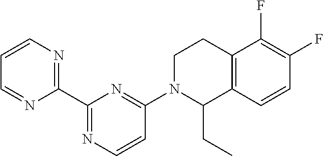 [SMILES: CCC1c2ccc(F)c(F)c2CCN1c1ccnc(-c2ncccn2)n1] | 2-([2,2′-bipyrimidin]-4-yl)-1-ethyl-5,6- difluoro-1,2,3,4-tetrahydroisoquinoline | 0.59 |
| 40 | 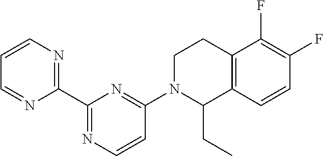 [SMILES: CCC1c2ccc(F)c(F)c2CCN1c1ccnc(-c2ncccn2)n1] | 2-([2,2′-bipyrimidin]-4-yl)-1-ethyl-5,6- difluoro-1,2,3,4-tetrahydroisoquinoline (single enantiomer I) | 0.5 |
| 41 | 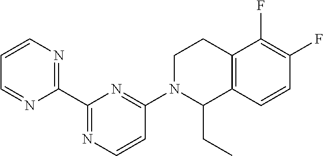 [SMILES: CCC1c2ccc(F)c(F)c2CCN1c1ccnc(-c2ncccn2)n1] | 2-([2,2′-bipyrimidin]-4-yl)-1-ethyl-5,6- difluoro-1,2,3,4-tetrahydroisoquinoline (single enantiomer II) | 2.6 |
| 42 | 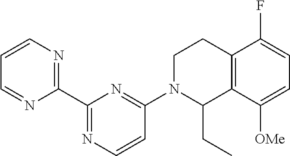 [SMILES: CCC1c2c(OC)ccc(F)c2CCN1c1ccnc(-c2ncccn2)n1] | 2-([2,2′-bipyrimidin]-4-yl)-1-ethyl-5- fluoro-8-methoxy-1,2,3,4- tetrahydroisoquinoline | 0.062 |
| 43 | 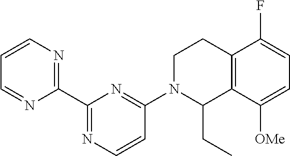 [SMILES: CCC1c2c(OC)ccc(F)c2CCN1c1ccnc(-c2ncccn2)n1] | 2-([2,2′-bipyrimidin]-4-yl)-1-ethyl-5- fluoro-8-methoxy-1,2,3,4- tetrahydroisoquinoline (single enantiomer I) | 0.06 |
| 44 | 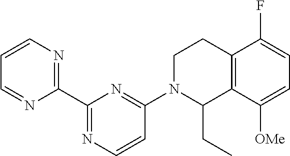 [SMILES: CCC1c2c(OC)ccc(F)c2CCN1c1ccnc(-c2ncccn2)n1] | 2-([2,2′-bipyrimidin]-4-yl)-1-ethyl-5- fluoro-8-methoxy-1,2,3,4- tetrahydroisoquinoline (single enantiomer II) | 7 |
| 45 | 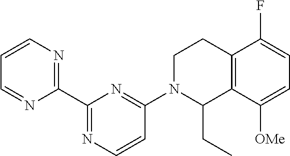 [SMILES: CCC1c2c(OC)ccc(F)c2CCN1c1ccnc(-c2ncccn2)n1] | 2-([2,2′-bipyrimidin]-4-yl)-1-ethyl-5- fluoro-8-methoxy-1,2,3,4- tetrahydroisoquinoline | 0.19 |
| 46 | 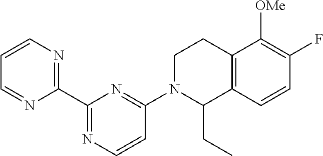 [SMILES: CCC1c2ccc(F)c(OC)c2CCN1c1ccnc(-c2ncccn2)n1] | 2-([2,2′-bipyrimidin]-4-yl)-1-ethyl-6- fluoro-5-methoxy-1,2,3,4- tetrahydroisoquinoline | 0.13 |
| 47 | 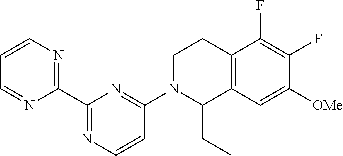 [SMILES: CCC1c2cc(OC)c(F)c(F)c2CCN1c1ccnc(-c2ncccn2)n1] | 2-([2,2′-bipyrimidin]-4-yl)-1-ethyl-5,6- difluoro-7-methoxy-1,2,3,4- tetrahydroisoquinoline | 0.48 |
| 48 | 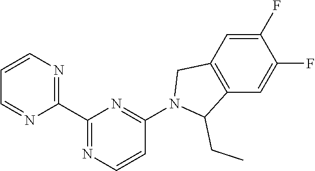 [SMILES: CCC1c2cc(F)c(F)cc2CN1c1ccnc(-c2ncccn2)n1] | 2-([2,2′-bipyrimidin]-4-yl)-1-ethyl-5,6- difluoroisoindoline | 0.092 |
| 49 | 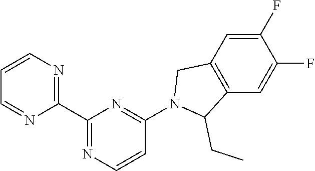 [SMILES: CCC1c2cc(F)c(F)cc2CN1c1ccnc(-c2ncccn2)n1] | 2-([2,2′-bipyrimidin]-4-yl)-1-ethyl-5,6- difluoroisoindoline (single enantiomer I) | 0.044 |
| 50 | 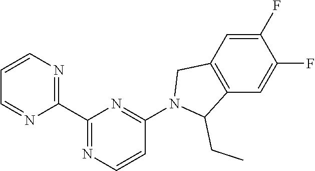 [SMILES: CCC1c2cc(F)c(F)cc2CN1c1ccnc(-c2ncccn2)n1] | 2-([2,2′-bipyrimidin]-4-yl)-1-ethyl-5,6- difluoroisoindoline (single enantiomer II) | 0.4 |
| 51 | 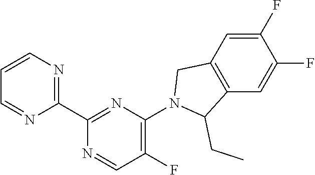 [SMILES: CCC1c2cc(F)c(F)cc2CN1c1nc(-c2ncccn2)ncc1F] | 1-ethyl-5,6-difluoro-2-(5-fluoro-[2,2′- bipyrimidin]-4-yl)isoindoline | 0.027 |
| 52 | 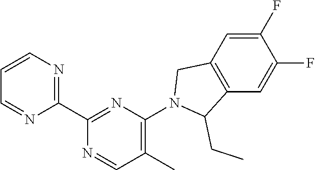 [SMILES: CCC1c2cc(F)c(F)cc2CN1c1nc(-c2ncccn2)ncc1C] | 1-ethyl-5,6-difluoro-2-(5-methyl-[2,2′- bipyrimidin]-4-yl)isoindoline | 0.043 |
| 53 | 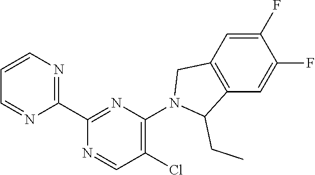 [SMILES: CCC1c2cc(F)c(F)cc2CN1c1nc(-c2ncccn2)ncc1Cl] | 2-(5-chloro-[2,2′-bipyrimidin]-4-yl)-1- ethyl-5,6-difluoroisoindoline | 0.03 |
| 54 | 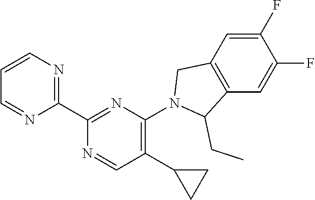 [SMILES: CCC1c2cc(F)c(F)cc2CN1c1nc(-c2ncccn2)ncc1C1CC1] | 2-(5-cyclopropyl-[2,2′-bipyrimidin]-4- yl)-1-ethyl-5,6-difluoroisoindoline | 0.22 |
| 55 | 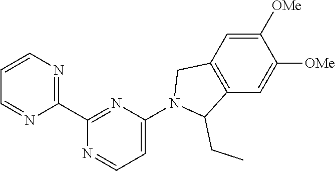 [SMILES: CCC1c2cc(OC)c(OC)cc2CN1c1ccnc(-c2ncccn2)n1] | 2-([2,2′-bipyrimidin]-4-yl)-1-ethyl-5,6- dimethoxyisoindoline | 0.024 |
| 56 | 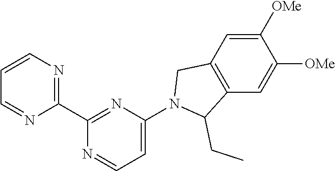 [SMILES: CCC1c2cc(OC)c(OC)cc2CN1c1ccnc(-c2ncccn2)n1] | 2-([2,2′-bipyrimidin]-4-yl)-1-ethyl-5,6- dimethoxyisoindoline (single enantiomer I) | 0.012 |
| 57 | 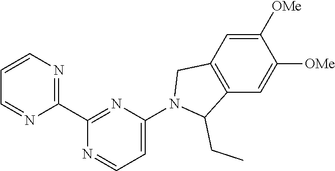 [SMILES: CCC1c2cc(OC)c(OC)cc2CN1c1ccnc(-c2ncccn2)n1] | 2-([2,2′-bipyrimidin]-4-yl)-1-ethyl-5,6- dimethoxyisoindoline (single enantiomer II) | 0.076 |
| 58 | 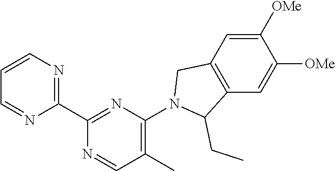 [SMILES: CCC1c2cc(OC)c(OC)cc2CN1c1nc(-c2ncccn2)ncc1C] | 1-ethyl-5,6-dimethoxy-2-(5-methyl- [2,2′-bipyrimidin]-4-yl)isoindoline | 0.002 |
| 59 | 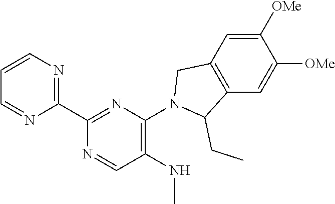 [SMILES: CCC1c2cc(OC)c(OC)cc2CN1c1nc(-c2ncccn2)ncc1NC] | 4-(1-ethyl-5,6-dimethoxyisoindolin-2- yl)-N-methyl-[2,2′-bipyrimidin]-5- amine | 0.13 |
| 60 | 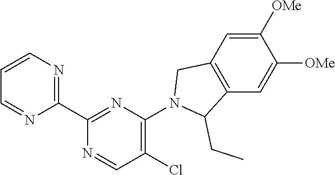 [SMILES: CCC1c2cc(OC)c(OC)cc2CN1c1nc(-c2ncccn2)ncc1Cl] | 2-(5-chloro-[2,2′-bipyrimidin]-4-yl)-1- ethyl-5,6-dimethoxyisoindoline | 0.003 |
| 61 | 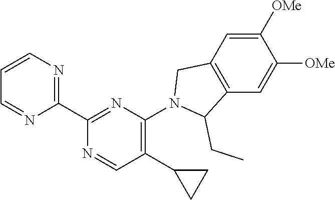 [SMILES: CCC1c2cc(OC)c(OC)cc2CN1c1nc(-c2ncccn2)ncc1C1CC1] | 2-(5-cyclopropyl-[2,2′-bipyrimidin]-4- yl)-1-ethyl-5,6-dimethoxyisoindoline | 0.014 |
| 62 | 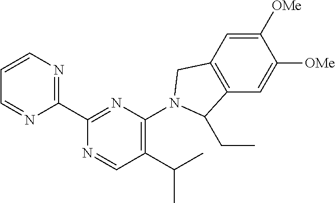 [SMILES: CCC1c2cc(OC)c(OC)cc2CN1c1nc(-c2ncccn2)ncc1C(C)C] | 1-ethyl-2-(5-isopropyl-[2,2′- bipyrimidin]-4-yl)-5,6- dimethoxyisoindoline | 1.0 |
| 63 | 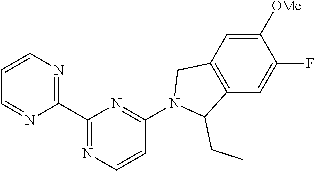 [SMILES: CCC1c2cc(F)c(OC)cc2CN1c1ccnc(-c2ncccn2)n1] | 2-([2,2′-bipyrimidin]-4-yl)-1-ethyl-6- fluoro-5-methoxyisoindoline | 0.021 |
| 64 | 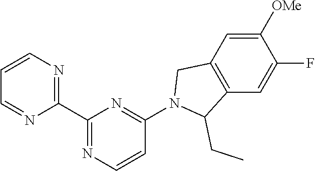 [SMILES: CCC1c2cc(F)c(OC)cc2CN1c1ccnc(-c2ncccn2)n1] | 2-([2,2′-Bipyrimidin]-4-yl)-1-ethyl-6- fluoro-5-methoxyisoindoline (single enantiomer I) | 0.012 |
| 65 | 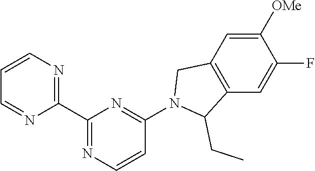 [SMILES: CCC1c2cc(F)c(OC)cc2CN1c1ccnc(-c2ncccn2)n1] | 2-([2,2′-Bipyrimidin]-4-yl)-1-ethyl-6- fluoro-5-methoxyisoindoline (single enantiomer II) | 0.23 |
| 66 | 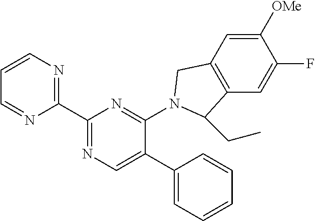 [SMILES: CCC1c2cc(F)c(OC)cc2CN1c1nc(-c2ncccn2)ncc1-c1ccccc1] | 1-ethyl-6-fluoro-5-methoxy-2-(5- phenyl-[2,2′-bipyrimidin]-4- yl)isoindoline (single enantiomer I) | 1.0 |
| 67 | 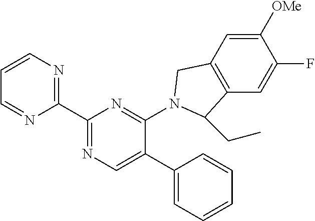 [SMILES: CCC1c2cc(F)c(OC)cc2CN1c1nc(-c2ncccn2)ncc1-c1ccccc1] | 1-ethyl-6-fluoro-5-methoxy-2-(5- phenyl-[2,2′-bipyrimidin]-4- yl)isoindoline (single enantiomer II) | 18 |
| 68 | 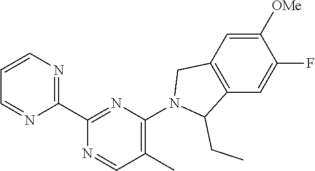 [SMILES: CCC1c2cc(F)c(OC)cc2CN1c1nc(-c2ncccn2)ncc1C] | 1-ethyl-6-fluoro-5-methoxy-2-(5- methyl-[2,2′-bipyrimidin]-4- yl)isoindoline (single enantiomer I) | 0.002 |
| 69 | 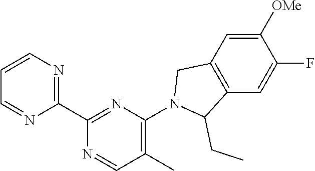 [SMILES: CCC1c2cc(F)c(OC)cc2CN1c1nc(-c2ncccn2)ncc1C] | 1-ethyl-6-fluoro-5-methoxy-2-(5- methyl-[2,2′-bipyrimidin]-4- yl)isoindoline (single enantiomer II) | 0.059 |
| 70 | 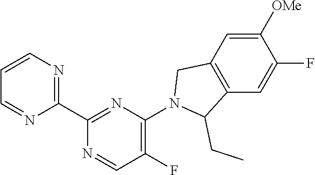 [SMILES: CCC1c2cc(F)c(OC)cc2CN1c1nc(-c2ncccn2)ncc1F] | 1-ethyl-6-fluoro-2-(5-fluoro-[2,2′- bipyrimidin]-4-yl)-5- methoxyisoindoline | 0.005 |
| 71 | 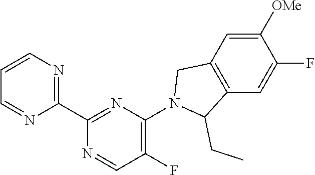 [SMILES: CCC1c2cc(F)c(OC)cc2CN1c1nc(-c2ncccn2)ncc1F] | 1-ethyl-6-fluoro-2-(5-fluoro-[2,2′- bipyrimidin]-4-yl)-5- methoxyisoindoline (single enantiomer I) | 0.003 |
| 72 | 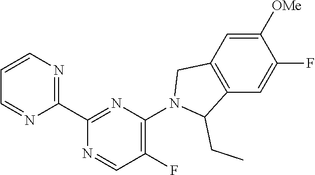 [SMILES: CCC1c2cc(F)c(OC)cc2CN1c1nc(-c2ncccn2)ncc1F] | 1-ethyl-6-fluoro-2-(5-fluoro-[2,2′- bipyrimidin]-4-yl)-5- methoxyisoindoline (single enantiomer II) | 0.067 |
| 73 | 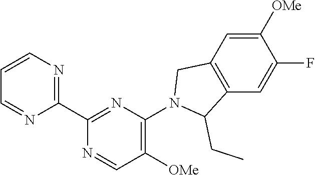 [SMILES: CCC1c2cc(F)c(OC)cc2CN1c1nc(-c2ncccn2)ncc1OC] | 1-ethyl-6-fluoro-5-methoxy-2-(5- methoxy-[2,2′-bipyrimidin]-4- yl)isoindoline (single enantiomer I) | 1.0 |
| 74 | 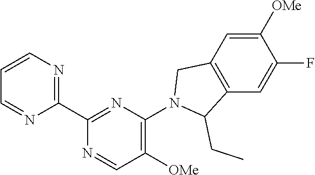 [SMILES: CCC1c2cc(F)c(OC)cc2CN1c1nc(-c2ncccn2)ncc1OC] | 1-ethyl-6-fluoro-5-methoxy-2-(5- methoxy-[2,2′-bipyrimidin]-4- yl)isoindoline (single enantiomer II) | 0.008 |
| 75 | 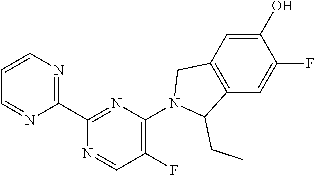 [SMILES: CCC1c2cc(F)c(O)cc2CN1c1nc(-c2ncccn2)ncc1F] | 1-ethyl-6-fluoro-2-(5-fluoro-[2,2′- bipyrimidin]-4-yl)isoindolin-5-ol | 0.70 |
| 76 | 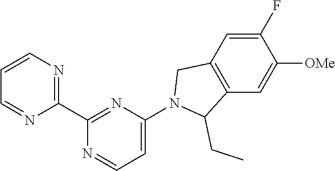 [SMILES: CCC1c2cc(OC)c(F)cc2CN1c1ccnc(-c2ncccn2)n1] | 2-([2,2′-bipyrimidin]-4-yl)-1-ethyl-5- fluoro-6-methoxyisoindoline | 0.12 |
| 77 | 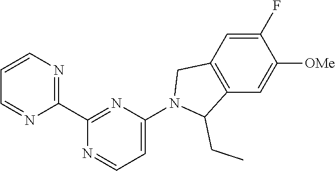 [SMILES: CCC1c2cc(OC)c(F)cc2CN1c1ccnc(-c2ncccn2)n1] | 2-([2,2′-bipyrimidin]-4-yl)-1-ethyl-5- fluoro-6-methoxyisoindoline (single enantiomer I) | 0.11 |
| 78 | 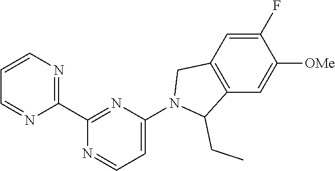 [SMILES: CCC1c2cc(OC)c(F)cc2CN1c1ccnc(-c2ncccn2)n1] | 2-([2,2′-bipyrimidin]-4-yl)-1-ethyl-5- fluoro-6-methoxyisoindoline (single enantiomer II) | 0.098 |
| 79 | 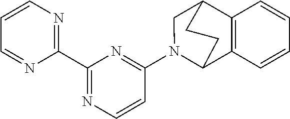 [SMILES: c1cnc(-c2nccc(N3CC4CCC3c3ccccc34)n2)nc1] | 10-([2,2′-bipyrimidin]-4-yl)-1,2,3,4- tetrahydro-1,4- (epiminomethano)naphthalene | 6.0 |
| 80 | 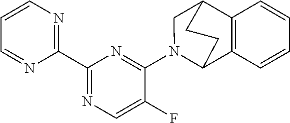 [SMILES: Fc1cnc(-c2ncccn2)nc1N1CC2CCC1c1ccccc12] | 10-(5-fluoro-[2,2′-bipyrimidin]-4-yl)- 1,2,3,4-tetrahydro-1,4- (epiminomethano)naphthalene | 0.70 |
| 81 | 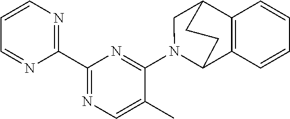 [SMILES: Cc1cnc(-c2ncccn2)nc1N1CC2CCC1c1ccccc12] | 10-(5-methyl-[2,2′-bipyrimidin]-4-yl)- 1,2,3,4-tetrahydro-1,4- (epiminomethano)naphthalene | 0.28 |
| 82 | 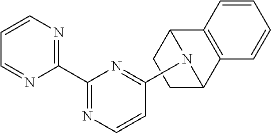 [SMILES: c1cnc(-c2nccc(N3C4CCC3c3ccccc34)n2)nc1] | 9-([2,2′-bipyrimidin]-4-yl)-1,2,3,4- tetrahydro-1,4-epiminonaphthalene | 9.0 |
| 83 | 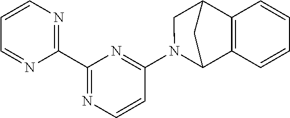 [SMILES: c1cnc(-c2nccc(N3CC4CC3c3ccccc34)n2)nc1] | 2-([2,2′-bipyrimidin]-4-yl)-1,2,3,4- tetrahydro-1,4-methanoisoquinoline | 5.0 |
| 84 | 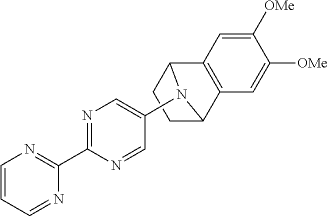 [SMILES: COc1cc2c(cc1OC)C1CCC2N1c1cnc(-c2ncccn2)nc1] | 9-([2,2′-bipyrimidin]-5-yl)-6,7- dimethoxy-1,2,3,4-tetrahydro-1,4- epiminonaphthalene | 4.0 |
| 85 | 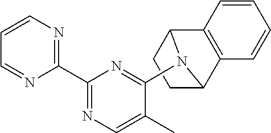 [SMILES: Cc1cnc(-c2ncccn2)nc1N1C2CCC1c1ccccc12] | 9-(5-methyl-[2,2′-bipyrimidin]-4-yl)- 1,2,3,4-tetrahydro-1,4- epiminonaphthalene | 3.0 |
| 86 | 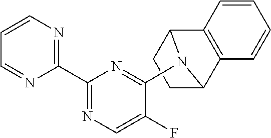 [SMILES: Fc1cnc(-c2ncccn2)nc1N1C2CCC1c1ccccc12] | 9-(5-fluoro-[2,2′-bipyrimidin]-4-yl)- 1,2,3,4-tetrahydro-1,4- epiminonaphthalene | 2.4 |
| 87 | 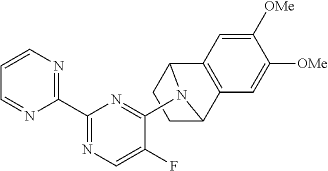 [SMILES: COc1cc2c(cc1OC)C1CCC2N1c1nc(-c2ncccn2)ncc1F] | 9-(5-fluoro-[2,2′-bipyrimidin]-4-yl)- 6,7-dimethoxy-1,2,3,4-tetrahydro-1,4- epiminonaphthalene | 1.0 |
| 88 | 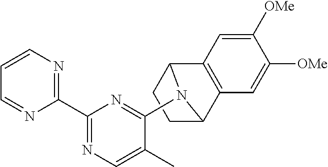 [SMILES: COc1cc2c(cc1OC)C1CCC2N1c1nc(-c2ncccn2)ncc1C] | 6,7-dimethoxy-9-(5-methyl-[2,2′- bipyrimidin]-4-yl)-1,2,3,4-tetrahydro- 1,4-epiminonaphthalene | 2.0 |
| 89 | 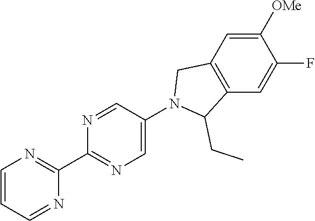 [SMILES: CCC1c2cc(F)c(OC)cc2CN1c1cnc(-c2ncccn2)nc1] | 2-([2,2′-bipyrimidin]-5-yl)-1-ethyl-6- fluoro-5-methoxyisoindoline (single enantiomer I) | 0.008 |
| 90 | 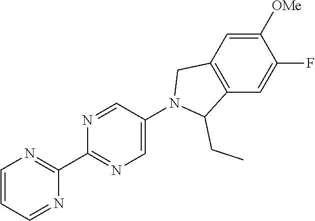 [SMILES: CCC1c2cc(F)c(OC)cc2CN1c1cnc(-c2ncccn2)nc1] | 2-([2,2′-bipyrimidin]-5-yl)-1-ethyl-6- fluoro-5-methoxyisoindoline (single enantiomer II) | 0.26 |
| 91 | 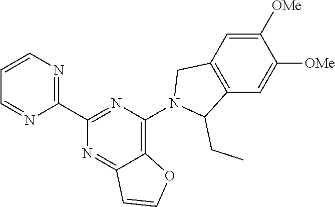 [SMILES: CCC1c2cc(OC)c(OC)cc2CN1c1nc(-c2ncccn2)nc2ccoc12] | 4-(1-ethyl-5,6-dimethoxyisoindolin-2- yl)-2-(pyrimidin-2-yl)furo[3,2- d]pyrimidine | 0.15 |
| 92 | 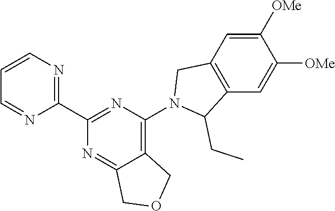 [SMILES: CCC1c2cc(OC)c(OC)cc2CN1c1nc(-c2ncccn2)nc2c1COC2] | 4-(1-ethyl-5,6-dimethoxyisoindolin-2- yl)-2-(pyrimidin-2-yl)-5,7- dihydrofuro[3,4-d]pyrimidine | 0.18 |
| 93 | 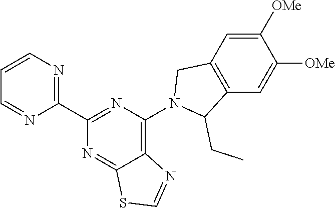 [SMILES: CCC1c2cc(OC)c(OC)cc2CN1c1nc(-c2ncccn2)nc2scnc12] | 7-(1-ethyl-5,6-dimethoxyisoindolin-2- yl)-5-(pyrimidin-2-yl)thiazolo[5,4- d]pyrimidine | 0.2 |
| 94 | 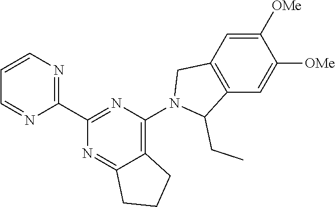 [SMILES: CCC1c2cc(OC)c(OC)cc2CN1c1nc(-c2ncccn2)nc2c1CCC2] | 4-(1-ethyl-5,6-dimethoxyisoindolin-2- yl)-2-(pyrimidin-2-yl)-6,7-dihydro-5H- cyclopenta[d]pyrimidine | 0.35 |
| 95 | 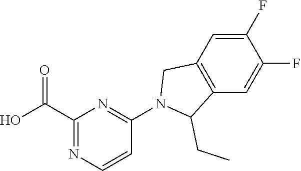 [SMILES: CCC1c2cc(F)c(F)cc2CN1c1ccnc(C(=O)O)n1] | 4-(1-ethyl-5,6-difluoroisoindolin-2- yl)pyrimidine-2-carboxylic acid | 2.0 |
| 96 | 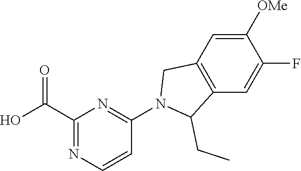 [SMILES: CCC1c2cc(F)c(OC)cc2CN1c1ccnc(C(=O)O)n1] | 4-(1-ethyl-6-fluoro-5- methoxyisoindolin-2-yl)pyrimidine-2- carboxylic acid | 1.2 |
| 97 | 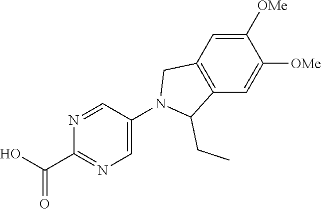 [SMILES: CCC1c2cc(OC)c(OC)cc2CN1c1cnc(C(=O)O)nc1] | 5-(1-ethyl-5,6-dimethoxyisoindolin-2- yl)pyrimidine-2-carboxylic acid | 5.0 |
| 98 | 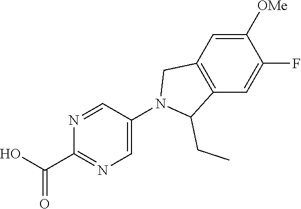 [SMILES: CCC1c2cc(F)c(OC)cc2CN1c1cnc(C(=O)O)nc1] | 5-(1-ethyl-6-fluoro-5- methoxyisoindolin-2-yl)pyrimidine-2- carboxylic acid | 0.24 |
| 99 | 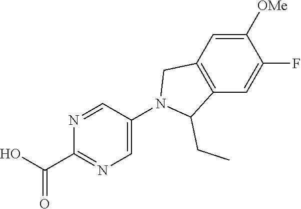 [SMILES: CCC1c2cc(F)c(OC)cc2CN1c1cnc(C(=O)O)nc1] | 5-(1-ethyl-6-fluoro-5- methoxyisoindolin-2-yl)pyrimidine-2- carboxylic acid (single enantiomer I) | 7.0 |
| 100 | 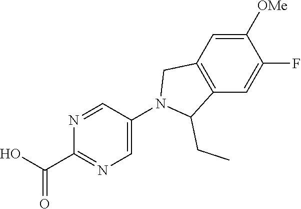 [SMILES: CCC1c2cc(F)c(OC)cc2CN1c1cnc(C(=O)O)nc1] | 5-(1-ethyl-6-fluoro-5- methoxyisoindolin-2-yl)pyrimidine-2- carboxylic acid (single enantiomer II) | 0.20 |
| 101 | 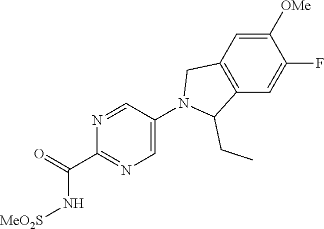 [SMILES: CCC1c2cc(F)c(OC)cc2CN1c1cnc(C(=O)NS(C)(=O)=O)nc1] | 5-(1-ethyl-6-fluoro-5- methoxyisoindolin-2-yl)-N- (methylsulfonyl)pyrimidine-2- carboxamide | 0.65 |
| 102 | 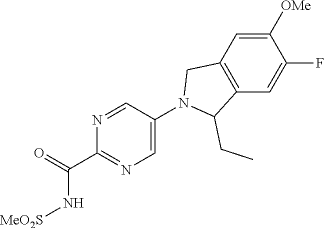 [SMILES: CCC1c2cc(F)c(OC)cc2CN1c1cnc(C(=O)NS(C)(=O)=O)nc1] | 5-(1-ethyl-6-fluoro-5- methoxyisoindolin-2-yl)-N- (methylsulfonyl)pyrimidine-2- carboxamide (single enantiomer I) | 0.62 |
| 103 | 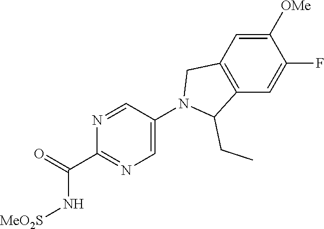 [SMILES: CCC1c2cc(F)c(OC)cc2CN1c1cnc(C(=O)NS(C)(=O)=O)nc1] | 5-(1-ethyl-6-fluoro-5- methoxyisoindolin-2-yl)-N- (methylsulfonyl)pyrimidine-2- carboxamide (single enantiomer II) | 0.60 |
| 104 | 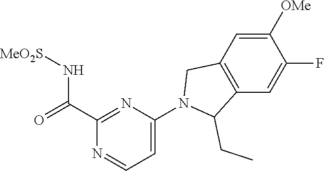 [SMILES: CCC1c2cc(F)c(OC)cc2CN1c1ccnc(C(=O)NS(C)(=O)=O)n1] | 4-(1-ethyl-6-fluoro-5- methoxyisoindolin-2-yl)-N- (methylsulfonyl)pyrimidine-2- carboxamide | 5.0 |
| 105 | 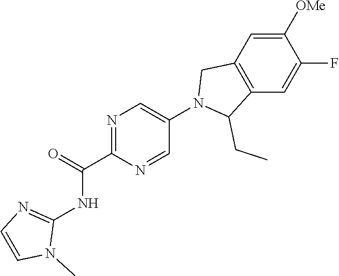 [SMILES: CCC1c2cc(F)c(OC)cc2CN1c1cnc(C(=O)Nc2nccn2C)nc1] | 5-(1-ethyl-6-fluoro-5- methoxyisoindolin-2-yl)-N-(1-methyl- 1H-imidazol-2-yl)pyrimidine-2- carboxamide | 6.0 |
| 106 | 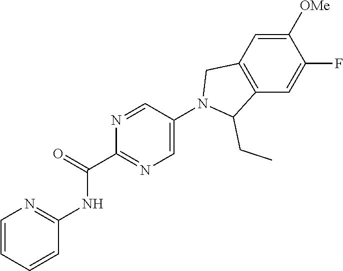 [SMILES: CCC1c2cc(F)c(OC)cc2CN1c1cnc(C(=O)Nc2ccccn2)nc1] | 5-(1-ethyl-6-fluoro-5- methoxyisoindolin-2-yl)-N-(pyridin-2- yl)pyrimidine-2-carboxamide | 2.0 |
| 107 | 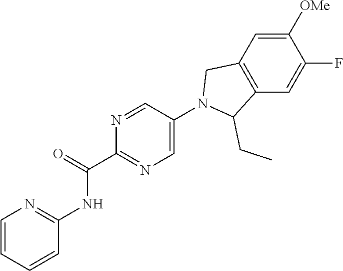 [SMILES: CCC1c2cc(F)c(OC)cc2CN1c1cnc(C(=O)Nc2ccccn2)nc1] | 5-(1-ethyl-6-fluoro-5- methoxyisoindolin-2-yl)-N-(pyridin-2- yl)pyrimidine-2-carboxamide (single enantiomer I) | 49 |
| 108 | 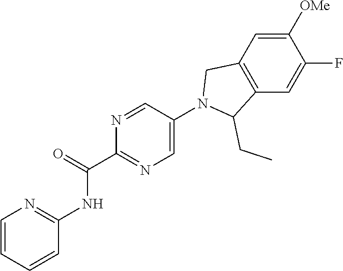 [SMILES: CCC1c2cc(F)c(OC)cc2CN1c1cnc(C(=O)Nc2ccccn2)nc1] | 5-(1-ethyl-6-fluoro-5- methoxyisoindolin-2-yl)-N-(pyridin-2- yl)pyrimidine-2-carboxamide (single enantiomer II) | 0.56 |
| 109 | 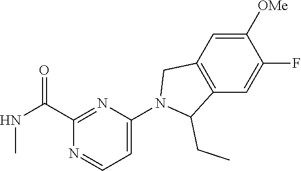 [SMILES: CCC1c2cc(F)c(OC)cc2CN1c1ccnc(C(=O)NC)n1] | 4-(1-ethyl-6-fluoro-5- methoxyisoindolin-2-yl)-N- methylpyrimidine-2-carboxamide (single enantiomer I) | 42 |
| 110 | 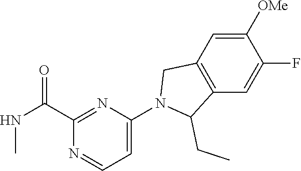 [SMILES: CCC1c2cc(F)c(OC)cc2CN1c1ccnc(C(=O)NC)n1] | 4-(1-ethyl-6-fluoro-5- methoxyisoindolin-2-yl)-N- methylpyrimidine-2-carboxamide (single enantiomer II) | 3.0 |
| 111 | 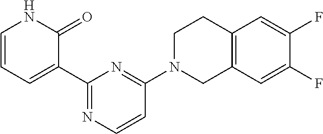 [SMILES: O=c1[nH]cccc1-c1nccc(N2CCc3cc(F)c(F)cc3C2)n1] | 3-(4-(6,7-difluoro-3,4- dihydroisoquinolin-2(1H)- yl)pyrimidin-2-yl)pyridin-2-ol | 46 |
| 112 | 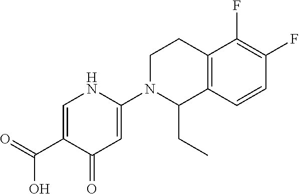 [SMILES: CCC1c2ccc(F)c(F)c2CCN1c1cc(=O)c(C(=O)O)c[nH]1] | 6-(1-ethyl-5,6-difluoro-3,4- dihydroisoquinolin-2(1H)-yl)-4-oxo- 1,4-dihydropyridine-3-carboxylic acid | 11 |
| 113 | 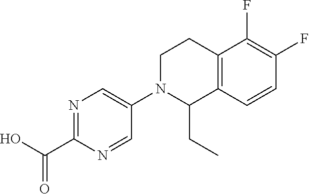 [SMILES: CCC1c2ccc(F)c(F)c2CCN1c1cnc(C(=O)O)nc1] | 5-(1-ethyl-5,6-difluoro-3,4- dihydroisoquinolin-2(1H)- yl)pyrimidine-2-carboxylic acid | 2.0 |
| 114 | 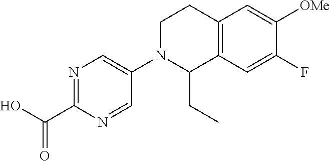 [SMILES: CCC1c2cc(F)c(OC)cc2CCN1c1cnc(C(=O)O)nc1] | 5-(1-ethyl-7-fluoro-6-methoxy-3,4- dihydroisoquinolin-2(1H)- yl)pyrimidine-2-carboxylic acid (single enantiomer I) | 0.057 |
| 115 | 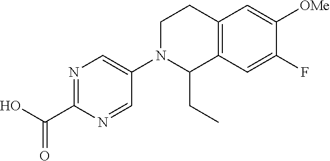 [SMILES: CCC1c2cc(F)c(OC)cc2CCN1c1cnc(C(=O)O)nc1] | 5-(1-ethyl-7-fluoro-6-methoxy-3,4- dihydroisoquinolin-2(1H)- yl)pyrimidine-2-carboxylic acid (single enantiomer II) | 6.0 |
| 116 | 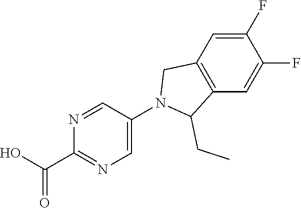 [SMILES: CCC1c2cc(F)c(F)cc2CN1c1cnc(C(=O)O)nc1] | 5-(1-ethyl-5,6-difluoroisoindolin-2- yl)pyrimidine-2-carboxylic acid (single enantiomer I) | 3.0 |
| 117 | 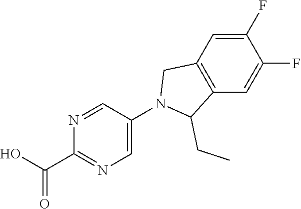 [SMILES: CCC1c2cc(F)c(F)cc2CN1c1cnc(C(=O)O)nc1] | 5-(1-ethyl-5,6-difluoroisoindolin-2- yl)pyrimidine-2-carboxylic acid (single enantiomer II) | 15 |
| 118 | 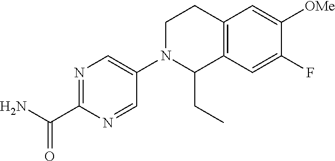 [SMILES: CCC1c2cc(F)c(OC)cc2CCN1c1cnc(C(N)=O)nc1] | 5-(1-ethyl-7-fluoro-6-methoxy-3,4- dihydroisoquinolin-2(1H)- yl)pyrimidine-2-carboxamide | 0.52 |
| 119 | 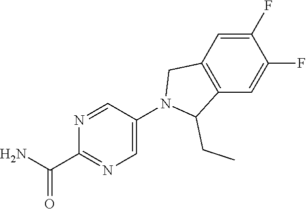 [SMILES: CCC1c2cc(F)c(F)cc2CN1c1cnc(C(N)=O)nc1] | 5-(1-ethyl-5,6-difluoroisoindolin-2- yl)pyrimidine-2-carboxamide | 42 |
| 120 | 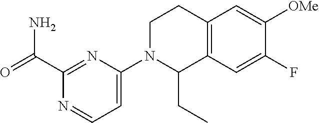 [SMILES: CCC1c2cc(F)c(OC)cc2CCN1c1ccnc(C(N)=O)n1] | 4-(1-ethyl-7-fluoro-6-methoxy-3,4- dihydroisoquinolin-2(1H)- yl)pyrimidine-2-carboxamide | 14 |
| 121 | 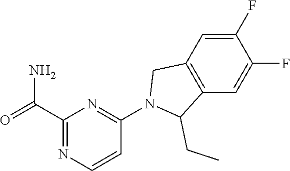 [SMILES: CCC1c2cc(F)c(F)cc2CN1c1ccnc(C(N)=O)n1] | 4-(1-ethyl-5,6-difluoroisoindolin-2- yl)pyrimidine-2-carboxamide | 18 |
| 122 | 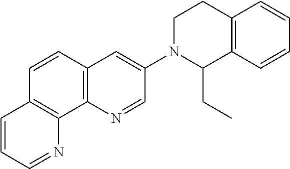 [SMILES: CCC1c2ccccc2CCN1c1cnc2c(ccc3cccnc32)c1] | 3-(1-ethyl-3,4-dihydroisoquinolin- 2(1H)-yl)-1,10-phenanthroline | 1.2 |
| 123 | 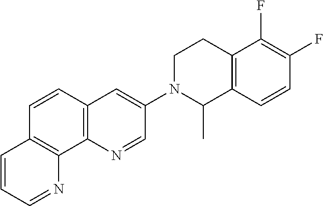 [SMILES: CC1c2ccc(F)c(F)c2CCN1c1cnc2c(ccc3cccnc32)c1] | 3-(5,6-difluoro-1-methyl-3,4- dihydroisoquinolin-2(1H)-yl)-1,10- phenanthroline | 0.44 |
| 124 | 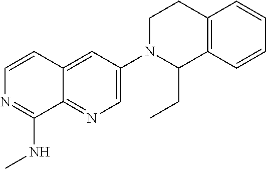 [SMILES: CCC1c2ccccc2CCN1c1cnc2c(NC)nccc2c1] | 3-(1-ethyl-3,4-dihydroisoquinolin- 2(1H)-yl)-N-methyl-1,7-naphthyridin- 8-amine | 22 |
| 125 | 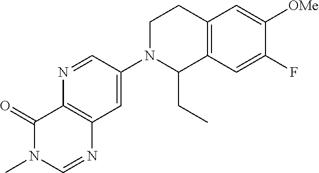 [SMILES: CCC1c2cc(F)c(OC)cc2CCN1c1cnc2c(=O)n(C)cnc2c1] | 7-(1-ethyl-7-fluoro-6-methoxy-3,4- dihydroisoquinolin-2(1H)-yl)-3- methylpyrido[3,2-d]pyrimidin-4(3H)- one | 1.6 |
| 126 | 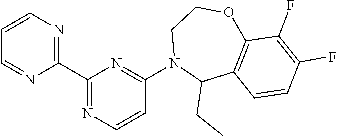 [SMILES: CCC1c2ccc(F)c(F)c2OCCN1c1ccnc(-c2ncccn2)n1] | 5-Ethyl-8,9-difluoro-4-(2-pyrimidin-2- ylpyrimidin-4-yl)-3,5-dihydro-2H-1,4- benzoxazepine | 28 |
| 127 | 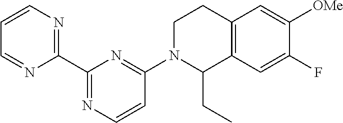 [SMILES: CCC1c2cc(F)c(OC)cc2CCN1c1ccnc(-c2ncccn2)n1] | 2-(2,2′-bipyrimidin-4-yl)-1-ethyl-7- fluoro-6-methoxy-1,2,3,4- tetrahydroisoquinoline | 0.020 |
| 128 | 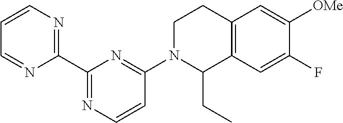 [SMILES: CCC1c2cc(F)c(OC)cc2CCN1c1ccnc(-c2ncccn2)n1] | 2-([2,2′-bipyrimidin]-4-yl)-1-ethyl-7- fluoro-6-methoxy-1,2,3,4- tetrahydroisoquinoline (single enantiomer I) | 0.082 |
| 129 | 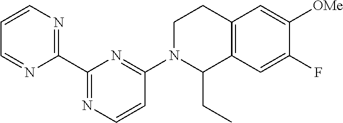 [SMILES: CCC1c2cc(F)c(OC)cc2CCN1c1ccnc(-c2ncccn2)n1] | 2-([2,2′-bipyrimidin]-4-yl)-1-ethyl-7- fluoro-6-methoxy-1,2,3,4- tetrahydroisoquinoline (single enantiomer II) | 0.015 |
| 130 | 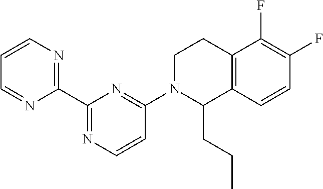 [SMILES: CCCC1c2ccc(F)c(F)c2CCN1c1ccnc(-c2ncccn2)n1] | 2-([2,2′-bipyrimidin]-4-yl)-5,6- difluoro-1-propyl-1,2,3,4- tetrahydroisoquinoline (single enantiomer I) | 0.097 |
| 131 | 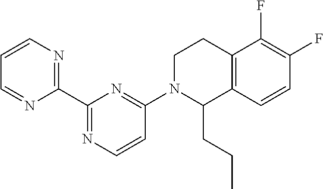 [SMILES: CCCC1c2ccc(F)c(F)c2CCN1c1ccnc(-c2ncccn2)n1] | 2-([2,2′-bipyrimidin]-4-yl)-5,6- difluoro-1-propyl-1,2,3,4- tetrahydroisoquinoline (single enantiomer II) | 2.0 |
| 132 | 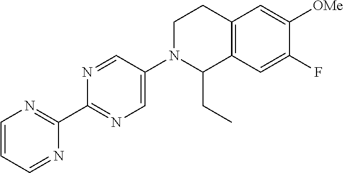 [SMILES: CCC1c2cc(F)c(OC)cc2CCN1c1cnc(-c2ncccn2)nc1] | 2-([2,2′-bipyrimidin]-5-yl)-1-ethyl-7- fluoro-6-methoxy-1,2,3,4- tetrahydroisoquinoline | 0.007 |
| 133 | 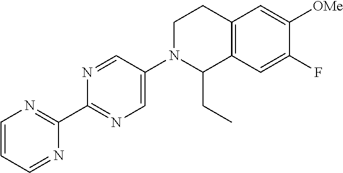 [SMILES: CCC1c2cc(F)c(OC)cc2CCN1c1cnc(-c2ncccn2)nc1] | 2-([2,2′-bipyrimidin]-5-yl)-1-ethyl-7- fluoro-6-methoxy-1,2,3,4- tetrahydroisoquinoline (single enantiomer I) | 0.003 |
| 134 | 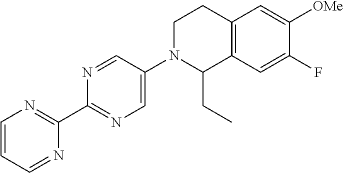 [SMILES: CCC1c2cc(F)c(OC)cc2CCN1c1cnc(-c2ncccn2)nc1] | 2-([2,2′-bipyrimidin]-5-yl)-1-ethyl-7- fluoro-6-methoxy-1,2,3,4- tetrahydroisoquinoline (single enantiomer II) | 0.054 |
| 135 | 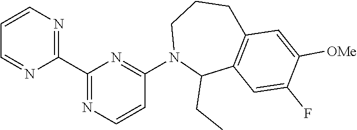 [SMILES: CCC1c2cc(F)c(OC)cc2CCCN1c1ccnc(-c2ncccn2)n1] | 2-([2,2′-bipyrimidin]-4-yl)-1-ethyl-8- fluoro-7-methoxy-2,3,4,5-tetrahydro- 1H-benzo[c]azepine | 1.0 |

### Enumerated Embodiments

The following exemplary embodiments are provided, the numbering of which is not to be construed as designating levels of importance.

Embodiment 1 provides a compound selected from the group consisting of:

(i) a compound of formula (Ia):

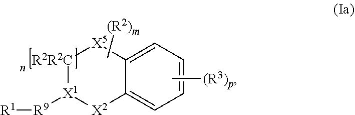

wherein in (Ia):

X¹ is N and X² is CR²R², or X² is NR⁴ and X¹ is CR⁴;

X⁵ is selected from the group consisting of O and CR²R², or one R² group from X⁵ can combine with one R² group of X² to form C₁-C₆ alkylene;

R¹ is selected from the group consisting of:

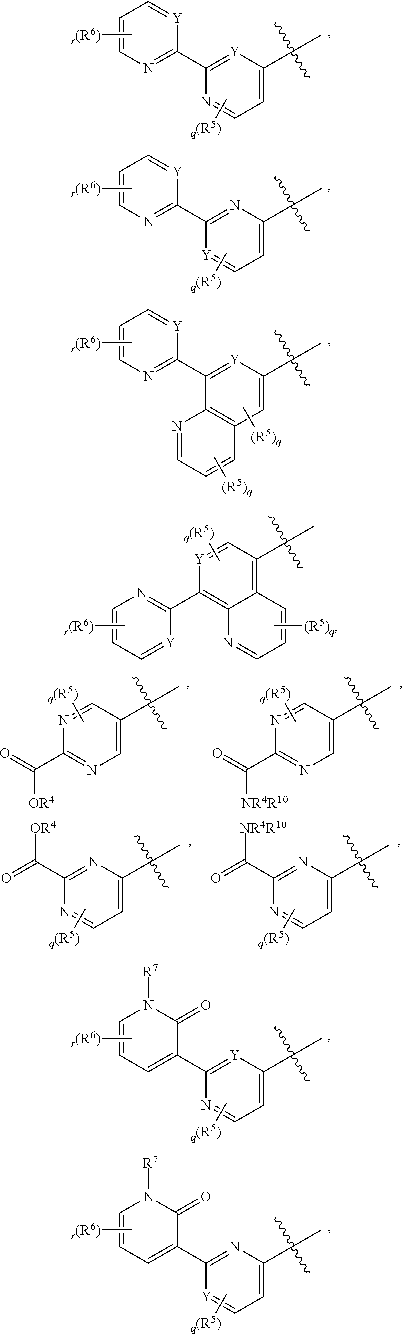

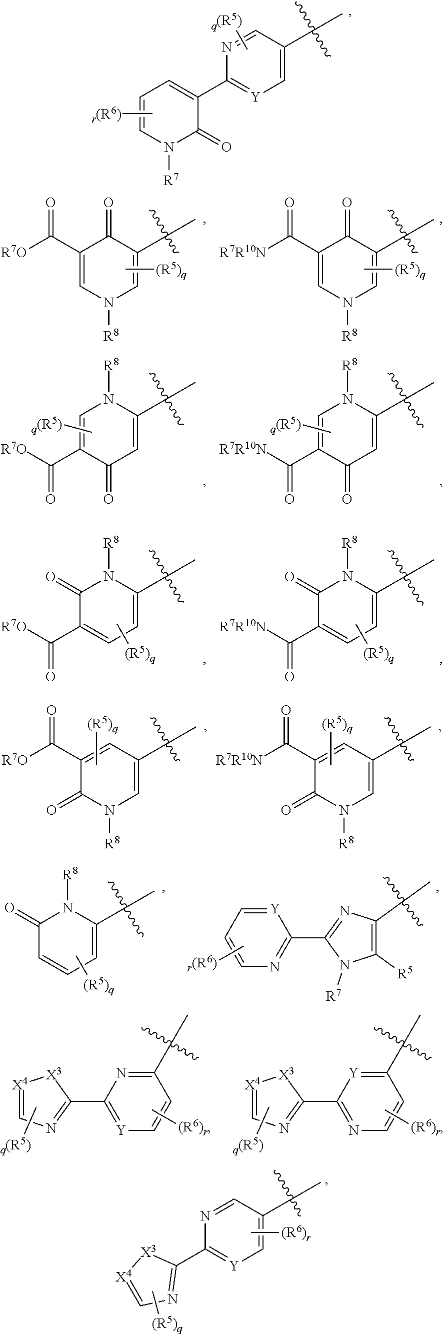

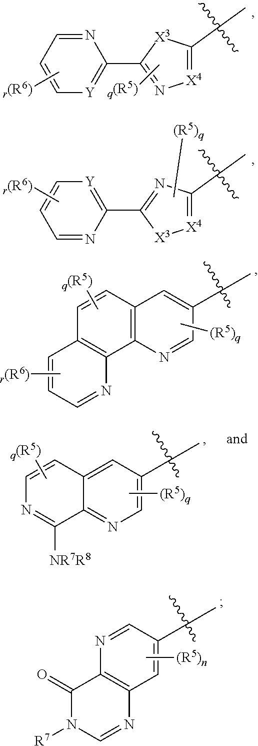

R⁹ is a bond if X¹ is CH, or R⁹ is selected from the group consisting of a bond and —C(═O)— if X¹ is N;

each occurrence of X³ is independently selected from the group consisting of NR⁷, O, and S;

each occurrence of X⁴ is independently selected from the group consisting of NR⁷ and CR⁵;

each occurrence of Y is independently selected from the group consisting of N and CR⁵;

each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)O₂C(═O)OR′, and —N(R′)(R′), wherein each occurrence of R′ is independently selected from the group consisting of H, optionally substituted C₁-C₆ alkyl, and optionally substituted C₃-C₈ cycloalkyl; or two R² combine with the carbon atom to which both of them are bound to form a substituent selected from the group consisting of C(═O) and optionally substituted 1,1-(C₃-C₈ cycloalkanediyl); or two R² bound to different carbon atoms combine to form an optionally substituted C₁-C₆ alkanediyl;

each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁴ is independently selected from the group consisting of H, optionally substituted C₁-C₆ alkyl, and optionally substituted C₃-C₈ cycloalkyl;

each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl; or two R⁵ bound to adjacent carbon atoms combine to form optionally substituted 5-7 membered carbocyclyl or heterocyclyl;

each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁷ is independently selected from the group consisting of H, optionally substituted C₁-C₆ alkyl, and optionally substituted C₃-C₈ cycloalkyl;

each occurrence of R⁸ is independently selected from the group consisting of H, optionally substituted C₁-C₆ alkyl, and optionally substituted C₃-C₈ cycloalkyl;

each occurrence of R¹⁰ is independently selected from the group consisting of H, optionally substituted C₁-C₆ alkyl, optionally substituted C₃-C₈ cycloalkyl, optionally substituted phenyl, optionally substituted heteroaryl, —S(═O)₂(optionally substituted C₁-C₆ alkyl), and —S(═O)₂(optionally substituted C₃-C₈ cycloalkyl);

m is 0, 1, 2, 3, or 4;

n is 0, 1, or 2;

p is 0, 1, 2, 3, or 4;

q is 0, 1, or 2;

r is 0, 1, 2, or 3;

(ii) a compound of formula (Ib):

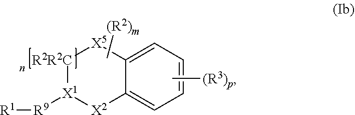

wherein in (Ib):

X¹ is N and X² is CR²R², or X² is NR⁴ and X¹ is CR⁴;

X⁵ is selected from the group consisting of O and CR²R², or one R² group from X⁵ can combine with one R² group of X² to form C₁-C₆ alkylene;

R¹ is

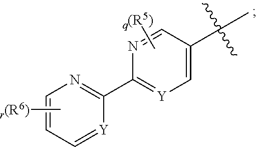

R⁹ is a bond if X¹ is CH, or R¹ is selected from the group consisting of a bond and —C(═O)— if X¹ is N;
wherein, if R⁹ is a bond, X¹ is N, X² is CHR², and X⁵ is CH₂, then n is not 1;

each occurrence of Y is independently selected from the group consisting of N and CR⁵;

each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)₀₋₂C(O)OR′, and —N(R′)(R′), wherein each occurrence of R′ is independently selected from the group consisting of H, optionally substituted C₁-C₆ alkyl, and optionally substituted C₃-C₈ cycloalkyl; or two R² combine with the carbon atom to which both of them are bound to form a substituent selected from the group consisting of C(═O) and optionally substituted 1,1-(C₃-C₈ cycloalkanediyl); or two R² bound to different carbon atoms combine to form an optionally substituted C₁-C₆ alkanediyl;

each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

each occurrence of R⁴ is independently selected from the group consisting of H, optionally substituted C₁-C₆ alkyl, and optionally substituted C₃-C₈ cycloalkyl;

each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl; or two R⁵ bound to adjacent carbon atoms combine to form optionally substituted 5-7 membered carbocyclyl or heterocyclyl;

each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;

m is 0, 1, 2, 3, or 4;

n is 0, 1, or 2;

p is 0, 1, 2, 3, or 4;

q is 0, 1, or 2;

r is 0, 1, 2, or 3;

or a salt, solvate, geometric isomer, stereoisomer, tautomer, and any mixtures thereof.

Embodiment 2 provides the compound of Embodiment 1, which is

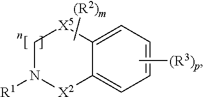

wherein X² is CR²R².

Embodiment 3 provides the compound of Embodiment 1, which is

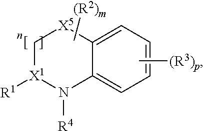

wherein X¹ is CR⁴.

Embodiment 4 provides the compound of any of Embodiments 1-3, wherein each occurrence of R⁴ is independently selected from the group consisting of H, methyl, ethyl, propyl, isopropyl, butyl, isobutyl, sec-butyl, and tert-butyl.

Embodiment 5 provides the compound of any of Embodiments 1-4, wherein R¹ is selected from the group consisting of:

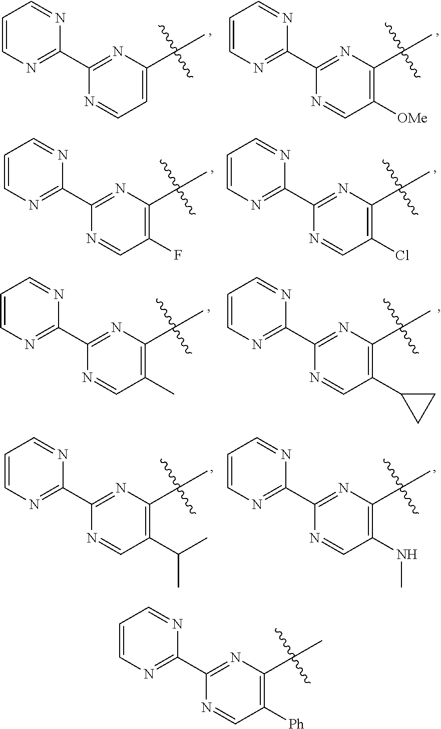

wherein Ph is optionally substituted,

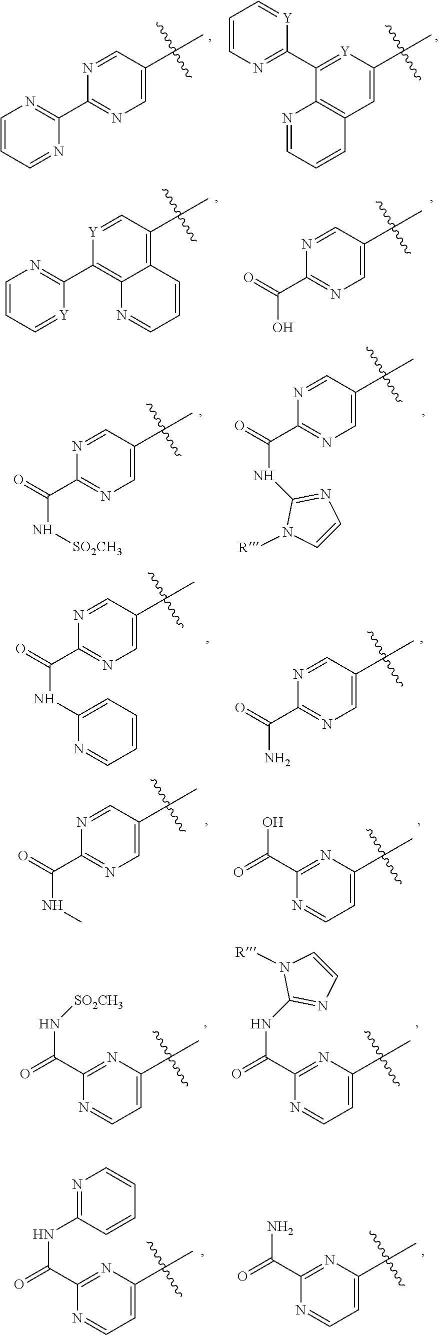

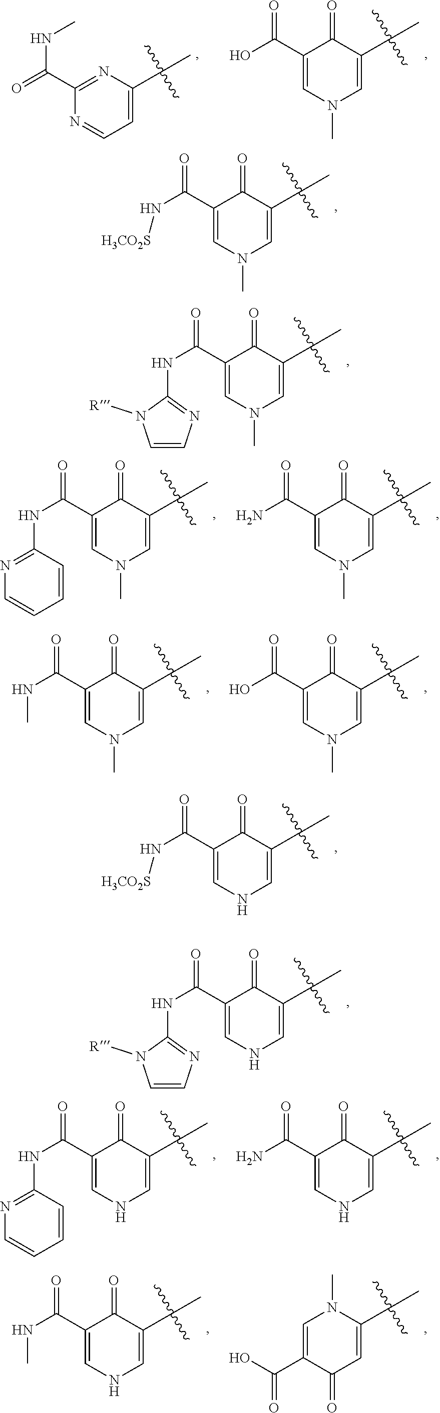

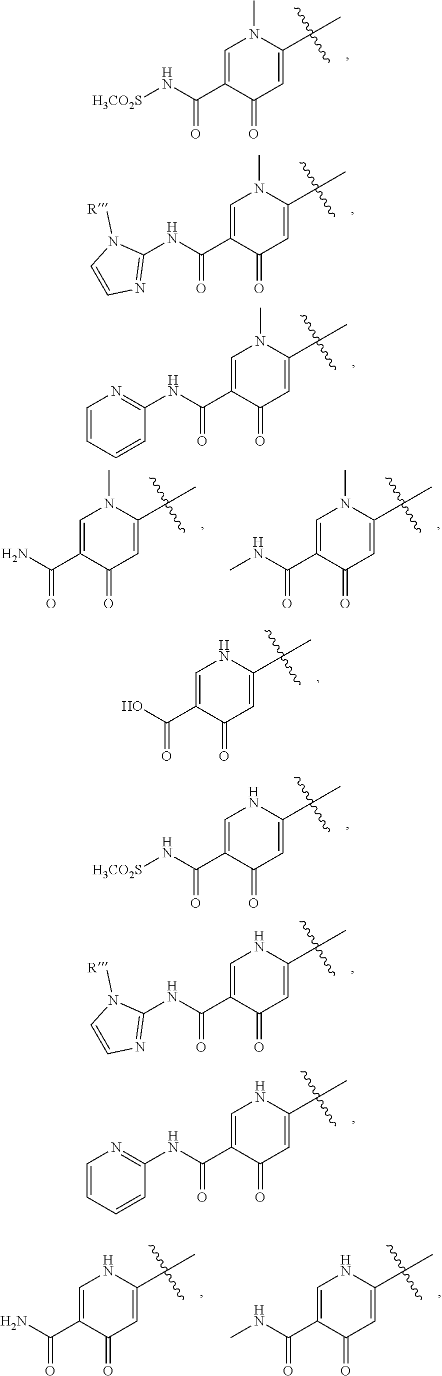

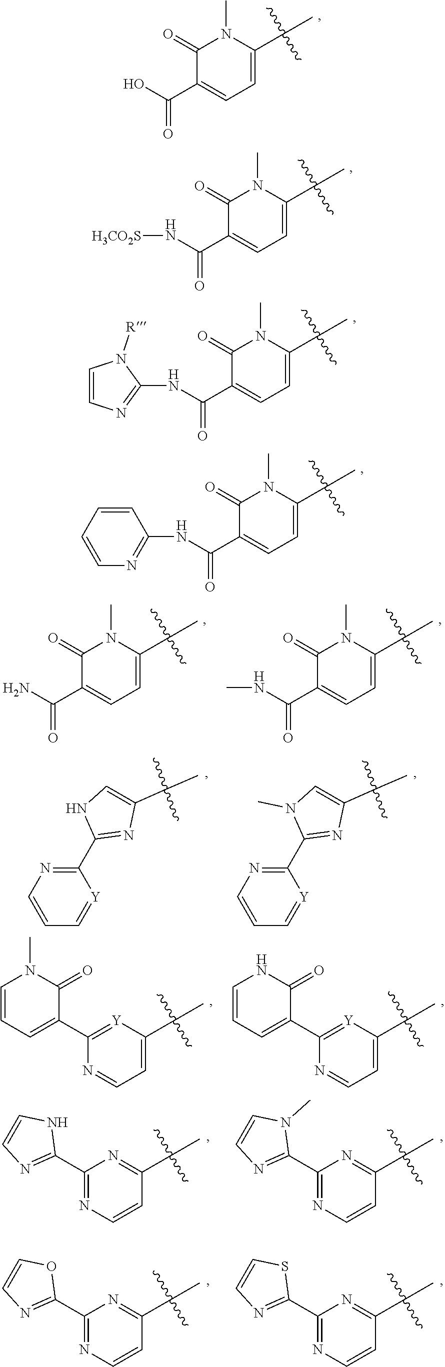

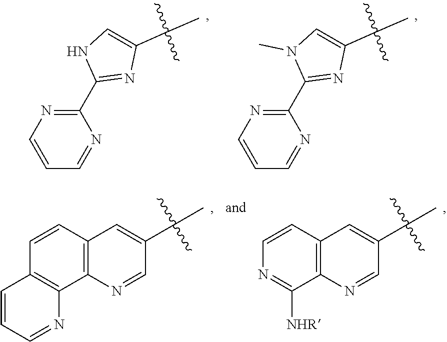

wherein each occurrence of R′″ is independently H, C₁-C₆ alkyl, or C₃-C₈ cycloalkyl.

Embodiment 6 provides the compound of any of Embodiments 1-5, wherein X² is selected from the group consisting of C═O, NH, N(CH₃), N(CH₂CH₃), N(CH(CH₃)₂), CH₂, CH(CH₃), CH(CH₂CH₃), CH(CH₂CH₂CH₃), CHCH(CH₃)₂, C(CH₃)₂, C(CH₃)(CH₂CH₃), C(CH₂CH₃)₂, 1,1-cyclopropanediyl, 1,1-cyclobutanediyl, 1,1-cyclopentanediyl, and 1,1-cyclohexanediyl.

Embodiment 7 provides the compound of any of Embodiments 1-6, wherein each occurrence of R² is independently selected from the group consisting of H and C₁-C₆ alkyl.

Embodiment 8 provides the compound of any of Embodiments 1-6, wherein (i) two R² combine with the carbon atom to which both of them are bound to form a substituent selected from the group consisting of C(═O), 1,1-cyclopropanediyl, 1,1-cyclobutanediyl, 1,1-cyclopentanediyl, and 1,1-cyclohexanediyl, or (ii) two R² bound to different carbon atoms combine to form —CH₂—, —CH₂CH₂—, —CH(CH₃)CH₂—, —CH₂CH₂CH₂—, or —CH₂CH₂CH₂CH₂—.

Embodiment 9 provides the compound of any of Embodiments 1-6 and 8, wherein two R² bound to different carbon atoms combine such that the compound of formula (I), (Ia), or (Ib) is

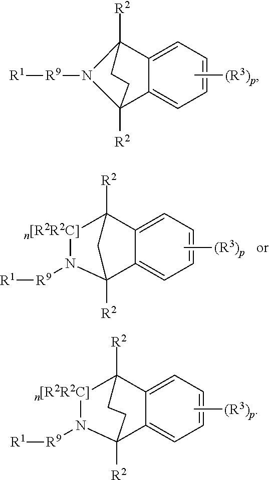

Embodiment 10 provides the compound of any of Embodiments 1-9, wherein each occurrence of R³ is such that the

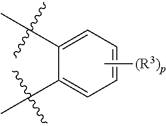

ring in (I), (Ia), or (Ib) is

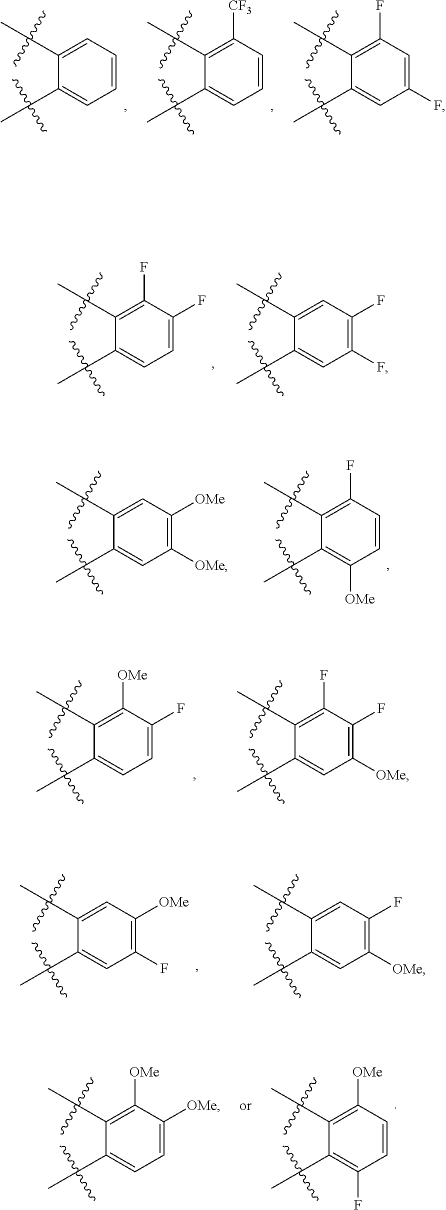

Embodiment 11 provides the compound of any of Embodiments 1-10, wherein two R⁵ bound to adjacent carbon atoms combine to form

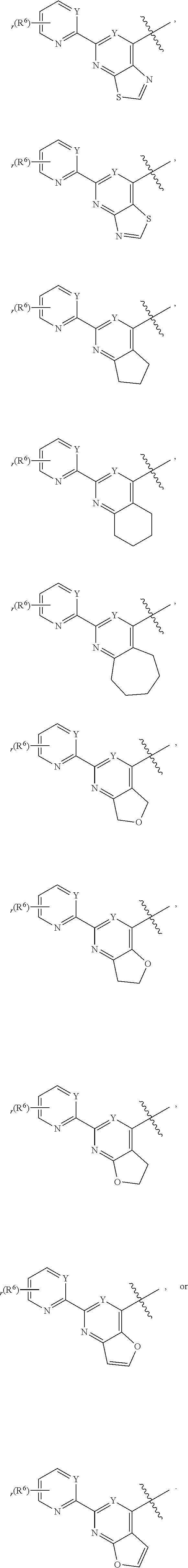

Embodiment 12 provides the compound of any of Embodiments 1-11, wherein each occurrence of alkyl, alkenyl, cycloalkyl, carbocyclyl, or heterocyclyl is independently optionally substituted with at least one substituent selected from the group consisting of C₁-C₆ alkyl, halo, —OR″, phenyl, and —N(R″)(R″), wherein each occurrence of R″ is independently H, C₁-C₆ alkyl, or C₃-C₈ cycloalkyl.

Embodiment 13 provides the compound of any of Embodiments 1-12, wherein each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR″, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

Embodiment 14 provides the compound of any of Embodiments 1-13, wherein each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR″, —N(R″)(R″), and C₁-C₆ alkoxycarbonyl, wherein each occurrence of R″ is independently H, C₁-C₆ alkyl or C₃-C₈ cycloalkyl.

Embodiment 15 provides the compound of any of Embodiments 1-14, which is selected from the group consisting of:
2-([2,2′-bipyrimidin]-4-yl)-5,7-difluoro-1,2,3,4-tetrahydroisoquinoline;
2-([2,2′-bipyrimidin]-4-yl)-5,6-difluoro-1,2,3,4-tetrahydroisoquinoline;
2-([2,2′-bipyrimidin]-5-yl)-4-methyl-1,2,3,4-tetrahydroisoquinoline;
2-([2,2′-bipyrimidin]-4-yl)-1-methyl-1,2,3,4-tetrahydroisoquinoline;
2-([2,2′-bipyrimidin]-4-yl)-3-ethyl-1,2,3,4-tetrahydroisoquinoline;
2-([2,2′-bipyrimidin]-4-yl)-1,2,3,4-tetrahydroisoquinoline;
2-([2,2′-bipyrimidin]-4-yl)-1-ethyl-1,2,3,4-tetrahydroisoquinoline;
2-([2,2′-bipyrimidin]-5-yl)-4-(trifluoromethyl)isoindoline;
2-([2,2′-bipyrimidin]-4-yl)-4-methyl-1,2,3,4-tetrahydroisoquinoline;
2-([2,2′-bipyrimidin]-5-yl)-4-methyl-3,4-dihydroisoquinolin-1(2H)-one;
2-([2,2′-bipyrimidin]-4-yl)-6,7-difluoro-1,2,3,4-tetrahydroisoquinoline;
2′-([2,2′-bipyrimidin]-5-yl)-6′,7′-dimethoxy-3′,4′-dihydro-2′H-spiro[cyclobutane-1,1′-isoquinoline];
2-([2,2′-bipyrimidin]-5-yl)-1-ethylisoindoline;
10-([2,2′-bipyrimidin]-5-yl)-1,2,3,4-tetrahydro-1,4-(epiminomethano)naphthalene;
2-([2,2′-bipyrimidin]-5-yl)-1,2,3,4-tetrahydro-1,4-methanoisoquinoline;
9-([2,2′-bipyrimidin]-5-yl)-1,2,3,4-tetrahydro-1,4-epiminonaphthalene;
2-([2,2′-bipyrimidin]-4-yl)-1-propyl-1,2,3,4-tetrahydroisoquinoline;
2-([2,2′-bipyrimidin]-4-yl)-5,6-difluoro-1-methyl-1,2,3,4-tetrahydroisoquinoline;
2-([2,2′-bipyrimidin]-4-yl)-1-ethyl-5,6-difluoro-1,2,3,4-tetrahydroisoquinoline;
2-([2,2′-bipyrimidin]-4-yl)-1-ethyl-5-fluoro-8-methoxy-1,2,3,4-tetrahydroisoquinoline;
2-([2,2′-bipyrimidin]-4-yl)-1-ethyl-5-fluoro-8-methoxy-1,2,3,4-tetrahydroisoquinoline;
2-([2,2′-bipyrimidin]-4-yl)-1-ethyl-6-fluoro-5-methoxy-1,2,3,4-tetrahydroisoquinoline;
2-([2,2′-bipyrimidin]-4-yl)-1-ethyl-5,6-difluoro-7-methoxy-1,2,3,4-tetrahydroisoquinoline;
2-([2,2′-bipyrimidin]-4-yl)-1-ethyl-5,6-difluoroisoindoline;
1-ethyl-5,6-difluoro-2-(5-fluoro-[2,2′-bipyrimidin]-4-yl)isoindoline;
1-ethyl-5,6-difluoro-2-(5-methyl-[2,2′-bipyrimidin]-4-yl)isoindoline;
2-(5-chloro-[2,2′-bipyrimidin]-4-yl)-1-ethyl-5,6-difluoroisoindoline;
2-(5-cyclopropyl-[2,2′-bipyrimidin]-4-yl)-1-ethyl-5,6-difluoroisoindoline;
2-([2,2′-bipyrimidin]-4-yl)-1-ethyl-5,6-dimethoxyisoindoline;
1-ethyl-5,6-dimethoxy-2-(5-methyl-[2,2′-bipyrimidin]-4-yl)isoindoline;
4-(1-ethyl-5,6-dimethoxyisoindolin-2-yl)-N-methyl-[2,2′-bipyrimidin]-5-amine;
2-(5-chloro-[2,2′-bipyrimidin]-4-yl)-1-ethyl-5,6-dimethoxyisoindoline;
2-(5-cyclopropyl-[2,2′-bipyrimidin]-4-yl)-1-ethyl-5,6-dimethoxyisoindoline;
1-ethyl-2-(5-isopropyl-[2,2′-bipyrimidin]-4-yl)-5,6-dimethoxyisoindoline;
2-([2,2′-bipyrimidin]-4-yl)-1-ethyl-6-fluoro-5-methoxyisoindoline;
1-ethyl-6-fluoro-5-methoxy-2-(5-phenyl-[2,2′-bipyrimidin]-4-yl)isoindoline;
1-ethyl-6-fluoro-5-methoxy-2-(5-methyl-[2,2′-bipyrimidin]-4-yl)isoindoline;
1-ethyl-6-fluoro-2-(5-fluoro-[2,2′-bipyrimidin]-4-yl)-5-methoxyisoindoline;
1-ethyl-6-fluoro-5-methoxy-2-(5-methoxy-[2,2′-bipyrimidin]-4-yl)isoindoline;
1-ethyl-6-fluoro-2-(5-fluoro-[2,2′-bipyrimidin]-4-yl)isoindolin-5-ol;
2-([2,2′-bipyrimidin]-4-yl)-1-ethyl-5-fluoro-6-methoxyisoindoline;
10-([2,2′-bipyrimidin]-4-yl)-1,2,3,4-tetrahydro-1,4-(epiminomethano)naphthalene;
10-(5-fluoro-[2,2′-bipyrimidin]-4-yl)-1,2,3,4-tetrahydro-1,4-(epiminomethano)naphthalene;
10-(5-methyl-[2,2′-bipyrimidin]-4-yl)-1,2,3,4-tetrahydro-1,4-(epiminomethano)naphthalene;
9-([2,2′-bipyrimidin]-4-yl)-1,2,3,4-tetrahydro-1,4-epiminonaphthalene;
2-([2,2′-bipyrimidin]-4-yl)-1,2,3,4-tetrahydro-1,4-methanoisoquinoline;
9-([2,2′-bipyrimidin]-5-yl)-6,7-dimethoxy-1,2,3,4-tetrahydro-1,4-epiminonaphthalene;
9-(5-methyl-[2,2′-bipyrimidin]-4-yl)-1,2,3,4-tetrahydro-1,4-epiminonaphthalene;
9-(5-fluoro-[2,2′-bipyrimidin]-4-yl)-1,2,3,4-tetrahydro-1,4-epiminonaphthalene;
9-(5-fluoro-[2,2′-bipyrimidin]-4-yl)-6,7-dimethoxy-1,2,3,4-tetrahydro-1,4-epiminonaphthalene;
6,7-dimethoxy-9-(5-methyl-[2,2′-bipyrimidin]-4-yl)-1,2,3,4-tetrahydro-1,4-epiminonaphthalene;
2-([2,2′-bipyrimidin]-5-yl)-1-ethyl-6-fluoro-5-methoxyisoindoline;
4-(1-ethyl-5,6-dimethoxyisoindolin-2-yl)-2-(pyrimidin-2-yl)furo[3,2-d]pyrimidine;
4-(1-ethyl-5,6-dimethoxyisoindolin-2-yl)-2-(pyrimidin-2-yl)-5,7-dihydrofuro[3,4-d]pyrimidine;
7-(1-ethyl-5,6-dimethoxyisoindolin-2-yl)-5-(pyrimidin-2-yl)thiazolo[5,4-d]pyrimidine;
4-(1-ethyl-5,6-dimethoxyisoindolin-2-yl)-2-(pyrimidin-2-yl)-6,7-dihydro-5H-cyclopenta[d]pyrimidine;
4-(1-ethyl-5,6-difluoroisoindolin-2-yl)pyrimidine-2-carboxylic acid;
4-(1-ethyl-6-fluoro-5-methoxyisoindolin-2-yl)pyrimidine-2-carboxylic acid;
5-(1-ethyl-5,6-dimethoxyisoindolin-2-yl)pyrimidine-2-carboxylic acid;
5-(1-ethyl-6-fluoro-5-methoxyisoindolin-2-yl)pyrimidine-2-carboxylic acid;
5-(1-ethyl-6-fluoro-5-methoxyisoindolin-2-yl)-N-(methylsulfonyl)pyrimidine-2-carboxamide;
4-(1-ethyl-6-fluoro-5-methoxyisoindolin-2-yl)-N-(methylsulfonyl)pyrimidine-2-carboxamide;
5-(1-ethyl-6-fluoro-5-methoxyisoindolin-2-yl)-N-(1-methyl-1H-imidazol-2-yl)pyrimidine-2-carboxamide;
5-(1-ethyl-6-fluoro-5-methoxyisoindolin-2-yl)-N-(pyridin-2-yl)pyrimidine-2-carboxamide;
4-(1-ethyl-6-fluoro-5-methoxyisoindolin-2-yl)-N-methylpyrimidine-2-carboxamide;
3-(4-(6,7-difluoro-3,4-dihydroisoquinolin-2(1H)-yl)pyrimidin-2-yl)pyridin-2-ol;
6-(1-ethyl-5,6-difluoro-3,4-dihydroisoquinolin-2(1H)-yl)-4-oxo-1,4-dihydropyridine-3-carboxylic acid;
5-(1-ethyl-5,6-difluoro-3,4-dihydroisoquinolin-2(1H)-yl)pyrimidine-2-carboxylic acid;
5-(1-ethyl-7-fluoro-6-methoxy-3,4-dihydroisoquinolin-2(1H)-yl)pyrimidine-2-carboxylic acid;
5-(1-ethyl-5,6-difluoroisoindolin-2-yl)pyrimidine-2-carboxylic acid;
5-(1-ethyl-7-fluoro-6-methoxy-3,4-dihydroisoquinolin-2(1H)-yl)pyrimidine-2-carboxamide;
5-(1-ethyl-5,6-difluoroisoindolin-2-yl)pyrimidine-2-carboxamide;
4-(1-ethyl-7-fluoro-6-methoxy-3,4-dihydroisoquinolin-2(1H)-yl)pyrimidine-2-carboxamide;
4-(1-ethyl-5,6-difluoroisoindolin-2-yl)pyrimidine-2-carboxamide;
3-(1-ethyl-3,4-dihydroisoquinolin-2(1H)-yl)-1,10-phenanthroline;
3-(5,6-difluoro-1-methyl-3,4-dihydroisoquinolin-2(1H)-yl)-1,10-phenanthroline;
3-(1-ethyl-3,4-dihydroisoquinolin-2(1H)-yl)-N-methyl-1,7-naphthyridin-8-amine;
7-(1-ethyl-7-fluoro-6-methoxy-3,4-dihydroisoquinolin-2(1H)-yl)-3-methylpyrido[3,2-d]pyrimidin-4(3H)-one;
5-Ethyl-8,9-difluoro-4-(2-pyrimidin-2-ylpyrimidin-4-yl)-3,5-dihydro-2H-1,4-benzoxazepine;
2-(2,2′-bipyrimidin-4-yl)-1-ethyl-7-fluoro-6-methoxy-1,2,3,4-tetrahydroisoquinoline;
2-([2,2′-bipyrimidin]-4-yl)-5,6-difluoro-1-propyl-1,2,3,4-tetrahydroisoquinoline; and
2-([2,2′-bipyrimidin]-4-yl)-1-ethyl-8-fluoro-7-methoxy-2,3,4,5-tetrahydro-1H-benzo[c]azepine.

Embodiment 16 provides a compound selected from the group consisting of
2-([2,2′-bipyrimidin]-5-yl)-5,7-difluoro-1,2,3,4-tetrahydroisoquinoline;
2-([2,2′-bipyrimidin]-5-yl)-5,6-difluoro-1,2,3,4-tetrahydroisoquinoline;
1-methyl-2-(2-pyrimidin-2-ylpyrimidin-5-yl)-3,4-dihydro-1H-isoquinoline;
2-([2,2′-bipyrimidin]-5-yl)-1-ethyl-1,2,3,4-tetrahydroisoquinoline;
2-([2,2′-bipyrimidin]-5-yl)-5,6-difluoro-1-methyl-1,2,3,4-tetrahydroisoquinoline;
2-([2,2′-bipyrimidin]-5-yl)-1-ethyl-6,7-difluoro-1,2,3,4-tetrahydroisoquinoline;
methyl 2-(2-([2,2′-bipyrimidin]-5-yl)-6,7-dimethoxy-1,2,3,4-tetrahydroisoquinolin-1-yl)acetate;
2-(2-([2,2′-bipyrimidin]-5-yl)-6,7-dimethoxy-1,2,3,4-tetrahydroisoquinolin-1-yl)acetic acid;
2-([2,2′-bipyrimidin]-5-yl)-1-ethyl-5,6-dimethoxy-1,2,3,4-tetrahydroisoquinoline;
2-([2,2′-bipyrimidin]-5-yl)-1-ethyl-5,6-difluoro-7-methoxy-1,2,3,4-tetrahydroisoquinoline;
2-([2,2′-bipyrimidin]-5-yl)-1-ethyl-5,6-difluoro-1,2,3,4-tetrahydroisoquinoline;
2-([2,2′-bipyrimidin]-5-yl)-1-ethyl-5-fluoro-8-methoxy-1,2,3,4-tetrahydroisoquinoline;
2-([2,2′-bipyrimidin]-5-yl)-1-ethyl-7-fluoro-6-methoxy-1,2,3,4-tetrahydroisoquinoline.

Embodiment 17 provides a pharmaceutical composition comprising at least one compound of any of Embodiments 1-16 and a pharmaceutically acceptable carrier.

Embodiment 18 provides the pharmaceutical composition of Embodiment 17, further comprising at least one additional agent useful for treating hepatitis virus infection.

Embodiment 19 provides the pharmaceutical composition of Embodiment 18, wherein the at least one additional agent comprises at least one selected from the group consisting of reverse transcriptase inhibitor; capsid inhibitor; cccDNA formation inhibitor; sAg secretion inhibitor; oligomeric nucleotide targeted to the Hepatitis B genome; and immunostimulator.

Embodiment 20 provides the pharmaceutical composition of Embodiment 19, wherein the oligomeric nucleotide comprises one or more siRNAs.

Embodiment 21 provides a method of treating or preventing hepatitis virus infection in a subject, the method comprising administering to the subject a therapeutically effective amount of at least one compound of any of Embodiments 1-16 or at least one pharmaceutical composition of any of Embodiments 17-20.

Embodiment 22 provides a method of reducing or minimizing levels of at least one selected from the group consisting of hepatitis B virus surface antigen (HBsAg), hepatitis B e-antigen (HBeAg), hepatitis B core protein, and pregenomic (pg) RNA, in a HBV-infected subject, the method comprising administering to the subject a therapeutically effective amount of at least one compound of any of Embodiments 1-16 or at least one pharmaceutical composition of any of Embodiments 17-20.

Embodiment 23 provides the method of any of Embodiments 21-22, wherein the at least one compound is administered to the subject in a pharmaceutically acceptable composition.

Embodiment 24 provides the method of any of Embodiments 21-23, wherein the subject is further administered at least one additional agent useful for treating the hepatitis virus infection.

Embodiment 25 provides the method of any of Embodiments 21-24, wherein the at least one additional agent comprises at least one selected from the group consisting of reverse transcriptase inhibitor; capsid inhibitor; cccDNA formation inhibitor; sAg secretion inhibitor; oligomeric nucleotide targeted to the Hepatitis B genome; and immunostimulator.

Embodiment 26 provides the method of Embodiment 25, wherein the oligomeric nucleotide comprises one or more siRNAs.

Embodiment 27 provides the method of Embodiment 24, wherein the subject is co-administered the at least one compound and the at least one additional agent.

Embodiment 28 provides the method of Embodiment 27, wherein the at least one compound and the at least one additional agent are coformulated.

Embodiment 29 provides the method of any of Embodiments 21-28, wherein the subject is infected with HBV or co-infected with HBV-hepatitis D virus (HDV).

Embodiment 30 provides the method of any of Embodiments 21-29, wherein the subject is a mammal.

Embodiment 31 provides the method of Embodiment 30, wherein the mammal is a human.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound selected from the group consisting of:
(i) a compound of formula (Ia): 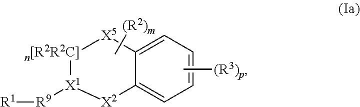
wherein in (Ia):
X¹ is N and X² is CR²R², or X² is NR⁴ and X is CR⁴;
X⁵ is selected from the group consisting of O and CR²R²,
or one R² group from X⁵ can combine with one R² group of X² to form C₁-C₆ alkylene;
R¹ is selected from the group consisting of: 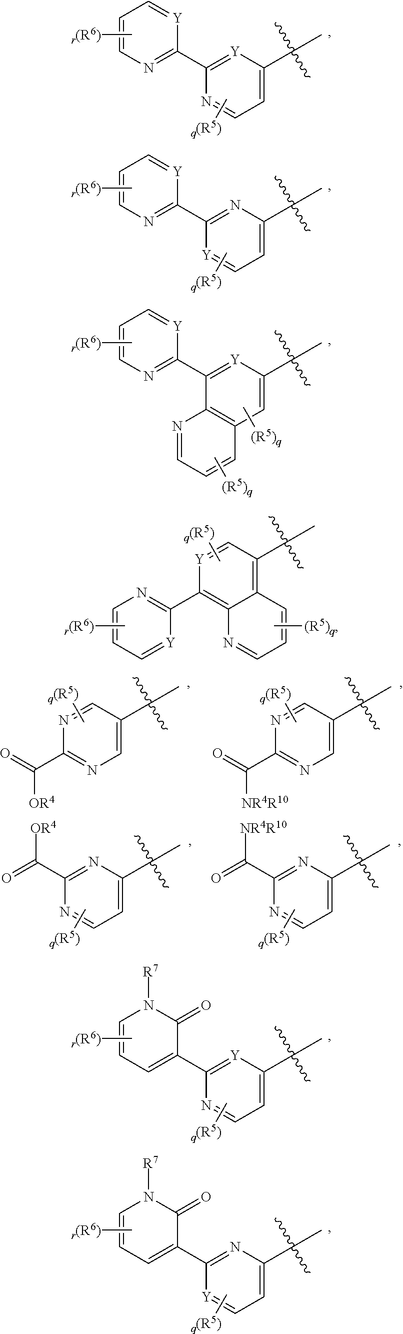 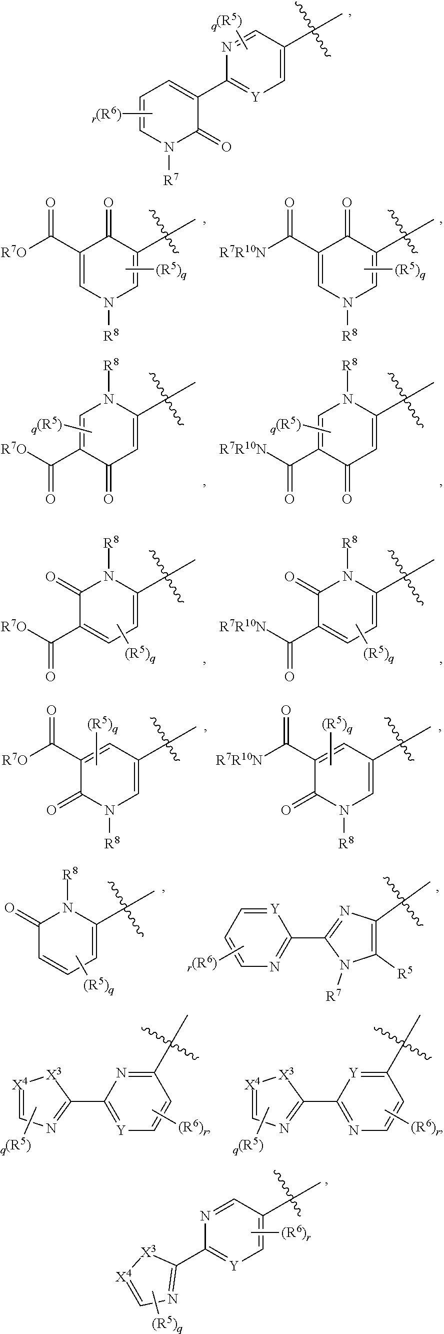 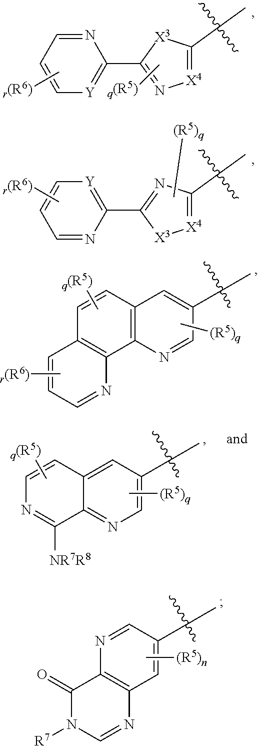
R⁹ is a bond if X¹ is CH, or R⁹ is selected from the group consisting of a bond and —C(═O)— if X¹ is N;
each occurrence of X³ is independently selected from the group consisting of NR⁷, O, and S;
each occurrence of X⁴ is independently selected from the group consisting of NR and CR⁵;
each occurrence of Y is independently selected from the group consisting of N and CR⁵;
each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)₀₋₂C(═O)OR′, and —N(R′)(R′), wherein each occurrence of R′ is independently selected from the group consisting of H, optionally substituted C₁-C₆ alkyl, and optionally substituted C₃-C₈ cycloalkyl;
or two R² combine with the carbon atom to which both of them are bound to form a substituent selected from the group consisting of C(═O) and optionally substituted 1,1-(C₃-C₈ cycloalkanediyl);
or two R² bound to different carbon atoms combine to form an optionally substituted C₁-C₆ alkanediyl;
each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
each occurrence of R⁴ is independently selected from the group consisting of H, optionally substituted C₁-C₆ alkyl, and optionally substituted C₃-C₈ cycloalkyl;
each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
or two R⁵ bound to adjacent carbon atoms combine to form optionally substituted 5-7 membered carbocyclyl or heterocyclyl;
each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
each occurrence of R⁷ is independently selected from the group consisting of H, optionally substituted C₁-C₆ alkyl, and optionally substituted C₃-C₈ cycloalkyl;
each occurrence of R⁸ is independently selected from the group consisting of H, optionally substituted C₁-C₆ alkyl, and optionally substituted C₃-C₈ cycloalkyl;
each occurrence of R¹⁰ is independently selected from the group consisting of H, optionally substituted C₁-C₆ alkyl, optionally substituted C₃-C₈ cycloalkyl, optionally substituted phenyl, optionally substituted heteroaryl, —S(═O)₂(optionally substituted C₁-C₆ alkyl), and —S(═O)₂(optionally substituted C₃-C₈ cycloalkyl);
m is 0, 1, 2, 3, or 4;
n is 0, 1, or 2;
p is 0, 1, 2, 3, or 4;
q is 0, 1, or 2;
r is 0, 1, 2, or 3;
(ii) a compound of formula (Ib): 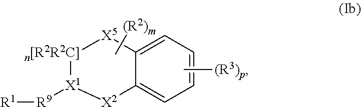
wherein in (Ib):
X¹ is N and X² is CR²R², or X² is NR⁴ and X¹ is CR⁴;
X⁵ is selected from the group consisting of 0 and CR²R²,
or one R² group from X⁵ can combine with one R² group of X² to form C₁-C₆ alkylene;
R¹ is 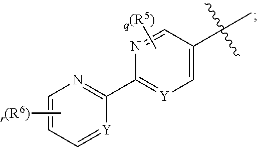
R⁹ is a bond if X¹ is CH, or R⁹ is selected from the group consisting of a bond and —C(═O)— if X¹ is N;
wherein, if R⁹ is a bond, X¹ is N, X² is CR², and X⁵ is CH₂, then n is not 1;
each occurrence of Y is independently selected from the group consisting of N and CR⁵;
each occurrence of R² is independently selected from the group consisting of H, optionally substituted C₁-C₆ alkyl, optionally substituted C₃-C₈ cycloalkyl, halo, C₁-C₆ haloalkyl, C₁-C₆ haloalkoxy, C₁-C₆ hydroxyalkyl, —OR′, —(CH₂)₀₋₂C(═O)OR′, and —N(R′)(R′), wherein each occurrence of R′ is independently selected from the group consisting of H, optionally substituted C₁-C₆ alkyl, and optionally substituted C₃-C₈ cycloalkyl;
or two R² combine with the carbon atom to which both of them are bound to form a substituent selected from the group consisting of C(═O) and optionally substituted 1,1-(C₃-C₈ cycloalkanediyl);
or two R² bound to different carbon atoms combine to form an optionally substituted C₁-C₆ alkanediyl;
each occurrence of R³ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
each occurrence of R⁴ is independently selected from the group consisting of H, optionally substituted C₁-C₆ alkyl, and optionally substituted C₃-C₈ cycloalkyl;
each occurrence of R⁵ is independently selected from the group consisting of H, optionally substituted C₁-C₆ alkyl, optionally substituted C₃-C₈ cycloalkyl, optionally substituted phenyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
or two R⁵ bound to adjacent carbon atoms combine to form optionally substituted 5-7 membered carbocyclyl or heterocyclyl;
each occurrence of R⁶ is independently selected from the group consisting of H, optionally substituted C₁-C₆ alkyl, optionally substituted C₃-C₈ cycloalkyl, halo, cyano, nitro, C₁-C₆ haloalkyl, C₁-C₆ haloalkoxy, C₁-C₆ hydroxyalkyl, —OR′, —SR′, —S(═O)R′, —S(O)₂R′, and —N(R′)(R′), wherein each occurrence of R′ is independently selected from the group consisting of H, optionally substituted C₁-C₆ alkyl, and optionally substituted C₃-C₈ cycloalkyl;
m is 0, 1, 2, 3, or 4;
n is 0, 1, or 2;
p is 0, 1, 2, 3, or 4;
q is 0, 1, or 2;
r is 0, 1, 2, or 3;
or a salt, solvate, geometric isomer, stereoisomer, tautomer, and any mixtures thereof.

2. The compound of claim 1, which is: 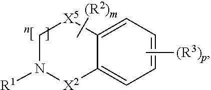
wherein X² is CR²R², or 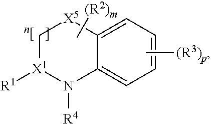
wherein X¹ is CR⁴.

3. (canceled)

4. The compound of claim 1, wherein each occurrence of R⁴ is independently selected from the group consisting of H, methyl, ethyl, propyl, isopropyl, butyl, isobutyl, sec-butyl, and tert-butyl.

5. The compound of claim 1, wherein R¹ is selected from the group consisting of: 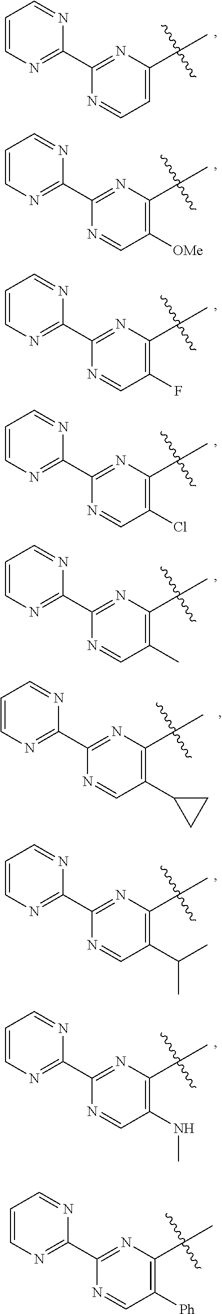
wherein Ph is optionally substituted 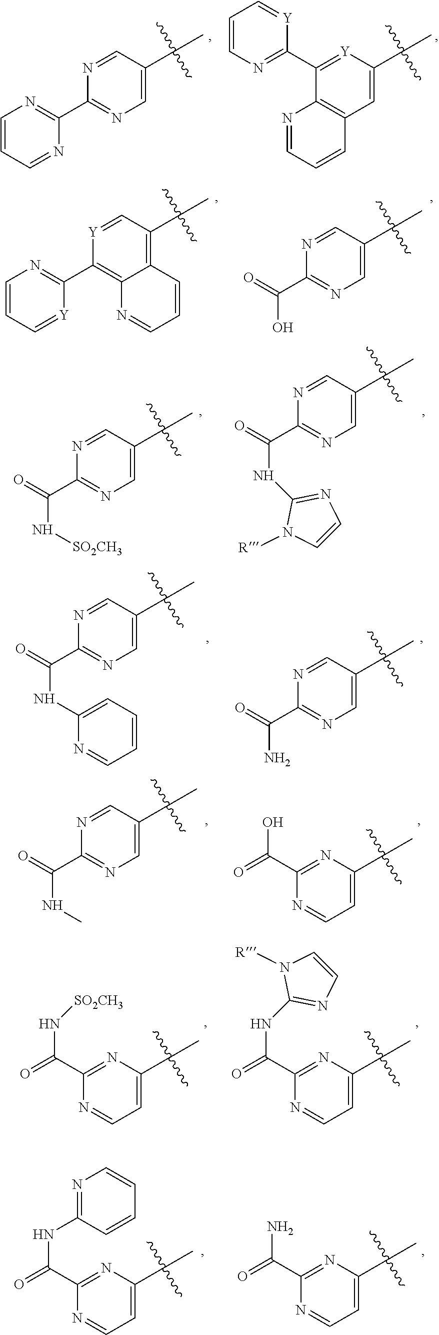 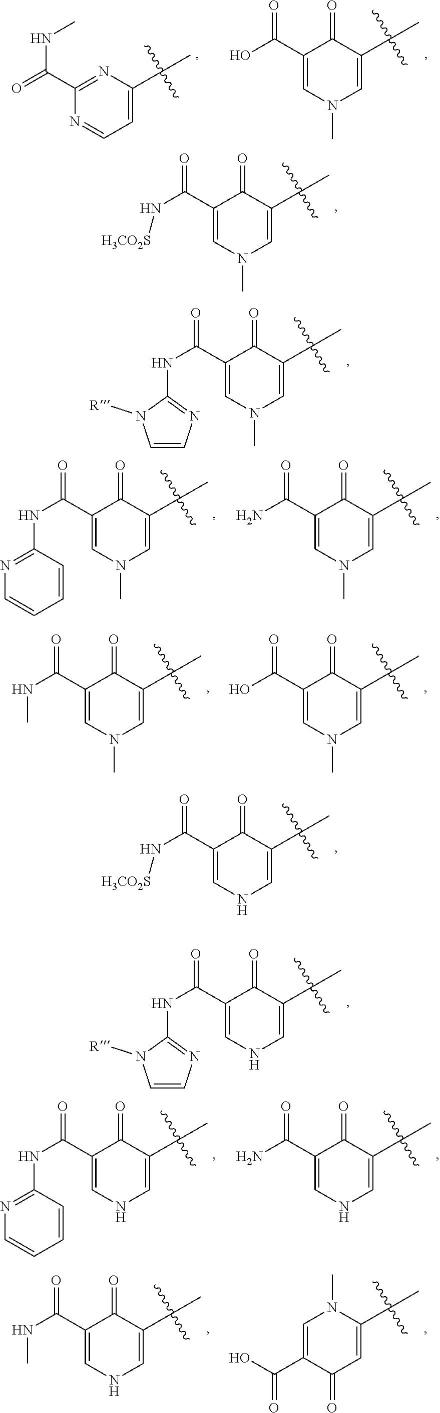 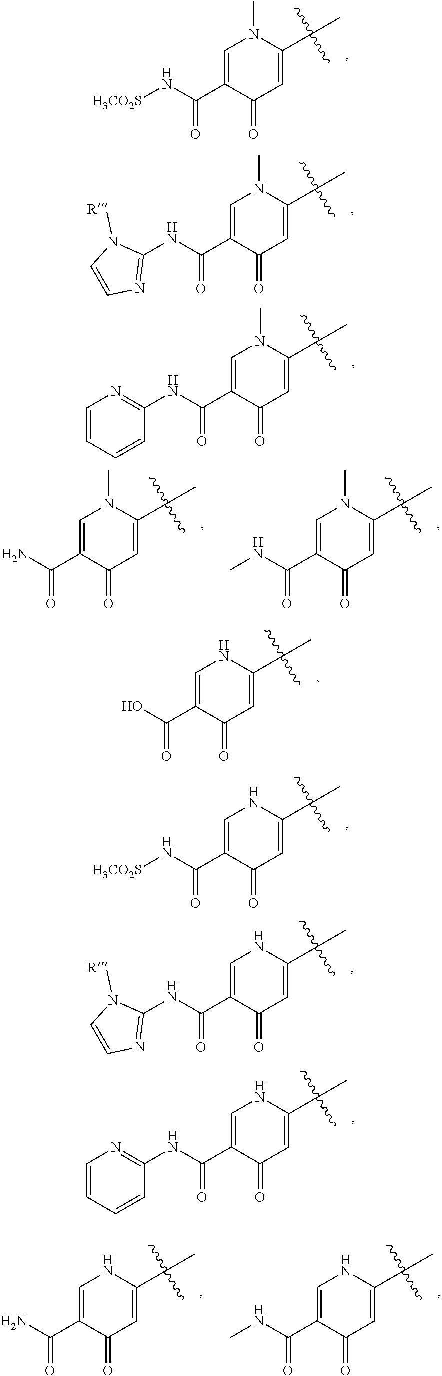 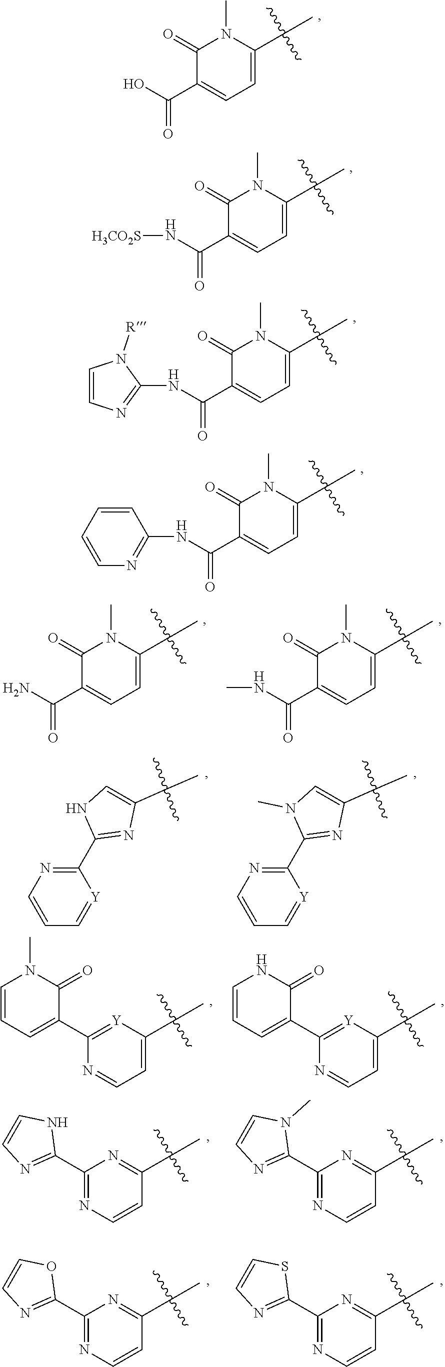 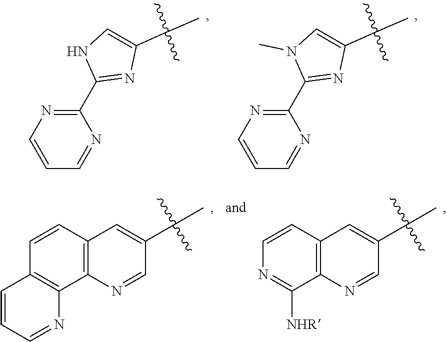
wherein each occurrence of R′″ is independently H, C₁-C₆ alkyl, or C₃-C₈ cycloalkyl.

6. The compound of claim 1, wherein X² is selected from the group consisting of C═O, NH, N(CH₃), N(CH₂CH₃), N(CH(CH₃)₂), CH₂, CH(CH₃), CH(CH₂CH₃), CH(CH₂CH₂CH₃), CHCH(CH₃)₂, C(CH₃)₂, C(CH₃)(CH₂CH₃), C(CH₂CH₃)₂, 1,1-cyclopropanediyl, 1,1-cyclobutanediyl, 1,1-cyclopentanediyl, and 1,1-cyclohexanediyl.

7. The compound of claim 1, wherein
(i) each occurrence of R² is independently selected from the group consisting of H and C₁-C₆ alkyl;
(ii) two R² combine with the carbon atom to which both of them are bound to form a substituent selected from the group consisting of C(═O), 1,1-cyclopropanediyl, 1,1-cyclobutanediyl, 1,1-cyclopentanediyl, and 1,1-cyclohexanediyl, or
(iii) two R² bound to different carbon atoms combine to form —CH₂—, —CH₂CH₂—, —CH(CH₃)CH₂—, —CH₂CH₂CH₂—, or —CH₂CH₂CH₂CH₂—.

8. (canceled)

9. The compound of claim 1, wherein two R² bound to different carbon atoms combine such that the compound of formula (I), (Ia), or (Ib) is

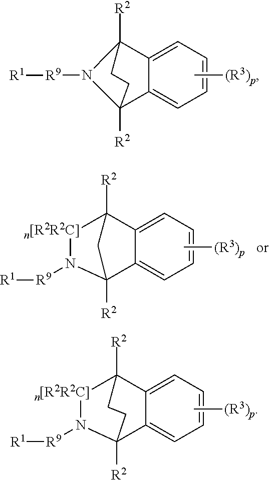

10. The compound of claim 1, wherein each occurrence of R³ is such that the 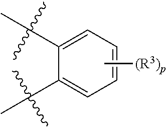
ring in (I), (Ia), or (Ib) is

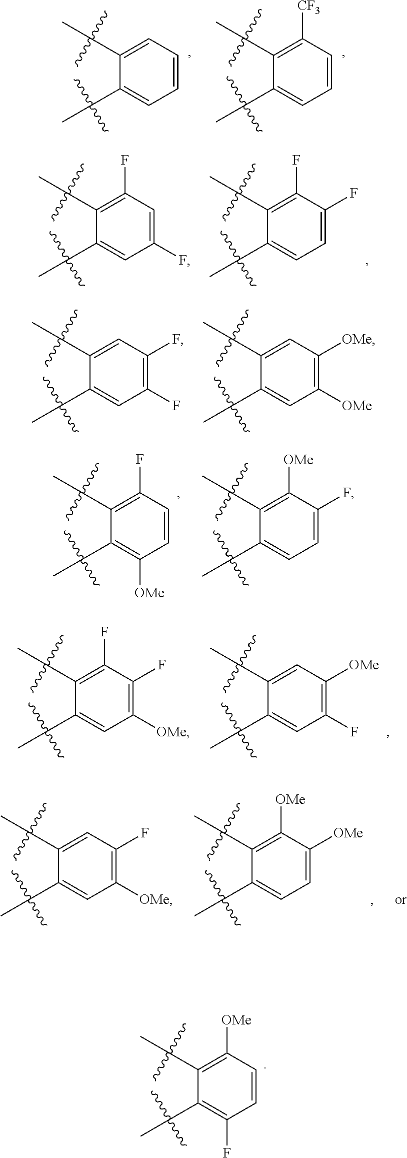

11. The compound of claim 1, wherein two R⁵ bound to adjacent carbon atoms combine to form

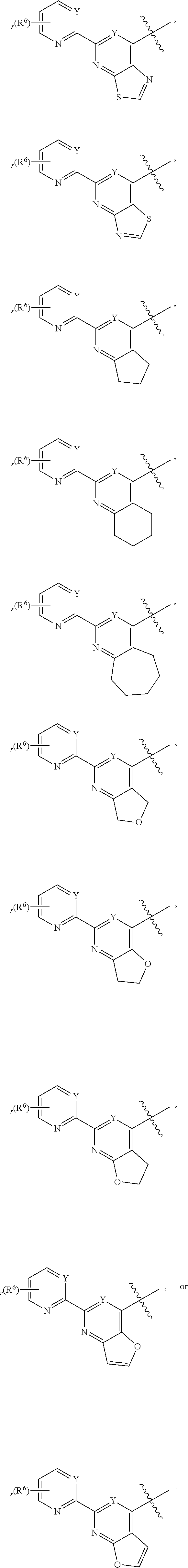

12. The compound of claim 1, wherein each occurrence of alkyl, alkenyl, cycloalkyl, carbocyclyl, or heterocyclyl is independently optionally substituted with at least one substituent selected from the group consisting of C₁-C₆ alkyl, halo, —OR″, phenyl, and —N(R″)(R″), wherein each occurrence of R″ is independently H, C₁-C₆ alkyl, or C₃-C₈ cycloalkyl.

13. The compound of claim 1, wherein each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR″, —N(R″)(R″), —NO₂, —S(═O)₂N(R″)(R″), acyl, and C₁-C₆ alkoxycarbonyl, wherein each occurrence of R″ is independently H, C₁-C₆ alkyl or C₃-C₈ cycloalkyl.

14. (canceled)

15. A compound of claim 1, which is selected from the group consisting of:

| | |
|---|---|
| 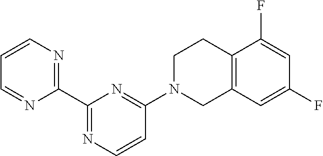 [SMILES: Fc1cc(F)c2c(c1)CN(c1ccnc(-c3ncccn3)n1)CC2] | 2-([2,2′-bipyrimidin]-4-yl)-5,7-difluoro-1,2,3,4- tetrahydroisoquinoline; |
| 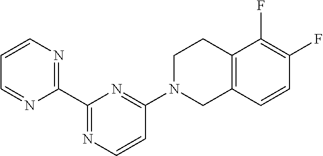 [SMILES: Fc1ccc2c(c1F)CCN(c1ccnc(-c3ncccn3)n1)C2] | 2-([2,2′-bipyrimidin]-4-yl)-5,6-difluoro-1,2,3,4- tetrahydroisoquinoline; |
| 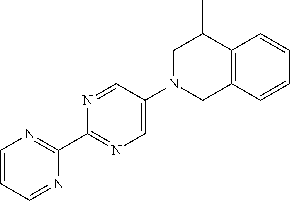 [SMILES: CC1CN(c2cnc(-c3ncccn3)nc2)Cc2ccccc21] | 2-([2,2′-bipyrimidin]-5-yl)-4-methyl-1,2,3,4- tetrahydroisoquinoline; |
| 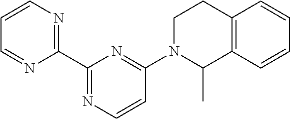 [SMILES: CC1c2ccccc2CCN1c1ccnc(-c2ncccn2)n1] | 2-([2,2′-bipyrimidin]-4-yl)-1-methyl-1,2,3,4- tetrahydroisoquinoline; |
| 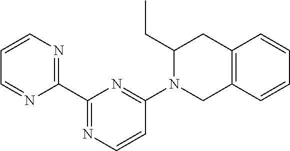 [SMILES: CCC1Cc2ccccc2CN1c1ccnc(-c2ncccn2)n1] | 2-([2,2′-bipyrimidin]-4-yl)-3-ethyl-1,2,3,4- tetrahydroisoquinoline; |
| 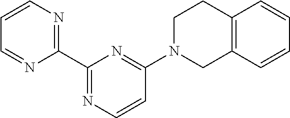 [SMILES: c1cnc(-c2nccc(N3CCc4ccccc4C3)n2)nc1] | 2-([2,2′-bipyrimidin]-4-yl)-1,2,3,4- tetrahydroisoquinoline; |
| 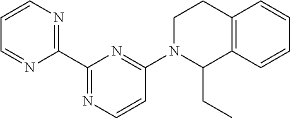 [SMILES: CCC1c2ccccc2CCN1c1ccnc(-c2ncccn2)n1] | 2-([2,2′-bipyrimidin]-4-yl)-1-ethyl-1,2,3,4- tetrahydroisoquinoline; |
| 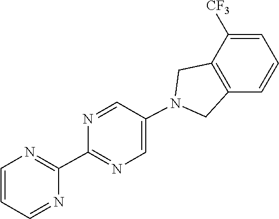 [SMILES: FC(F)(F)c1cccc2c1CN(c1cnc(-c3ncccn3)nc1)C2] | 2-([2,2′-bipyrimidin]-5-yl)-4- (trifluoromethyl)isoindoline; |
| 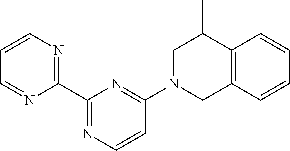 [SMILES: CC1CN(c2ccnc(-c3ncccn3)n2)Cc2ccccc21] | 2-([2,2′-bipyrimidin]-4-yl)-4-methyl-1,2,3,4- tetrahydroisoquinoline; |
| 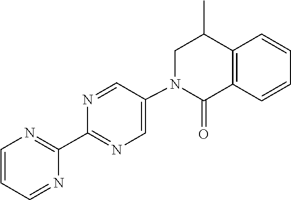 [SMILES: CC1CN(c2cnc(-c3ncccn3)nc2)C(=O)c2ccccc21] | 2-([2,2′-bipyrimidin]-5-yl)-4-methyl-3,4- dihydroisoquinolin-1(2H)-one; |
| 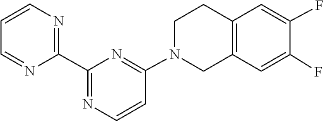 [SMILES: Fc1cc2c(cc1F)CN(c1ccnc(-c3ncccn3)n1)CC2] | 2-([2,2′-bipyrimidin]-4-yl)-6,7-difluoro-1,2,3,4- tetrahydroisoquinoline; |
| 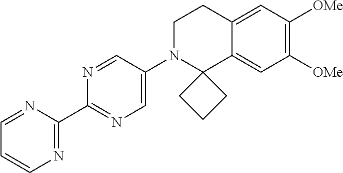 [SMILES: COc1cc2c(cc1OC)C1(CCC1)N(c1cnc(-c3ncccn3)nc1)CC2] | 2′-([2,2′-bipyrimidin]-5-yl)-6′,7′-dimethoxy-3′,4′- dihydro-2′H-spiro[cyclobutane-1,1′-isoquinoline]; |
| 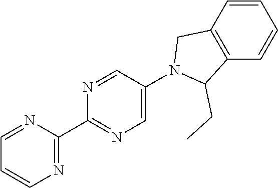 [SMILES: CCC1c2ccccc2CN1c1cnc(-c2ncccn2)nc1] | 2-([2,2′-bipyrimidin]-5-yl)-1-ethylisoindoline; |
| 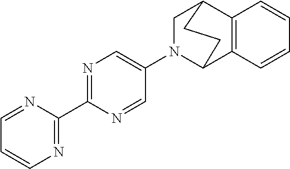 [SMILES: c1cnc(-c2ncc(N3CC4CCC3c3ccccc34)cn2)nc1] | 10-([2,2′-bipyrimidin]-5-yl)-1,2,3,4-tetrahydro-1,4- (epiminomethano)naphthalene; |
| 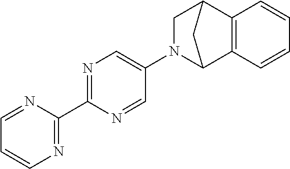 [SMILES: c1cnc(-c2ncc(N3CC4CC3c3ccccc34)cn2)nc1] | 2-([2,2′-bipyrimidin]-5-yl)-1,2,3,4-tetrahydro-1,4- methanoisoquinoline; |
| 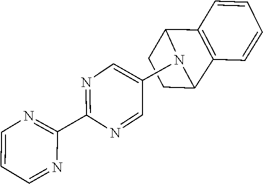 [SMILES: c1cnc(-c2ncc(N3C4CCC3c3ccccc34)cn2)nc1] | 9-([2,2′-bipyrimidin]-5-yl)-1,2,3,4-tetrahydro-1,4- epiminonaphthalene; |
| 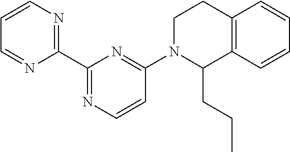 [SMILES: CCCC1c2ccccc2CCN1c1ccnc(-c2ncccn2)n1] | 2-([2,2′-bipyrimidin]-4-yl)-1-propyl-1,2,3,4- tetrahydroisoquinoline; |
| 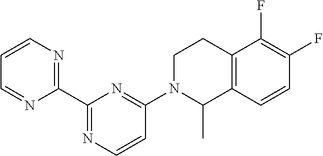 [SMILES: CC1c2ccc(F)c(F)c2CCN1c1ccnc(-c2ncccn2)n1] | 2-([2,2′-bipyrimidin]-4-yl)-5,6-difluoro-1-methyl- 1,2,3,4-tetrahydroisoquinoline; |
| 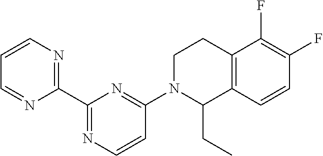 [SMILES: CCC1c2ccc(F)c(F)c2CCN1c1ccnc(-c2ncccn2)n1] | 2-([2,2′-bipyrimidin]-4-yl)-1-ethyl-5,6-difluoro- 1,2,3,4-tetrahydroisoquinoline; |
| 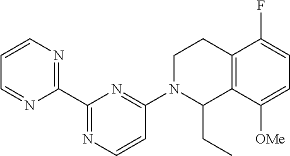 [SMILES: CCC1c2c(OC)ccc(F)c2CCN1c1ccnc(-c2ncccn2)n1] | 2-([2,2′-bipyrimidin]-4-yl)-1-ethyl-5-fluoro-8- methoxy-1,2,3,4-tetrahydroisoquinoline; |
| 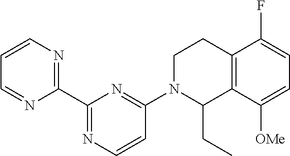 [SMILES: CCC1c2c(OC)ccc(F)c2CCN1c1ccnc(-c2ncccn2)n1] | 2-([2,2′-bipyrimidin]-4-yl)-1-ethyl-5-fluoro-8- methoxy-1,2,3,4-tetrahydroisoquinoline; |
| 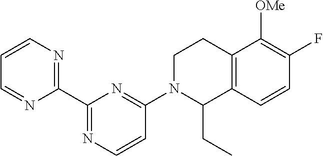 [SMILES: CCC1c2ccc(F)c(OC)c2CCN1c1ccnc(-c2ncccn2)n1] | 2-([2,2′-bipyrimidin]-4-yl)-1-ethyl-6-fluoro-5- methoxy-1,2,3,4-tetrahydroisoquinoline; |
| 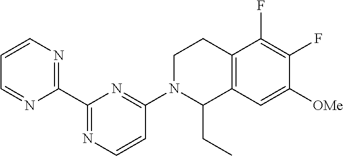 [SMILES: CCC1c2cc(OC)c(F)c(F)c2CCN1c1ccnc(-c2ncccn2)n1] | 2-([2,2′-bipyrimidin]-4-yl)-1-ethyl-5,6-difluoro-7- methoxy-1,2,3,4-tetrahydroisoquinoline; |
| 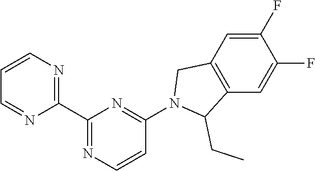 [SMILES: CCC1c2cc(F)c(F)cc2CN1c1ccnc(-c2ncccn2)n1] | 2-([2,2′-bipyrimidin]-4-yl)-1-ethyl-5,6- difluoroisoindoline; |
| 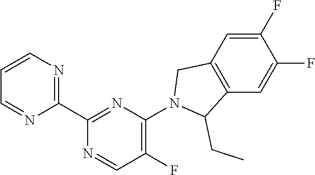 [SMILES: CCC1c2cc(F)c(F)cc2CN1c1nc(-c2ncccn2)ncc1F] | 1-ethyl-5,6-difluoro-2-(5-fluoro-[2,2′-bipyrimidin]-4- yl)isoindoline; |
| 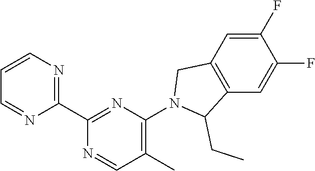 [SMILES: CCC1c2cc(F)c(F)cc2CN1c1nc(-c2ncccn2)ncc1C] | 1-ethyl-5,6-difluoro-2-(5-methyl-[2,2′-bipyrimidin]-4- yl)isoindoline; |
| 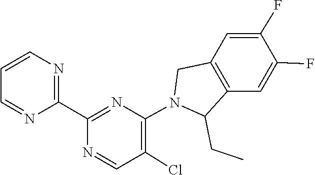 [SMILES: CCC1c2cc(F)c(F)cc2CN1c1nc(-c2ncccn2)ncc1Cl] | 2-(5-chloro-[2,2′-bipyrimidin]-4-yl)-1-ethyl-5,6- difluoroisoindoline; |
| 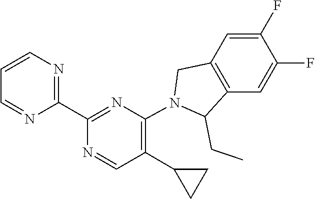 [SMILES: CCC1c2cc(F)c(F)cc2CN1c1nc(-c2ncccn2)ncc1C1CC1] | 2-(5-cyclopropyl-[2,2′-bipyrimidin]-4-yl)-1-ethyl-5,6- difluoroisoindoline; |
| 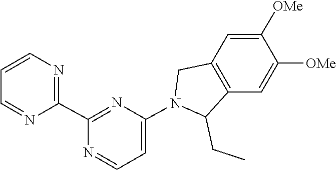 [SMILES: CCC1c2cc(OC)c(OC)cc2CN1c1ccnc(-c2ncccn2)n1] | 2-([2,2′-bipyrimidin]-4-yl)-1-ethyl-5,6- dimethoxyisoindoline; |
| 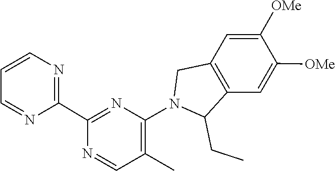 [SMILES: CCC1c2cc(OC)c(OC)cc2CN1c1nc(-c2ncccn2)ncc1C] | 1-ethyl-5,6-dimethoxy-2-(5-methyl-[2,2′-bipyrimidin]- 4-yl)isoindoline; |
| 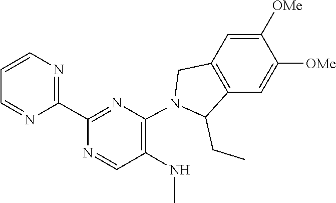 [SMILES: CCC1c2cc(OC)c(OC)cc2CN1c1nc(-c2ncccn2)ncc1NC] | 4-(1-ethyl-5,6-dimethoxyisoindolin-2-yl)-N-methyl- [2,2′-bipyrimidin]-5-amine; |
| 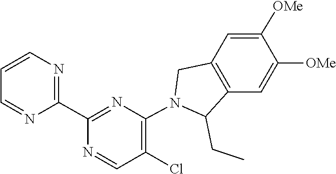 [SMILES: CCC1c2cc(OC)c(OC)cc2CN1c1nc(-c2ncccn2)ncc1Cl] | 2-(5-chloro-[2,2′-bipyrimidin]-4-yl)-1-ethyl-5,6- dimethoxyisoindoline; |
| 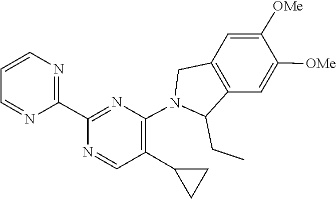 [SMILES: CCC1c2cc(OC)c(OC)cc2CN1c1nc(-c2ncccn2)ncc1C1CC1] | 2-(5-cyclopropyl-[2,2′-bipyrimidin]-4-yl)-1-ethyl-5,6- dimethoxyisoindoline; |
| 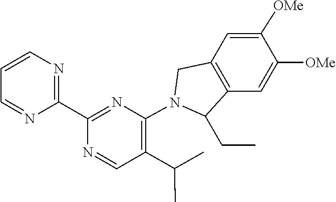 [SMILES: CCC1c2cc(OC)c(OC)cc2CN1c1nc(-c2ncccn2)ncc1C(C)C] | 1-ethyl-2-(5-isopropyl-[2,2′-bipyrimidin]-4-yl)-5,6- dimethoxyisoindoline; |
| 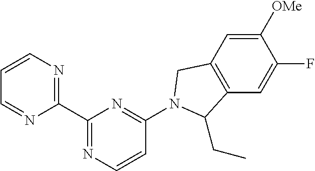 [SMILES: CCC1c2cc(F)c(OC)cc2CN1c1ccnc(-c2ncccn2)n1] | 2-([2,2′-bipyrimidin]-4-yl)-1-ethyl-6-fluoro-5- methoxyisoindoline; |
| 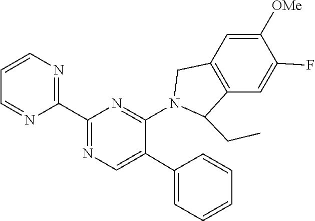 [SMILES: CCC1c2cc(F)c(OC)cc2CN1c1nc(-c2ncccn2)ncc1-c1ccccc1] | 1-ethyl-6-fluoro-5-methoxy-2-(5-phenyl-[2,2′- bipyrimidin]-4-yl)isoindoline; |
| 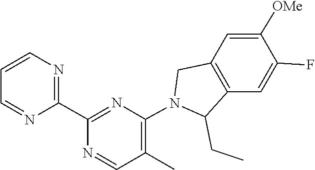 [SMILES: CCC1c2cc(F)c(OC)cc2CN1c1nc(-c2ncccn2)ncc1C] | 1-ethyl-6-fluoro-5-methoxy-2-(5-methyl-[2,2′- bipyrimidin]-4-yl)isoindoline; |
| 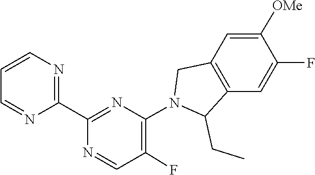 [SMILES: CCC1c2cc(F)c(OC)cc2CN1c1nc(-c2ncccn2)ncc1F] | 1-ethyl-6-fluoro-2-(5-fluoro-[2,2′-bipyrimidin]-4-yl)- 5-methoxyisoindoline; |
| 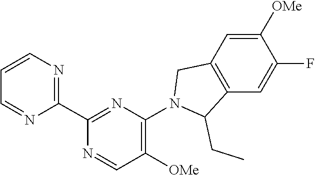 [SMILES: CCC1c2cc(F)c(OC)cc2CN1c1nc(-c2ncccn2)ncc1OC] | 1-ethyl-6-fluoro-5-methoxy-2-(5-methoxy-[2,2′- bipyrimidin]-4-yl)isoindoline; |
| 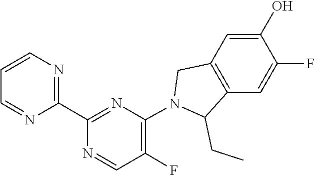 [SMILES: CCC1c2cc(F)c(O)cc2CN1c1nc(-c2ncccn2)ncc1F] | 1-ethyl-6-fluoro-2-(5-fluoro-[2,2′-bipyrimidin]-4- yl)isoindolin-5-ol; |
| 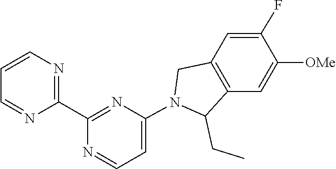 [SMILES: CCC1c2cc(OC)c(F)cc2CN1c1ccnc(-c2ncccn2)n1] | 2-([2,2′-bipyrimidin]-4-yl)-1-ethyl-5-fluoro-6- methoxyisoindoline; |
| 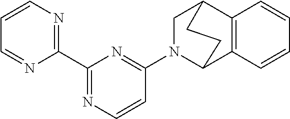 [SMILES: c1cnc(-c2nccc(N3CC4CCC3c3ccccc34)n2)nc1] | 10-([2,2′-bipyrimidin]-4-yl)-1,2,3,4-tetrahydro-1,4 (epiminomethano)naphthalene; |
| 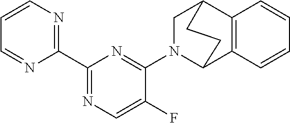 [SMILES: Fc1cnc(-c2ncccn2)nc1N1CC2CCC1c1ccccc12] | 10-(5-fluoro-[2,2′-bipyrimidin]-4-yl)-1,2,3,4- tetrahydro-1,4-(epiminomethano)naphthalene; |
| 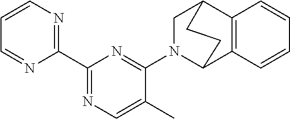 [SMILES: Cc1cnc(-c2ncccn2)nc1N1CC2CCC1c1ccccc12] | 10-(5-methyl-[2,2′-bipyrimidin]-4-yl)-1,2,3,4- tetrahydro-1,4-(epiminomethano)naphthalene; |
| 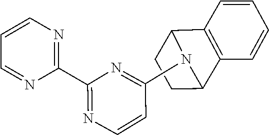 [SMILES: c1cnc(-c2nccc(N3C4CCC3c3ccccc34)n2)nc1] | 9-([2,2′-bipyrimidin]-4-yl)-1,2,3,4-tetrahydro-1,4- epiminonaphthalene; |
| 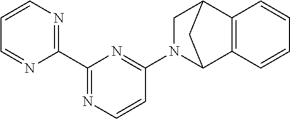 [SMILES: c1cnc(-c2nccc(N3CC4CC3c3ccccc34)n2)nc1] | 2-([2,2′-bipyrimidin]-4-yl)-1,2,3,4-tetrahydro-1,4- methanoisoquinoline; |
| 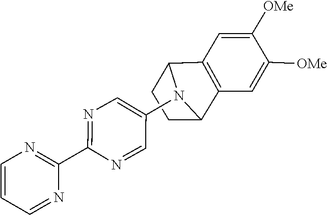 [SMILES: COc1cc2c(cc1OC)C1CCC2N1c1cnc(-c2ncccn2)nc1] | 9-([2,2′-bipyrimidin]-5-yl)-6,7-dimethoxy-1,2,3,4- tetrahydro-1,4-epiminonaphthalene; |
| 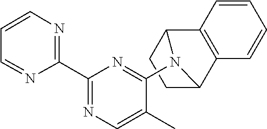 [SMILES: Cc1cnc(-c2ncccn2)nc1N1C2CCC1c1ccccc12] | 9-(5-methyl-[2,2′-bipyrimidin]-4-yl)-1,2,3,4- tetrahydro-1,4-epiminonaphthalene; |
| 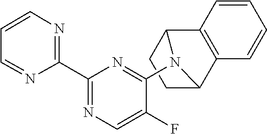 [SMILES: Fc1cnc(-c2ncccn2)nc1N1C2CCC1c1ccccc12] | 9-(5-fluoro-[2,2′-bipyrimidin]-4-yl)-1,2,3,4- tetrahydro-1,4-epiminonaphthalene; |
| 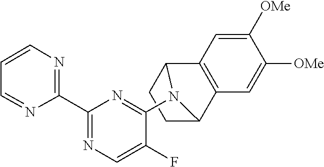 [SMILES: COc1cc2c(cc1OC)C1CCC2N1c1nc(-c2ncccn2)ncc1F] | 9-(5-fluoro-[2,2′-bipyrimidin]-4-yl)-6,7-dimethoxy- 1,2,3,4-tetrahydro-1,4-epiminonaphthalene; |
| 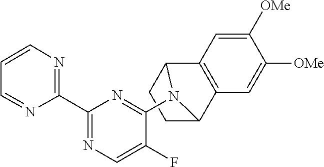 [SMILES: COc1cc2c(cc1OC)C1CCC2N1c1nc(-c2ncccn2)ncc1F] | 6,7-dimethoxy-9-(5-methyl-[2,2′-bipyrimidin]-4-yl)- 1,2,3,4-tetrahydro-1,4-epiminonaphthalene; |
| 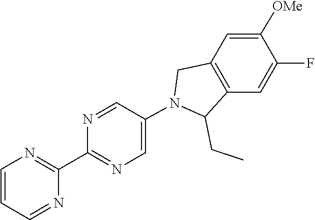 [SMILES: CCC1c2cc(F)c(OC)cc2CN1c1cnc(-c2ncccn2)nc1] | 2-([2,2′-bipyrimidin]-5-yl)-1-ethyl-6-fluoro-5- methoxyisoindoline; |
| 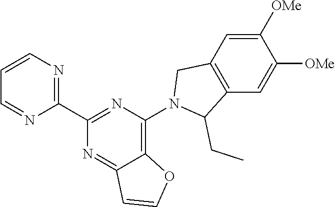 [SMILES: CCC1c2cc(OC)c(OC)cc2CN1c1nc(-c2ncccn2)nc2ccoc12] | 4-(1-ethyl-5,6-dimethoxyisoindolin-2-yl)-2- (pyrimidin-2-yl)furo[3,2-d]pyrimidine; |
| 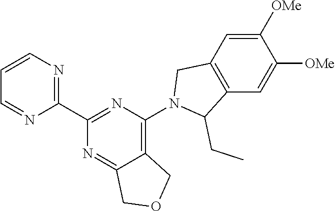 [SMILES: CCC1c2cc(OC)c(OC)cc2CN1c1nc(-c2ncccn2)nc2c1COC2] | 4-(1-ethyl-5,6-dimethoxyisoindolin-2-yl)-2- (pyrimidin-2-yl)-5,7-dihydrofuro[3,4-d]pyrimidine; |
| 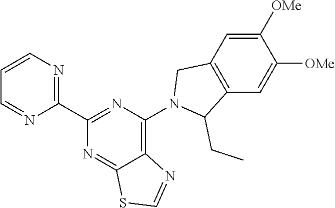 [SMILES: CCC1c2cc(OC)c(OC)cc2CN1c1nc(-c2ncccn2)nc2scnc12] | 7-(1-ethyl-5,6-dimethoxyisoindolin-2-yl)-5- (pyrimidin-2-yl)thiazolo[5,4-d]pyrimidine; |
| 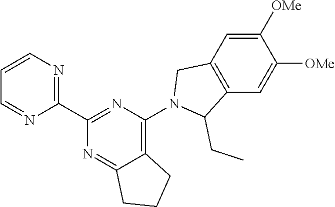 [SMILES: CCC1c2cc(OC)c(OC)cc2CN1c1nc(-c2ncccn2)nc2c1CCC2] | 4-(1-ethyl-5,6-dimethoxyisoindolin-2-yl)-2- (pyrimidin-2-yl)-6,7-dihydro-5H- cyclopenta[d]pyrimidine; |
| 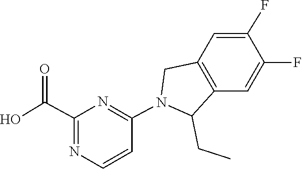 [SMILES: CCC1c2cc(F)c(F)cc2CN1c1ccnc(C(=O)O)n1] | 4-(1-ethyl-5,6-difluoroisoindolin-2-yl) pyrimidine-2-carboxylic acid; |
| 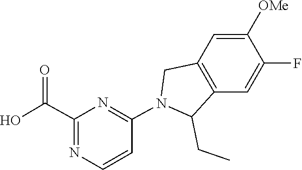 [SMILES: CCC1c2cc(F)c(OC)cc2CN1c1ccnc(C(=O)O)n1] | 4-(1-ethyl-6-fluoro-5-methoxyisoindolin-2- yl)pyrimidine-2-carboxylic acid; |
| 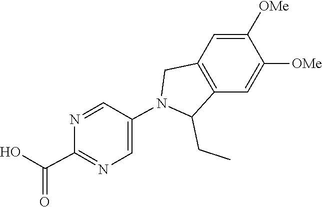 [SMILES: CCC1c2cc(OC)c(OC)cc2CN1c1cnc(C(=O)O)nc1] | 5-(1-ethyl-5,6-dimethoxyisoindolin-2-yl) pyrimidine-2-carboxylic acid; |
| 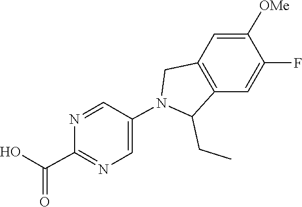 [SMILES: CCC1c2cc(F)c(OC)cc2CN1c1cnc(C(=O)O)nc1] | 5-(1-ethyl-6-fluoro-5-methoxyisoindolin-2- yl)pyrimidine-2-carboxylic acid; |
| 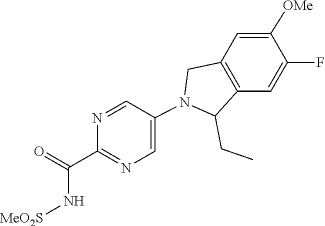 [SMILES: CCC1c2cc(F)c(OC)cc2CN1c1cnc(C(=O)NS(C)(=O)=O)nc1] | 5-(1-ethyl-6-fluoro-5-methoxyisoindolin-2-yl)-N- (methylsulfonyl)pyrimidine-2-carboxamide; |
| 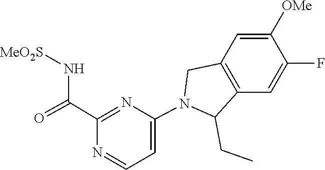 [SMILES: CCC1c2cc(F)c(OC)cc2CN1c1ccnc(C(=O)NS(C)(=O)=O)n1] | 4-(1-ethyl-6-fluoro-5-methoxyisoindolin-2-yl)-N- (methylsulfonyl)pyrimidine-2-carboxamide; |
| 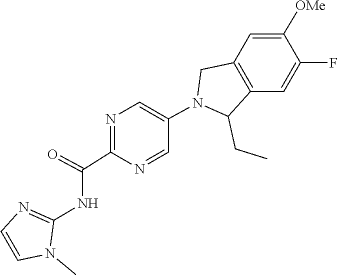 [SMILES: CCC1c2cc(F)c(OC)cc2CN1c1cnc(C(=O)Nc2nccn2C)nc1] | 5-(1-ethyl-6-fluoro-5-methoxyisoindolin-2-yl)-N-(1- methyl-1H-imidazo1-2-yl)pyrimidine-2-carboxamide; |
| 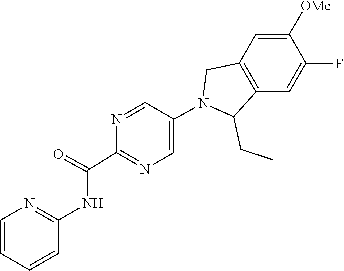 [SMILES: CCC1c2cc(F)c(OC)cc2CN1c1cnc(C(=O)Nc2ccccn2)nc1] | 5-(1-ethyl-6-fluoro-5-methoxyisoindolin-2-yl)-N- (pyridin-2-yl)pyrimidine-2-carboxamide; |
| 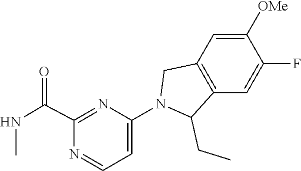 [SMILES: CCC1c2cc(F)c(OC)cc2CN1c1ccnc(C(=O)NC)n1] | 4-(1-ethyl-6-fluoro-5-methoxyisoindolin-2-yl)-N- methylpyrimidine-2-carboxamide; |
| 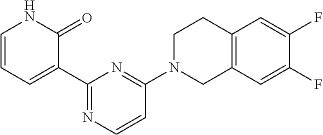 [SMILES: O=c1[nH]cccc1-c1nccc(N2CCc3cc(F)c(F)cc3C2)n1] | 3-(4-(6,7-difluoro-3,4-dihydroisoquinolin-2(1H)- yl)pyrimidin-2-yl)pyridin-2-ol; |
| 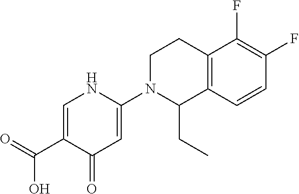 [SMILES: CCC1c2ccc(F)c(F)c2CCN1c1cc(=O)c(C(=O)O)c[nH]1] | 6-(1-ethyl-5,6-difluoro-3,4-dihydroisoquinolin-2(1H)- yl)-4-oxo-1,4-dihydropyridine-3-carboxylic acid; |
| 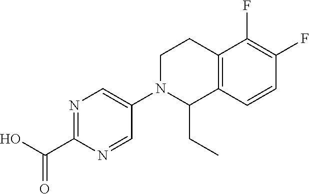 [SMILES: CCC1c2ccc(F)c(F)c2CCN1c1cnc(C(=O)O)nc1] | 5-(1-ethyl-5,6-difluoro-3,4-dihydroisoquinolin-2(1H)- yl)pyrimidine-2-carboxylic acid; |
| 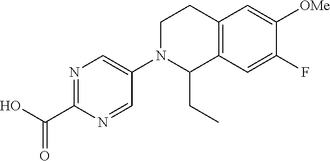 [SMILES: CCC1c2cc(F)c(OC)cc2CCN1c1cnc(C(=O)O)nc1] | 5-(1-ethyl-7-fluoro-6-methoxy-3,4- dihydroisoquinolin-2(1H)-yl)pyrimidine-2- carboxylic acid; |
| 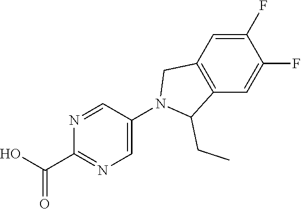 [SMILES: CCC1c2cc(F)c(F)cc2CN1c1cnc(C(=O)O)nc1] | 5-(1-ethyl-5,6-difluoroisoindolin-2-yl)pyrimidine-2- carboxylic acid; |
| 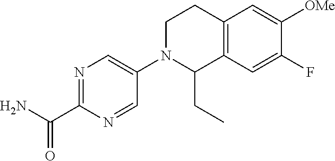 [SMILES: CCC1c2cc(F)c(OC)cc2CCN1c1cnc(C(N)=O)nc1] | 5-(1-ethyl-7-fluoro-6-methoxy-3,4- dihydroisoquinolin-2(1H)-yl)pyrimidine-2- carboxamide; |
| 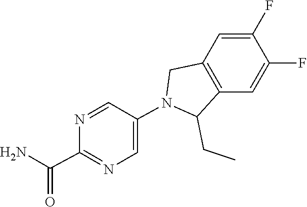 [SMILES: CCC1c2cc(F)c(F)cc2CN1c1cnc(C(N)=O)nc1] | 5-(1-ethyl-5,6-difluoroisoindolin-2-yl)pyrimidine-2- carboxamide; |
| 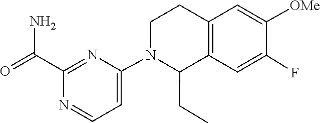 [SMILES: CCC1c2cc(F)c(OC)cc2CCN1c1ccnc(C(N)=O)n1] | 4-(1-ethyl-7-fluoro-6-methoxy-3,4- dihydroisoquinolin-2(1H)-yl)pyrimidine-2- carboxamide; |
| 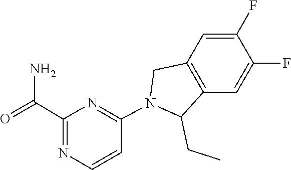 [SMILES: CCC1c2cc(F)c(F)cc2CN1c1ccnc(C(N)=O)n1] | 4-(1-ethyl-5,6-difluoroisoindolin-2-yl)pyrimidine-2- carboxamide; |
| 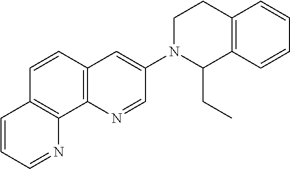 [SMILES: CCC1c2ccccc2CCN1c1cnc2c(ccc3cccnc32)c1] | 3-(1-ethyl-3,4-dihydroisoquinolin-2(1H)-yl)-1,10- phenanthroline; |
| 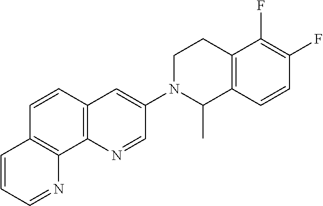 [SMILES: CC1c2ccc(F)c(F)c2CCN1c1cnc2c(ccc3cccnc32)c1] | 3-(5,6-difluoro-1-methyl-3,4-dihydroisoquinolin- 2(1H)-yl)-1,10-phenanthroline; |
| 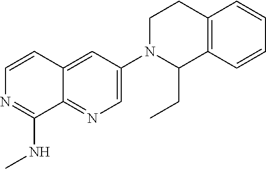 [SMILES: CCC1c2ccccc2CCN1c1cnc2c(NC)nccc2c1] | 3-(1-ethyl-3,4-dihydroisoquinolin-2(1H)-yl)-N- methyl-1,7-naphthyridin-8-amine; |
| 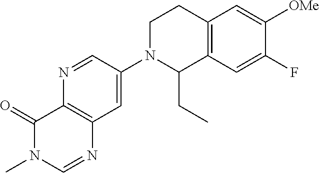 [SMILES: CCC1c2cc(F)c(OC)cc2CCN1c1cnc2c(=O)n(C)cnc2c1] | 7-(1-ethyl-7-fluoro-6-methoxy-3,4- dihydroisoquinolin-2(1H)-yl)-3-methylpyrido[3,2- d]pyrimidin-4(3H)-one; |
| 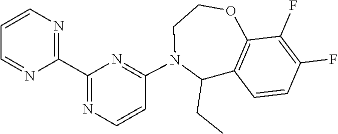 [SMILES: CCC1c2ccc(F)c(F)c2OCCN1c1ccnc(-c2ncccn2)n1] | 5-Ethyl-8,9-difluoro-4-(2-pyrimidin-2-ylpyrimidin-4- yl)-3,5-dihydro-2H-1,4-benzoxazepine; |
| 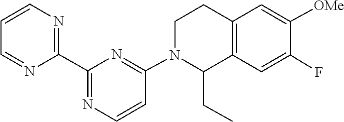 [SMILES: CCC1c2cc(F)c(OC)cc2CCN1c1ccnc(-c2ncccn2)n1] | 2-(2,2′-bipyrimidin-4-yl)-1-ethyl-7-fluoro-6-methoxy- 1,2,3,4-tetrahydroisoquinoline; |
| 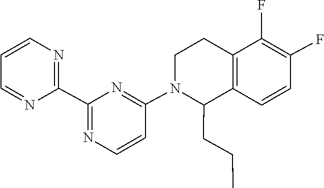 [SMILES: CCCC1c2ccc(F)c(F)c2CCN1c1ccnc(-c2ncccn2)n1] | 2-([2,2′-bipyrimidin]-4-yl)-5,6-difluoro-1-propyl- 1,2,3,4-tetrahydroisoquinoline; and |
| 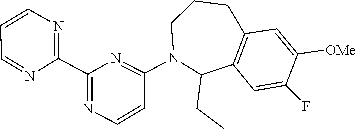 [SMILES: CCC1c2cc(F)c(OC)cc2CCCN1c1ccnc(-c2ncccn2)n1] | 2-([2,2′-bipyrimidin]-4-yl)-1-ethyl-8-fluoro-7- methoxy-2,3,4,5-tetrahydro-1H-benzo[c]azepine. |

16. A compound selected from the group consisting of:

| | |
|---|---|
| 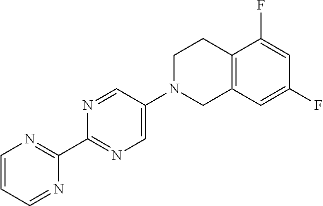 [SMILES: Fc1cc(F)c2c(c1)CN(c1cnc(-c3ncccn3)nc1)CC2] | 2-([2,2′-bipyrimidin]-5-yl)-5,7-difluoro-1,2,3,4- tetrahydroisoquinoline; |
| 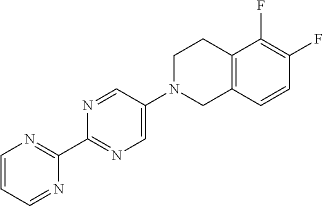 [SMILES: Fc1ccc2c(c1F)CCN(c1cnc(-c3ncccn3)nc1)C2] | 2-([2,2′-bipyrimidin]-5-yl)-5,6-difluoro-1,2,3,4- tetrahydroisoquinoline; |
| 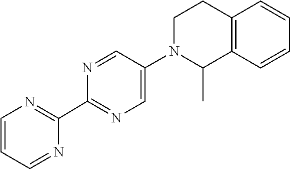 [SMILES: CC1c2ccccc2CCN1c1cnc(-c2ncccn2)nc1] | 1-methyl-2-(2-pyrimidin-2-ylpyrimidin-5-yl)-3,4- dihydro-1H-isoquinoline; |
| 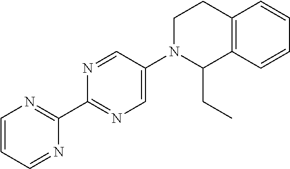 [SMILES: CCC1c2ccccc2CCN1c1cnc(-c2ncccn2)nc1] | 2-([2,2′-bipyrimidin]-5-yl)-1-ethyl-1,2,3,4- tetrahydroisoquinoline; |
| 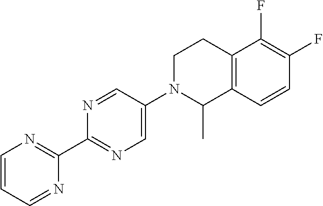 [SMILES: CC1c2ccc(F)c(F)c2CCN1c1cnc(-c2ncccn2)nc1] | 2-([2,2′-bipyrimidin]-5-yl)-5,6-difluoro-1-methyl- 1,2,3,4-tetrahydroisoquinoline; |
| 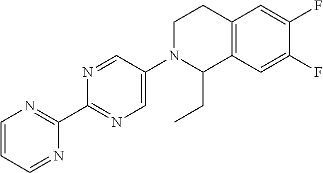 [SMILES: CCC1c2cc(F)c(F)cc2CCN1c1cnc(-c2ncccn2)nc1] | 2-([2,2′-bipyrimidin]-5-yl)-1-ethyl-6,7-difluoro- 1,2,3,4-tetrahydroisoquinoline; |
| 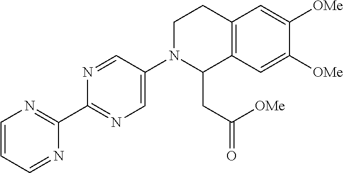 [SMILES: COC(=O)CC1c2cc(OC)c(OC)cc2CCN1c1cnc(-c2ncccn2)nc1] | methyl 2-(2-([2,2′-bipyrimidin]-5-yl)-6,7- dimethoxy-1,2,3,4-tetrahydroisoquinolin-1- yl)acetate; |
| 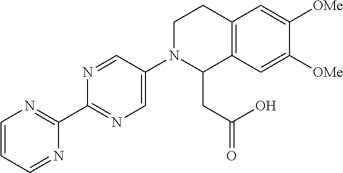 [SMILES: COc1cc2c(cc1OC)C(CC(=O)O)N(c1cnc(-c3ncccn3)nc1)CC2] | 2-(2-([2,2′-bipyrimidin]-5-yl)-6,7-dimethoxy- 1,2,3,4-tetrahydroisoquinolin-1-yl)acetic acid; |
| 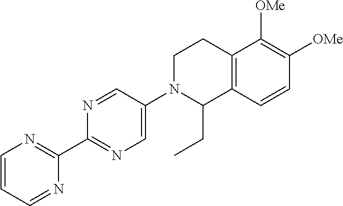 [SMILES: CCC1c2ccc(OC)c(OC)c2CCN1c1cnc(-c2ncccn2)nc1] | 2-([2,2′-bipyrimidin]-5-yl)-1-ethyl-5,6-dimethoxy- 1,2,3,4-tetrahydroisoquinoline; |
| 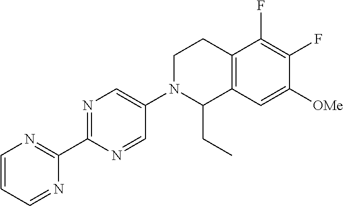 [SMILES: CCC1c2cc(OC)c(F)c(F)c2CCN1c1cnc(-c2ncccn2)nc1] | 2-([2,2′-bipyrimidin]-5-yl)-1-ethyl-5,6-difluoro-7- methoxy-1,2,3,4-tetrahydroisoquinoline; |
| 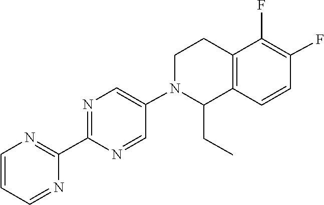 [SMILES: CCC1c2ccc(F)c(F)c2CCN1c1cnc(-c2ncccn2)nc1] | 2-([2,2′-bipyrimidin]-5-yl)-1-ethyl-5,6-difluoro- 1,2,3,4-tetrahydroisoquinoline; |
| 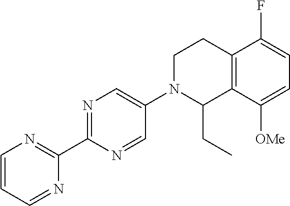 [SMILES: CCC1c2c(OC)ccc(F)c2CCN1c1cnc(-c2ncccn2)nc1] | 2-([2,2′-bipyrimidin]-5-yl)-1-ethyl-5-fluoro-8- methoxy-1,2,3,4-tetrahydroisoquinoline; and |
| 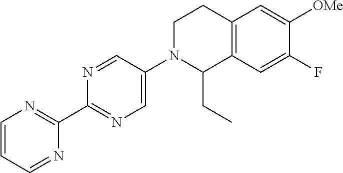 [SMILES: CCC1c2cc(F)c(OC)cc2CCN1c1cnc(-c2ncccn2)nc1] | 2-([2,2′-bipyrimidin]-5-yl)-1-ethyl-7-fluoro-6- methoxy-1,2,3,4-tetrahydroisoquinoline. |

17. A pharmaceutical composition comprising at least one compound of claim 1 and a pharmaceutically acceptable carrier, optionally further comprising at least one additional agent useful for treating hepatitis B virus (HBV) infection.

18. (canceled)

19. The pharmaceutical composition of claim 17, wherein the at least one additional agent comprises at least one selected from the group consisting of reverse transcriptase inhibitor; capsid inhibitor; cccDNA formation inhibitor; sAg secretion inhibitor; oligomeric nucleotide targeted to the Hepatitis B genome; and immunostimulator.

20. The pharmaceutical composition of claim 19, wherein the oligomeric nucleotide comprises one or more siRNAs.

21. A method of treating or ameliorating hepatitis B virus HBV infection in a subject, the method comprising administering to the subject a therapeutically effective amount of at least one compound of claim 1.

22. A method of reducing or minimizing levels of at least one selected from the group consisting of hepatitis B virus surface antigen (HBsAg), hepatitis B e-antigen (HBeAg), hepatitis B core protein, and pregenomic (pg) RNA, in a hepatitis B virus (HBV)-infected subject, the method comprising administering to the subject a therapeutically effective amount of at least one compound of claim 1.

23. (canceled)

24. The method of claim 21, wherein the subject is further administered at least one additional agent useful for treating the hepatitis B virus (HBV) infection.

25. The method of claim 24, wherein the at least one additional agent comprises at least one selected from the group consisting of reverse transcriptase inhibitor; capsid inhibitor; cccDNA formation inhibitor; sAg secretion inhibitor; oligomeric nucleotide targeted to the Hepatitis B genome; and immunostimulator.

26. The method of claim 25, wherein the oligomeric nucleotide comprises one or more siRNAs.

27. The method of claim 24, wherein the subject is co-administered the at least one compound and the at least one additional agent.

28. The method of claim 27, wherein the at least one compound and the at least one additional agent are coformulated.

29. The method of claim 21, wherein the subject is co-infected with HBV-hepatitis D virus (HDV).

30. The method of claim 21, wherein the subject is a mammal.

31. The method of claim 30, wherein the mammal is a human.
